(19) United States
(12) Patent Application Publication
(10) Pub. No.: US 2024/0158776 A1
(43) Pub. Date: May 16, 2024

(54) NANOCAGED ENZYMES WITH ENHANCED CATALYTIC ACTIVITY AND INCREASED STABILITY

(71) Applicant: ARIZONA BOARD OF REGENTS ON BEHALF OF ARIZONA STATE UNIVERSITY, Scottsdale, AZ (US)

(72) Inventors: Jinglin FU, Tempe, AZ (US); Zhao ZHAO, Boston, MA (US); Neal WOODBURY, Tempe, AZ (US); Hao YAN, Chandler, AZ (US)

(21) Appl. No.: 18/481,955

(22) Filed: Oct. 5, 2023

Related U.S. Application Data
(63) Continuation of application No. 16/812,225, filed on Mar. 6, 2020.
Application No. 16/812,225 is a continuation of application No. 15/649,351, filed on Jul. 13, 2017, now Pat. No. 10,669,534.
(60) Provisional application No. 62/361,884, filed on Jul. 13, 2016.

(51) Int. Cl. C12N 11/04; C12N 11/06; C12N 11/10; C12N 15/11

(52) U.S. Cl. CPC C12N 11/04; C12N 11/06; C12N 11/10; C12N 15/11; B82Y 5/00; C12N 2310/532

(57) ABSTRACT

The present disclosure describes a nanoparticle comprising a three dimensional DNA nanocage and a payload biological macromolecule, and methods of assembly thereof.

### CROSS-REFERENCE TO RELATED APPLICATIONS

This application is a Continuation of U.S. application Ser. No. 16/812,225 filed Mar. 6, 2020, which is a Continuation of U.S. application Ser. No. 15/649,351 filed Jul. 13, 2017, which claims the benefit of U.S. Provisional Application No. 62/361,884, filed Jul. 13, 2016, which are both hereby incorporated by reference in its entirety for all purposes.

### STATEMENT REGARDING FEDERALLY SPONSORED RESEARCH

This invention was made with government support under W911NF-11-1-0137 and W911NF-12-1-0420 awarded by the Army Research Office. The government has certain rights in the invention.

### SEQUENCE LISTING

The instant application contains a Sequence Listing which has been filed electronically in XML format and is hereby incorporated by reference in its entirety. Said XML copy, created on March 6, 2020, is named G8118-00403.xml and is 1,250,732 bytes in size.

### BACKGROUND

Common micro- and nanoscale subcellular compartments are formed from either lipids or proteins and include mitochondria, lysosomes, peroxisomes, carboxysomes and other metabolosomes, as well as multi-enzyme complexes. Compartments increase the overall activity and specificity of the encapsulated enzyme pathways by maintaining a high local concentration of enzymes and substrates, promoting substrate channeling and protecting their content from damage, as well as by segregating potentially damaging reactions from the cytosol. Spatial confinement is also an important aspect for chaperone-assisted folding of linear polypeptides into active tertiary and quaternary conformations, as well as for preventing proteins from aggregating under cellular stress conditions. A better understanding of the effects of spatial confinement on protein function will not only enhance the fundamental knowledge of cellular organization and metabolism but also increase the ability to translate biochemical pathways into a variety of noncellular applications, ranging from diagnostics and drug delivery to the production of high-value chemicals and smart materials. Over the past few decades, artificial enzymatic particles have been created using compartmentalization by virus-like protein particles, liposomes or polymersomes and chemical crosslinking. However, severe obstacles to a broader application remain, including low encapsulation yield of large proteins because of steric hindrance, insufficient access of substrates to the encapsulated enzymes, aggregation of vesicle shells and limited control over the spatial arrangement of proteins within the compartments.

### SUMMARY

In a first aspect, provided herein is a nanocage, where the nanocage comprises a three dimensional body comprising a plurality of structural members comprising DNA, wherein internal surfaces of the plurality of structural members form an inner cavity. The DNA can be M13 viral DNA. Architectural arrangement of the structural members in the three dimensional body can form a honeycomb lattice. Architectural arrangement of the structural members in the three dimensional body can form a square lattice. In some cases, the architectural arrangement of the structural members in the three dimensional body can form a single-walled square lattice. In other cases, the architectural arrangement of the structural members in the three dimensional body can form a double-walled square lattice. The three dimensional body can be smaller than 100nm×100 nm×100 nm. The three dimensional body can be smaller than 75nm×50 nm×50 nm. The inner cavity of the three dimensional body can measure less than 50 nm×50 nm×50nm. The three dimensional body can further comprise at least one nanopore. The at least one nanopore can have a diameter of about 1 nm to about 5nm. The at least one nanopore has a diameter of about 1.5 nm to about 3 nm. The three dimensional body can comprise between 0.10 to 0.30 DNA helices per nm². The three dimensional body can comprise between 0.11 to 0.17 DNA helices per nm².

In another aspect, provided herein is a nanoparticle comprising a nanocage comprising a plurality of structural members comprising DNA in a three-dimensional lattice, wherein internal surfaces of the plurality of structural members form an inner cavity; and one or more payload molecules bound to internal surfaces of the inner cavity. The payload molecules can comprise enzymes, nucleic acids, polypeptides, antibodies, phospholipids, or any combination thereof. The inner cavity can encapsulate two payload molecules. The one or more payload molecules can be covalently linked to internal surfaces of the inner cavity. The nanocage can be configured to prevent proteolytic degradation of the trapped payload molecule. The nanocage can be configured to enhance the activity of the trapped payload molecule.

In another aspect, provided herein is a method of making a nanoparticle, where the method comprises trapping a payload macromolecule in an open half cage; and assembling two half cages into a closed nanocage; wherein the closed nanocage has an inner cavity; wherein the closed nanocage has nanopores; and wherein the resulting nanoparticle comprises a closed nanocage comprising nanopores and an inner cavity comprising one or more biological macromolecules. The half cage can comprise DNA. The DNA can be M13 viral DNA. The half cage comprising DNA can be constructed by folding full-length M13 viral DNA. The half cage can comprise a base and two adjoined side walls protruding from the base. The biological macromolecule can be covalently linked to the half cage. Two half cages can be assembled into a closed nanocage by adding short bridge DNA strands.

### BRIEF DESCRIPTION OF DRAWINGS

The patent or patent application file contains at least one drawing in color. Copies of this patent or patent application publication with color drawings will be provided by the Office upon request and payment of the necessary fee.

The present invention will be better understood and features, aspects and advantages other than those set forth above will become apparent when consideration is given to the following detailed description thereof. Such detailed description makes reference to the following drawings, wherein:

FIGS. 1A-1C show design and characterization of DNA nanocage-encapsulated enzymes with controlled stoichiometry. (A) Schematic representations of the assembly of a DNA nanocage encapsulating a pair of GOx (orange) and HRP (green) enzymes. Individual enzymes were first attached to half-cages, followed by the addition of linker strands (red) to combine the two halves into a full-cage. Small pores of honeycomb shape (˜2.5 nm d.i.) were designed on the bottom of cages to facilitate the diffusion of substrate molecules in an out of the cage. (B) Negatively stained TEM images of DNA cages containing a single GOx (shown as less stained dots) and (C) a pair of GOx and HRP (shown as less stained dots). Scale bar, 50 nm.

FIGS. 2A-2E show single-molecule fluorescence characterization of enzyme encapsulation. (A) Schematic illustration of single-molecule fluorescence co-localization of Cy3-labelled protein with Cy5-labelled cage using TIRF microscopy. DNA cages were captured on the surface by biotin-streptavidin interaction. (B) Representative field of view of enzyme-encapsulating cages under TIRF microscope. Examples of Cy3-Cy5 co-localization are highlighted using a pair of rectangles. Scale bar, 10 μm. (C) Quantified encapsulation yield for six different enzymes. The total number of molecules analyzed for each protein is shown in Table 3. The error bars represent the standard deviation obtained from the analysis of two to four movies of the sample from the same batch. (D) Fluorophore photobleaching trajectories with one, two, and three photobleaching steps. Photobleaching steps were quantitatively analyzed by fitting the trajectories by HMM in QUB program. (E) Photobleaching statistics for Cy3-labelled proteins encapsulated within half-cages (Half[G6pDH]) or full-cages (Full[G6pDH]), as well as for an unencapsulated protein control (G6pDH). HMM, hidden-Markov modelling.

FIGS. 3A-3B show activity characterization of encapsulated GOx/HRP pairs. (A) Schematic representation of the GOx/HRP cascade. (B) Normalized cascade activities for a GOx/HRP pair encapsulated within a full-cage (Full[G0x/HRP]), two individual full-cages (Full[G0x]+Full[HRP]) and two individual half-cages (Half[G0x]+Half[HRP]), as well as unencapsulated enzyme pairs with and without the presence of DNA cages. Assay conditions: 1 nM enzyme or 1 nM enzyme-DNA cage, 1 mM glucose and 2 mM ABTS in TBS buffer (pH 7.5), and monitoring absorbance at 410 nm. Error bars were generated as the standard deviation of at least three replicates.

FIGS. 4A-4C show mechanistic study of the activity enhancement of DNA nanocage-encapsulated enzymes. (A) Relationship between turnover rate enhancement factor after encapsulation against enzyme molecular weight (fitted using one-phase decay function). (B) Nanocage-encapsulated G6pDH activity change after incubation with different amount of NaCl. Assay conditions: 0.5 nM enzyme-DNA cage, incubation with 1 mM glucose-6-phosphate and 1 mM NAD+in TBS buffer (pH 7.5), and monitoring absorbance at 340 nm. (C) Normalized kcat and K_{M} values of free G6pDH and G6pDH that is encapsulated within different DNA cage: SH(G6pDH), a honeycomb lattice origami with a single layer; SS(G6pDH), a square-lattice origami with a single layer; and DS(G6pDH), a square-lattice origami with two layers. k cat and K_{M} values of caged enzymes are normalized to that of free enzymes. Error bars were generated as the standard deviation of at least three replicates.

FIGS. 5A-5G show single-molecule kinetics of nanocage-encapsulated enzymes. (A) Schematic of the experimental TIRF set up for characterizing G6pDH encapsulated within a full-cage (Full[G6pDH]) and a half-cage (Half[G6pDH]), as well as an unencapsulated control. (B) A PMS/resazurin-coupled fluorescence assay used to characterize the activity of G6pDH. NAD+ is first reduced to NADH by G6pDH, followed by PMS- catalyzed electron transfer from NADH to resazurin, producing a strongly fluorescent resorufin, which has an excitation/emission maximum at 544/590 nm. (C) TIRFM snapshots captured before and after the injection of substrate G6p. In presence of G6p, the field of view showed increased fluorescence due to the formation of resorufin (compare the boxed regions). Fluorescent beads (very bright spots present in both +G6p and −G6p images) were used as reference markers to correct for the drift. Scale bars, 10 μm. (D) Real-time traces of fluorescence spikes (resorufin production) for enzymes without and with the addition of G6p substrate. Ten single-molecule traces for each condition were concatenated. (E) Statistics of spike frequency, (F) fraction of active molecules, and (G) overall observed enzyme activity for G6pDH. The number of active molecules analyzed is denoted by ‘n’ in (E). The standard deviations for the spike frequency were calculated after randomly assigning the active molecules into three groups; those for the fractions of active molecules were calculated from three to four independent movies, and those for the normalized overall activity were estimated from the propagation of errors. All experiments were carried out at room temperature in 1×TBS buffer, pH 7.5, in the presence of 1 mM Mg2+ and 10% (w/v) PEG 8000.

FIGS. 6A-6C show protection of nanocaged enzymes against protease-mediated degradation and aggregation. (A) Schematic representation illustrating how a DNA cage may block access of big proteins such as a protease to the interior of the cage, but still allow the penetration of small molecules. (B) Relative enzyme activity of encapsulated GOx/HRP pairs (Full [G0x/HRP]) and free GOx/HRP pairs (free GOx/HRP) before and after the addition of trypsin. Trypsin digestion conditions: enzyme or enzyme-DNA cage was incubated with 1,000× excess trypsin for 24 h at 37° C. Assay conditions: 0.5 nM enzyme or 0.5 nM enzyme-DNA cage, incubation with 1 mM glucose and 2 mM ABTS in 1 x TBS buffer (pH 7.5), and monitoring absorbance at 410 nm. (C) Relative activity data for free G6pDH and Full[G6pDH] (0.5 nM) with trypsin digestion for 0, 1, 4, 8 and 24 h. Digestion by incubation sample with 1,000 times amount of trypsin at 37° C. in 1× TBS buffer (pH 7.5). Error bars were generated as the standard deviation of at least three replicates.

FIG. 7 illustrates an exemplary SH full-cage having a honeycomb lattice arrangement, presented in cross-sectional view and 3D view.

FIG. 8 illustrates an exemplary SS cage having a square lattice arrangement, presented in cross-sectional view and 3D view.

FIG. 9 illustrates an exemplary DS cage having a square lattice arrangement, presented in cross-sectional view and 3D view.

FIG. 10 shows a representative TEM image of the half-cage structure (scale bar: 50 nm).

FIG. 11 shows a representative TEM image of the full-cage structure (scale bar: 50 nm).

FIG. 12 shows an agarose gel electrophoresis (AGE) to characterize the full-cage structure (lane 1: MI3 DNA, lane 2: half-cage; lane 3: full-cage). According to the gel band intensity, the assembly yield of the full-cage was higher than 90%.

FIG. 13 shows a schematic illustration of the SPDP conjugation chemistry used for protein-DNA conjugation.

FIGS. 14A-14F show quantification of fluorescent dye-labeled enzyme-DNA conjugates using UV-Vis absorbance spectroscopy. (A) Cy3-labeled HRP-TTTTTCCCTCCCTCC (SEQ ID NO:1393) with an average dye-to-protein ratio of 1.8; (B) Cy3-labeled GOx-TTTTTCCCTCCCTCC (SEQ ID NO:1393) with an average dye-to-protein ratio of 1.5; (C) Cy3-labeled G6pDH-TTTTTCCCTCCCTCC (SEQ ID NO:1393) with an average dye-to-protein ratio of 1.6; (D) Alexa Fluor 647-labeled MDH-TTTTTGGCTGGCTGG (SEQ ID NO:1394) with an average dye-to-protein ratio of 1.2; (E) Alexa Fluor 647-labeled LDH-TTTTTGGCTGGCTGG (SEQ ID NO:1394) with an average dye-to-protein ratio of 1.7; (F) Cy3-labeled (β-Gal)-TTTTTCCCTCCCTCC (SEQ ID NO:1393) with an average dye-to-protein ratio of 0.6.

FIGS. 15A-15B shows two different designs for the cage structure with different encapsulation yields (see FIG. 16 and FIG. 17), assembled with GOx. (A) Cage with closed-wall design. (B) Cage with open-wall design.

FIG. 16 shows TEM image of full-cages with closed-wall design (FIG. 15A) encapsulating GOx. An encapsulation yield of 38% was estimated from similar images containing about 230 DNA cages by dividing the number of cages with a discernible protein inside by the total number of the cages counted (yellow arrow indicates DNA cage with enzyme inside).

FIG. 17 shows TEM image of full-cage with open-wall design (FIG. 15B) encapsulating GOx. An encapsulation yield of 77% was estimated from similar images containing about 300 DNA cages by dividing the number of cages with a discernible protein inside by the total number of cages counted (yellow arrow indicates DNA cage with enzyme inside).

FIG. 18 shows TEM image for HRP-GOx enzyme pairs encapsulated in DNA full-cage. Despite variable quality of staining across the field of view, the inner cavity of many nanocages appeared to contain two bright spots, which we interpreted as intact HRP-GOx enzyme pairs (yellow arrow indicates DNA cage with enzyme pair inside).

FIG. 19 shows native AGE characterization of a DNA nanocage encapsulating a GOx/HRP pair. GOx and HRP were conjugated with Cy3 and Cy5, respectively. Lane 1 (from left): half-cage assembled with G0x-Cy3, lane 2: half-cage assembled with HRP-Cy5, lanes 3 and 4: full-cage with GOx/HRP. “EB” indicates ethidium bromide staining of the gel to visualize all DNA bands.

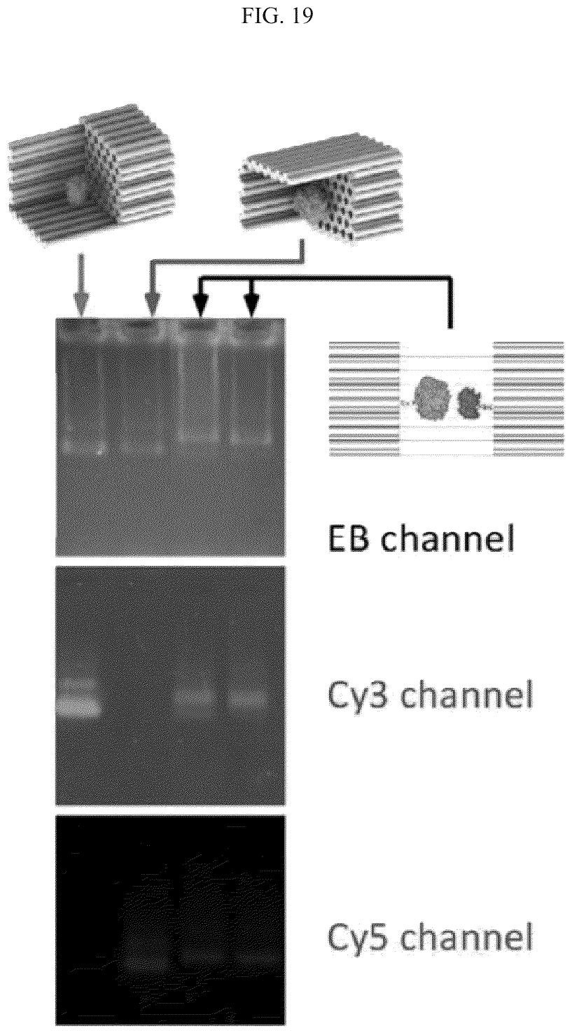

FIG. 20 shows raw activity data for a set of DNA cage-encapsulated enzymes. 1: Full[H+G], a full cage-encapsulated GOx and HRP; 2: Full[H] +Full[G], a full cage-encapsulated HRP and a full cage-encapsulated GOx; 3: half[H] +half[G], a half cage-encapsulated HRP and a half-cage encapsulated GOx; 4: Full +H +G, a full cage incubated with a pair of free HRP and GOx; 5: H +G fresh control, a fresh solution of free HRP and GOx; 6: H+G annealing control, a solution of free HRP and GOx that is incubated using the same thermal program as the DNA cage-encapsulated enzymes; 7: substrate background control. Assay conditions: 1 nM enzyme or enzyme-encapsulating DNA cage, with 1 mM Glucose, 2 mM ABTS in pH 7.5, lx TBS buffer. Absorbance is monitored at 410 nm.

FIG. 21 shows determination of the Michaelis-Menten constants for enzymes-HRP. Raw activity data for free enzyme solution of DNA-conjugated HRP (0.5 nM) with H₂O₂ concentration varied from 1 μM to 1000 μM, and 2 mM ABTS, monitoring absorbance at 410 nm. Error bars were calculated from the standard deviation of at least three replicates.

FIG. 22 shows determination of the Michaelis-Menten constants for enzymes-HRP. Raw activity data for DNA cage-encapsulating HRP (0.5 nM) with H₂O₂ varied from 1μM to 1000 μM and 2 mM ABTS, monitoring absorbance at 410 nm. Error bars were calculated from the standard deviation of at least three replicates.

FIG. 23 shows ABTS standard curve to calculate k_{cat} value (Y=0.01359x+0.01214).

FIG. 24 shows a Michaelis-Menten plot of HRP encapsulated within a full-cage (Full-Cage[HRP], red circles), compared with that of free HRP (HRP control, black squares) using H₂O₂ as the substrate. The solid lines represent fits of the Michaelis-Menten model to the data. Enzyme assay conditions: 0.5 nM enzyme or DNA-cage-encapsulated enzyme, 2 mM ABTS with different concentrations of H₂O₂ ranging from 1 μM to 1000 μM, in 1× TBS buffer (pH 7.5, 1 mM MgCl₂), absorbance monitored at 410 nm. The table lists the fit parameters. Full-cage encapsulation of the enzyme caused a 2-fold increase in K_{M} and an about 9-fold increase in k_{cat}.

FIG. 25 shows raw activity data measurement of Full-Cage [HRP] (0.5 nM) with ABTS concentration varied from 10 μM to 3000 μM and 2000 μM H₂O₂, monitoring absorbance at 410 nm. Error bars were calculated from the standard deviation of at least three replicates.

FIG. 26 shows raw activity data measurement of free DNA-conjugated HRP (0.5 nM) with ABTS concentration varied from 10 μM to 3000 μM, and 2000 μM H202, monitoring absorbance at 410 nm. Error bars were calculated from the standard deviation of at least three replicates.

FIG. 27 shows a Michaelis-Menten plot for HRP encapsulated within a full-cage (AB-HRP, red circles), compared with that of free HRP enzyme (HRP control, black squares) using ABTS as the substrate. The solid lines represent fits of the Michaelis-Menten model to the data. Enzyme assay conditions: 0.5 nM enzyme or full-cage-encapsulated enzyme, 2000 μM H₂O₂ with different concentrations of ABTS, ranging from 10 μM to 3000 μM, in 1× TBS buffer (pH 7.5, 1 mM MgCl₂), monitoring absorbance at 410 nm. The table lists the fit parameters. DNA encapsulation of the enzyme caused no change in K_{M} and a −9-fold increase in k_{cat}.

FIG. 28 shows a TEM image for the purified DNA full-cage with only HRP enzyme inside. Scale bar: 100 nm. The majority of cages showed one lighter spot inside the cavity, representing the enzyme. Despite variable quality of staining across the field of view, the inner cavity of many nanocages appeared to contain one bright spot, which we interpreted as intact one HRP enzyme (yellow arrow indicates DNA cage with enzyme inside).

FIG. 29 shows raw activity date for free DNA-conjugated GOx (0.5 nM) with different concentrations of glucose ranging from 1 mM to 200 mM. 2 mM ABTS and 100 nM HRP were used to quickly convert H₂O₂ to detectable signal that was monitored at 410 nm. Error bars were calculated from the standard deviation of at least three replicates.

FIG. 30 shows raw activity data for DNA cage-encapsulating GOx (0.5 nM) with different concentrations of glucose ranging from 1 mM to 200 mM. 2 mM ABTS and 100 nM HRP were used to quickly convert H₂O₂ to detectable signal that was monitored at 410 nm. Error bars were calculated from the standard deviation of at least three replicates.

FIG. 31 shows a Michaelis-Menten plot of GOx inside the full-cage (AB-GOx, red circles), compared with that of free GOx enzyme (GOx control, black squares) using glucose as the substrate. The solid lines represent fits of the Michaelis-Menten model to the data. Enzyme assay conditions: 0.5 nM enzyme or DNA cage encapsulated enzyme, 2 mM ABTS, 100 nM HRP with different concentrations of glucose ranging from 1 mM to 200 mM, in 1× TBS buffer (pH 7.5, 1 mM MgCl2) monitoring absorbance at 410 nm. The table lists the fit parameters. DNA encapsulation of the enzyme caused a 2-fold decrease in K_{M} and a 5-fold increase in k_{cat}.

FIG. 32 shows a TEM image of the purified DNA full-cage with only GOx inside (yellow arrow indicates DNA cage with enzyme inside).

FIG. 33 determination of the Michaelis-Menten constants for enzymes-G6pDH. Raw activity data for free DNA-modified G6pDH (0.5 nM) with 10-2500 μM NAD+and 1 mM glucose 6-phosphate, monitoring absorbance at 340 nm. Error bars were calculated from the standard deviation of at least three replicates.

FIG. 34 shows raw activity data for Full-Cage [G6pDH] (0.5 nM) with 10-2500 μM NAD+ and 1 mM glucose 6-phosphate, monitoring absorbance at 340 nm. Error bars were calculated from the standard deviation of at least three replicates.

FIG. 35 shows NADH absorbance standard curve to calculate k cat (Y=0.001951X+0.1694).

FIG. 36 shows a Michaelis-Menten plot of Full-Cage[G6pDH] (red circles) compared with that of free G6pDH (black square), using NAD+as the varying substrate. The solid lines represent fits of the Michaelis-Menten model to the data. Enzyme assay conditions: 0.5 nM enzyme or DNA cage-encapsulated enzyme, 1 mM glucose 6-phosphate, with different concentrations of NAD+ranging from 10 μM to 2500 μM, in 1× TBS buffer (pH 7.5, 1 mM MgCl₂), monitoring absorbance at 340 nm. The table lists the fit parameters. Full-cage encapsulation of the enzyme caused little change in K_{M} and a 5-fold increase in k cat . Error bars were calculated from the standard deviation of at least three replicates.

FIG. 37 shows raw activity data for free DNA-modified G6pDH (0.5 nM) with glucose 6-phosphate varied from 10 μM to 2500 μM, and 1 mM NAD+, monitoring at 340 nm. Error bars were calculated from the standard deviation of at least three replicates.

FIG. 38 shows raw activity data for Full-Cage [G6pDH] (0.5 nM) with glucose 6-phosphate varied from 10 μM to 2500 μM, and 1 mM NAD+, monitoring absorbance at 340 nm. Error bars were calculated from the standard deviation of at least three replicates.

FIG. 39 shows a Michaelis-Menten plot of Full-Cage [G6pDH] (red circles), compared with that of the free G6pDH enzyme (black squares), using glucose 6-phosphate as the substrate. The solid lines represent fits of the Michaelis-Menten model to the data. Enzyme assay conditions: 0.5 nM enzyme or DNA cage-encapsulated enzyme, 1 mM NAO+, with different concentration of glucose-6-phosphate ranging from 10 μM to 2000 μM, in 1× TBS buffer (pH 7.5, 1 mM MgCl₂) monitoring absorbance at 340 nm. The table lists the fitting parameters. DNA encapsulation of the enzyme caused a 1.4-fold increase in K_{M} and a 4-fold increase in k_{cat}.

FIG. 40 shows a TEM image of AGE-purified DNA full-cages with G6pDH inside (yellow arrow indicates DNA cage with enzyme inside).

FIG. 41 shows raw activity data for free DNA-modified MDH (0.5 nM) with 5-1000 μM NADH and 2 mM OAA, monitoring absorbance at 340 nm. Error bars were calculated from the standard deviation of at least three replicates.

FIG. 42 shows raw activity data for Full-Cage [MDH] (0.5 nM) with 5-1000 μM NADH and 2 mM OAA, monitoring absorbance at 340 nm. Error bars were calculated from the standard deviation of at least three replicates.

FIG. 43 shows a Michaelis-Menten plot of Full-Cage [MDH] (red circles), compared with that of free MOH (black squares) using NADH as the varying substrate. The solid lines represent fits of the Michaelis-Menten model to the data. Enzyme assay conditions: 0.5 nM enzyme or DNA cage-encapsulated enzyme, 2 mM OAA, with different concentration of NADH ranging from 5 μM to 1000 μM, in HEPES buffer (pH 7.5, 1 mM MgCl₂) monitoring absorbance at 340 nm. The table lists the fit parameters. DNA encapsulation of the enzyme caused a 1.5-fold increase in K_{M} and a 9-fold increase in k_{cat}.

FIG. 44 shows a TEM image for DNA full-cages with MDH inside (yellow arrow indicates DNA cage with enzyme inside).

FIG. 45 shows determination of the Michaelis-Menten constants for enzymes-LDH. Raw activity for free DNA-modified LDH (0.5 nM) with 5-250 μM NADH and 2 mM pyruvate, monitoring absorbance at 340 nm. (Error bars were calculated from the standard deviation of at least three replicates)

FIG. 46 shows raw activity data for full-cage [LDH] (0.5 nM) with 5-250 μM NADH and 2 mM pyruvate, monitoring absorbance at 340 nm. (Error bars were calculated from the standard deviation of at least three replicates)

FIG. 47 shows a Michaelis-Menten plot of Full-Cage [LDH] (red circles), compared with that of free LDH (black squares), using NADH as the varying substrate. The solid lines represent fits of the Michaelis-Menten model to the data. Enzyme assay condition: 0.5 nM enzyme or DNA cage encapsulated enzyme, 2 mM pyruvate, with different concentration of NADH ranging from 5 μM to 200 μM, in HEPES buffer (pH 7.5, 1 mM MgCl₂) monitoring absorbance at 340 nm. The table lists the fit parameters. DNA encapsulation of the enzyme caused a 2.4-fold increase in K_{M} and a 4-fold increase in k_{cat}.

FIG. 48 shows determination of the Michaelis-Menten constants for enzymes -β-Gal. Raw activity data for free DNA-modified β-Gal (0.5 nM) with different concentration of, ranging from 10 μM to 600 μM RBG, monitoring fluorescence at 590 nm (excitation 532 nm). Error bars were calculated from the standard deviation of at least three replicates.

FIG. 49 shows raw activity for full-cage β-Gal (0.5 nM) with different concentration of, ranging from 10 μM to 600 μM RBG, monitoring fluorescence at 590 nm (excitation 532 nm). Error bars were calculated from the standard deviation of at least three replicates.

FIG. 50 shows a Michaelis-Menten plot of full-cage β-Gal (red circle), compared with that of the fresh free MDH enzyme (blue square) using RBG as the substrate. The solid line is the fitting curve using the Michaelis-Menten model. Enzyme assay condition: 0.5 nM enzyme or DNA cage encapsulated enzyme, with different concentration of RBG, ranging from 10 μM to 600 μM, in TBS buffer (pH 7.5, 1 mM MgCl₂) monitoring fluorescence at 532/590 nm. The table lists the fitting parameters. DNA encapsulation of the enzyme caused a −1.6-fold increase in K_{M} and a -81% decrease in k_{cat} Error bars were calculated from the standard deviation of at least three replicates.

FIG. 51 shows a TEM image for the DNA full-cages with β-Gal inside (yellow arrow indicates DNA cage with enzyme inside).

FIGS. 52A-52E show control experiments in which DNA cages were incubated with enzyme substrates. (A) Red curve: 1 nM Cage was incubated with 1 mM glucose and 2 mM ABTS (GOx/HRP substrates) in 1× TBS, pH 7.5; Black: Autocatalysis of 1 mM glucose and 2 mM ABTS (GOx/HRP substrates) in 1× TBS, pH 7.5. (B) Red: 0.5 nM Cage was incubated with 1 mM glucose-6-phosphate and 1 mM NAO+ (G6pDH substrates) in 1× TBS, pH 7.5; Black: Autocatalysis of 1 mM glucose-6-phosphate and 1 mM NAO+ in 1× TBS, pH 7.5. (C) Red: 0.5 nM Cage was incubated with 2 mM pyruvate and 0.25 mM NAOH (LOH substrates) in 1× TBS, pH 7.5; Black: Autocatalysis of2 mM pyruvate and 0.25 mM NAOH in 1× TBS, pH 7.5. (D) Red: 0.5 nM Cage was incubated with 2 mM oxaloacetate (OAA) and 1 mM NAOH (MOH substrates) in 1× TBS, pH 7.5; Black: Autocatalysis of2 mM OAA and 1 mM NAOH in 1× TBS, pH 7.5. (E) Red: 0.5 nM Cage was incubated with 100 μM resorufin beta-D-galactopyranoside (RBG, β-Gal substrate) in 1× TBS, pH 7.5; Black: Autocatalysis of 100 μM RBG in 1× TBS, pH 7.5, 532 nm (excitation)/590 nm (emission). Error bars were calculated from the standard deviation of at least three replicates. All above results indicate that DNA cages at our experimental concentrations do not significantly catalyze substrate conversion.

FIG. 53 shows a test of the nonspecific adsorption of enzymes onto a plastic 96-well plate. Enzyme concentrations are quantified by UV-VIS spectrometer using the following extinction coefficients: HRP (E₄₀₅ₙₘ—100,000 M−1 cm−1), GOx (E₂₈₀ₙₘ—267,200 M−1 cm−1), G6pDH (E₂₈₀ₙₘ—118,450 M−1 cm−1), β-Gal (E₂₈₀ₙₘ—972,093 M−1 cm−1), LDH (E₂₈₀ₙₘ—202,640 M−1 cm−1), MDH (E₂₈₀ₙₘ—19,600 M−1 cm−1). The UV-Vis absorbance of 100μL of each enzyme solution was measured before adding to the plates, as well as after one hour incubation within the plates in the dark. These conditions are the same as those of the enzyme activity assay. As shown in the Figure, all enzyme solutions showed only a very slight decrease in absorbance after incubation in the plates, suggesting very weak nonspecific adsorption of enzymes onto the plastic plates. Error bars were calculated from the standard deviation of at least three replicates.

FIG. 54 shows testing for nonspecific adsorption of low nanomolar concentrations of enzymes onto plastic 96-well plates was tested using Cy3-labeled HRP. 100 μL of 10 nM Cy3-labeled HRP was assayed for fluorescence intensity, and then the plate was incubated inside a plate reader for one hour. The remaining fluorescence intensity was tested again. A slight increase of fluorescence intensity was observed, possibly due to the buffer evaporation during the incubation. This result suggests that there is very little nonspecific adsorption of Cy3-HRP onto the 96-well plate. Error bars were calculated from the standard deviation of at least three replicates.

FIG. 55 shows the crystal structure of β-Gal which shows its dimensions to be 17 nm×14 nm (left) (Jacobson, R. H. et al. Nature 369, 761-766 (1994)). Dynamic Light Scattering measures a hydrodynamic diameter of 14-18 nm.

FIG. 56 shows inhibition of β-Gal activity by 100-mer polyphosphate (Poly(P)100) in solution. Assay condition: 0.25 nM β-Gal and 100 μM RBG in pH 7.4, 50 mM HEPES buffer. For inhibition assay, β-Gal was first incubated with Poly(P)100 for half an hour, then RBG substrate was added before measuring the activity. The control β-Gal was run at the same condition except for the incubation with buffer for half an hour. The activity of β-Gal was significantly inhibited by 1000 μM Poly(P)100. Error bars were calculated from the standard deviation of at least three replicates.

FIGS. 57A-57E show TEM images of DNA cages after 1 h incubation with a) GOx-HRP enzymatic reaction (conditions: 50 mM HEPES, pH 7.5, 1 mM MgCl₂, 1 mM glucose, 2mM ABTS, 1 nM GOx-HRP, 0.5nM DNA cage), b) G6pDH enzyme reaction (conditions: 50 mM HEPES, pH 7.5, 1 mM MgCl₂, 1 mM glucose-6-phosphate, 1 mM NAD\ 1 nM G6pDH, 0.5nM DNA cage), c) MDH enzyme reaction (conditions: 50 mM HEPES, pH 7.5, 1 mM MgCl_{2, 2} mM OAA, 1 mM NADH, 1 nM MDH, 0.5nM DNA cage), d) LDH enzyme reaction (conditions: 50 mM HEPES, pH 7.5,1 mM MgCl₂, 2mM pyruvate, 1 mM NADH, 1 nM LDH, 0.5nM DNA cage), e) (3-gal enzyme reaction (conditions: 50 mM HEPES, pH 7.5, 1 mM MgCl₂, 1 mM RBG 1 nM Beta-gal, 0.5nM DNA cage). (Scale bars: 50 nm)

FIG. 58 shows raw enzyme activity data of single G6pDH molecules. Representative fluorescence-time traces of free-, half-cage and full-cage G6pDH. Five representative molecules are shown for each sample. The fluorescence intensity of enzyme reaction on the microscope slide was recorded for 5 min at 35 ms time resolution. The average spikes per molecule for different samples are compared in FIG. 5. All experiments were carried out at room temperature in 1×TBS buffer in presence of 1 mM Mg2+, pH 7 .5 (Table 5).

FIGS. 59A-59D show enzyme activity data of single β-Gal molecules. (A) Representative raw fluorescence-time traces of free-, half-cage and full-cage β-Gal. Five representative molecules are shown for each sample. The fluorescence intensity of enzyme reaction on the microscope slide was recorded for 5 min at 35 ms time resolution. (B, C, D) Statistics of spike frequency, fraction of active molecules, and overall observed enzyme activity. The number of active molecules analyzed is denoted by ‘n’ in (B). The standard deviations for spike frequency and fraction of active molecules were calculated after randomly assigning the active molecules into three groups. The standard deviation for the normalized overall activity was estimated from the propagation of errors. All experiments were carried out at room temperature in Ix TBS buffer, pH 7.5 in presence of 1 mM Mg²⁺ and 10% (w/v) PEG 8000.

FIG. 60 shows representative intensity-time traces (black) of full-cage enzyme after background correction and Hidden Markov Model (HMM) idealization to a two-state model (red). The fluorescence-time traces of the enzyme reaction on the microscope slide were recorded at 35 ms time resolution over 5 min.

FIGS. 61A-61D show titrations showing the effects of (A) NaCl, (B) KC1, (C) NH4Cl and (D) Triethylammonium acetate (TEAA) on the activity of free G6pDH. Assay conditions: 0.5 nM enzyme was incubated with a series of ion concentrations from low to high. Enzyme activity was monitored by absorbance at 340 nm with the addition of 1 mM Glucose-6-phosphate and 1mM NAO+in 1× TBS buffer (pH 7.5). The results show that high concentration of salts containing small cations such as Na+, K+and NH4+s ignificantly reduce the activity of G6pDH, possibly due to the chaotropic ion effect that disrupts hydrogen-bonded water structures as reported in the previous studies (Zhao, H. Journal of Molecular Catalysis B: Enzymatic 2005, 37, 16; Leberman, R. and Soper, A. K. Nature 1995, 378, 364.). Conversely, the salt containing a bulky organic cation (kosmotropic), triethylammonium, does not strongly inhibit enzyme activity, even at high concentrations. Error bars were calculated from the standard deviation of at least three replicates.

FIG. 62 shows comparison of G6pDH activity inside three different DNA full-cages, compared with that of free G6pDH, using NAO+as the varying substrate. The SH, SS and DS cages are described in the main text. Enzyme assay conditions: 0.5 nM enzyme or DNA-cage-encapsulated enzyme, 1 mM glucose 6-phosphate, with different concentration of NAO+ ranging from 10 μM to 2500 μM, in 1× TBS buffer (pH 7.5, 1 mM MgCl₂) monitoring absorbance at 340 nm. The table lists the fit parameters. Encapsulation of the enzyme in different DNA full-cages caused a 1.2- to 1.5-fold increase in K_{M} and a 5- to 9-fold increase in k_{cat}.

FIG. 63 The relative activity of a GOx/HRP pair when attached to a variety of DNA scaffolds: enzyme wildtypes (GOx/HRP), ssDNA (GOx/HRP-ssDNA), 2D rectangular DNA origami (GOx/HRP-2D origami), separate 3D half cages (Half[GOx]/Half1HRPD, separate full cages (Full[GOx]/FullIHRPD and the same full cage (Full [GOx/HRP]). Enzyme activity is positively correlated to the density of DNA helices within the scaffolds, and partially or fully caged enzymes exhibit activity several-fold higher than that of free and unconjugated enzymes. Error bars were calculated from the standard deviation of at least three replicates. The value for GOx/HRP-2D origami is extracted from our previously published article (Fu, J. et al. JACS 2012, 134, 5516-5519). We concluded that the boosted activities of Full[GOx/HRP] cannot be simply attributed to a single factor of DNA density or close proximity, but may be induced by both of the high DNA density and close proximity within a DNA cage.

FIG. 64 shows raw activity data for full-cage [HRP/GOx] (0.5 nM) before and after trypsin digestion for 24 hours at 37° C. in 1×TBS buffer (pH 7.5).

FIG. 65 shows raw activity data of a free pair of HRP and GOx (0.5 nM) before and after trypsin digestion for 24 hours at 37° C. in 1×TBS buffer (pH 7.5).

FIG. 66 shows raw activity data for free G6pDH (0.5nM) before and after trypsin digestion for 1 h at 37° C. in 1×TBS buffer (pH 7.5). Error bars were calculated from the standard deviation of at least three replicates.

FIG. 67 shows raw activity data for Full[G6pDH] (0.5nM) time course trypsin digestion from 0 h to 24 h at 3 7° C. Error bars were calculated from the standard deviation of at least three replicates.

### DETAILED DESCRIPTION

The present disclosure describes a three dimensional nanocage assembly to encapsulate a biological macromolecule and methods of nanocage assembly. DNA nanostructures have emerged as promising molecular scaffolds to organize biomolecules at the nanoscale based on their programmable, sequence-driven self-assembly. For example, multi-enzyme cascades have been assembled on DNA nanostructures with precise control over the spatial arrangement to enhance catalytic activity by substrate channeling. Conversely, self-assembling DNA nanoboxes and -cages have shown promise in the delivery of macromolecular payloads such as antibodies and enzymes. Tubular DNA nanostructures have also been used to construct efficient enzyme cascade nanoreactors. The present invention is based at least in part on the inventors' development of a simple and robust strategy for encapsulating metabolic enzymes in DNA-templated nanocages, where nanoparticles comprising the nanocaged enzymes are obtained with high assembly yield and controlled packaging stoichiometry.

Accordingly, in a first aspect, provided herein is a nanoparticle useful for the transport and administration of therapeutic agents, bioactive compounds, biomolecular reagents, biocatalysts, and other molecular compounds of interest, referred to generally herein as payload molecules (e.g., nucleic acids, polypeptides, enzymes, antibodies, or phospholipids). As used herein, the term “nanoparticle” refers to a structural composition comprising a full closed nanocage and at least one payload biological macromolecule tapped within the inner cavity of the nanocage. As used herein, “nanocage” may refer to a three dimensional body comprising an inner cavity. The three dimensional body of the nanocage is an assembly of a plurality of structural members. The internal surfaces of the structural members form the edges of the inner cavity. In one embodiment, these structural members are, tubular, rod like or linear, and may be constructed using nucleic acids. In another embodiment, the structural members are assemblies of double stranded DNA. A full closed nanocage may be formed by the assembly of two half cages.

The nanocage may be assembled using any means known in the art in which a nano-scale structure if formed. Assembly methods include, but are not limited to, DNA origami, or assembly using liposomes, polymersomes, or virus-like particles. For example, nanocages can be assembled by genetic fusion, chemical crosslinking, surface co-immobilization, and encapsulation within polymer vesicles, or virus-like particles. As used herein, “DNA origami” may refer to an assembly technique that folds a single-stranded DNA template into a 2 or 3 dimensional target structure by annealing it with short staple strands. In one embodiment, the body of the nanocage comprises between 0.10 to 0.30 DNA helices per nm². In another embodiment, the body of the nanocage comprises between 0.11 to 0.17 DNA helices per nm².

In one embodiment, the DNA used to assembly the body of the nanocage is M13mp18 single-stranded DNA. M13mp18 DNA is a circular, single-stranded virus DNA of approximately 7249 nucleotides in length and was isolated from M13mp18,a M13 lac phage vector comprising single HindIII, SphI, SbfI, PstI, SalI (AccI/ HincII), XbaI, BamHI, SmaI (XmaI), KpnI (Acc65I), Sad and EcoRI sites within the gene encoding β-Galactosidase. Generally, M13mp18 DNA is useful as a standard and has been tested as a template in the dideoxy-nucleotide termination method of sequencing DNA. Detailed sequences are available at neb.com/products/n4040-ml3mp18-single-stranded-dna#pd-description on the World Wide Web.

Other single-stranded circular DNA that can be used to fold a DNA nanocage include, without limitation, p′7308, p′7560, p7704, p8064, p8634, and pEGFP.

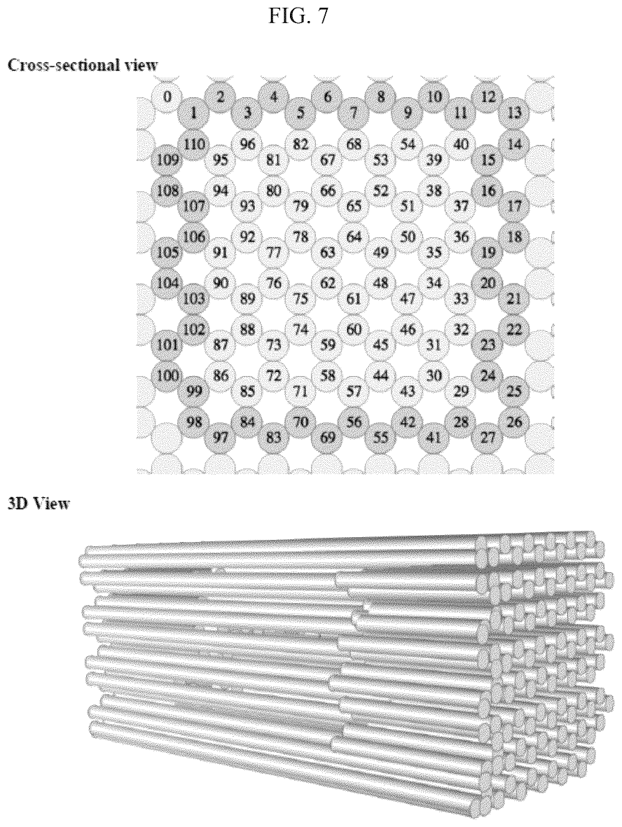

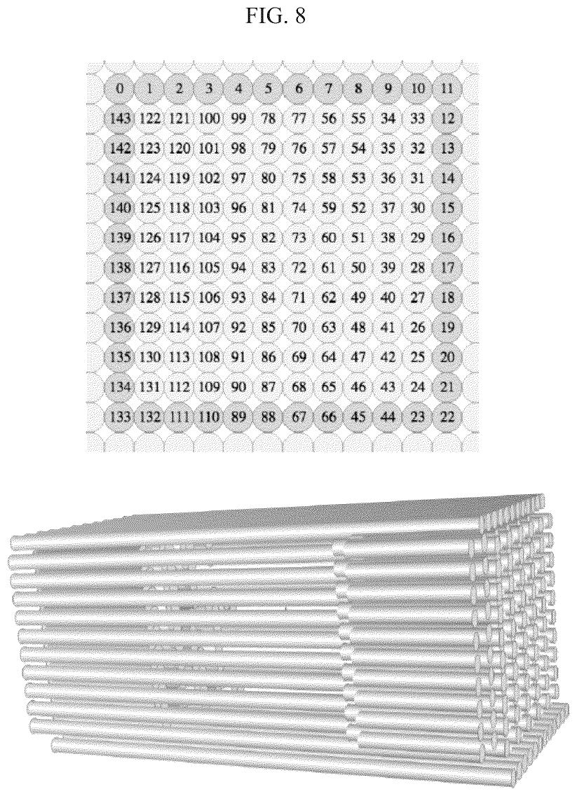

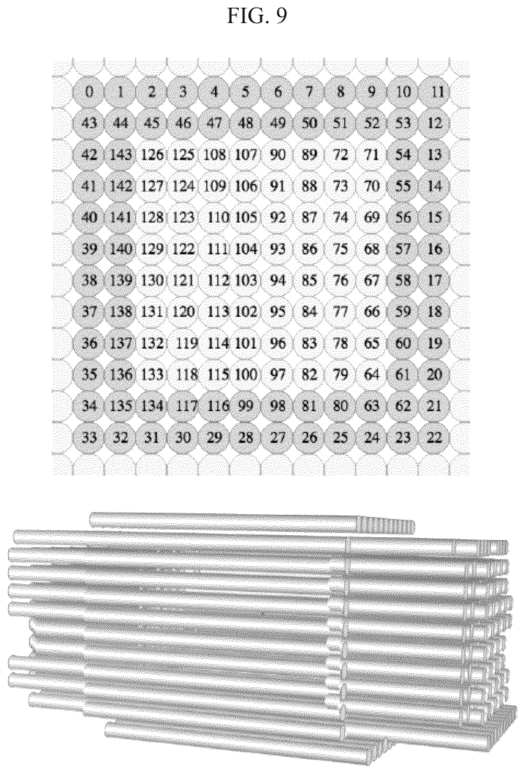

The nanocage may be formed in any architecture compatible with the chosen method of assembly. In one embodiment, the architecture of the structural members forms a square lattice, wherein the structural members and arranged in columns and rows. In another embodiment, the architecture of the structural members forms a honeycomb lattice (see FIG. 7). The architecture, assembly and three dimensional lattice of the nanocage may also accommodate variations in the number of and thickness of the walls of the nanocage. In one embodiment, the nanocage has a single walled square lattice arrangement as shown in FIG. 8, where there is a single layer of structural element between the inner cavity and the exterior of the nanocage. In one embodiment, the nanocage has a double walled square lattice arrangement as shown in FIG. 9, where there is a double layer of structural element between the inner cavity and the exterior of the nanocage. In another embodiment, the nanocage is multi-walled with several layers of structural elements between the inner cavity and the exterior of the nanocage.

The nanocage may be any size to accommodate the encapsulation of the enzyme or macromolecule of interest. In one embodiment, the dimensions of the nanocage are less than 100 nm×100 nm×100 nm. In another embodiment, the dimensions of the nanocage are less than 75 nm×50 nm×50 nm. In another embodiment, the dimensions of the nanocage are about 40-70 nm×15-40 nm×15-40 nm.

The inner cavity of the nanocage is a hollow, open space enclosed within the nanocage to contain the macromolecule or enzyme of interest. The inner cavity will have dimensions smaller than those of the nanocage. In one embodiment, the dimensions of the inner cavity are less than 50 nm×50 nm×50 nm. In one embodiment, the dimensions of the inner cavity are less than 30 nm×30 nm×20 nm.

In some cases, a nanoparticle as provided herein encapsulates a biological macromolecule within the inner cavity of the nanocage. Any biological macromolecule having any purpose or function can be encapsulated as payload within a nanocage thereby forming a nanoparticle. Exemplary biological macromolecules include, without limitation, proteins, enzymes, antibodies, protein complexes, phospholipids, nucleic acids, and combinations thereof. In one embodiment, the biological macromolecule is an enzyme. The design and structure of the nanocage may be changed and adjusted to accommodate a variety of enzymes with any size, shape, morphology or function. In one embodiment the enzyme has a molecular weight between 10-600 kilodaltons (kDa). Preferably, the nanocage accommodates enzymes having a molecular weight equal to or less than about 600 kDa.

Without being limited to one particular theory or practice, the nanocage structure may be configured for a variety of functions in regards to the encapsulated enzyme or macromolecule. In embodiments in which the macromolecule is an enzyme, the nanocage may be configured such that the catalytic activity of the enzyme may be tested while the enzyme is encapsulated within the nanocage. The nanocage may also be configured such that the catalytic activity of the enzyme is enhanced when encapsulated within the inner cavity. The nanocage may also be configured such that the enzyme is stabilized against protease digestion or proteolytic degradation when encapsulated within the inner cavity.

The nanocage structure and assembly may be designed and assembled into a nanoparticle to accommodate a variety of macromolecular configurations within the inner cavity. For example, a single nanocage may encapsulate a payload including but not limited to, a single biological macromolecule, a pair of biological macromolecules, a plurality of biological macromolecule, assemblies of biological macromolecules, multi-component complexes of biological macromolecules, or combinations thereof. When the nanoparticle comprises a payload of multiple biological macromolecules, the macromolecules may be the same, or they may be an assembly of two or more different macromolecules.

A nanocage may comprise one or more nanopores. As used herein, “nanopore” may refer to a nano-scale passage, pore or opening in the nanocage. The nanopore may be configured to allow the passage of small molecule substrates, solvents, enzyme substrates and products and the like into and out of the nanocage. The nanopores are sized such that the enzyme encapsulated within the nanocage cannot escape. In one embodiment, the nanocage comprises at least one nanopore. In another embodiment, the nanocage comprises 1-200 nanopores. In another embodiment, the nanocage comprises 10-75 nanopores. The size of the nanopores is determined by the interaction, arrangement and architecture of the structural members of the body of the nanocage, such that nanopores may be formed in the gaps between the structural members. In one embodiment, the nanopores are between 1 and 5 nm in diameter. In another embodiment, the nanopores are between 1.5 and 3 nm in diameter.

In some embodiments, the enzyme may be non-covalently linked to the internal surface of the nanocage. In another embodiment, the enzyme may be covalently connected to the internal surfaces of the nanocage. In one non-limiting, exemplary embodiment, succinimidyl 3-(2-pyridyldithio) propionate (SPDP) chemistry may be used to crosslink a surface lysine residue on the biological macromolecule to a thiol-modified oligonucleotide. Other useful methods include, without limitation, aptamer-protein noncovalent interactions, NTA-hexahistidine interactions, click chemistry, disulfide and maleimide coupling, and SPDP and SMCC (N-Succinimidyl 3-(2-pyridyldithio)-propionate) cross-linking.

A half-cage of the body of the nanocage may be assembled utilizing DNA origami. DNA structures can be designed with caDNAno and single strand DNA may be used as the scaffold. To form the half-cage, single strand DNA may be mixed with corresponding staple strands and annealed. Excess staple strands may be removed by filtration.

An enzyme molecule may be attached to the open half-cage by any appropriate covalent or non-covalent chemistry such as, for example, include, without limitation, aptamer-protein noncovalent interactions, NTA—hexahistidine interactions, click chemistry, disulfide and maleimide coupling, and SPDP and SMCC (N-Succinimidyl 3-(2-pyridyldithio)-propionate) cross-linking.

In some cases, two half-cages of the body of the nanocage are assembled by linking together half-cages. Linking may occur by incubating half-cages with DNA linkers. For example, DNA linkers may hybridize with sticky ends extending from the edge of “DNA half-cages.” Preferably, DNA linkers are complimentary to these sticky ends, and can be varied for different DNA cage sequences.

The present invention has been described in terms of one or more preferred embodiments, and it should be appreciated that many equivalents, alternatives, variations, and modifications, aside from those expressly stated, are possible and within the scope of the invention.

### Example 1

The embodiment described here demonstrates a simple and robust strategy for the DNA nanocage-templated encapsulation of metabolic enzymes with high assembly yield and controlled packaging stoichiometry. With such an approach in hand, the hypothesis that the recently described, chaperone-like stabilizing impact of polyphosphate on metabolic protein enzymes together with the cryptic RNA binding properties of many enzymes may lead to beneficial effects when enzymes are surrounded by DNA nanocages, is tested.

### Methods

The design and characterization of DNA half-cages and full-cages.

DNA origami half-cage and structures were designed with caDNAno, each used one M13mp18 ssDNA as the scaffold. Detailed design schemes are shown in FIGS. 7-9. One or both of the half-cages contained single-stranded probe strands (4 in each half-cage) extended toward the inside of the cage for binding with the DNA conjugated enzymes. Two of the half-cages can be linked together to form a fully enclosed full-cage though 24 linker strands. To form each of the half-cages, the M13mp18 ssDNA was mixed with the corresponding staples at a 1:10 molar ratio in 1×TAE-Mg2+ buffer (40 mM Tris, 20 mM acetic acid, 2 mM EDTA and 12.5 mM magnesium acetate, pH 8.0), annealed from 80° C. to 4° C. for 37 h. The excess staple strands were removed by the filtration of the DNA cages solution using 100-kD Amicon filter with 1×TAE-Mg2+ buffer for three times. To form a full-cage, 24 single-stranded DNA linkers were incubated with the two purified half-cages at a molar ratio of 5:1 for three hours at room temperature, in order to connect the two half-cages together.

Enzyme-DNA cage assembly.

A 15-fold molar excess of oligonucleotide-conjugated enzyme was incubated with the DNA half-cage containing capture strands. Protein assembly was performed using an annealing protocol in which the temperature was gradually decreased from 37° C. to 4° C. over 2 h and then held constant at 4° C. using an established procedure. Two Enzyme-attached half cages were then assembled into a full cage by adding DNA linkers as described above. The DNA caged-enzymes were further purified by agarose gel electrophoresis to remove excess free enzymes.

Preparation, purification, and characterization of protein-DNA conjugates.

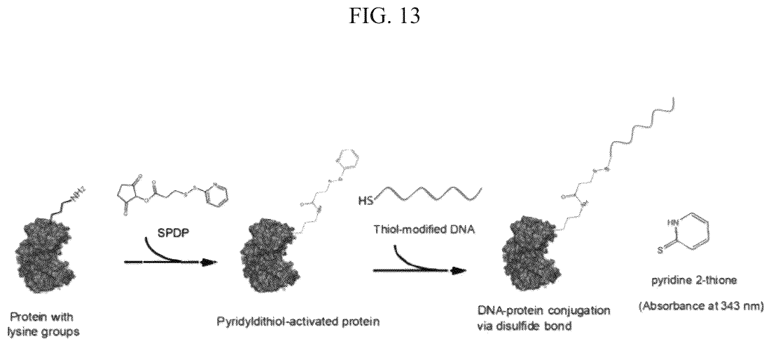

Protein-DNA conjugation- as shown in FIG. 13, SPDP conjugation chemistry was used to couple enzymes to oligonucleotides as reported previously. Enzymes (GOx, HRP, G6pDH, LDH, MDH and β-Gal) were first conjugated with SPDP at enzyme-to-SPDP ratios of 1:5, 1:20, 1:3, 1:5, 1:5, and 1:5, respectively, in HEPES buffer (50 mM HEPES, pH 8.5) for 1 h at room temperature. Different values of SPDP-to-Protein ratio were used due to the varied number of accessible surface lysine residues for each protein. Excess SPDP was removed by washing with 50 mM HEPES buffer using Amicon centrifugal filters (30 kD cutoff). The SPDP coupling efficiency was evaluated by monitoring the increase in absorbance at 343 nm due to the release of pyridine-2-thione (extinction coefficient: 8080 M⁻¹ cm⁻¹).

TCEP-treated thiolated DNA (/5ThioC6-/-TTTTTCCCTCCCTCC (SEQ ID NO:1393) (P1), or /5ThioC6-D/-TTTTTGGCTGGCTGG (SEQ ID NO:1394) (P2) was incubated with the SPDP-modified enzymes at an enzyme-to-DNA ratio of 1:10 in 50 mM HEPES buffer (pH 7.4) for 1 h in the dark. Excess unreacted oligonucleotide was removed by ultrafiltration using Amicon 30 kD cutoff filters: washing one time with 50 mM HEPES (pH 7.4) containing 1 M NaCl and three times with 50 mM HEPES (pH 7.4). The high salt concentration in the first washing buffer helps remove DNA nonspecifically bound to the surface of the protein due to electrostatic interactions.

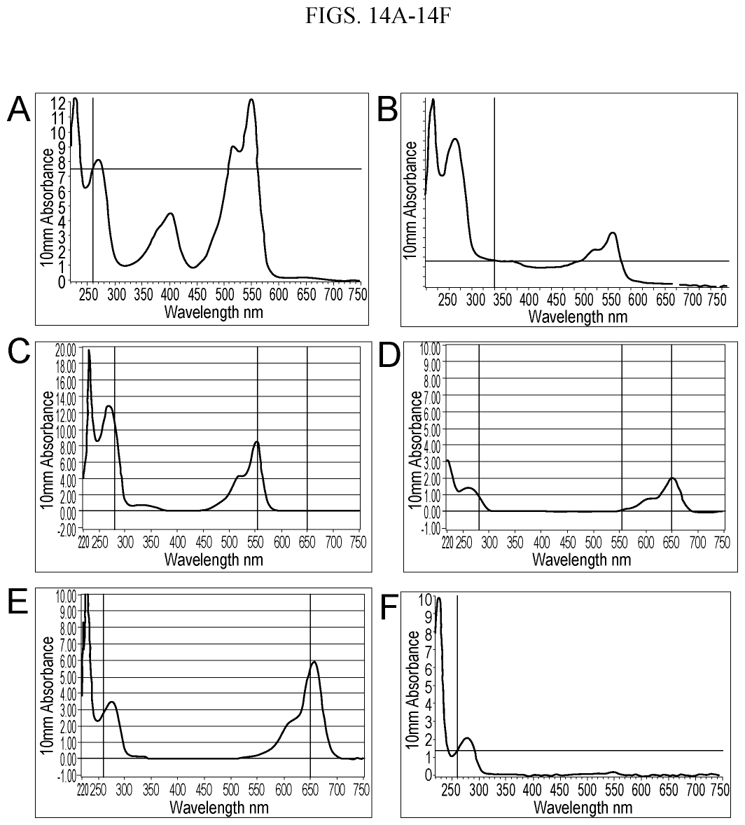

The absorbance values at 260 nm and 280 nm (A260 and A280) were recorded to quantify the enzyme-DNA complex concentrations and the labeling ratios using a Nanodrop spectrophotometer (Thermo Scientific) (FIGS. 14A-14F and Table 2). Extinction coefficients of DNA oligonucleotides were received from IDT-DNA, and extinction coefficients of enzymes were obtained from published data.

Dye labeling of DNA-conjugated proteins: The DNA-conjugated proteins were further labeled with spectrally distinct fluorescent dyes, which allow us to use native gel electrophoresis and single-molecule fluorescence to confirm the encapsulation of proteins within DNA nanocages. NHS-ester-modified dyes were reacted with the purified DNA-conjugated proteins from the above steps at a 20:1 ratio in 50 mM HEPES buffer, pH 8.5. Cy3 was directly labeled to the lysine residues on the protein surface. Excess dyes were then removed using 3-kD cutoff Amicon filters. The UV-Vis absorbance spectra of the purified dye-labeled proteins are shown in FIGS. 14A-14F and were used together with the extinction coefficients of the dye (150,000 M⁻¹ cm⁻¹ for Cy3 at 546 nm; 250,000 M⁻¹ cm⁻¹ for Alexa647 at 647 nm) and of the protein-DNA conjugates to quantify the concentration and labeling ratio of the dye-labeled proteins.

Conjugate proteins to Cy3-labeled DNA: In order to perform the single-molecule enzyme activity assay, selected enzymes (G6pDH and β-Gal) were conjugated to a Cy3-labeled DNA. First, NHS-ester-modified dyes were reacted with the 3 ‘-amine of oligonucleotides at a 20:1 ratio in 50 mM HEPES buffer, pH 8.5. Excess dyes were then removed using 3-kD cutoff Amicon filters. Dye-modified oligonucleotides were then conjugated to proteins via the 5 ’-thiol using the SPDP chemistry described above. Fast Protein Liquid Chromatography (FPLC) was used to purify the protein-DNA-Cy3 conjugates for removing excess DNA-Cy3, and characterized with the UV-Vis absorbance spectra.

### Enzyme-DNA Cage Assembly, Purification, and Characterization

The purified DNA half-cage containing capture strands was mixed with one of several enzyme-DNA conjugates at a 1:15 cage:enzyme ratio and annealed from 37° C. to 4° C. over 2 h in 1×TAE-Mg²⁺ buffer (containing 12.5 mM Mg(OAc)2). Twenty-four single-stranded DNA linkers were mixed with the two purified half-cages at a 5:1 linker: cage ratio to connect the two half-cages together by incubating at room temperature for 3 h. Agarose gel electrophoresis (2%, 1×TAE-Mg21 was employed to remove excess free enzymes (70V, 2h). The band of the DNA cage containing the enzyme was cut from the gel and extracted using a Freeze 'N Squeeze column (Bio-Rad). The DNA origami concentration was quantified by measuring the absorbance at 260 nm (A260) using an extinction coefficient of 0.109 nM⁻¹cm⁻¹.

### DNA Sequences of the Designed Nanocages

Sequences of staple strands in the SH Full-Cage-Left cage are listed in SEQ ID NOs: 1-210.

Sequences of staple strands in the SH-right cage are listed in SEQ ID NOs:211-420.

AB-Linker strands are listed in SEQ ID NOs:421-444.

SH-probe strands are listed in SEQ ID NOs:445-450. The BOLD portions of the sequences are complementary to the ssDNA conjugated to the enzymes, and are located in the Left (SEQ ID NOs:445-447, top) and Right (SEQ ID NOs:448-450, bottom) half-cages.

34[53]
ATGACCATAAATCGCCTGATAAAT GGAGGGAGGG
48[53]
TGTGTCGAAATCCCTCAGAACCGC GGAGGGAGGG
62[53]
CACCCTCAGAGCGCAGCACCGTAA GGAGGGAGGG
51[117]
TTTAGGCAGAGGCATTCAACGCCAACATGTAA CCAGCCAGCC
61[117]
CGAACAAAGTTACCAGAAAGTAAGCAGATAGC CCAGCCAGCC
75[117]
GTAAGCGTCATACATGTGAATTTACCGTTCCA CCAGCCAGCC

Sequences of staple strands in the SS-left half-cage are listed in SEQ ID NOs:451-669.

Sequences of staple strands in the SS-right half-cage are listed in SEQ ID NOs:670-890.

SS-linker strands are listed in SEQ ID NOs:891-908.

SS-probes are listed in SEQ ID NOs:909-914. The BOLD portions of the sequences are complementary to the ssDNA conjugated to the enzymes, and are located in the Left (SEQ ID NOs:909-911, top) and Right (SEQ ID NOs:912-914, bottom) half-cages.

94[44]
GATATAAGTATAGTGACACAGACAGCCCTCATGGAGGGAGGG
104[50]
CTTTTGATGATGTCAGTGCCTTGGAGGGAGGG
110[44]
CATTGACAGGAGGATTTAAGCGTCATACATGGGGAGGGAGGG
87[115]
GCAAGCAAATCAGGCTTATTTTGCACCCAGCTCCAGCCAGCC
93[109]
ACAATTTTATCCAGAGCCTAATCCAGCCAGCC
103[115]
GTAAGCAGATAGCTATAATAGAAAATTCATATCCAGCCAGCC

DNA sequences for DS Full-Cage design, cross sectional view

Sequences of staple strands in the DS-left half-cage are listed in SEQ ID NOs:915-1134.

Sequences of staple strands in the DS-right half-cage are listed in SEQ ID NOs:1135-1362.

DS-linker strands are listed in SEQ ID NOs:1363-1386.

DS-probes are listed in SEQ ID NOs:1387-1392. The BOLD portions of the sequences are complementary to the ssDNA conjugated to the enzymes, and are located in the Left (SEQ ID NOs:1387-1389, top) and Right (SEQ ID NOs:1390-1392, bottom) half-cages.

64[71]
ATTCATTTCAATTACCCGCGCAGAGGCGAATTTTTTGGAGGGAGGG
74[76]
TCAGATGATGGCAACAATAACTTTTGGAGGGAGGG
76[66]
ATTATCATTTTTTATCATCATATTCCTGATTATTTTGGAGGGAGGG
34[149]
TTCTGTGCAAAAGAAGGCACCAGGCTGACCGTAATCTTGACAAGAACCGGA
TTTTC CAGCCAGCC
67[136]
GCAAAAGACGGTGTACAGACCTTTTCCAGCCAGCC
73[131]
GCATCAAAAAGATTAAGAGGAACTTCAAATATCGCGTTTTAATTTTCCAGC
CAGCC

Single-molecule fluorescence microscopy.

All single-molecule measurements were performed at room temperature using a total internal reflection fluorescence (TIRF) microscope on PEGylated fused silica microscope slides. To passivate the microscope slides and functionalize the surface with biotin for selective immobilization of nanocages, a biotin- and PEG-coated surface was prepared by silylation with APTES, followed by incubation with a 1:10 mixture of biotin-PEG-SVA 5k:mPEG-SVA 5k as described previously. A flow channel was constructed as described elsewhere. To prepare the surface for enzyme or nanocage binding, a solution of 0.2 mg/mL streptavidin in T50 buffer (50 mM Tris-HCl, pH 8.0, 50 mM NaCl, 1 mM EDTA) was injected in to the flow channel, incubated for 10 min, and the excess streptavidin was flushed out thoroughly first with T50, then with 1× TAE-Mg2+.

Yield estimation by TIRF colocalization: All single-molecule measurements were performed at room temperature using a total internal reflection fluorescence (TIRF) microscope on PEGylated fused silica microscope slides. To passivate the microscope slides and functionalize the surface with biotin for selective immobilization of nanocages, a biotin- and PEG-coated surface was prepared by silylation with APTES, followed by incubation with a 1: 10 mixture of biotin-PEG-SVA 5k:mPEG-SVA 5k as described previously³. A flow channel was constructed as described elsewhere 3 . To prepare the surface for enzyme or nanocage binding, a solution of 0.2 mg/mL streptavidin in TSO buffer (50 mM Tris-HCl, pH 8.0, 50 mM NaCl, 1 mM EDTA) was injected in to the flow channel, incubated for 10 min, and the excess streptavidin was flushed out thoroughly first with TSO, then with 1×TAE-Mg.

The right half of the DNA origami cage was labeled with Cy5 dye inside the cavity, via hybridization of Cy5-labeled DNA to complementary handles incorporated into the structure. Each of the ssDNA conjugated enzymes (HRP, GOx, G6pD, LDH, MDH and β-Gal) was covalently labeled with Cy3 as described in section 3 (Cy3-Enzyme-5′-TTTTTCCCTCCCTCC, SEQ ID NO:1393), and then linked to the left half of the DNA origami cage via hybridization with complementary handles. Because Cy3 was directly labeled onto the enzyme surface, any observed Cy3 signal of the immobilized DNA nanocages came from the encapsulated enzymes. Linker strands were added to a 1:1 mixture of the two half-cages to encapsulate the enzymes in a full-cage. To capture DNA-modified enzymes in the absence of nanocage (as control) the microscope slide was incubated with 10-20 nM biotin-modified complementary DNA oligonucleotide (5′-biotin-TTTTTGGAGGGAGGG, SEQ ID NO:1395) for 3 min, followed by 10 min incubation with 20-50 pM enzyme sample in 1× TAE-Mg buffer. Excess enzyme was flushed out with 400 uL buffer (channel volume 30 μL). For the nanocage experiments, the samples were diluted to 20-50 pM in 1× TAE-Mg and immobilized on the streptavidin-coated PEG surface for 1 min, and the excess sample was flushed out with 400 (IL of 1×TAE-Mg. The DNA-modified enzymes were imaged with illumination at 532 nm (15 W/cm2), and the nanocage-encapsulated enzymes were imaged with simultaneous illumination at both 532 nm (15 W/cm2) and 640 nm (40 W/cm2) as described. Particle-finding and colocalization analysis were performed using custom-written scripts in IDL and MATLAB, using a threshold of 150 counts per frame for particle identification (typical particles showed 500-1,000 counts per frame in each detection channel). The enzyme encapsulation yield, defined as the fraction of assembled nanocages containing enzyme(s), was estimated by dividing N_{caloc} by the total number of particles containing a right half-cage, N_{right} (Table 3).

Estimation of enzyme copy number per nanocage: The number of enzyme copies per nanocage (N_{enz}) was determined by single-molecule photobleaching (SMPB). First, the number of Cy3 photobleaching steps was determined separately for unencapsulated as well as half-cage and full-cage-encapsulated enzymes. For this, the donor channel data of all single molecules were idealized in QuB (http://www.qub.buffalo.edu) using a six-state model. The histogram of the photobleaching steps was then acquired using a custom-written MATLAB script.

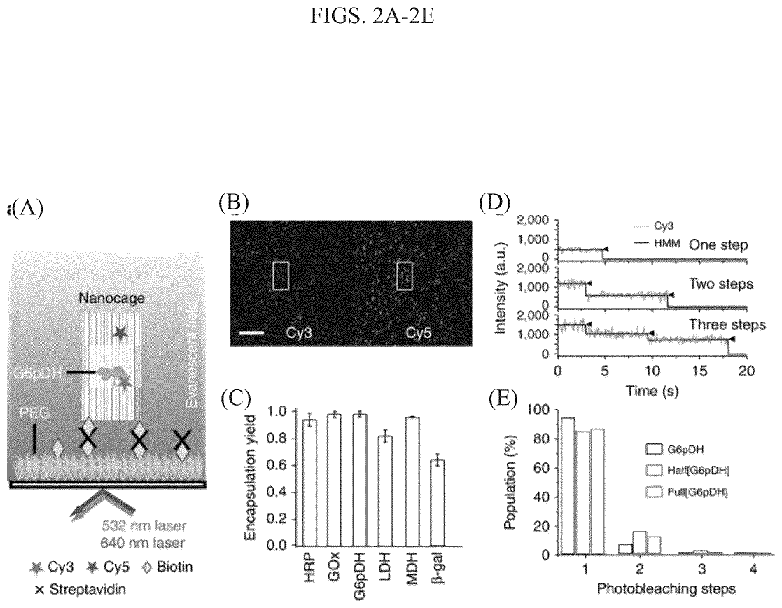

Representative intensity traces exhibiting one, two, and three photobleaching steps are shown in FIG. 2D (more than three photobleaching steps were rarely seen). Finally, the number of enzyme molecules per cage was estimated by dividing the mean number of Cy3 photobleaching steps of the full-cage (μ_{cy3_Encap}) by the mean number of Cy3 photobleaching steps for the unencapsulated enzyme (μ_{cy3_Unencap}). Results are summarized in Table 4.

### Single-Molecule Enzymology

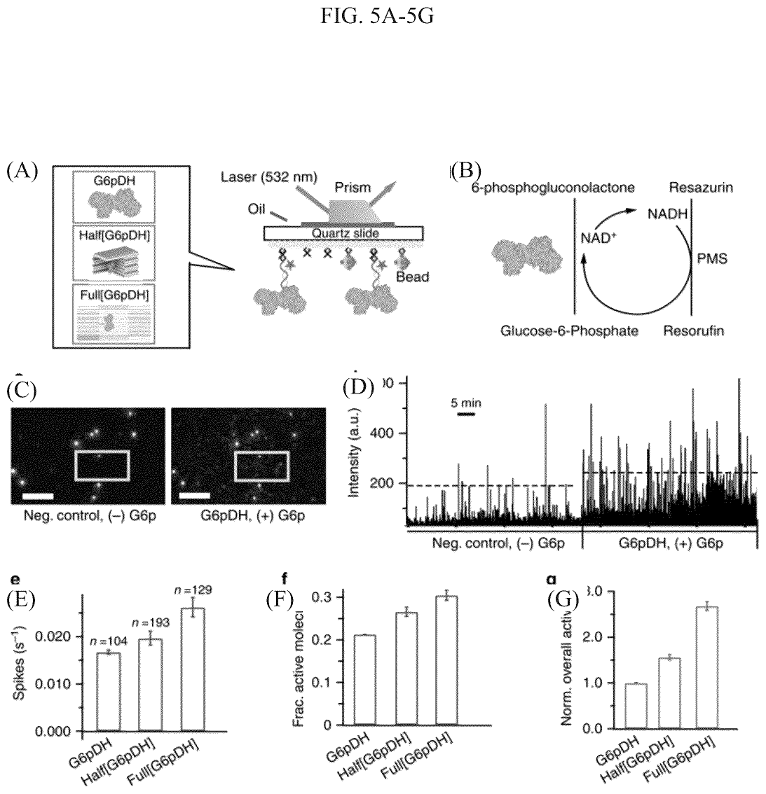

Single-molecule enzyme activity assay: Prior to single-molecule activity measurement, streptavidin-modified slides were incubated for 2 min with neutravidin-coated fluorescent beads (Invitrogen, 0.04 p.m diameter, excitation/emission; 550/605 nm) at 106-fold dilution and the excess flushed out with 1× TBS buffer. These beads (5-8 per field of view) were used as fiducial markers to correct for drift of the microscope stage and/or slide (FIGS. 5A and 5C). Following complete photobleaching of Cy3 in a field of view, the activity of single unencapsulated or nanocage-encapsulated enzyme molecules was imaged on the same field of view. During analysis of the movies, the coordinates of the initial photobleaching movie were registered with those of subsequent movies using the fiducial markers (visible throughout all sequential movies) in a custom-written MATLAB script. This approach allowed us to infer the locations (x- and y-coordinates) of all individual enzymes/nanocages in the field of view even after bleaching Cy3, and to monitor enzyme turnovers (resorufin formation) at these specific coordinates.

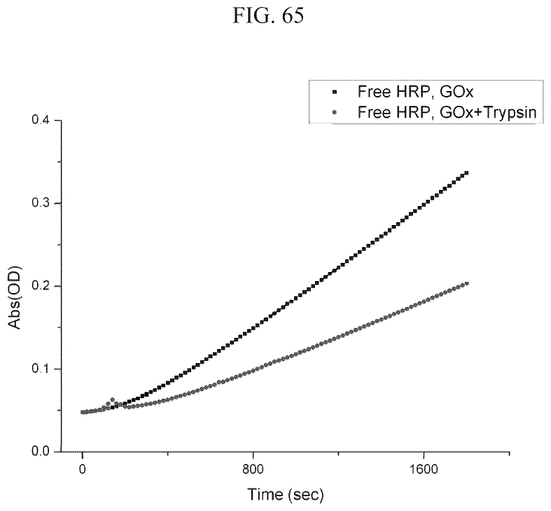

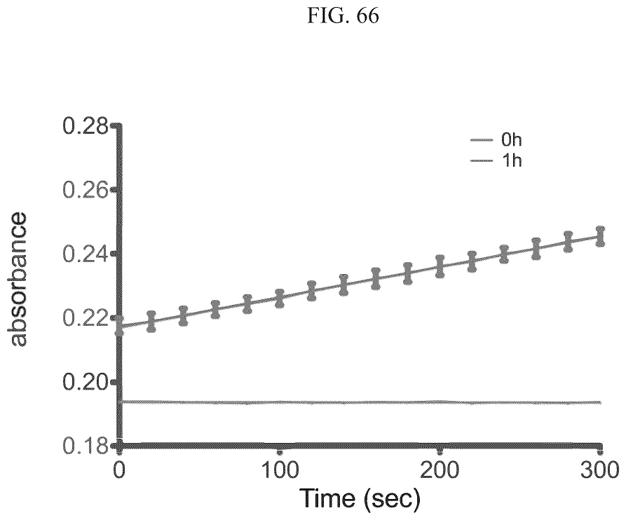

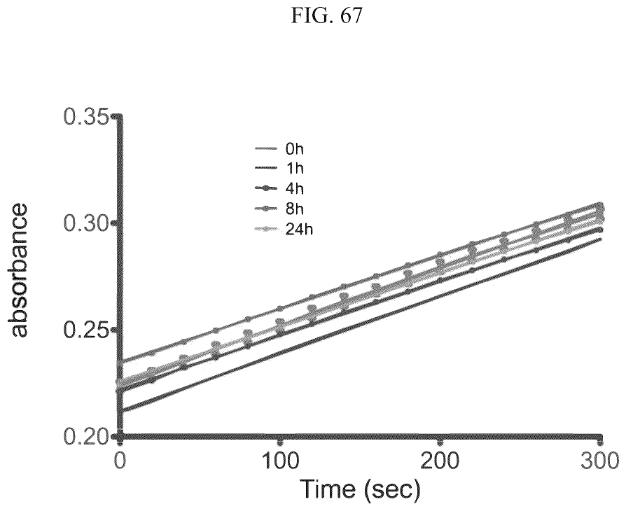

To image enzyme activity, 300 μL of substrate solution in 1× TBS buffer (pH 7.5, 1 mM Mg²⁺, and 10% (w/v) PEG8000) (Table 5) was injected into the flow channel. Movies were recorded for 5 min (9,091 frames) at 35 ms frame exposure time immediately after injecting the substrate solution. In case of G6pDH, the activity was measured in the same field of view under identical laser illumination and microscope settings, with or without glucose-6-phosphate (G6p) (FIG. 5c). Enzyme activity for β-Gal was measured similarly using a 500 nM solution of resorufin (3-D-galactopyranoside (RBG) as substrate, which is hydrolyzed by β-Gal into fluorescent resorufin. Fluorescence fluctuations over time were measured for unencapsulated enzyme as well as half- and full-cage-encapsulated enzyme (FIG. 65 and FIG. 66), and the fluorescence time traces were analyzed for intensity spikes using custom-written MATLAB script. The script allowed us to measure the background intensity of single-molecule traces and set a threshold (mean +8 standard deviations) to subtract from the raw intensity. Since we often observed one or two spikes above this intensity threshold in the control experiments, only those molecules with 2:4 spikes were counted as active molecules (FIG. 67) and considered for burst analysis. Due to the low concentration of resazurin (Table 5), the criteria we used to determine the fraction of active molecules might have excluded some molecules that are not highly active.

Burst analysis: Burst analysis was carried out using a modified Rank Surprise (RS) method6 recently utilized to analyze the binding of fluorescent DNA probes to a riboswitch. Briefly, Interspike Intervals (ISIs) were determined by calculating the time in between individual fluorescent spikes for each molecule (FIG. 67). The RS method was used to demarcate the start and end points of bursts after collecting ISIs for all molecules. Only intensity spikes characterized by an ISIs of greater than 3 seconds were considered part of a burst; any other intensity spikes are counted as non-bursts.

Comparing bulk and single-molecule enzyme activity: Unlike our single-molecule assay, the bulk measurement of enzyme activity cannot explicitly determine the fraction of active enzyme molecules present in the solution (it is well known that a fraction of enzyme molecules loses their activity during oligonucleotide conjugation, buffer exchange and the purification process). However, the observed bulk activity is contributed not only by enzyme turnover rate but also by the fraction of enzyme molecules that are still active. Both of these contributing factors need to be accounted for to directly compare the single-molecule enzyme activity with the bulk measurements. Therefore, in the single-molecule experiment, the overall activity of free, half-cage and full-cage enzymes were calculated by multiplying the turnover rate with the fraction of active molecules for the given sample.

Bulk solution enzyme assay.

A 96-well-plate reader was used to monitor enzyme activity through absorbance changes of the samples. The enzyme samples and substrates were loaded in the wells of the 96-well plate with a final concentration of caged enzymes 0.5 nM in 1× TBS (Tris buffered saline with 1 mM MgC12, pH 7.5) for most assays.

Enzymes and substrates: Glucose-6-phosphate dehydrogenase (G6pDH, Leuconostoc mesenteroides), malic dehydrogenase (MDH, porcine heart), lactate dehydrogenase (LDH, rabbit muscle), glucose oxidase (GOx, Aspergillus niger), horseradish peroxidase (HRP) and β-galactosidase (β-Gal, E. coli) were purchased from Sigma (St. Louis, MO). Pyruvate, oxaloacetate (OAA), glucose 6-phosphate (G6P), glucose, resorufin β-D-glucopyranoside (RBG), β-nicotinamide adenine dinucleotide (NAD), resazurin (RESA) and phenazine methosulfate (PMS) were obtained from Sigma-Aldrich. ABTS (2,2′-Azino-bis[3-ethylbenzothiazoline-6-sulfonic acid] diammonium salt) was purchased from Pierce (Rockford, IL), polyphosphate (100) is ordered from Kerafast.
DNA strands:

Single-stranded MI3mp18 DNA was purchased from New England Biolabs. Staple strand oligonucleotides were obtained from Integrated DNA Technologies (IDT) on 96-well plates and used without further purification. Thiol-modified DNA oligonucleotides were also purchased from IDT, and were purified by denaturing PAGE before use.

Crosslinking reagents:

N-Succinimidyl 3-(2-pyridyldithio)propionate (SPDP) and tris(2-carboxyethyl)phosphine (TCEP) were obtained from Pierce. Dimethyl sulfoxide (DMSO) was purchased from Sigma.

### Buffers:

Phosphate buffered saline (PBS), HEPES sodium salt, Tris buffered saline (TBS), Tris base, acetic acid, EDTA, and magnesium acetate were purchased from Sigma. 1×TAE/Mg²⁺ buffer (pH 8.0) is prepared by 40 mM Tris, 20 mM acetic acid, 2 mM EDTA and 12.5 mM magnesium acetate.

Dye-labeling reagents:

NHS-Cy3, Cy5 amine reactive dyes were purchased from GE Healthcare Life Sciences. NHS-AlexaFluor®555 and AlexaFluor®647 amine reactive dyes were obtained from Life Technologies.

Amicon centrifugal filters were purchased from Millipore.

PEG 8000 was purchased from Promega.

Surface PEGylating reagents:

APTES (3-Aminopropyl)triethoxysilane was purchased from Sigma-Aldrich. mPEG-SV A 5k and biotin-PEG-SY A 5k were obtained from Laysan Bio, Inc.

TEM imaging:

TEM grids (400 mesh, copper grid coated with ultrathin carbon, Ted Pella) were glow discharged (Emitech K1 OOX). 2 μl concentrated samples were deposited onto the grids for 1 min, washed with 10 (11 DI water for 5 sec, stained with 10 (11 1% uranyl formate twice (2 sec for the first time and 15 sec for the second time), and imaged using Philips CMI2 transmission electron microscope.

Enzyme activity assay:

A 96-well-plate reader was used to monitor enzyme activity through absorbance changes of the samples. The enzyme samples and substrates were loaded in the wells of the 96-well plate with a final concentration of caged enzymes of 0.5 nM in 1×TBS (Tris buffered saline with 1 mM MgCl₂, pH 7.5) for most assays. The DNA cage concentration was determined by the A260 value as described above. For a typical GOx and HRP assay, 1 mM Glucose and 2 mM ABTS was used as substrate and enzyme activity was measured by monitoring the increase in absorbance at 410 nm (ABTs-1). For a typical G6pDH assay, 1 mM G6P and 1 mM NAD+were used as substrates, and enzyme activity was measured by monitoring the increased absorbance at 340 nm due to the reduction of NAD+to NADH. For a typical LDH assay, 2 mM pyruvate and 1 mM NADH were used as substrates, and enzyme activity was measured by monitoring the decreased absorbance at 340 nm due to the oxidation of NADH to NAD+. For a typical MDH assay, 2 mM OAA and 1 mM NADH were used as substrates, and enzyme activity was measured by monitoring the decrease in absorbance at 340 nm. For a typical β-Gal assay, 100 μM RBG was used as substrate and enzyme activity was measured by monitoring fluorescence intensity, with excitation at 532 nm and emission at 590 nm.

Trypsin assay:

Enzyme activity was measured after incubation with or without trypsin (1 μM) at 37° C. for 24 h in 1× TAE-10 mM Mg buffer (pH 8.0). Activity assay conditions: 1 mM Glucose, 1 mM ABTS, 1 nM of free GOx and HRP in pH 7.5, 1× TBS buffer containing 1 mM MgCl₂, and monitoring absorbance at 410 nm. In the DNA cage experiment, all conditions were the same except for incubating 1 nM DNA cage-encapsulated GOx and HRP with trypsin.

### Results

Enzyme encapsulation strategy.

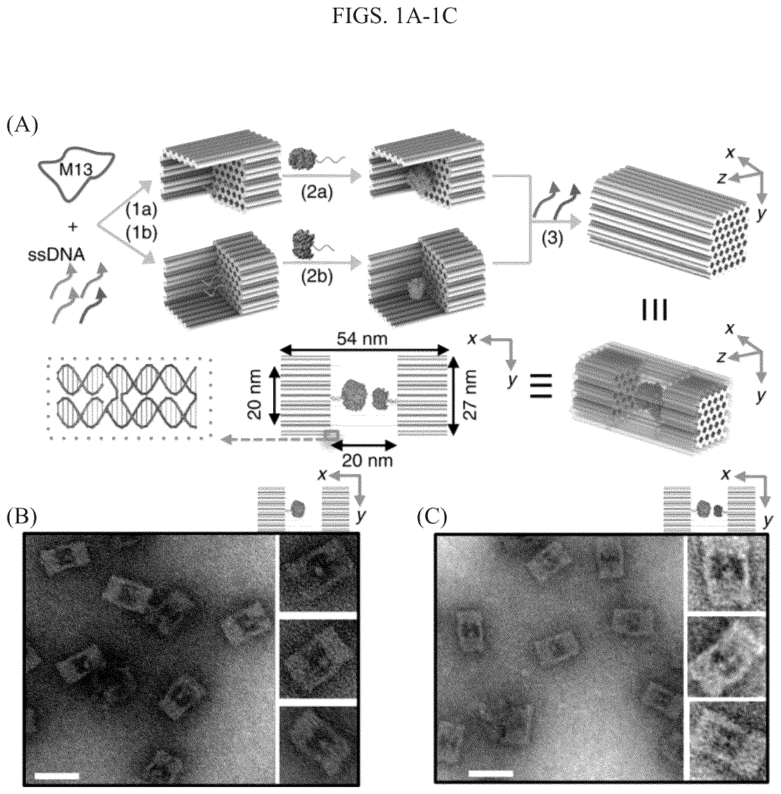

As shown in FIG. 1A, the current embodiment of the approach for enzyme encapsulation within DNA nanocages involves two steps: 1) the attachment of an individual enzyme into an open half-cage and 2) the assembly of two half-cages into a full (closed) nanocage. DNA half-cages were constructed by folding a full-length M13 viral DNA29 into the indicated shape based on a honeycomb lattice using the DNA origami technique; a shape with two open sides was chosen to improve accessibility of the internal cavity to large proteins. Two half-cages were then linked into a full-cage by adding 24 short bridge DNA strands that hybridize with the complementary ssDNA sequences extending from the edges of either half-cage. The DNA full-cage is 54 nm×27 nm×26 nm with designed inner cavity dimensions of 20 nm×20 nm×17 nm. By design, 42 small nanopores (each 2.5 nm in diameter) were introduced on each of the top and bottom surfaces of the DNA nanocage to permit the diffusion of small molecules (e.g., enzyme substrates) across the DNA walls (FIG. 7).

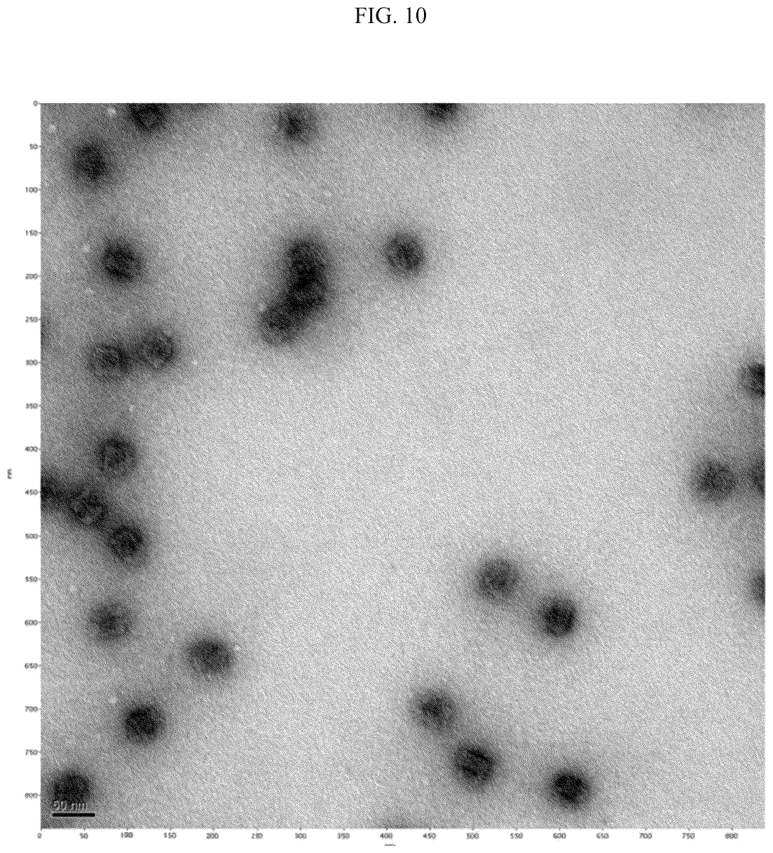

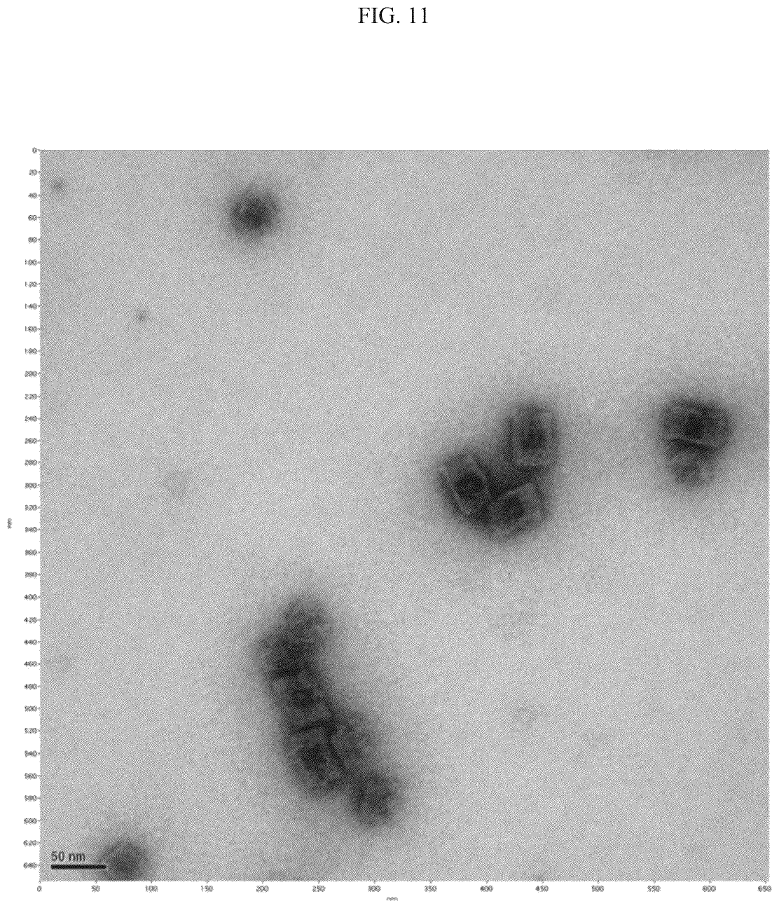

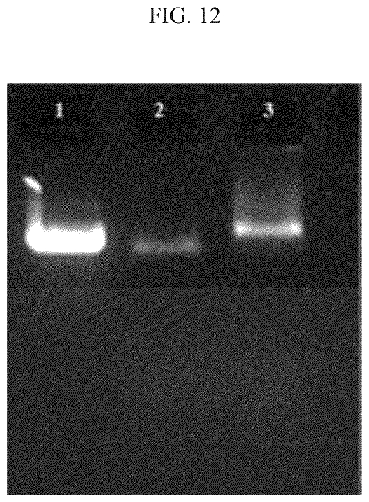

The formation of half and full DNA nanocages was first characterized using transmission electron microscopy (TEM) (FIG. 10 and FIG. 11) and gel electrophoresis (FIG. 12), which indicate a nearly 100% yield for half-cages and a more than 90% yield for full-cages. To capture target enzymes into a half-cage, a previously reported succinimidyl 3-(2-pyridyldithio) propionate (SPDP) chemistry was used to crosslink a lysine residue on the protein surface to a thiol-modified oligonucleotide. Two anchor probes of complementary sequence were displayed on the bottom of the half-cage cavity to capture a DNA-modified enzyme via sequence-specific DNA hybridization.

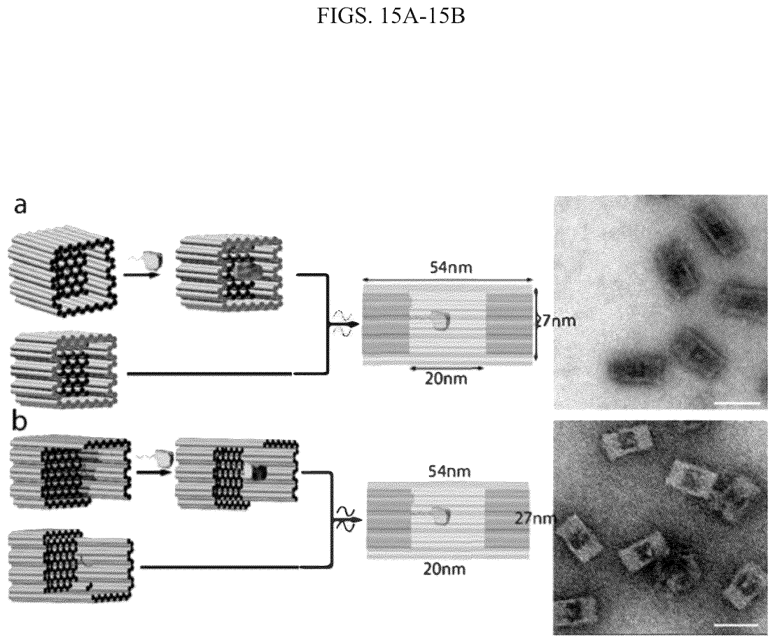

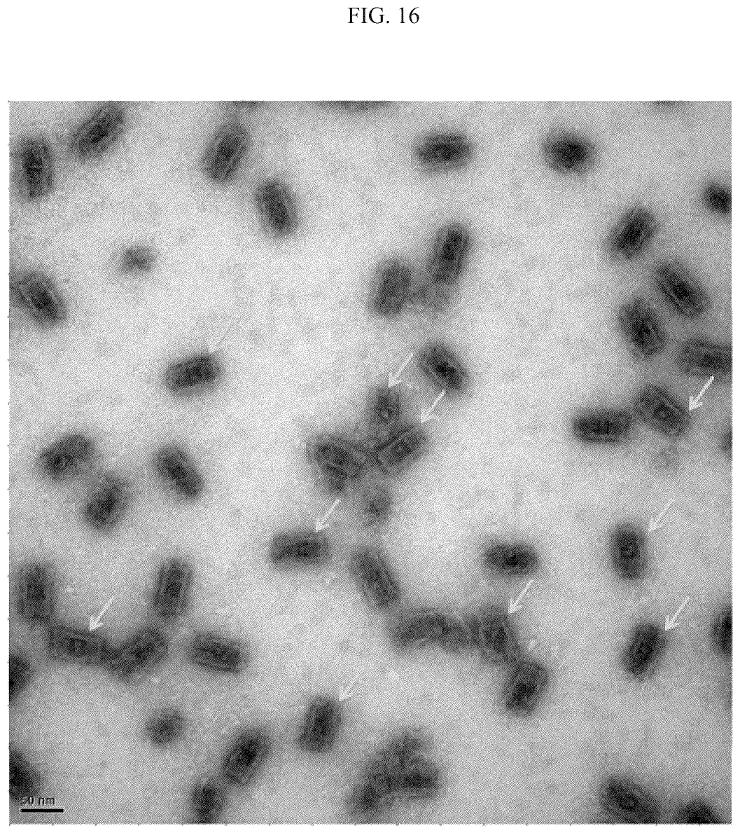

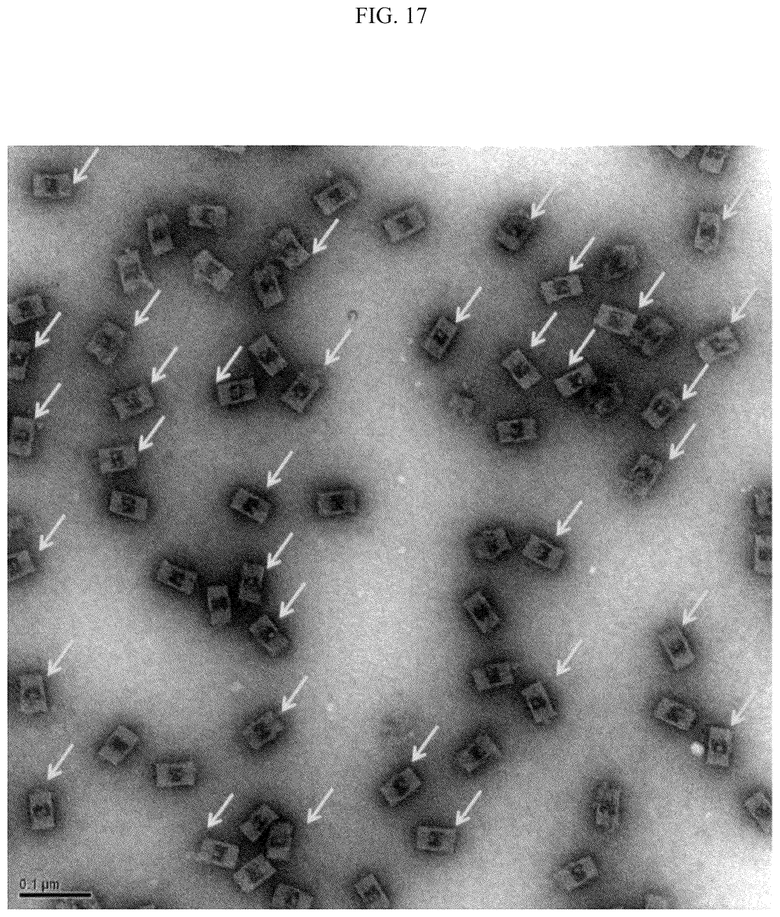

As a demonstration of an enzyme cascade, a glucose oxidase (GOx)-attached half-cage was incubated with a horseradish peroxidase (HRP)-attached half—cage at a stoichiometric ratio of 1:1, followed by the addition of bridge strands into solution to assemble a full DNA nanocage containing a GOx/HRP pair. The inner cavity of a full nanocage is of sufficient size to encapsulate this enzyme pair (GOx is 10 nm32 and HRP 5 nm in diameter33). Unencapsulated enzyme and excess short DNA strands were removed using agarose gel electrophoresis (AGE). Details of the enzyme-DNA conjugation and optimization of the assembly are shown in FIGS. 13, 14A-14F, 15A-15B, 16, 17, 18, Table 2.

Characterization of enzyme encapsulation.

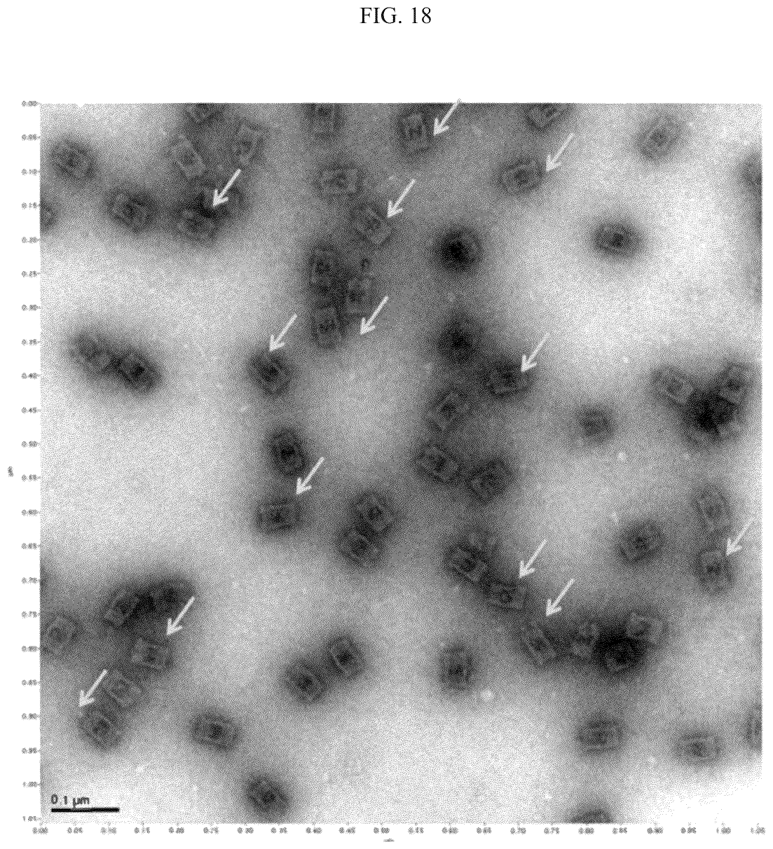

To verify the presence of both enzymes within a DNA nanocage, the co-localization of a Cy3-labeled GOx (green emission) and a Cy5-labeled HRP (red emission) was quantified by dual-color fluorescence gel electrophoresis where a gel band with overlapped green and red color was identified (FIG. 18). By comparison, the GOx-containing half-cage (Half[GOx]) shows the presence of only Cy3 (green), whereas a HRP-half-cage (Half[HRP]) shows the presence of only Cy5 (red). In addition, negatively-stained TEM images were used to visualize DNA cages upon stoichiometrically controlled encapsulation of a single GOx (FIG. 1B) or a single GOx/HRP pair (FIG. 1C), where GOx and HRP were visible as brighter spots within the cage. To quantitatively analyze the yield of DNA nanocage encapsulation, two-color total internal reflection fluorescence (TIRF) microscopy34 (FIG. 2A) was used to characterize the fluorescence co-localization of a Cy3-labeled enzyme and a Cy5-labeled nanocage (FIG. 2B). Six different enzymes were tested and characterized for encapsulation, ranging from the smallest HRP (44 kD)35, malic dehydrogenase (MDH, 70 kD)36, glucose-6-phosphate dehydrogenase (G6pDH, 100 kD)37, lactic dehydrogenase (LDH, 140 kD)38 and GOx (160 kD)39 to the largest β-galactosidase (β-Gal, 450 kD)40. All six enzymes were successfully encapsulated within full DNA nanocages with high yields, ranging from 64-98% (FIG. 2C and Table 3). The relatively low yield of β-Gal (64%) may be due to its large size (16 nm in diameter), which is comparable to the inner diameter of the nanocage (20 nm), likely resulting in steric hindrance for encapsulation. To evaluate how many copies of the same enzyme were encapsulated per DNA nanocage, single-molecule fluorescence photobleaching (SMPB) was used to count the number of photobleaching of Cy3 fluorophores per cage (FIG. 2D). The number of copies of each enzyme per cage was estimated by normalizing the number of Cy3 fluorophores per DNA nanocage with the average number of Cy3 labels per free enzyme. A majority of nanocage-encapsulated enzymes showed only one- or two-step photobleaching of Cy3, similar to the photobleaching of single free enzymes (FIG. 2E). These results suggest that most nanocages (90%) contain exactly one enzyme per cage, as expected (FIG. 2E and Table 4).

Activity characterization of nano-caged enzymes.

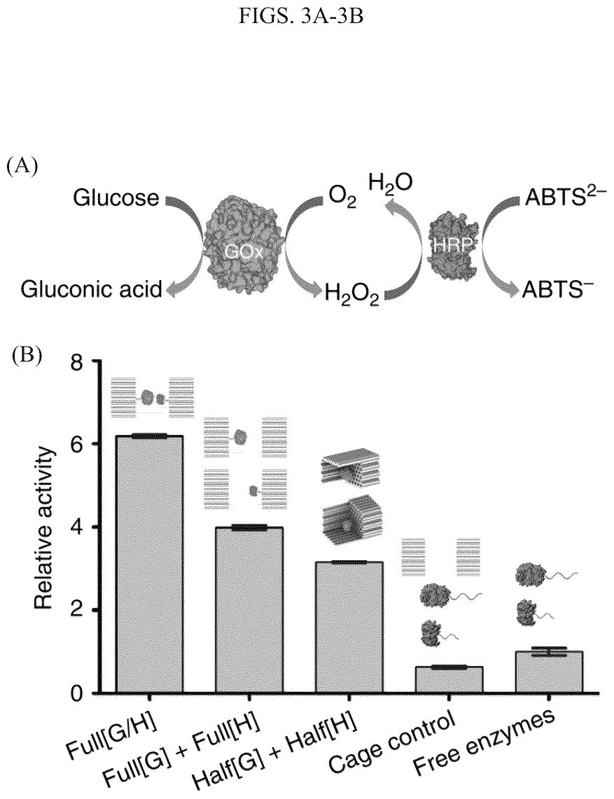

To evaluate the effect of DNA nanocages on enzyme activity, an encapsulated GOx/HRP pair was tested (FIG. 3A). This pair of enzymes catalyzes a reaction cascade beginning with the oxidation of glucose by GOx to generate hydrogen peroxide (H₂O₂). H₂O₂ is subsequently used by HRP to oxidize ABTS, producing a strong colorimetric signal. As shown in FIG. 3B, the overall activity of a co-assembled GOx/HRP cage (Full[GOx/HRP]) is 8-fold higher than that of a control enzyme pair incubated with the same cage but without encapsulation. Two plausible effects are hypothesized which could contribute to such a significant activity enhancement: 1) The proximity effect that brings the two enzymes close together and facilitates their substrate transfer, as described previously; and/or 2) the unique environment provided by the high charge density of DNA helices within a nanocage.

To separate the proximity effect from the charge density effect, control experiments of DNA nanocages encapsulating only a single GOx or HRP enzyme are designed, which clearly do not allow for substrate channeling between two proximal enzymes. For example, an equimolar mixture of two separate nanocages encapsulating either a single GOx or a single HRP (Full[GOx]+Full[HRP]) exhibited an 4-fold increase in overall activity compared to the unencapsulated control enzymes. Similarly, an equimolar mixture of two half-cages encapsulating either a single GOx or a single HRP already showed an increase in overall activity by 3-fold. Since there was no proximity effect in the case of two enzymes encapsulated into two different nanocages, the local environment modified by a DNA nanocage appears to be more important for the observed activity enhancement. Similarly, a half-cage was almost as effective in activity enhancement (3-fold) as a full-cage, suggesting that enzyme access to substrate does not play a role in this enhancement. Interestingly, a similar enhancement was reported previously upon conjugation of enzymes to a giant multi-branched DNA scaffold, without further explanation.

To test the generality of nanocage activity observations, the activity of six different enzymes upon encapsulation within DNA nanocages are evaluated. As shown in Table 1, five of them (GOx, HRP, G6pDH, MDH, and LDH) exhibited higher activity in nanocages than the free enzyme, with enhancements ranging from 3- to 10-fold.

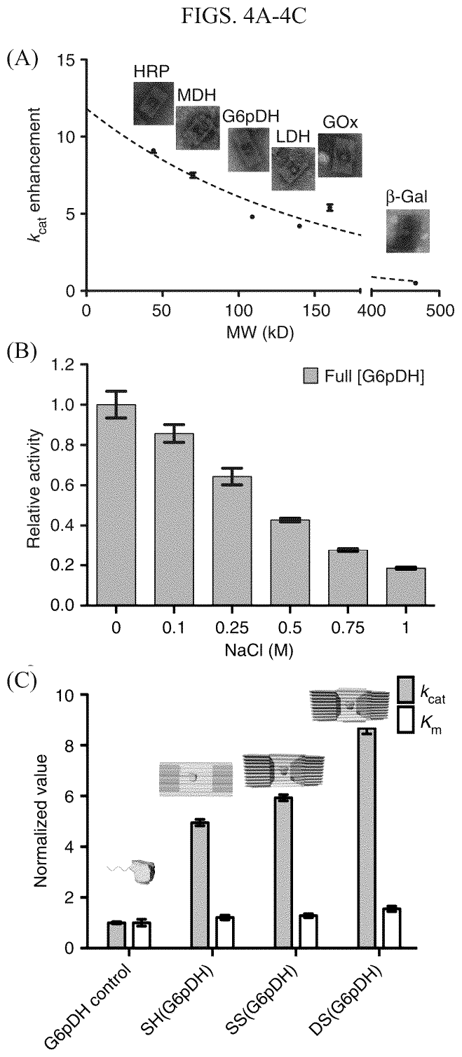

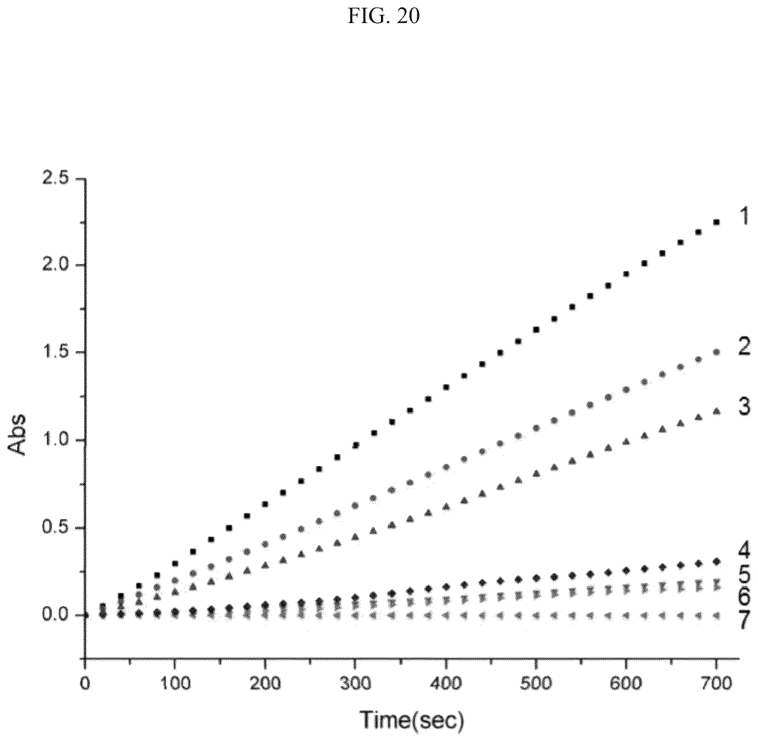

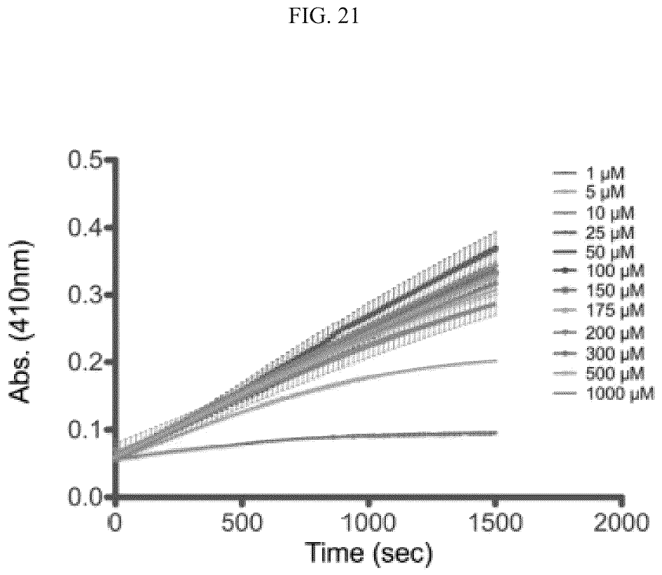

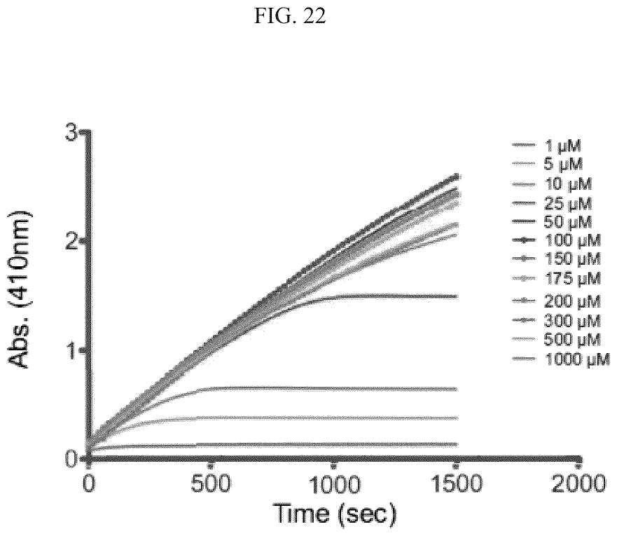

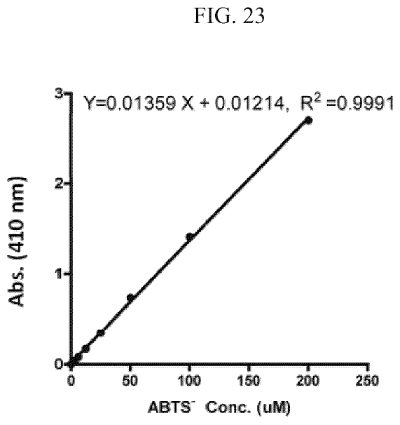

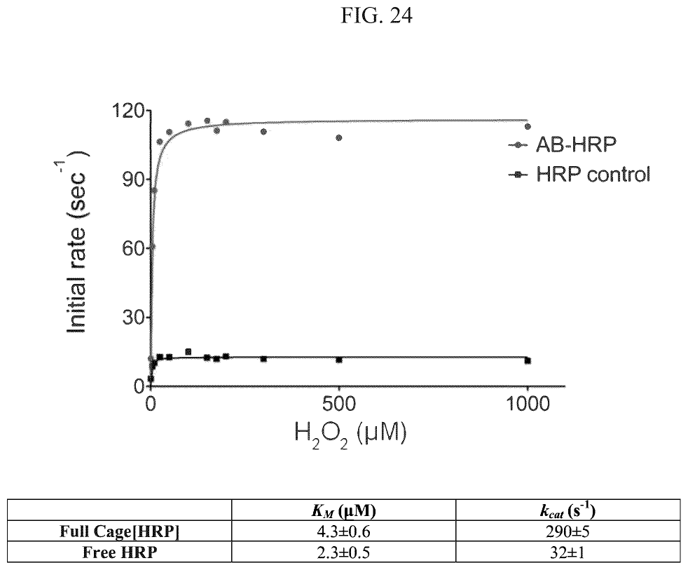

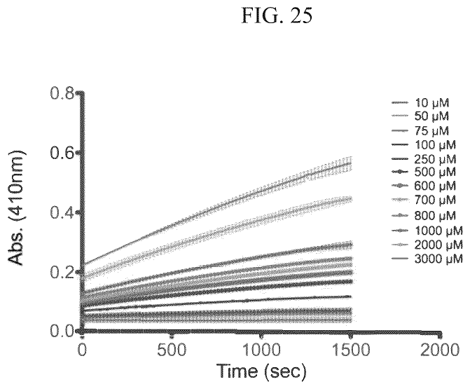

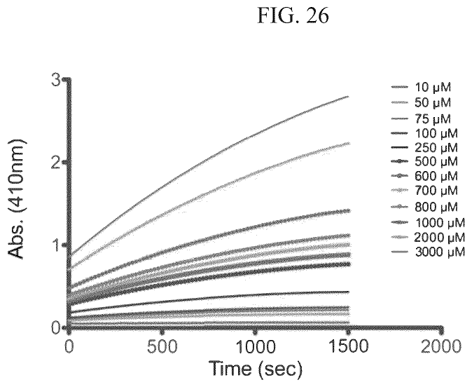

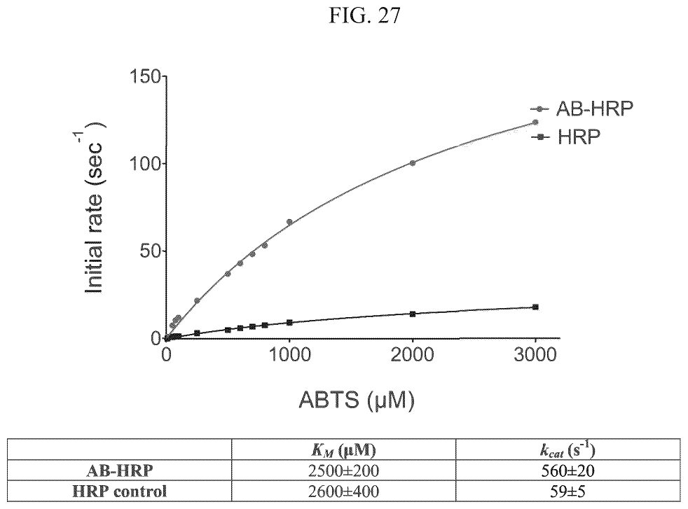

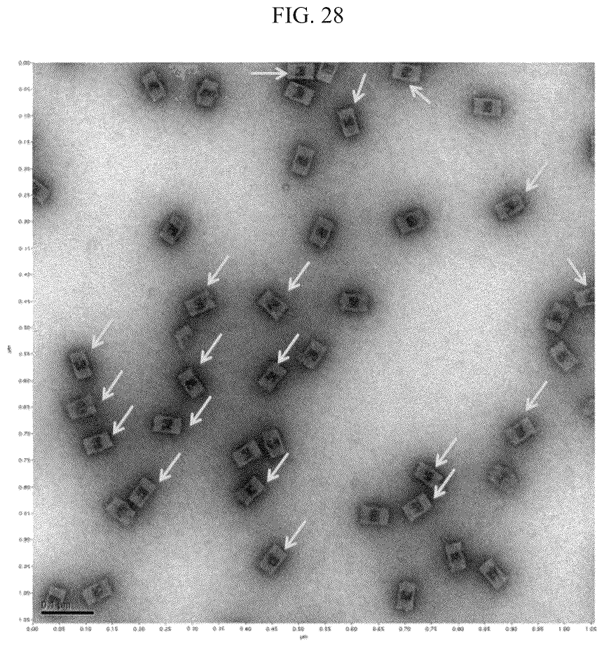

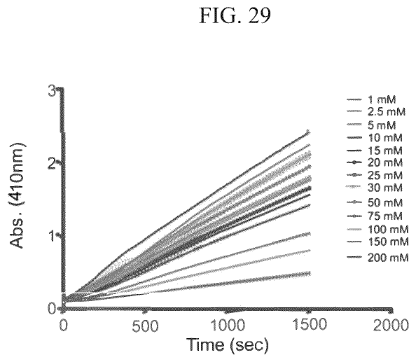

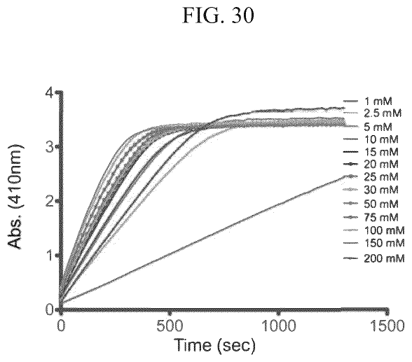

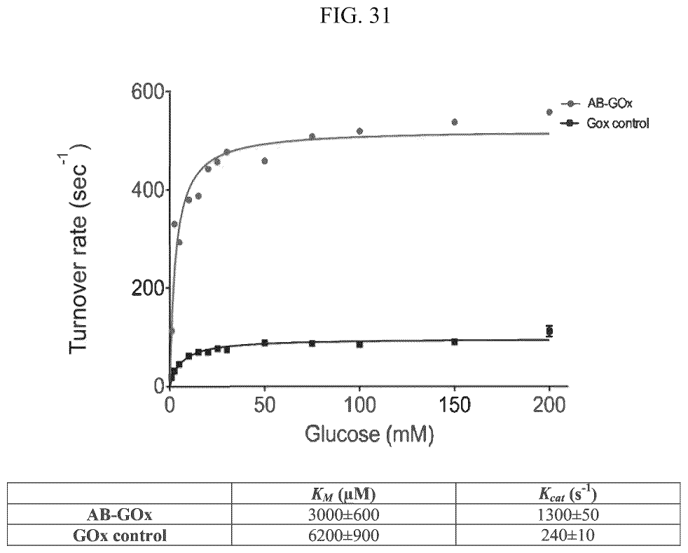

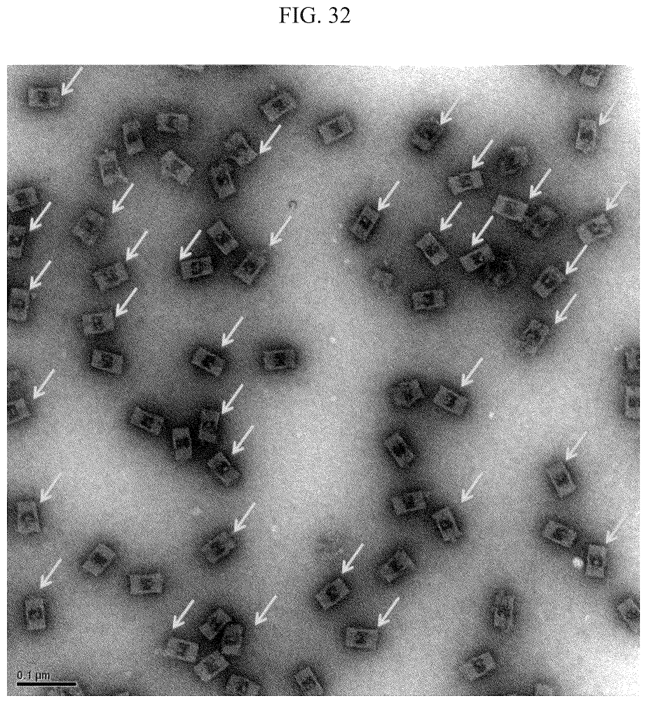

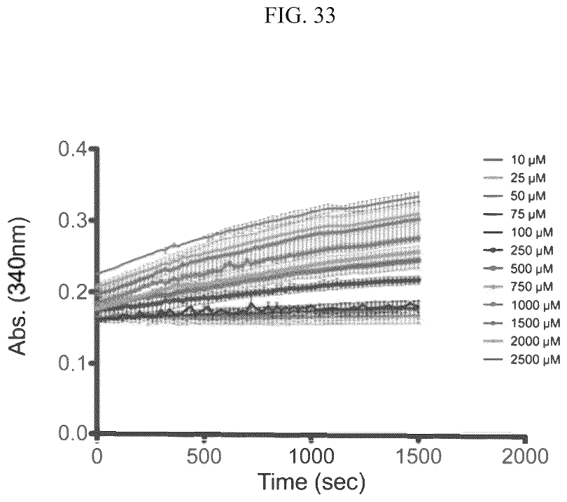

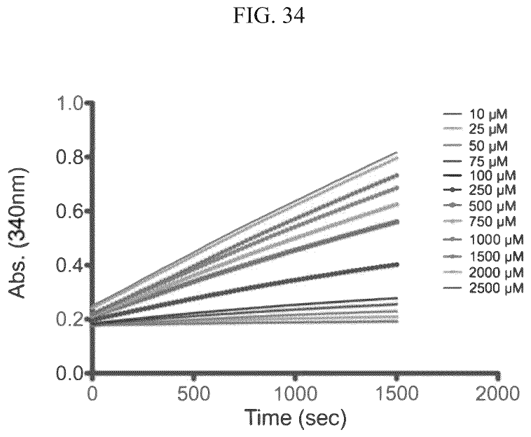

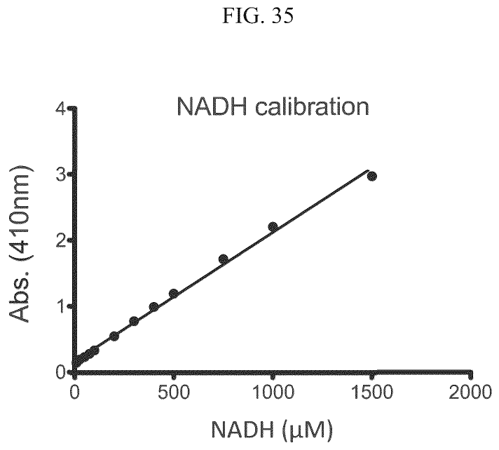

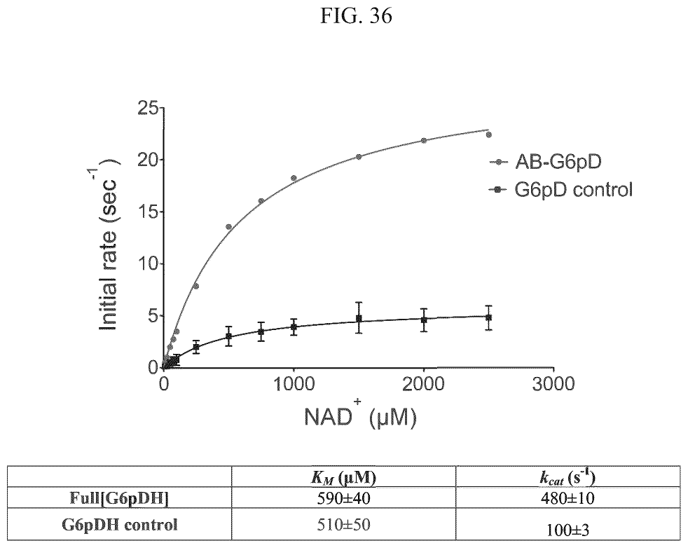

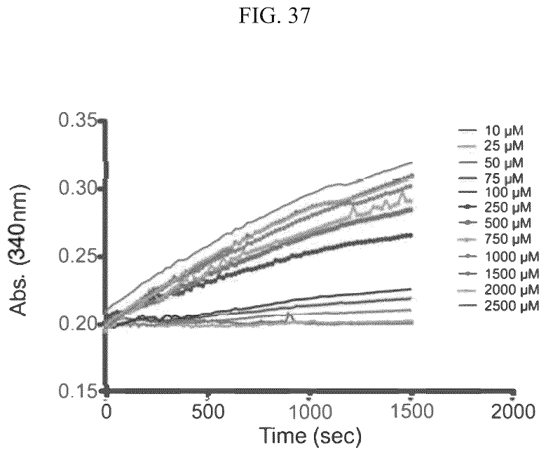

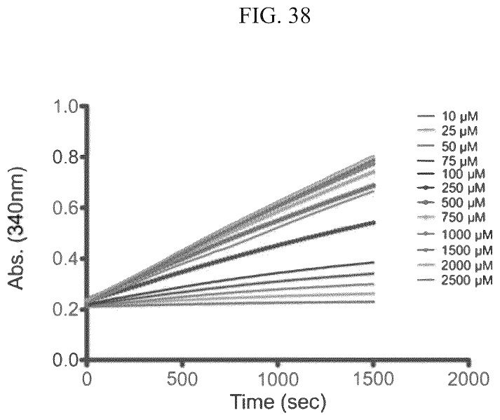

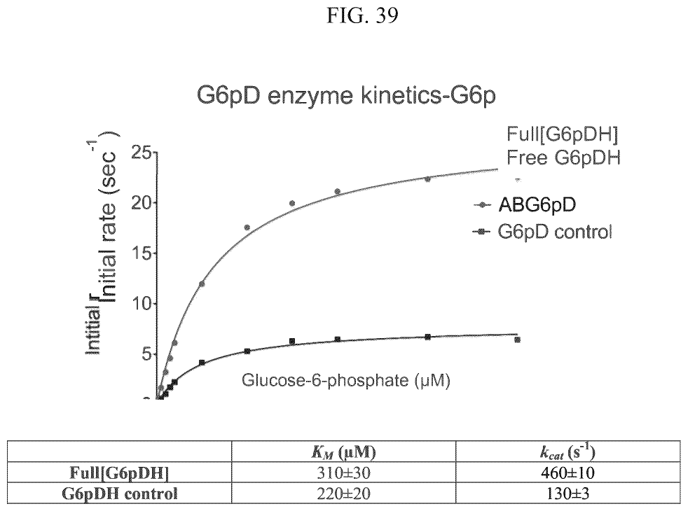

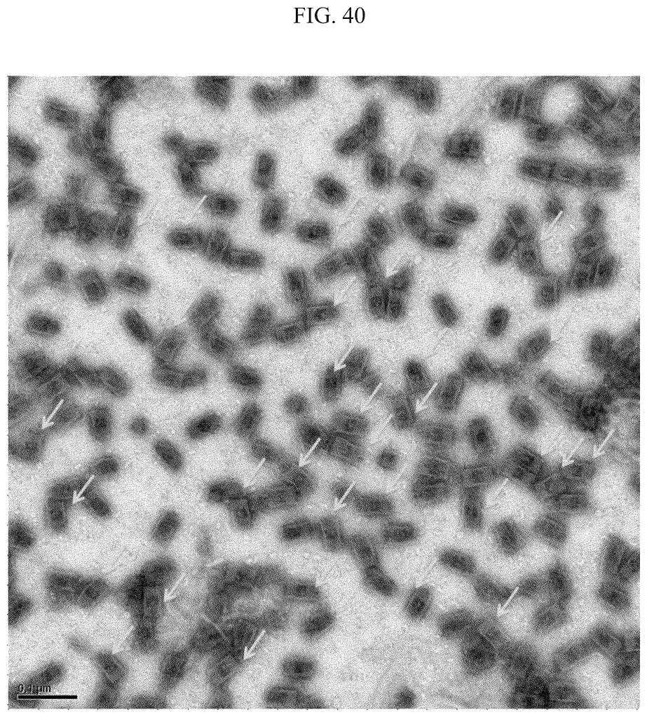

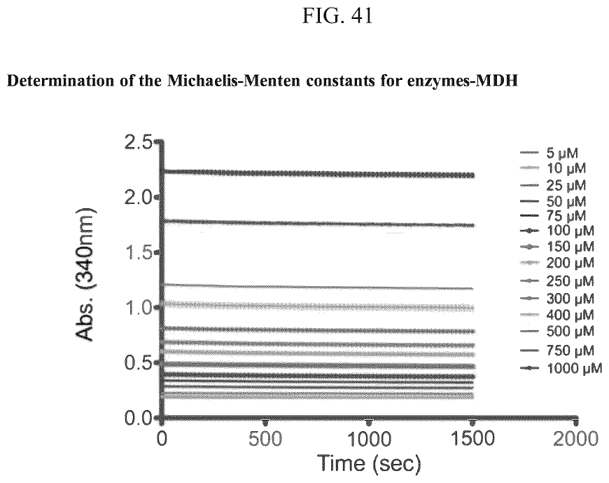

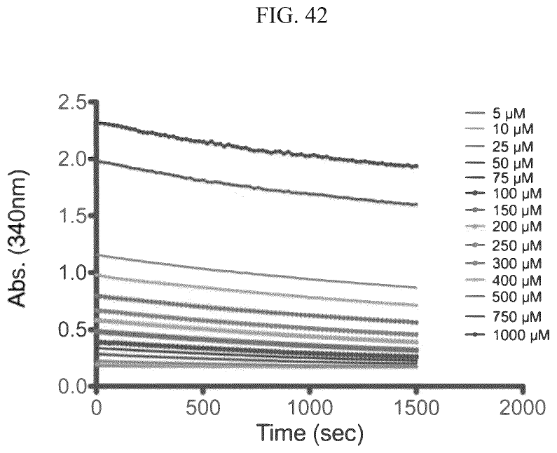

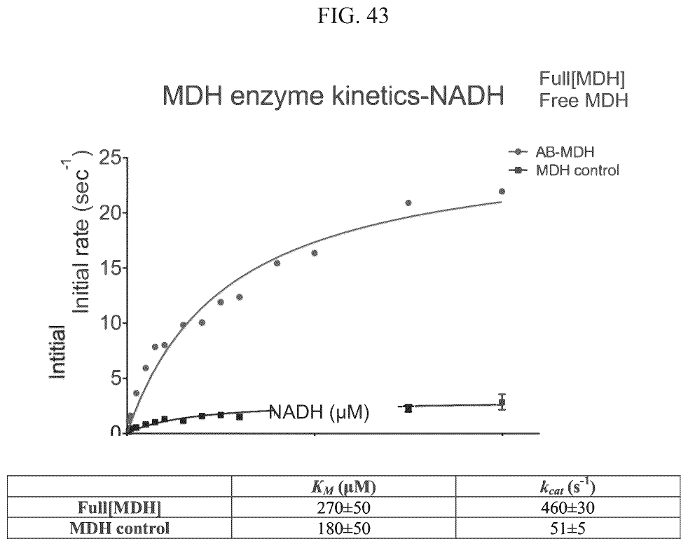

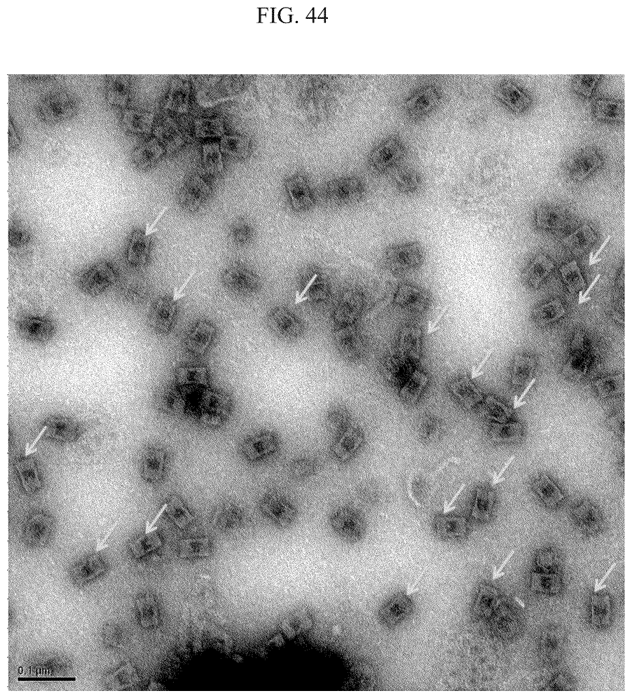

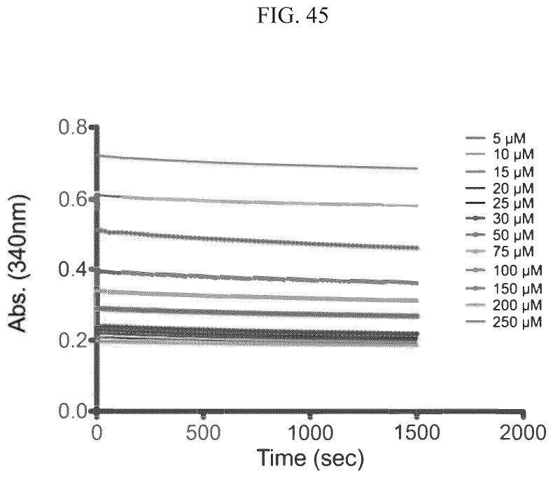

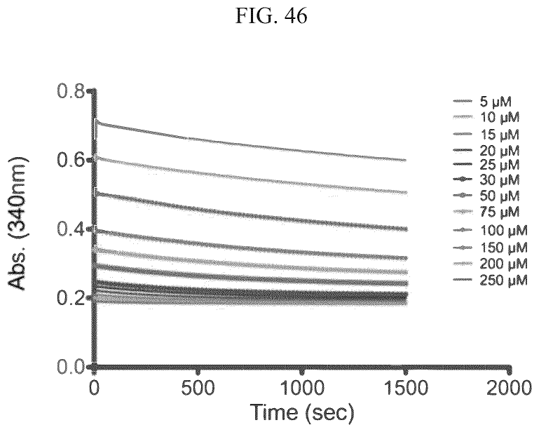

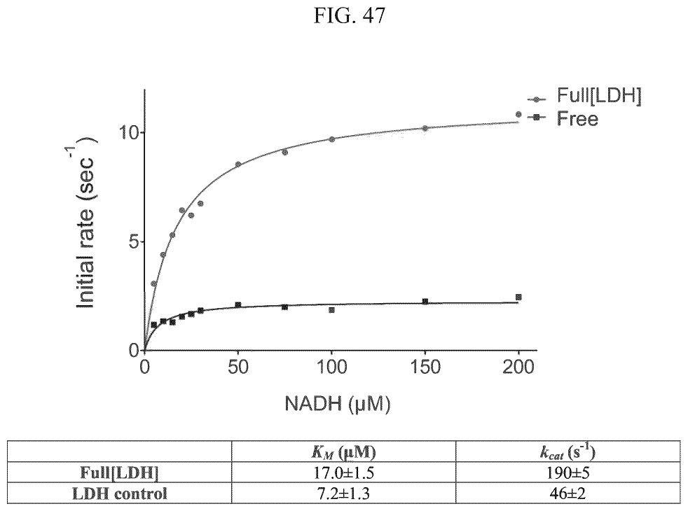

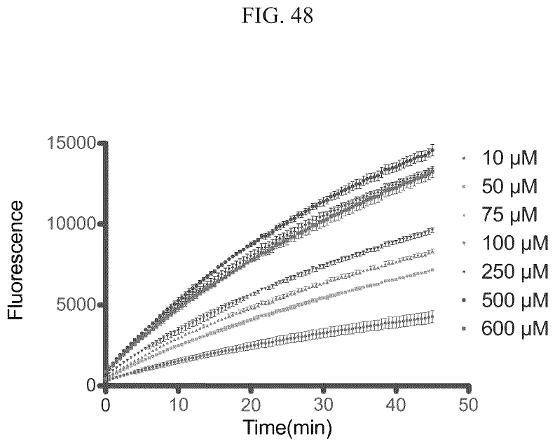

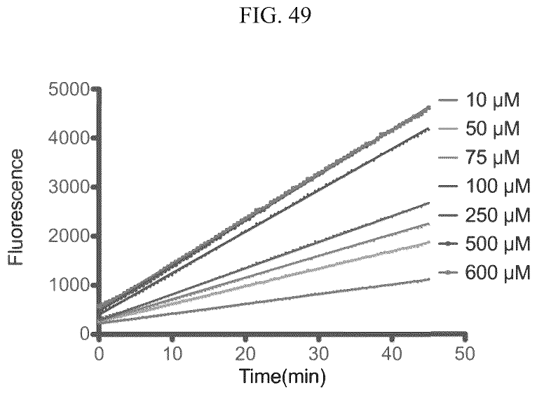

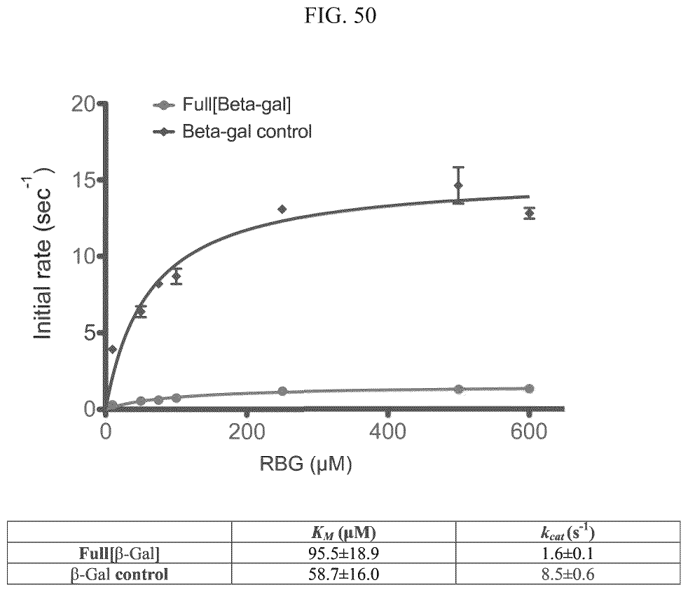

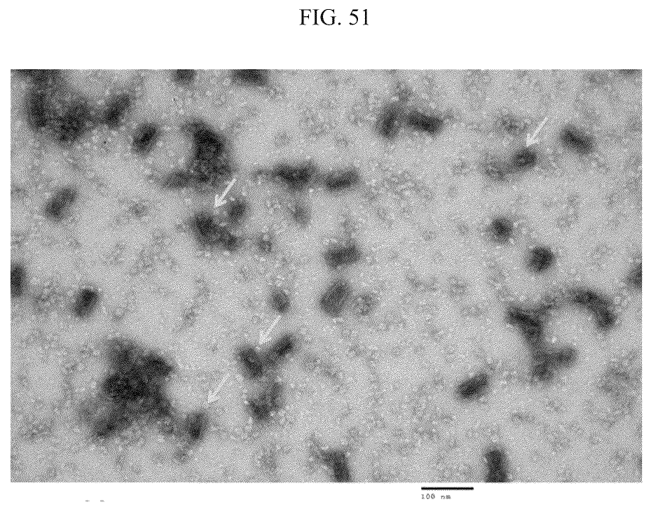

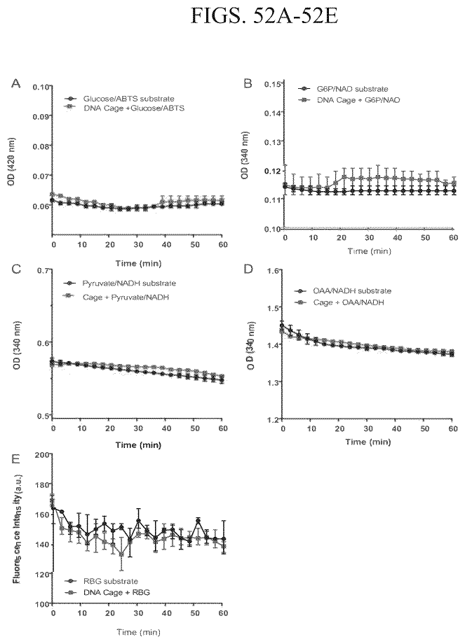

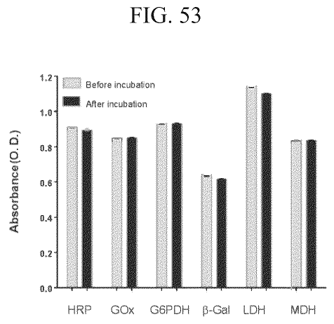

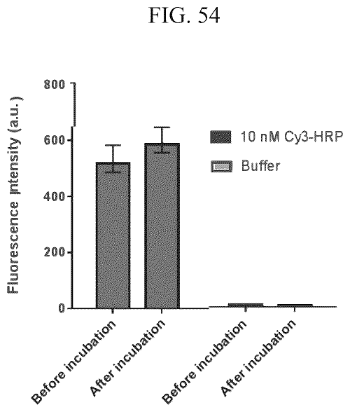

Detailed kinetic analyses show that the K_{M} (the Michaelis-Menten constant) varies little between encapsulated and free enzyme for most substrates (ranging from 0.5 to 2.4-fold of the free enzyme), suggesting that the porous DNA cages do not substantially hinder diffusion of small-molecule substrates. In contrast, a large increase in turnover number (k cat) was observed for these five enzymes (ranging from 3.5- to 9.6-fold of the free enzyme), suggesting an inherently higher catalytic activity of the proteins. For all the raw kinetics data, please see FIGS. 20-54. An inverse correlation was observed between enhanced turnover and size of the encapsulated enzyme (FIG. 4A). That is, the smaller HRP (44 kD) and MDH (70 kD) exhibited relatively large increases in turnover number of 9.6±0.4 and 9.0±0.7 fold, respectively, whereas the larger enzymes G6pDH, LDH, and GOx exhibited smaller enhancements of 4.7±0.1 fold, 4.1±0.1 fold, and 5.4±0.2 fold, respectively. No correlation was observed between enhancement and isoelectric point (pI), despite the wide range of pI values for these enzymes (ranging from 4.2 to 10.0).

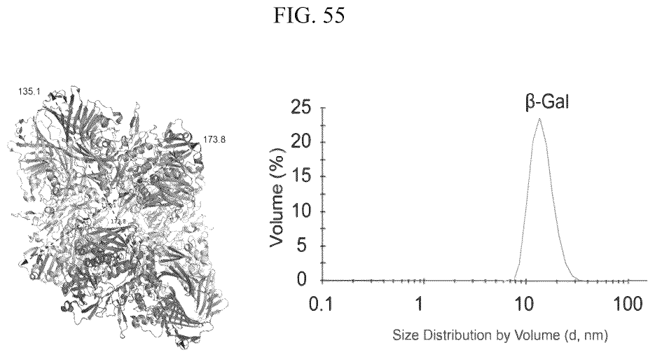

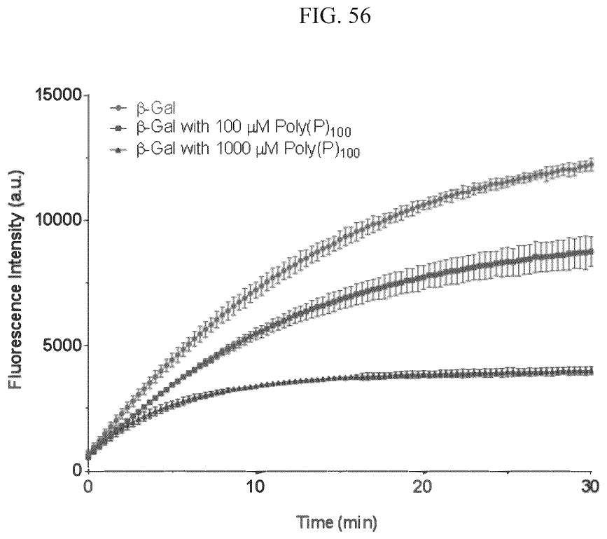

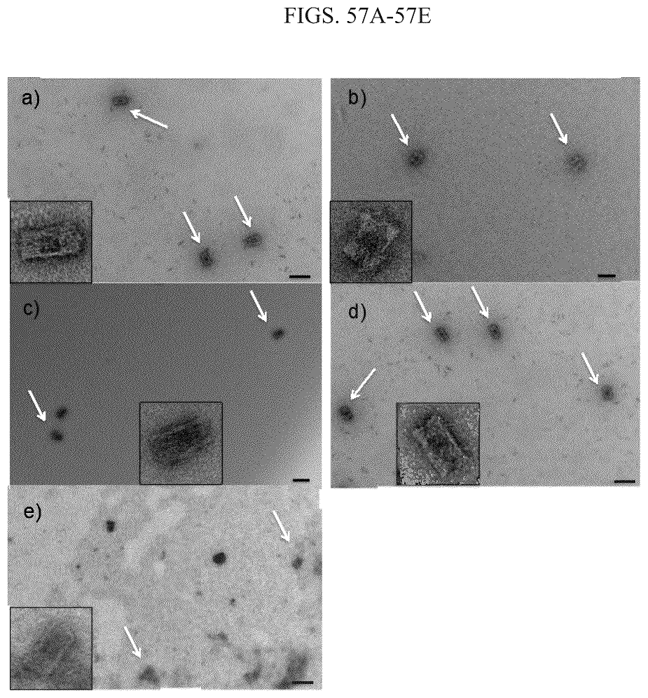

In contrast to these five enzymes, β-Gal is strongly inhibited upon encapsulation, possibly due to its large size (16 nm in diameter, FIG. 55) that is comparable to the inner cavity diameter (20 nm) of the DNA nanocage. Alternatively, the β-Gal orientation may be unfavorable and block binding of substrate to the active site. Notably, in a control experiment polyphosphate inhibited the activity of β-Gal (FIG. 56), suggesting that the local high density of backbone phosphates of the DNA nanocage might be responsible for the decrease in activity of β-Gal. The DNA cages retained their structural integrity during the enzymatic reactions (FIGS. 57A-57E).

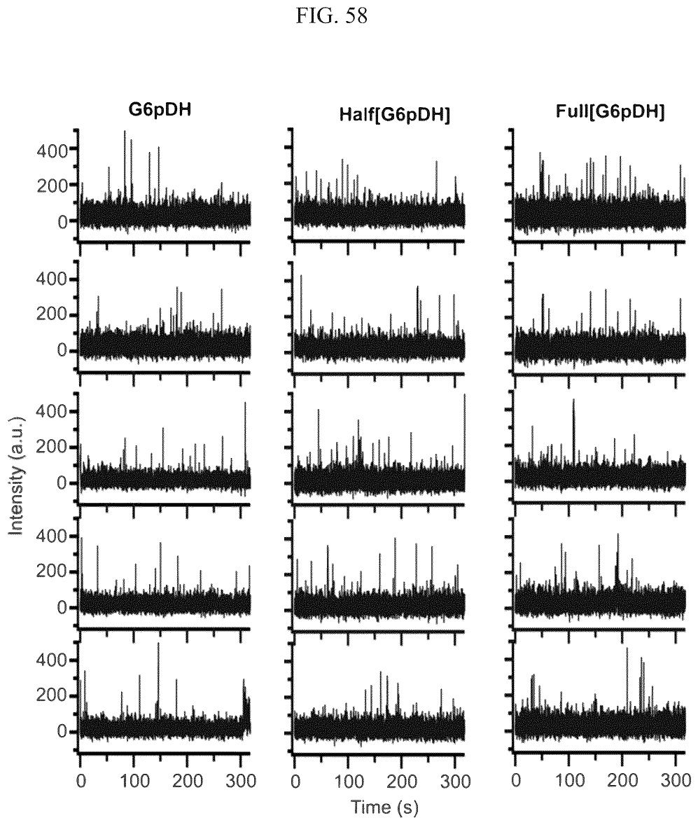

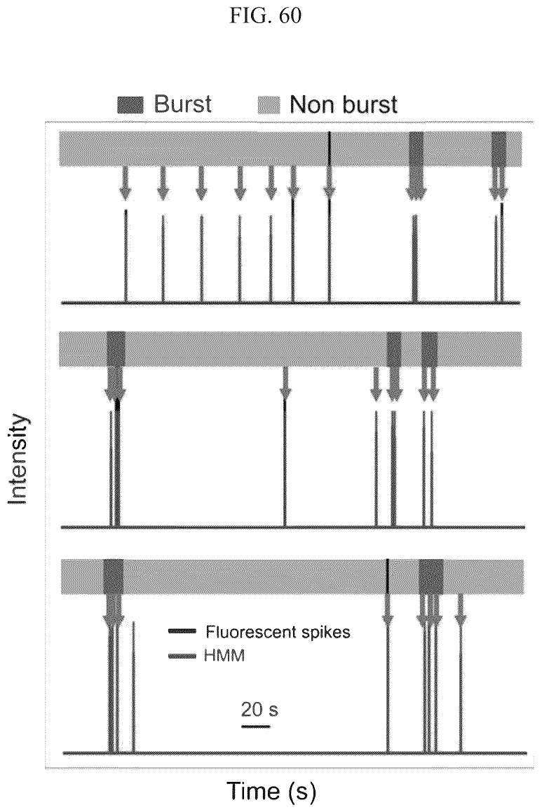

To gain more detailed mechanistic insight into the enhancement of catalytic turnover, a novel single-molecule fluorescence assay to characterize the activity of individual enzymes with and without encapsulation was applied (FIG. 5). As shown in FIGS. 5A and 5B, TIRF microscopy is used to record the repetitive turnover of substrates by individual G6pDH enzymes over time; coupling with a PMS/resazurin reaction allowed us to detect stochastic fluctuations of enzyme turnover rates via transient spikes in intensity from the generation of the fluorescent product resorufin (FIGS. 5C-5D and FIGS. 58, 59A-59D, and 60). Such fluctuations have been observed for various enzymes before and are thought to be induced by the conformational switching between more and less active sub-states.

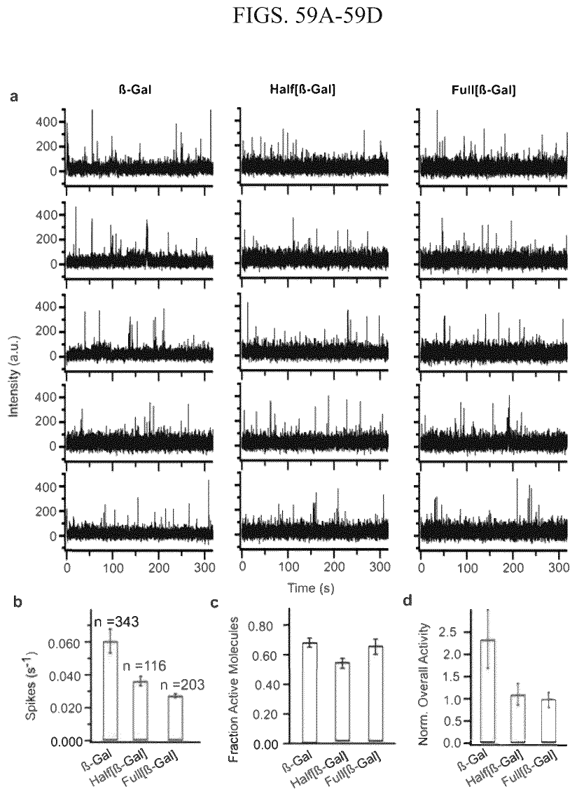

Compared to a control without substrate, more frequent fluorescent spikes were observed with the addition of glucose-6-phosphate substrate (FIGS. 5C and 5D). The average spike frequency was increased from 0.016±0.001 s−1 for unencapsulated enzymes, to 0.019±0.001 s−1 for the half-cage and 0.026±0.002 s−1 for the full-cage (FIG. 5E). Further analysis suggested that the fraction of active enzyme molecules was increased from 20.3% for unencapsulated enzymes to 26.6% for the half-cage and 30.5% for the full-cage (FIG. 5F). Taken together, the 1.6-fold higher spike frequency and the 1.5-fold increase in the fraction of active enzymes yield a 2.5-fold increase in G6pDH activity for the encapsulated compared to the unencapsulated enzyme (FIG. 5G), comparable to the 4-fold enhancement observed in the bulk assay. Conversely, a similar analysis of β-Gal activity showed a 3-fold lower activity of the full-cage enzyme (2.3±0.5 fold lower in spike frequency compared to free enzyme whereas the fractions of active enzymes (65%) were similar) compared to unencapsulated enzyme (FIGS. 59A-59D), also consistent with the bulk measurement.

The activity enhancement for DNA cage-encapsulated enzymes is consistent with recent reports of enhanced enzyme activity upon attachment to a long double-stranded DNA molecule (XIDNA), a 2D rectangular DNA origami, or a DNA scaffold that bound to enzyme substrates, and further suggests that it may be a widespread effect of enzyme-DNA interactions. Several mechanisms have been previously proposed to explain these observed enhancements, including micro-environment composed of giant and ordered DNA molecules, molecular crowding and the substrates affinity to DNA scaffolds. We further suggested that the negatively charged phosphate backbones of DNA might also contribute to the activity enhancement. DNA is a negatively charged biopolymer due to its closely spaced backbone phosphates (leading to a linear negative charge density of 0.6 e/A). Thus, upon encapsulation within a DNA nanocage, an enzyme is exposed to an environment full of negative charges that may resemble the relative abundance of polyanionic molecules and surfaces (including RNA and phospholipid membranes) within the cell. Phosphate is a known kosmotropic anion that increases the extent of hydrogen-bonded water structures (termed high-density or structured water). A DNA nanocage is thus expected to attract a strongly bound hydration layer of hydrogen-bonded water molecules inside its cavity. Multiple studies have described that proteins are more stable and active in a highly ordered, hydrogen-bonded water environment, possibly due to stabilization of the hydrophobic interactions of a folded protein through an increase in the solvent entropy penalty upon unfolding.

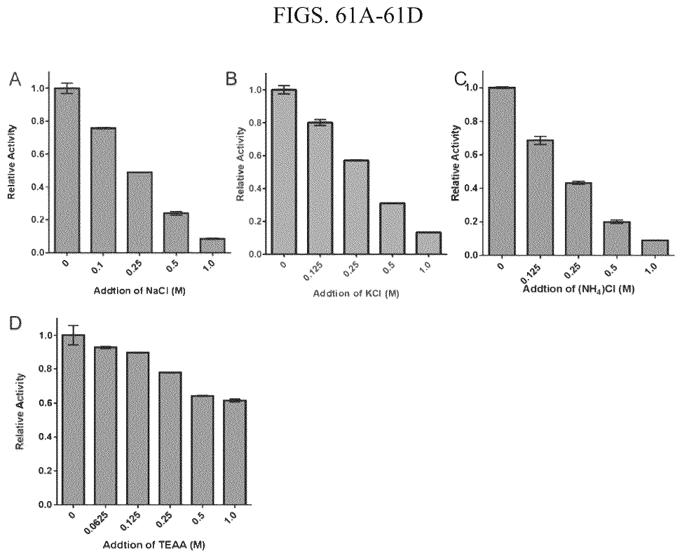

Consistent with this model, polyphosphate has been shown to act as a generic chaperone stabilizing a variety of enzymes. To further test whether this mechanism is at work in our nanocages, we titrated the concentration of NaCl (known to consist of chaotropic ions) for the purpose of interrupting hydrogen-bonded water molecules. Consistent with our hypothesis, the activity of encapsulated enzymes significantly decreased with increasing NaCl concentration (reduced to 25% activity with 1 M NaC1 as shown in FIG. 4B. A high concentration of Na+ can shield the negative charge on the DNA surface, thus disrupting the surface-bound hydration layer. As a control, we observed that the bulky kosmotropic cation, triethylammonium, had a much less pronounced effect on enzymatic activity (FIGS. 61A-61D). This model also allowed us to rationalize why we observed smaller enzymes to be more activated than larger enzymes: namely, because their higher surface-to-volume ratio predicts a stronger impact of the hydration layer.

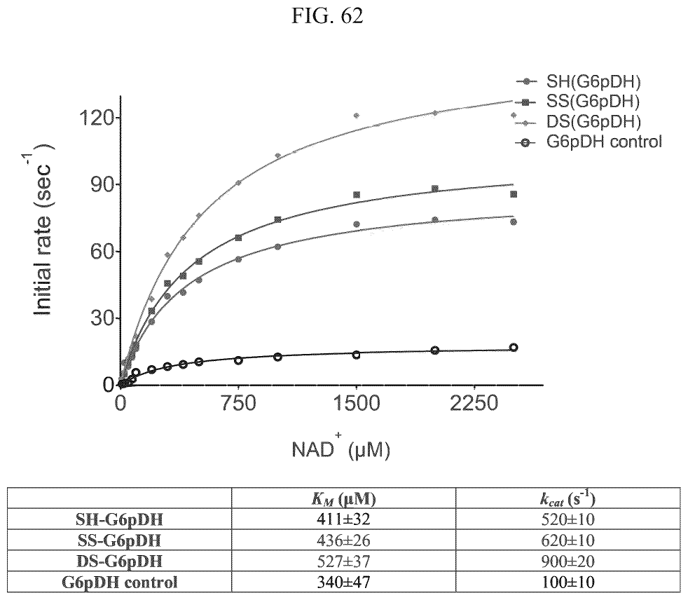

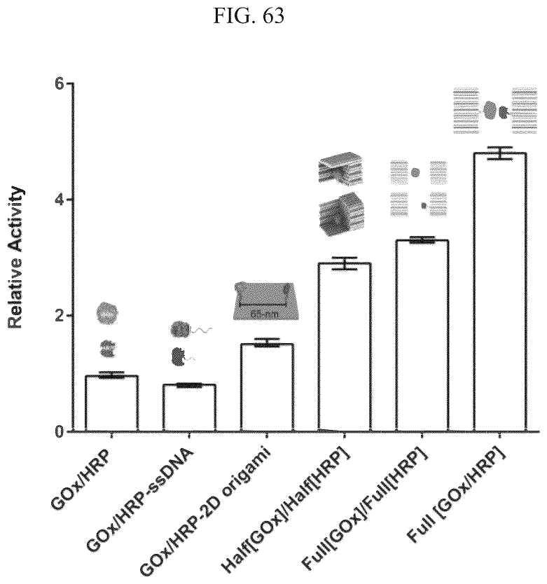

To further test this model, we investigated the effect of DNA helix density on the encapsulated enzyme activity. As shown in FIG. 4C, we designed three nanocages with walls that systematically increase the density of DNA helices, including: 1) a single-layer honeycomb pattern (SH) with 2-3 nm pores between helices; 2) a single-layer square pattern (SS) with smaller 0.5-1 nm pores between helices, and 3) a double-layer square pattern (DS). The helix density at the top and bottom surfaces thus increased from 0.12 helices per nm2 for SH to 0.16 helices per nm2 for the SS and DS designs. The k_{cat} of G6pDH encapsulated in the SH-cage was 4.7-fold higher than that of the free enzyme. As the density of DNA helices was increased, the k cat of encapsulated G6pDH raised to 6-fold for the SS-cage and 8-fold for the DS-cage compared to the free enzyme control. A slight increase in K_{M} values was also observed from the SH-cage to the SS- and DS-cages, possibly due to a decrease in substrate diffusion through the DNA walls of these more tightly packed structures. For example, the K_{M} value of G6pDH increased from 41111M in the SH-cage to 43611M in the SS-cage and 527 μM in the DS-cage (FIG. 62 and FIG. 4C). Additional studies showed that activities of attached enzymes were enhanced by increasing the helix packing density for various 1D, 2D and 3D DNA scaffolds (FIG. 63). These observations suggest that encapsulated enzymes exhibit higher activity within densely packed DNA cages, consistent with our model that the highly ordered, hydrogen-bonded water environment near closely spaced phosphate groups are responsible for this effect.

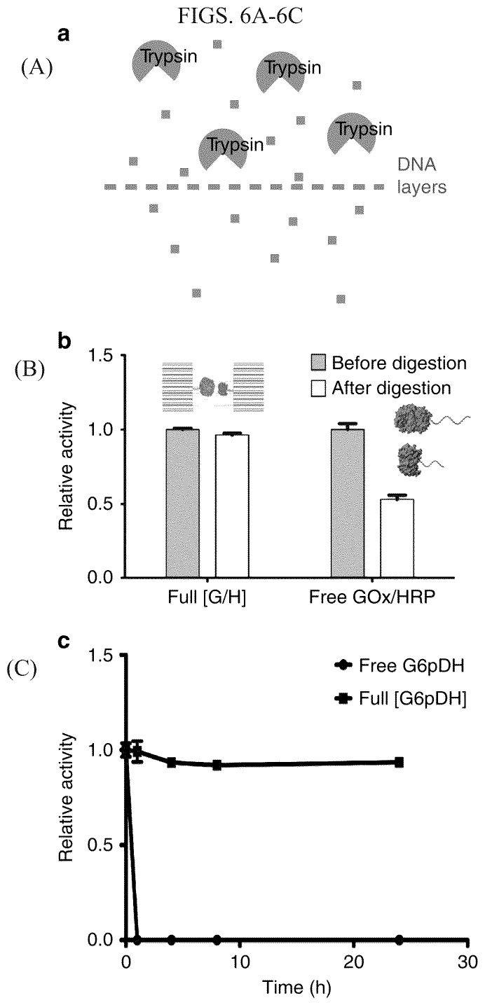

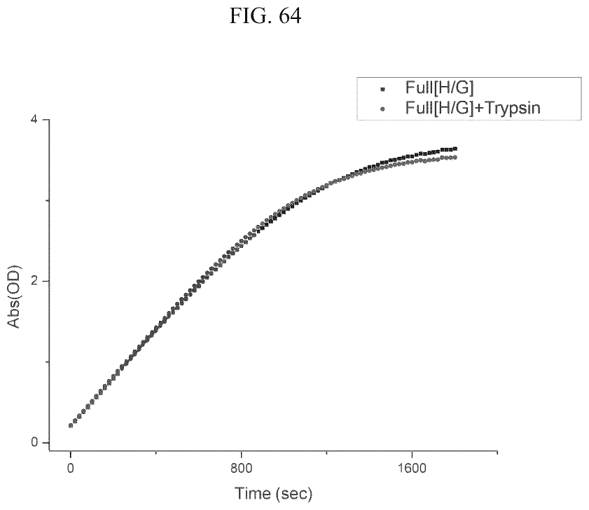

Nanocaged enzymes are protected from proteolysis. Self-assembled DNA nanostructures previously were found to be more resistant against nuclease degradation than single- or double-stranded DNA molecules. Similarly, DNA nanocages should protect encapsulated enzymes from deactivation and aggregation under challenging biological conditions. As shown in FIG. 6, encapsulated GOx/HRP was highly resistant to digestion by trypsin (FIG. 6B), and retained more than 95% of its initial activity after incubation with trypsin for 24 h (FIG. 6C). A time-course experiment was also performed to demonstrate the stability of caged enzymes against Trypsin digestion (FIG. 6C and FIGS. 64-67). In contrast, free GOx/HRP only retained 50% of its initial activity after a similar incubation with trypsin. This result demonstrated the potential utility of DNA nanocages for protecting encapsulated proteins from biological degradation.

TABLE 1
Enzyme kinetic data (values of K_{M} and k_{cat}) for each individual enzyme encapsulated inside
a DNA full-cage in comparison with the values for the free enzymes in solution.

| | | Molecular | | Free enzyme | | Encapsulated enzyme | |
|---|---|---|---|---|---|---|---|
| Enzyme | pI | weight | Substrate | K_{M} (μM) | k_{cat} (s−1) | K_{M} (μM) | k_{cat} (s−1) |
| GOx | 4.2 | 160 kDa | Glucose | 6,200 ± 900 | 240 ± 10 | 3,000 ± 600 | 1,300 ± 50 |
| HRP | 8.8 | 44 kDa | H₂O₂ | 2.3 ± 0.5 | 32 ± 1 | 4.3 ± 0.6 | 290 ± 5 |
| | | | ABTS | 2,600 ± 400 | 59 ± 5 | 2,500 ± 200 | 560 ± 20 |
| G6pDH | 4.3 | 100 kDa | Glucose-6- | 220 ± 20 | 130 ± 3 | 310 ± 30 | 460 ± 10 |
| | | | phosphate | | | | |
| | | | NAD+ | 510 ± 50 | 100 ± 3 | 590 ± 40 | 480 ± 10 |
| MDH | 10.0 | 70 kDa | NADH | 180 ± 50 | 51 ± 5 | 270 ± 50 | 460 ± 30 |
| LDH | 5.0 | 140 kDa | NADH | 7.2 ± 1.3 | 46 ± 2 | 17.0 ± 1.5 | 190 ± 5 |
| β-Gal | 4.1 | 465 kDa | RBG | 58.7 ± 16.0 | 8.5 ± 0.6* | 95.5 ± 18.9 | 1.6 ± 0.1* |

ABTS, 2,2′-azino-bis(3-ethylbenzothiazoline-6-sulphonic acid); GOx, glucose oxidase; HRP, horseradish peroxidase; LDH, lactic acid dehydrogenase; MDH, malic dehydrogenase; pI isoelectric point.
The pI values of the enzymes were obtained from brenda-enzymes.org
*k_{cat} values for β-Gal groups were not calibrated

TABLE 2
Estimation of the concentration and DNA labeling ratio of the purified DNA-conjugated enzymes by measuring
absorbance at 260 and 280 nm. Concentration of HRP-P1-Cy3 was estimated by the unique absorbance at 405 nm.

| | | ε260 | ε260 | | | ε260 | ε260 | | | | | DNA-to- | Protein | |
|---|---|---|---|---|---|---|---|---|---|---|---|---|---|---|
| | A260/ | (M⁻¹ | (M⁻¹ | | A260/ | (M⁻¹ | (M⁻¹ | | A260/ | | | Protein | Conc. | Dye |
| DNA | A280 | cm⁻¹) | cm⁻¹) | Protein | A280 | cm⁻¹) | cm⁻¹) | Sample | A280 | A260 | A280 | Ratio | (μM) | (μM) |
| P1- | 1.27 | 115200 | 90709 | GOx | 0.63 | 168336 | 267200 | GOx-P1- | 1.18 | 13.50 | 14.10 | 3.09 | 25.77 | 37.00 |
| Cy3 | | | | | | | | Cy3 | | | | | | |
| P1- | 1.27 | 115200 | 90709 | β-Gal | 0.59 | 573534.9 | 972093 | β-Gal-P1- | 0.63 | 1.34 | 2.11 | 0.74 | 2.03 | 1.10 |
| Cy3 | | | | | | | | Cy3 | | | | | | |
| P1- | 1.27 | 115200 | 90709 | G6pDH | 0.52 | 61594 | 118450 | G6pDH- | 1.00 | 11.15 | 11.17 | 2.30 | 34.17 | 53 |
| Cy3 | | | | | | | | P1-Gy3 | | | | | | |
| P2- | 1.60 | 130100 | 81313 | MDH | 0.72 | 14112 | 19600 | MDH-P2- | 1.49 | 1.47 | 0.99 | 1.63 | 6.49 | 8 |
| AF647 | | | | | | | | AF647 | | | | | | |
| P2- | 1.60 | 130100 | 81313 | LDH | 0.57 | 115504.8 | 202640 | LDH-P2- | 0.83 | 2.83 | 3.41 | 0.84 | 12.59 | 22 |
| AF647 | | | | | | | | AF647 | | | | | | |

A₂₆₀ (DNA-protein) = ε₂₆₀ (protein) * Conc. (protein) + ε₂₆₀ (DNA) * Conc. (DNA)
A₂₈₀ (DNA-protein) = ε₂₈₀ (protein) * Conc. (protein) + ε₂₈₀ (DNA) * Conc. (DNA)
$Ratio ( \frac{DNA}{protein} ) = \frac{Conc . ( DNA )}{Conc . ( protein )}$

TABLE 3
Enzyme encapsulation efficiency calculation. Enzyme encapsulation
was calculated by taking the ratio of the number of colocalized
molecules (i.e., both enzyme and right half-cage) to the total number
of molecules containing the right half-cage. N is the number of particles
analyzes, N_{coloc} is the number of particles containing both fluorophores,
and N_{right} is the number of particles showing evidence of the
right half-cage.

| | N | N_{coloc} | N_{right} | N_{coloc}/N_{right} |
|---|---|---|---|---|
| HRP | 176 | 156 | 165 | 0.94 |
| GOx | 205 | 197 | 201 | 0.98 |
| G6pDH | 218 | 209 | 214 | 0.98 |
| LDH | 1229 | 826 | 1008 | 0.82 |
| MDH | 363 | 335 | 348 | 0.96 |
| β-Gal | 284 | 115 | 179 | 0.64 |

TABLE 4
Calculation of enzyme copies per DNA nanocage. The percentage of molecules
exhibiting a given number Cy3 photobleaching steps “Cy3 Steps” for both the
encapsulated and unencapsulated enzymes are provided. The mean number of enzymes
per cage (N_{enz}) was calculated by taking the ratio of μ_{Cy3}_Encap to
μ_{Cy3}_Unencap. N is the total number of particles analyzed.

| | | Cy3 Steps | | | | Cy3 Steps | | | | |
|---|---|---|---|---|---|---|---|---|---|---|
| | | (% molecules) | | | | (% molecules) | | | | |
| | N | One | Two | Three | μ_{Cy3}_Encap | One | Two | Three | μ_{Cy3}_Unencap | N_{enz} |
| HRP | 176 | 86 | 13 | 1 | 1.15 | 92 | 8 | 0 | 1.08 | 1.0 |
| G6pDH | 218 | 87 | 10 | 3 | 1.16 | 93 | 7 | 0 | 1.07 | 1.1 |
| β-Gal | 284 | 93 | 6 | 1 | 1.08 | 88 | 9 | 3 | 1.15 | 0.9 |

TABLE 5
Conditions for the single-molecule enzyme activity assay

| Solution | Concentration | |
|---|---|---|
| 10X TBS, pH 7.5 | 1X | |
| Resazurin Glucose-6-phosphate | 50 | nM |
| (G6p) | 1 | nM |
| Phenazine Methosulfate (PMS) | 12.5 | μM |
| Mg²⁺ (MgCl₂) | 1 | mM |
| NAD⁺ | 1 | mM |
| PEG 8000 | 10% | (w/v) |

### Discussion

In summary, we have developed a method for using a DNA nanocage to efficiently encapsulate enzymes with high yield. Using single-molecule characterization, we were able to quantify the copies of encapsulated enzymes per cage with demonstrated one enzyme per cage. Upon encapsulation, five of six tested metabolic enzymes exhibit turnover numbers 4- to 10-fold higher than that of the free enzyme. Conversely, the K_{M} values remain similar between encapsulated enzymes and free enzymes, indicating an uninterrupted diffusion of small-molecule substrates and products through the nanopores in the DNA cage. Application of a novel single-molecule enzyme assay showed that both the fraction of active enzyme molecules and their individual turnover numbers increase as a consequence of encapsulation.

It is therefore proposed, without being bound to any particular theory or mechanism of action, that the unique local environment created within a DNA nanocage, particularly the high density of negatively charged phosphate groups, enhances the activity of encapsulated enzymes, where the tightly bound, highly structured water layers on DNA surface may stabilize the active enzyme conformations. This effect appears consistent with recent independent evidence that many conserved metabolic enzymes are stabilized by polyphosphate and associate non-specifically with nucleic acids through cryptic binding sites thus taking advantage of the high polyanionic DNA and RNA contents of the cell. DNA nanocages therefore may serve as a molecular tool to precisely sculpt the properties of the local environment of enzymes in smart-material and biotechnological application. DNA nanocages also demonstrated their value in protecting encapsulated enzymes from biological degradation through proteases.

### REFERENCES

1. Chen, A. H. & Silver, P. A. Designing biological compartmentalization. Trends. Cell. Biol. 12, 662-670 (2012).
2. Hurtley, S. Location, Location, Location. Science 326, 1205 (2009).
3. Kertelf, C. A., Heinhorst, S. & Cannon, G. C. Bacterial microcompartments. Annu. Rev. Microbiol. 64, 391-408 (2010).
4. Kerfelf, C. A., Sawaya, M. R., Tanaka, S., Nguyen, C. V., Phillips, M., Beeby, M. & Yeates, T. O. Protein structures forming the shell of primitive bacterial organelles. Science 309, 936-938 (2005).
5. Graff, A., Winterhalter, M. & Meier, W. Nanoreactors from polymer-stabilized liposomes. Langmuir 17, 919-923 (2001).
6. Hartl, F. U. Molecular chaperones in cellular protein folding Nature 381, 571-580 (1996).
7. Comellas-Aragones, M. et al. A virus-based single-enzyme nanoreactor. Nature Nanotech. 2, 635-639 (2007).
8. Liu, Y. et al. Biomimetic enzyme nanocomplexes and their use as antidotes and preventive measures for alcohol intoxication. Nature Nanotech. 8, 187-192 (2013).
9. Sang, L. & Coppens, M. Effects of surface curvature and surface chemistry on the structure and activity of protein adsorbed in nanopores. Phys. Chem. Chem. Phys. 13, 6689-6698 (2011).
10. Vriezema, D. M., Aragones, M. C., Elemans, J., Cornelissen, J., Rowan, A. E. & Nolte, R. J. M. Self-assembled nanoreactors. Chem. Rev. 105, 1445-1490 (2005).
11. Bruns, N. & Tiller, J. C. Amphiphilic network as nanoreactor for enzymes in organic solvents. Nano Lett. 5, 45-48 (2005).
12. Betancor , L., and Luckarift, H. R. Bioinspired enzyme encapsulation for biocatalysis. Trends. Biotechnol. 26, 566-572 (2008).
13. Fiedler, J. D., Brown. S. D., Lau. J. & Finn. M. G. RNA-directed packaging of enzymes within virus-like particles. Angew. Chem. Int. Ed. 49, 9648-9651 (2010).
14. Douglas, S. M., Dietz, H., Liedl, T., Hogberg, B., Graf, F. & Shih, W. M. Self-assembly of DNA into nanoscale three-dimensional shapes. Nature 459, 414-418 (2009).
15. Han, D., Pal, S., Nangreave, J., Deng, Z., Liu, Y. & Yan, H. DNA origami with complex curvatures in three-dimensional space. Science 332, 342-346 (2011).
16. Ke, Y., Ong, L. L., Shih, W. M. & Yin, P. Three-dimensional structures self-assembled from DNA bricks. Science, 338, 1177-1183 (2012).
17. Kuzyk, A. et al. DNA-based self-assembly of chiral plasmonic nanostructures with tailored optical response. Nature 483, 311-314 (2012).
18. Langecker, M., Arnaut, V., Martin, T., List, J., Renner, S., Mayer, M., Dietz, H., & Simmel, F. Synthetic lipid membrane channels by designed DNA nanostructures. Science 338, 932-936 (2012).
19. Fu, J., Liu, M., Liu, Y., Woodbury, N. W. & Yan, H. Interenzyme substrate diffusion for an enzyme cascade organized on spatially addressable DNA nanostructure. J. Am. Chem. Soc. 134, 5516-5519 (2012).
20. Fu, J., Liu, M., Liu, Y. & Yan, H. Spatially-Interactive Biomolecular Networks Organized by Nucleic Acid Nanostructures, Acc. Chem. Res. 45, 1215-1226 (2012).
21. Wilner, O . I., Weizmann, Y., Gill, R., Lioubashevski, O., Freeman, R. & Willner, I. Enzyme cascades activated on topologically programmed DNA scaffolds. Nature Nanotechnol. 4, 249-254 (2009).
22. Andersen. E. S. et al. Self-assembly of a nanoscale DNA box with a controllable lid. Nature 459, 73-76 (2009).
23. Douglas, S. M., Bachelet, I., Church, G. M. A logic-gated nanorobot for targeted transport of molecular payloads. Science 335, 831-834 (2012).
24. Juul, S., et al. Temperature-Controlled Encapsulation and Release of an Active Enzyme in the Cavity of a Self-Assembled DNA Nanocage. ACS Nano 7, 9724-9734 (2013).
25. Fu, Y., et al. Single-Step Rapid Assembly of DNA Origami Nanostructures for Addressable Nanoscale Bioreactors. J. Am. Chem. Soc. 135, 696-702 (2013).
26. Linko, V., Eerikainen, M. & Kostiainen, M. A modular DNA origami-based enzyme cascade nanoreactor. Chem. Commun. 51, 5351-5354 (2015).
27. Gray M. J., et al. Polyphosphate is a primordial chaperone. Mol. Cell. 53, 689-699 (2014).
28. Cie§la, J. Metabolic enzymes that bind RNA: yet another level of cellular regulatory network? Acta Biochim Pol. 53, 11-32 (2006).
29. Bellot, G., McClintock, M. A., Lin, C. X., Shih, W. M. Recovery of intact DNA nanostructures after agarose gel-based separation. Nat. Methods. 8, 192-194 (2011).
30. Liu, M., Fu, J., Hejesen, C., Yang, Y., Woodbury, N. W., Gothelf, K., Liu, Y., and Yan, H. A DNA Tweezer-Actuated Enzyme Nanoreactors, Nat. Commun. 6, 712-719 (2013).
31. Fu, J., Yang, Y. R., Johnson-Buck, A., Liu, Y., Walter, N. G., Woodbury, N. W., and Yan, H. Multi-enzyme complexes on DNA scaffolds capable of substrate channeling with an artificial swinging arm, Nature Nanotechnol. 9, 531-536 (2014).
32. Hecht, H. J., Kalisz, K., Hendle, J., Schmid, R. D., Schomburg, D. Crystal structure of glucose oxidase from Aspergillus niger refined at 2-3 A resolution. J. Mol. Biol. 229, 153-172 (1993).
33. Henriksen, A., Schuller, D. J., Gajhede, M. Structural interactions between horseradish peroxidase C and the substrate benzhydroxamic acid determined by X-ray crystallography. Biochemistry 37, 8054-8060 (1998).
34. Widom, J. R., Dhakal, S., Heinicke, L. A. & Walter, N. G. Single-molecule tools for enzymology, structural biology, systems biology and nanotechnology: an update. Arch. Toxicol. 88, 1965-1985 (2014).
35. Veitch, N. C. Horseradish peroxidase: a modern view of a classic enzyme. Phytochemistry 65, 249-259 (2004).
36. Chapman, A. D., Cortes, A., Dafforn, T. R., Clarke, A. R. & Brady, R. L. Structural basis of substrate specificity in malate dehydrogenases: crystal structure of a ternary complex of porcine cytoplasmic malate dehydrogenase, alpha-ketomalonate and tetrahydoNAD. J Mol Biol. 285, 703-712 (1999).
37. Rowland, P., Basak, A. K., Gover, S., Levy, H. R. & Adams, M. J. The three-dimensional structure of glucose 6-phosphate dehydrogenase from Leuconostoc mesenteroides refined at 2.0 A resolution. Structure 15, 1073-1087 (1994).
38. Lovell, S. L. & Winzor, D. J. Effects of phosphate on the dissociation and enzymic stability of rabbit muscle lactate dehydrogenase. Biochemistry 13, 3527-3531 (1974).
39. Hecht, H. J., Kalisz, H. M., Hendle, J., Schmid, R. D. & Schomburg D. Crystal structure of glucose oxidase from Aspergillus niger refined at 2.3 A resolution. J Mol Biol. 229, 153-172 (1993).
40. Jacobson, R. H., Zhang, X. J., DuBose, R. F. & Matthews, B. W. Three-dimensional structure of beta-galactosidase from E. coli. Nature 369, 761-766 (1994).
41. Erkelenz, M., Kuo, C. H. & Niemeyer, C. M. DNA-Mediated Assembly of Cytochrome P450 BM3 Subdomains, J. Am. Chem. Soc. 133, 16111-16118 (2011).
42. Rudiuk, S., Venancio-Marques, A. & Baigl, D. Enhancement and modulation of enzymatic activity through higher-order structural changes of giant DNA-protein multibranch conjugates. Angew. Chem. Int. Ed. 51, 12694-12698 (2012).
43. English, B. P., et al. Ever-fluctuating single enzyme molecules: Michaelis-Menten equation revisited. Nat. Chem. Bio. 2, 87-94 (2006).
44. Liu, B., Baskin, R. J. & Kowalczykowski, S. C. NA unwinding heterogeneity by RecBCD results from static molecules able to equilibrate. Nature 500, 482-485 (2013).
45. Ramanathan, A., Savol, A., Burger, V., Chennubhotla, C. S. & Agarwal, P. K. Protein Conformational Populations and Functionally Relevant Substates. Acc. Chem. Res. 47, 149-156 (2014).
46. Hammes, G. G., Benkovic, S. J. & Hammes-Schiffer, S. Flexibility, Diversity, and Cooperativity: Pillars of Enzyme Catalysis. Biochemistry 50, 10422-10430 (2011).
47. Ramanathan, A. & Agarwal P. K. Evolutionarily Conserved Linkage between Enzyme Fold, Flexibility, and Catalysis. PLoS Biol. 9, 1-17 (2011).
48. Timm, C. & Niemeyer, C. M. Assembly and Purification of Enzyme-Functionalized DNA Origami Structures. Angew. Chem. Int. Ed. 54, 6745-6750 (2015).
49. Lin, J. & Wheeldon, I. Kinetic Enhancements in DNA—Enzyme Nanostructures Mimic the Sabatier Principle ACS Catal. 3, 560-564 (2013).
50. Gao, Y., Roberts, C. C., Zhu, J., Lin, J., Chang, C. A. & Wheeldon, I. Tuning Enzyme Kinetics through Designed Intermolecular Interactions Far from the Active Site. ACS Catal. 5, 2149-2153 (2015).
51. Zhao, H. Effects of ions and other compatible solutes on enzyme activity, and its implication for biocatalysis using ionic liquids. J. Mol. Catal. B-Enzym. 37, 16-25 (2005).
52. Moelberta, S., Normandb, B. & Rios, P. D. L. Kosmotropes and chaotropes: modelling preferential exclusion, binding and aggregate stability. Biophys. Chem. 112, 45-57 (2004).
53. N Leberman, R. & Soper, A. K. Effect of high salt concentrations on water structure. Nature 378, 364-366 (1995).
54. N Jana, B., Pal, S., Maiti. P. K., Lin. S., Hynes, J. T. & Bagchi, B. Entropy of Water in the Hydration Layer of Major and Minor Grooves of DNA. J. Phys. Chem. B 110, 19611-19618 (2006).
55. N Chuprina, V. P., Heinemann, U., Nurislamov, A. A., Zielenkiewicz, P., Dickerson, R. E. & Saenger W. Molecular dynamics simulation ofthe hydration shell ofa B-DNA decamer reveals two main types of minor-groove hydration depending on groove width. Proc. Nati. Acad. Sci. USA 88, 593-597 (1991).
56. Zhao, H., Olubajo, O., Song, Z., Sims, A. L., Person, T. E., Lawal, R. A. & Holley, L. A. Effect of kosmotropicity of ionic liquids on the enzyme stability in aqueous solutions. Bioorg. Chem. 34, 15-25 (2006).
57. Timasheff, S. N. Protein—solvent preferential interactions, protein hydration, and the modulation of biochemical reactions by solvent components. Proc. Natl. Acad. Sci. 99, 9721-9726 (2002).
58. N Levy, Y. & Onuchic, J. N. Water and proteins: A love—hate relationship. Proc. Natl. Acad. Sci. USA 101, 3325-3326 (2004)
59. N Grey, M. J. et al. Polyphosphate is a Primordial Chaperone. Mol. Cell. 53, 689-699 (2014).
60. Marcus, Y. Effects of ions on the structure of water: structure making and breaking. Chem. Rev. 109, 1346-1370 (2009).
61. Mei, Q., Wei, X., Su, F., Liu, Y., Yongbull, C., Johnson, R., Lindsay, S., Yan, H., Meidrum, D. Stability of DNA origami nanoarrays in cell lysate. Nano Lett. 11, 1477-1482 (2011).
62. Jiang, Q., Song, C., Nangreave, J., Liu, X., Lin, L., Qiu, Z., Wang, Z., Zou, G., Liang, X., Yan, H., Ding, B. DNA origami as a carrier for circumvention of drug resistance. J. Am. Chem. Soc. 134, 13396-13403 (2012).
63. Castello, A. et al. Insights into RNA Biology from an Atlas of Mammalian mRNA-Binding Proteins. Cell 149, 1393-1406 (2012)
64. Wong, C. M., Wong, K. H. and Chen, X. D. Glucose oxidase: natural occurrence, function, properties and industrial application. Appl. Microbiol. Biotechnol. 78, 927-38 (2008).
65. Guo, S., Cao, R., Lu, A., Zhou, Q., Lu, T., Ding, X., Li, C. and Huang, X. One of the possible mechanisms for the inhibition effect of Tb(III) on peroxidase activity in horseradish (Armoracia rusticana) treated with Tb(III). J. Biol. Inorg. Chem. 13, 587-597 (2008).
66. Sung, J. Y. & Lee, Y. N. Isoforms of glucose 6-phosphate dehydrogenase in Deinococcus radiophilus. J. Microbiol. 45, 318-325 (2007).
67. Horikiri, S., Aizawa, Y., Kai, T., Amachi, S., Shinoyama, H. and Fujii, T. Electron acquisition system constructed from an NAD-independent D-lactate dehydrogenase and cytochrome c2 in Rhodopseudomonas palustris No. 7. Biosci. Biotechnol. Biochem. 68, 516-522 (2004).
68. Eanes, R.Z., and Kun, E. Separation and characterization of aconitate hydratase isoenzymes from pig tissues. Biochim. Biophys. Acta 227, 204-210 (1971).
69. Fu, J., Liu, M., Liu, Y., Woodbury, N. W. & Yan, H. Interenzyme Substrate Diffusion for an Enzyme Cascade Organized on Spatially Addressable DNA Nanostructures. J. Am. Chem. Soc. 134, 5516-5519 (2012).
70. Liu, M., Fu, J., Hejesen, C., Yang, Y., Woodbury, N. W., Gothelf, K., Liu, Y. & Yan, H. A DNA Tweezer-Actuated Enzyme Nanoreactor. Nature Commun. 4, 1-5 (2013).
71. Abelson, J. et al. Conformational dynamics of single pre-mRNA molecules during in vitro splicing Nat. Struct. Mal. Biol. 17, 504-512 (2010).
72. Michelotti, N. et al. A bird's eye view tracking slow nanometer-scale movements of single molecular nano-assemblies. Methods Enzymol. 475, 121-148 (2010).
73. Blanco, M. & Walter, N. G. Analysis of Complex Single-Molecule FRET Time Trajectories. Method. Enzymol. 472, 153-178 (2010).
74. Gourevitch, B. & Eggermont, J. J. A nonparametric approach for detection of bursts in spike trains. Journal of Neuroscience Methods 160, 349-358 (2007).
75. Rinaldi, A. J., Lund, P. E., Blanco, M. R. & Walter, N. G. The Shine-Dalgamo sequence of riboswitch-regulated single mRNAs shows ligand-dependent accessibility bursts. Nat. Commun., 8976 (2015).

We claim:

1-19. (canceled)

20. A method of increasing the activity of an enzyme or antibody, the method comprising:
trapping an enzyme or antibody in a first open half cage comprising M13 viral DNA;
presenting a second open half cage which does not comprise an enzyme or antibody;
assembling the first and second open half cages into a closed nanocage having an inner cavity and nanopores;
wherein the inner cavity comprises the enzyme or antibody as a trapped enzyme or antibody respectively, and
wherein the enzyme or antibody is less than 465 kDa in size.

21. The method of claim 20, wherein the activity is selected from increased catalytic activity, increased potency, or increased resistance to proteolytic digestion.

22. The method of claim 21, wherein the activity is increased resistance to proteolytic digestion by trypsin.

23. The method of claim 21, wherein the increased resistance to proteolytic digestion is more than 95% of the initial activity of the enzyme or antibody after incubation with trypsin for 24 h relative to that of respective free enzyme or antibody.

24. The method of claim 21, wherein the increased catalytic activity is more than six-fold higher for the trapped enzyme or antibody relative to that of the respective free enzyme or antibody.

25. The method of claim 20, wherein the trapped enzyme or antibody has a mean average cross-sectional size which is less than half the longest width of the inner cavity.

26. The method of claim 20, wherein the closed nanocage comprises a plurality of structural members, each of which independently comprises all or a portion of M13 viral DNA, in a three-dimensional lattice,
wherein internal surfaces of the plurality of structural members form an inner cavity which is defined by at least three pairs of opposite walls,
wherein a first structural member of the plurality of structural members is linked to a second structural member of the plurality of structural members by short bridge DNA strands,
wherein the at least three pairs of opposite walls comprises a first pair, a second pair, and a third pair of opposite walls, and
wherein each of the first pair of opposite walls comprises a thickness which is different from the other pairs of opposite walls.

27. The method of claim 20, wherein the nanocage comprises:
a plurality of structural members comprising a single-stranded circular DNA sequence selected from p7308, p7560, p7704, p8064, p8634, and pEGFP, wherein internal surfaces of the plurality of structural members form an inner cavity which is defined by at least three pairs of opposite walls, wherein architectural arrangement of the structural members in the three dimensional body forms an arrangement selected from the group consisting of a honeycomb lattice, a single-walled square lattice, and a double-walled square lattice, and
wherein the at least three pairs of opposite walls comprises a first pair, a second pair, and a third pair of opposite walls, and
wherein each of the first pair of opposite walls comprises a thickness which is different from the other pairs of opposite walls.

28. The method of claim 26, wherein the dimensions of the three dimensional body is less than 100 nm×100 nm×100 nm.

29. The method of claim 26, wherein the dimensions of the three dimensional body is less than 75 nm×50 nm×50 nm in size.

30. The method of claim 26, wherein the dimensions of the inner cavity of the three dimensional body is less than 50 nm×50 nm×50 nm.

31. The method of claim 26, wherein the three dimensional body further comprises at least one nanopore.

32. The method of claim 26, wherein the at least one nanopore has a diameter of about 1 nm to about 5 nm.

33. The method of claim 26, wherein the three dimensional body comprises between 0.10 to 0.30 DNA helices per nm².

34. The method of claim 26, wherein the three dimensional body comprises between 0.11 to 0.17 DNA helices per nm².


SEQUENCE LISTING (1395 sequences)

SEQ ID NO: 1 - DNA, 23 bp, synthetic construct
  source 1..23: mol_type = other DNA
ggtggagaggcggtttgcgtttt

SEQ ID NO: 2 - DNA, 23 bp, synthetic construct
  source 1..23: mol_type = other DNA
cgagttgggtaacgccaggtttt

SEQ ID NO: 3 - DNA, 16 bp, synthetic construct
  source 1..16: mol_type = other DNA
tttttcgccattcagg

SEQ ID NO: 4 - DNA, 26 bp, synthetic construct
  source 1..26: mol_type = other DNA
ttttgccagctttcatcaacattcgt

SEQ ID NO: 5 - DNA, 34 bp, synthetic construct
  source 1..34: mol_type = other DNA
tttttggagcaaacaagagaatcggaagattagc

SEQ ID NO: 6 - DNA, 34 bp, synthetic construct
  source 1..34: mol_type = other DNA
ttttgggagaagcctttatttcaaaaagggacag

SEQ ID NO: 7 - DNA, 46 bp, synthetic construct
  source 1..46: mol_type = other DNA
ggtggcatcaattcatgggcgcgacctgtttgtataagcaaatttt

SEQ ID NO: 8 - DNA, 26 bp, synthetic construct
  source 1..26: mol_type = other DNA
atataaagtagtagatgggcgctttt

SEQ ID NO: 9 - DNA, 26 bp, synthetic construct
  source 1..26: mol_type = other DNA
aatcatactaatagtagtagcatttt

SEQ ID NO: 10 - DNA, 31 bp, synthetic construct
  source 1..31: mol_type = other DNA
gctgtcatagcaccgagctcgaattcgtttt

SEQ ID NO: 11 - DNA, 32 bp, synthetic construct
  source 1..32: mol_type = other DNA
tttttgaggactaaagactttcaacactaagg

SEQ ID NO: 12 - DNA, 37 bp, synthetic construct
  source 1..37: mol_type = other DNA
cggttttgctttgcgctagtgagctaactcacatttt

SEQ ID NO: 13 - DNA, 34 bp, synthetic construct
  source 1..34: mol_type = other DNA
ttttgaaggattaggattagcggtagcaacgcga

SEQ ID NO: 14 - DNA, 27 bp, synthetic construct
  source 1..27: mol_type = other DNA
ttttaaaagggcgacattcaaccaggc

SEQ ID NO: 15 - DNA, 37 bp, synthetic construct
  source 1..37: mol_type = other DNA
tgactaatatgtttgatgtttgccccagcaggctttt

SEQ ID NO: 16 - DNA, 27 bp, synthetic construct
  source 1..27: mol_type = other DNA
ttttaggcttatccggtattctagttt

SEQ ID NO: 17 - DNA, 48 bp, synthetic construct
  source 1..48: mol_type = other DNA
ctcaacaagtatcacataatttattaaagttccagtttggaacatttt

SEQ ID NO: 18 - DNA, 24 bp, synthetic construct
  source 1..24: mol_type = other DNA
ttttagagtccacactagaaaatt

SEQ ID NO: 19 - DNA, 25 bp, synthetic construct
  source 1..25: mol_type = other DNA
ttttgaaaatcctcagagagatttt

SEQ ID NO: 20 - DNA, 25 bp, synthetic construct
  source 1..25: mol_type = other DNA
ttttattgggcggagccaccatttt

SEQ ID NO: 21 - DNA, 22 bp, synthetic construct
  source 1..22: mol_type = other DNA
ttttaattgcgaaacaactttt

SEQ ID NO: 22 - DNA, 25 bp, synthetic construct
  source 1..25: mol_type = other DNA
ttttaatcatggctcattcagtttt

SEQ ID NO: 23 - DNA, 22 bp, synthetic construct
  source 1..22: mol_type = other DNA
ttttgttttcccagtcattttt

SEQ ID NO: 24 - DNA, 31 bp, synthetic construct
  source 1..31: mol_type = other DNA
ttttatcgtaaccgtggcaaagcgccatttt

SEQ ID NO: 25 - DNA, 31 bp, synthetic construct
  source 1..31: mol_type = other DNA
ttttatttaaattgtggccttcctgtatttt

SEQ ID NO: 26 - DNA, 32 bp, synthetic construct
  source 1..32: mol_type = other DNA
ttttgagacagtcaaatgcctgagagtctttt

SEQ ID NO: 27 - DNA, 39 bp, synthetic construct
  source 1..39: mol_type = other DNA
ttttagcctcagagcataaagcttaatacttttgctttt

SEQ ID NO: 28 - DNA, 30 bp, synthetic construct
  source 1..30: mol_type = other DNA
ttttaacatccaatattaagcaataatttt

SEQ ID NO: 29 - DNA, 29 bp, synthetic construct
  source 1..29: mol_type = other DNA
ttttaaatggtcaataagctgaaaatttt

SEQ ID NO: 30 - DNA, 29 bp, synthetic construct
  source 1..29: mol_type = other DNA
ttttattcccaatgatacatttcgctttt

SEQ ID NO: 31 - DNA, 28 bp, synthetic construct
  source 1..28: mol_type = other DNA
ttttaaatatgcaactaacagttgtttt

SEQ ID NO: 32 - DNA, 26 bp, synthetic construct
  source 1..26: mol_type = other DNA
ttttgcggatggcctcaacatgtttt

SEQ ID NO: 33 - DNA, 53 bp, synthetic construct
  source 1..53: mol_type = other DNA
ttttgtttaccagacgacgataatagcaaaaaatcattgagaaaggccgtttt

SEQ ID NO: 34 - DNA, 32 bp, synthetic construct
  source 1..32: mol_type = other DNA
ttttacataacgccaaatcataaccctctttt

SEQ ID NO: 35 - DNA, 27 bp, synthetic construct
  source 1..27: mol_type = other DNA
ttttagaaagatactaatgcagatttt

SEQ ID NO: 36 - DNA, 29 bp, synthetic construct
  source 1..29: mol_type = other DNA
ttttggaagaaaaatctattacaggtttt

SEQ ID NO: 37 - DNA, 29 bp, synthetic construct
  source 1..29: mol_type = other DNA
ttttgaattacctgtcaggacgttgtttt

SEQ ID NO: 38 - DNA, 26 bp, synthetic construct
  source 1..26: mol_type = other DNA
ttttgaataaggtaaattgggctttt

SEQ ID NO: 39 - DNA, 31 bp, synthetic construct
  source 1..31: mol_type = other DNA
ttttcaccctcagcaggctacagaggctttt

SEQ ID NO: 40 - DNA, 28 bp, synthetic construct
  source 1..28: mol_type = other DNA
ttttatattcggtttgcgggatcgtttt

SEQ ID NO: 41 - DNA, 29 bp, synthetic construct
  source 1..29: mol_type = other DNA
ttttgataccgatagtcataaccgatttt

SEQ ID NO: 42 - DNA, 25 bp, synthetic construct
  source 1..25: mol_type = other DNA
ttttaattgtacttaaacagctttt

SEQ ID NO: 43 - DNA, 27 bp, synthetic construct
  source 1..27: mol_type = other DNA
ttttaataattttttaaggagcctttt

SEQ ID NO: 44 - DNA, 26 bp, synthetic construct
  source 1..26: mol_type = other DNA
ttttcaacagttaggaattgcgaatt

SEQ ID NO: 45 - DNA, 30 bp, synthetic construct
  source 1..30: mol_type = other DNA
ttttcggaacctatgactcctcaagatttt

SEQ ID NO: 46 - DNA, 28 bp, synthetic construct
  source 1..28: mol_type = other DNA
ttttgtcagtgccccccctgcctatttt

SEQ ID NO: 47 - DNA, 28 bp, synthetic construct
  source 1..28: mol_type = other DNA
ttttcatacatggcttttaacgggtttt

SEQ ID NO: 48 - DNA, 29 bp, synthetic construct
  source 1..29: mol_type = other DNA
ttttcattaaagcttccagtaagcgtttt

SEQ ID NO: 49 - DNA, 28 bp, synthetic construct
  source 1..28: mol_type = other DNA
ttttaggttgaggcagataaatcctttt

SEQ ID NO: 50 - DNA, 29 bp, synthetic construct
  source 1..29: mol_type = other DNA
ttttccctcagagagcattgacaggtttt

SEQ ID NO: 51 - DNA, 30 bp, synthetic construct
  source 1..30: mol_type = other DNA
ttaagtttattttgagcgccaaagactttt

SEQ ID NO: 52 - DNA, 28 bp, synthetic construct
  source 1..28: mol_type = other DNA
ttttatacatacaacaccacggaatttt

SEQ ID NO: 53 - DNA, 28 bp, synthetic construct
  source 1..28: mol_type = other DNA
ttttgaactggcatgaacgtagaaattt

SEQ ID NO: 54 - DNA, 29 bp, synthetic construct
  source 1..29: mol_type = other DNA
ttttcaaagttacgaatacccaaaatttt

SEQ ID NO: 55 - DNA, 27 bp, synthetic construct
  source 1..27: mol_type = other DNA
ttttgcaatagctatcatagccgaatt

SEQ ID NO: 56 - DNA, 29 bp, synthetic construct
  source 1..29: mol_type = other DNA
ttttaacccacaaaacaatgaaatatttt

SEQ ID NO: 57 - DNA, 32 bp, synthetic construct
  source 1..32: mol_type = other DNA
ttttcgagaacaagcaatcagatatagatttt

SEQ ID NO: 58 - DNA, 29 bp, synthetic construct
  source 1..29: mol_type = other DNA
ttttagaaaccaataccgcactcattttt

SEQ ID NO: 59 - DNA, 29 bp, synthetic construct
  source 1..29: mol_type = other DNA
ttttacgcgcctgtttcgagcatgttttt

SEQ ID NO: 60 - DNA, 29 bp, synthetic construct
  source 1..29: mol_type = other DNA
ttttataaagtacagctaatgcagatttt

SEQ ID NO: 61 - DNA, 29 bp, synthetic construct
  source 1..29: mol_type = other DNA
tttttgagaatcgccataagagaattttt

SEQ ID NO: 62 - DNA, 29 bp, synthetic construct
  source 1..29: mol_type = other DNA
ttttagcctgtttgtagggcttaattttt

SEQ ID NO: 63 - DNA, 49 bp, synthetic construct
  source 1..49: mol_type = other DNA
gactccaataaacaccagggaagcgcataagtcagcggcaaatgcagca

SEQ ID NO: 64 - DNA, 28 bp, synthetic construct
  source 1..28: mol_type = other DNA
gaaccatcgtaaagcactaaacttgacg

SEQ ID NO: 65 - DNA, 28 bp, synthetic construct
  source 1..28: mol_type = other DNA
tagggttccgaaatagggtaaacaaatc

SEQ ID NO: 66 - DNA, 44 bp, synthetic construct
  source 1..44: mol_type = other DNA
ggtcaaaagaatagagggcgaaaaaccgtaaataagagaattaa

SEQ ID NO: 67 - DNA, 23 bp, synthetic construct
  source 1..23: mol_type = other DNA
ataaaaggaacacccaccaccgg

SEQ ID NO: 68 - DNA, 42 bp, synthetic construct
  source 1..42: mol_type = other DNA
gggaaaggggcgctggcaagtcgctgcgcgtaaccttgacga

SEQ ID NO: 69 - DNA, 28 bp, synthetic construct
  source 1..28: mol_type = other DNA
agcggtccttttcaccctcagatttagc

SEQ ID NO: 70 - DNA, 35 bp, synthetic construct
  source 1..35: mol_type = other DNA
gtagctctttagagtcggaacatggcccactacgt

SEQ ID NO: 71 - DNA, 28 bp, synthetic construct
  source 1..28: mol_type = other DNA
cggccaacgctttcttttctgaatggct

SEQ ID NO: 72 - DNA, 28 bp, synthetic construct
  source 1..28: mol_type = other DNA
ctgcattgacgggcagagagtatccctt

SEQ ID NO: 73 - DNA, 42 bp, synthetic construct
  source 1..42: mol_type = other DNA
gcacgtaagctaaacaggaggttttataatcagtggtaaaag

SEQ ID NO: 74 - DNA, 45 bp, synthetic construct
  source 1..45: mol_type = other DNA
gcccgcgcggggtttttcacgctgggtggttgagtgttgaacgtg

SEQ ID NO: 75 - DNA, 23 bp, synthetic construct
  source 1..23: mol_type = other DNA
gtcgtttttccagagtaatcttg

SEQ ID NO: 76 - DNA, 28 bp, synthetic construct
  source 1..28: mol_type = other DNA
tggggtgtgtgtgacaaatcactcgaac

SEQ ID NO: 77 - DNA, 28 bp, synthetic construct
  source 1..28: mol_type = other DNA
aggccatgagcgggtaacgtgctggtca

SEQ ID NO: 78 - DNA, 42 bp, synthetic construct
  source 1..42: mol_type = other DNA
agtctgtcacttgcctgagtaatccagaacaatatacgctca

SEQ ID NO: 79 - DNA, 35 bp, synthetic construct
  source 1..35: mol_type = other DNA
cacgaagtgtccgattaggttgctaccacacggcg

SEQ ID NO: 80 - DNA, 49 bp, synthetic construct
  source 1..49: mol_type = other DNA
gaggatcaaacgacaattgctcagtttgtaggtcaaaatgtgaataatt

SEQ ID NO: 81 - DNA, 28 bp, synthetic construct
  source 1..28: mol_type = other DNA
gcaggtcatccgcttaaagtggaaacct

SEQ ID NO: 82 - DNA, 42 bp, synthetic construct
  source 1..42: mol_type = other DNA
ttgcacgtcaggatgtatcggggcggatcgtcggaaccaata

SEQ ID NO: 83 - DNA, 46 bp, synthetic construct
  source 1..46: mol_type = other DNA
tgcaaggactgttggtgccggaaaccagcatctgcccttttgttaa

SEQ ID NO: 84 - DNA, 28 bp, synthetic construct
  source 1..28: mol_type = other DNA
gaaatattggtaatgaagaacacaccga

SEQ ID NO: 85 - DNA, 28 bp, synthetic construct
  source 1..28: mol_type = other DNA
atcgtctatttacacagagatagcgcac

SEQ ID NO: 86 - DNA, 46 bp, synthetic construct
  source 1..46: mol_type = other DNA
ctgcgcacgattaacgttgtacccgggttgtttcccctaatgcact

SEQ ID NO: 87 - DNA, 21 bp, synthetic construct
  source 1..21: mol_type = other DNA
cgatcggaaagggggccaagc

SEQ ID NO: 88 - DNA, 32 bp, synthetic construct
  source 1..32: mol_type = other DNA
attcacccattttgtaccgccataacatccat

SEQ ID NO: 89 - DNA, 18 bp, synthetic construct
  source 1..18: mol_type = other DNA
cggcaccgacgacataga

SEQ ID NO: 90 - DNA, 42 bp, synthetic construct
  source 1..42: mol_type = other DNA
aagataacccttctagccctaattaaaaacgctgagagctca

SEQ ID NO: 91 - DNA, 42 bp, synthetic construct
  source 1..42: mol_type = other DNA
agaatacgtctttaaccagcaaacaccgcctgcaaaaatcta

SEQ ID NO: 92 - DNA, 43 bp, synthetic construct
  source 1..43: mol_type = other DNA
tggtcggtgtcatataataggacagaccagaaaaaatctaaag

SEQ ID NO: 93 - DNA, 42 bp, synthetic construct
  source 1..42: mol_type = other DNA
aaacaggaacaaaccctcaggtccagccctcttcgctggatt

SEQ ID NO: 94 - DNA, 26 bp, synthetic construct
  source 1..26: mol_type = other DNA
aacaacctgaccgtgacttcaaatat

SEQ ID NO: 95 - DNA, 42 bp, synthetic construct
  source 1..42: mol_type = other DNA
acgaaccatgcgcgaactgatgacctgatggccaattggcag

SEQ ID NO: 96 - DNA, 39 bp, synthetic construct
  source 1..39: mol_type = other DNA
cgcgtctaaacgttagtttcaacgagtaaactttgtttt

SEQ ID NO: 97 - DNA, 42 bp, synthetic construct
  source 1..42: mol_type = other DNA
aatcagccagcagcaatcaacaattgaggatttagaaggaga

SEQ ID NO: 98 - DNA, 42 bp, synthetic construct
  source 1..42: mol_type = other DNA
aagcatctcaatatatatctttcaatagataatacacaattc

SEQ ID NO: 99 - DNA, 42 bp, synthetic construct
  source 1..42: mol_type = other DNA
agttgtgtaccccgtttgttattttttattctccgtgtcgcc

SEQ ID NO: 100 - DNA, 26 bp, synthetic construct
  source 1..26: mol_type = other DNA
gtaaaacatcagaacagaaaacgaga

SEQ ID NO: 101 - DNA, 42 bp, synthetic construct
  source 1..42: mol_type = other DNA
atctaaactggtcagttggcaaaatgaacagtgccataccga

SEQ ID NO: 102 - DNA, 39 bp, synthetic construct
  source 1..39: mol_type = other DNA
aggtcattcaccattcatttgactgcggtaacggattga

SEQ ID NO: 103 - DNA, 42 bp, synthetic construct
  source 1..42: mol_type = other DNA
gagagataccgttctagctgatgcctgagaacccttggaagg

SEQ ID NO: 104 - DNA, 21 bp, synthetic construct
  source 1..21: mol_type = other DNA
atgccgtattagacatcattt

SEQ ID NO: 105 - DNA, 49 bp, synthetic construct
  source 1..49: mol_type = other DNA
gacaacttttaaaaaattatccatcaatataatcctgattgtaccagaa

SEQ ID NO: 106 - DNA, 42 bp, synthetic construct
  source 1..42: mol_type = other DNA
tgcggtaaatgcaataaattagggtagctcaatcataaaagg

SEQ ID NO: 107 - DNA, 42 bp, synthetic construct
  source 1..42: mol_type = other DNA
ggagcgggtttgagtaacatttttacaaatttgaggaaggtt

SEQ ID NO: 108 - DNA, 39 bp, synthetic construct
  source 1..39: mol_type = other DNA
gaccctgaaatcggaaagaataaaccaagtaagagttca

SEQ ID NO: 109 - DNA, 40 bp, synthetic construct
  source 1..40: mol_type = other DNA
cctacaagataaaaatttttagtaatgttttgccagaggg

SEQ ID NO: 110 - DNA, 49 bp, synthetic construct
  source 1..49: mol_type = other DNA
gttagaacaaaattgtagattttcagaggctttgcttgtaccaacatat

SEQ ID NO: 111 - DNA, 42 bp, synthetic construct
  source 1..42: mol_type = other DNA
tgaataacatatatttaacaaagaaaccttggattatacttc

SEQ ID NO: 112 - DNA, 42 bp, synthetic construct
  source 1..42: mol_type = other DNA
gcatagcgtctacgagggaataccacagcatagttaaaaaac

SEQ ID NO: 113 - DNA, 35 bp, synthetic construct
  source 1..35: mol_type = other DNA
aagaagtgtaggtaatattcactacaaaggtaatc

SEQ ID NO: 114 - DNA, 24 bp, synthetic construct
  source 1..24: mol_type = other DNA
ggtaatagtaaaccaacctaaaac

SEQ ID NO: 115 - DNA, 47 bp, synthetic construct
  source 1..47: mol_type = other DNA
tgaatccccctcaaatgttcaatatgaagattcacgcaagacattat

SEQ ID NO: 116 - DNA, 42 bp, synthetic construct
  source 1..42: mol_type = other DNA
tattagtttgataaaacggctcatacaatgattcgaggatac

SEQ ID NO: 117 - DNA, 49 bp, synthetic construct
  source 1..49: mol_type = other DNA
caggtttatctttaaacagttaagcccctgttaaacatcaaaagcgagt

SEQ ID NO: 118 - DNA, 49 bp, synthetic construct
  source 1..49: mol_type = other DNA
ccctgacaaaagattaagagaaatatttaaaaacagatgaacggctatc

SEQ ID NO: 119 - DNA, 37 bp, synthetic construct
  source 1..37: mol_type = other DNA
aaagcggatttaaattgttgatatagcatgtattttt

SEQ ID NO: 120 - DNA, 35 bp, synthetic construct
  source 1..35: mol_type = other DNA
cccgaaaaatgggaaggggacgcttctgggaaggg

SEQ ID NO: 121 - DNA, 14 bp, synthetic construct
  source 1..14: mol_type = other DNA
cgcgttttaattga

SEQ ID NO: 122 - DNA, 42 bp, synthetic construct
  source 1..42: mol_type = other DNA
ttgacgtaactgacgagtatgggaagtgagaaaccgcccaga

SEQ ID NO: 123 - DNA, 28 bp, synthetic construct
  source 1..28: mol_type = other DNA
cagaccgtacctttggccagtgatgtgc

SEQ ID NO: 124 - DNA, 42 bp, synthetic construct
  source 1..42: mol_type = other DNA
gaggaagcaaggatattatcaagacgttagttctaaagcctc

SEQ ID NO: 125 - DNA, 37 bp, synthetic construct
  source 1..37: mol_type = other DNA
gggccttgctacgccagctggcgtgcgggcagctttc

SEQ ID NO: 126 - DNA, 26 bp, synthetic construct
  source 1..26: mol_type = other DNA
gaaggcaatgtttaataaatattcat

SEQ ID NO: 127 - DNA, 24 bp, synthetic construct
  source 1..24: mol_type = other DNA
gaaagaggcaaaagagggttgata

SEQ ID NO: 128 - DNA, 42 bp, synthetic construct
  source 1..42: mol_type = other DNA
gataaccatcggcttgctactggtacagtgccagtatgggca

SEQ ID NO: 129 - DNA, 42 bp, synthetic construct
  source 1..42: mol_type = other DNA
tcatcttaagtacaacggaacaatcgtcgactggaagtgcaa

SEQ ID NO: 130 - DNA, 32 bp, synthetic construct
  source 1..32: mol_type = other DNA
taccaagcgcgaaacatgacccccagcgatta

SEQ ID NO: 131 - DNA, 26 bp, synthetic construct
  source 1..26: mol_type = other DNA
ccatatgcgaaaactttaatcattgt

SEQ ID NO: 132 - DNA, 42 bp, synthetic construct
  source 1..42: mol_type = other DNA
aagaaaaaagatcagctatattcagaaagcgagaaaagaaac

SEQ ID NO: 133 - DNA, 42 bp, synthetic construct
  source 1..42: mol_type = other DNA
tacttaggaaccgagtgtacacgagcttcaaaggatgggaag

SEQ ID NO: 134 - DNA, 18 bp, synthetic construct
  source 1..18: mol_type = other DNA
acggtcaatgtcagaagc

SEQ ID NO: 135 - DNA, 44 bp, synthetic construct
  source 1..44: mol_type = other DNA
gagatggtttaatttcaaggctgtagttagagcataagaggtca

SEQ ID NO: 136 - DNA, 28 bp, synthetic construct
  source 1..28: mol_type = other DNA
taggcacatgaacgactgaccgacttta

SEQ ID NO: 137 - DNA, 14 bp, synthetic construct
  source 1..14: mol_type = other DNA
ccacaacgcctgta

SEQ ID NO: 138 - DNA, 28 bp, synthetic construct
  source 1..28: mol_type = other DNA
gaccttccattaccaattgttgactcta

SEQ ID NO: 139 - DNA, 23 bp, synthetic construct
  source 1..23: mol_type = other DNA
acccacacatcaaactattgcct

SEQ ID NO: 140 - DNA, 49 bp, synthetic construct
  source 1..49: mol_type = other DNA
tgaacgagggggttttgtattaaggattgagtcatatgagaacgcccaa

SEQ ID NO: 141 - DNA, 49 bp, synthetic construct
  source 1..49: mol_type = other DNA
tccattaatacgtaatgcctaaatagcgaggtttaacgtcaggggtaaa

SEQ ID NO: 142 - DNA, 35 bp, synthetic construct
  source 1..35: mol_type = other DNA
aacggagtaccaagttacaaggcggagaggaagtt

SEQ ID NO: 143 - DNA, 49 bp, synthetic construct
  source 1..49: mol_type = other DNA
aagacattcatcatcagacaacattacgttaaccattatctgcgattcc

SEQ ID NO: 144 - DNA, 28 bp, synthetic construct
  source 1..28: mol_type = other DNA
gccgtcgagaatacactaaagcaactac

SEQ ID NO: 145 - DNA, 24 bp, synthetic construct
  source 1..24: mol_type = other DNA
taagtatagccccaccgtcaccga

SEQ ID NO: 146 - DNA, 26 bp, synthetic construct
  source 1..26: mol_type = other DNA
tttagtaccgccaccctacttaacac

SEQ ID NO: 147 - DNA, 42 bp, synthetic construct
  source 1..42: mol_type = other DNA
ccgccaccgcgacctgctcctgagatttgtatcatcaaaaat

SEQ ID NO: 148 - DNA, 41 bp, synthetic construct
  source 1..41: mol_type = other DNA
ttttcggtttgctccaacacgttgcgagtagcttgcccaaa

SEQ ID NO: 149 - DNA, 28 bp, synthetic construct
  source 1..28: mol_type = other DNA
gggatagtgagtttcgtcaaaaacatgt

SEQ ID NO: 150 - DNA, 45 bp, synthetic construct
  source 1..45: mol_type = other DNA
gaagattggcccagagcagccctttaataagcaacgccgccaacg

SEQ ID NO: 151 - DNA, 14 bp, synthetic construct
  source 1..14: mol_type = other DNA
gcattccacagaca

SEQ ID NO: 152 - DNA, 28 bp, synthetic construct
  source 1..28: mol_type = other DNA
gtaacgaaaatgaacagtcggtaaagcc

SEQ ID NO: 153 - DNA, 46 bp, synthetic construct
  source 1..46: mol_type = other DNA
ctagaatcaacgagccggaagcacacaattaagaaccactccaaca

SEQ ID NO: 154 - DNA, 42 bp, synthetic construct
  source 1..42: mol_type = other DNA
tgatattctgggccgcttcgctgagcccacgtgcgccgtata

SEQ ID NO: 155 - DNA, 35 bp, synthetic construct
  source 1..35: mol_type = other DNA
cattaagctcagtaccaaatcgcgcagaagacgga

SEQ ID NO: 156 - DNA, 38 bp, synthetic construct
  source 1..38: mol_type = other DNA
atgataaacacaatagaaaagaaatttatttggtatta

SEQ ID NO: 157 - DNA, 42 bp, synthetic construct
  source 1..42: mol_type = other DNA
agagcgtaaaaggtgaattatggaataggtgtaggcgtaagt

SEQ ID NO: 158 - DNA, 24 bp, synthetic construct
  source 1..24: mol_type = other DNA
cttgagccatttcgggaggttttg

SEQ ID NO: 159 - DNA, 26 bp, synthetic construct
  source 1..26: mol_type = other DNA
caatgtgagtcaccgtactcaggagg

SEQ ID NO: 160 - DNA, 32 bp, synthetic construct
  source 1..32: mol_type = other DNA
attagcaaggccggaacagtagcaccattacc

SEQ ID NO: 161 - DNA, 38 bp, synthetic construct
  source 1..38: mol_type = other DNA
aggctctgaatccttatacgcaatatagatataaacaa

SEQ ID NO: 162 - DNA, 42 bp, synthetic construct
  source 1..42: mol_type = other DNA
gtttggtagaaaccatcgatacaccaccctcatctcacagaa

SEQ ID NO: 163 - DNA, 24 bp, synthetic construct
  source 1..24: mol_type = other DNA
tcagtagcgacaacgagcgtcttt

SEQ ID NO: 164 - DNA, 42 bp, synthetic construct
  source 1..42: mol_type = other DNA
cgtttgccagccctcatagagccccagtacaaactaggcgca

SEQ ID NO: 165 - DNA, 14 bp, synthetic construct
  source 1..14: mol_type = other DNA
tcaaatagcagcct

SEQ ID NO: 166 - DNA, 38 bp, synthetic construct
  source 1..38: mol_type = other DNA
accttgagcggttaagcccggaattatgcgttatacaa

SEQ ID NO: 167 - DNA, 28 bp, synthetic construct
  source 1..28: mol_type = other DNA
accggaagccgccaccagtgaaatgaat

SEQ ID NO: 168 - DNA, 38 bp, synthetic construct
  source 1..38: mol_type = other DNA
agaccagagcctgaacatagacggggcgttatgaccta

SEQ ID NO: 169 - DNA, 23 bp, synthetic construct
  source 1..23: mol_type = other DNA
aagattgccctttcctcggccag

SEQ ID NO: 170 - DNA, 49 bp, synthetic construct
  source 1..49: mol_type = other DNA
tattgaaaattacatttaatagcgaaatggagggaaggtaaaaattatt

SEQ ID NO: 171 - DNA, 42 bp, synthetic construct
  source 1..42: mol_type = other DNA
acctcacaatgttaatgttgagtaaataagttttgatgtgaa

SEQ ID NO: 172 - DNA, 33 bp, synthetic construct
  source 1..33: mol_type = other DNA
aagccttaaatcgagtgaataattttccattcc

SEQ ID NO: 173 - DNA, 38 bp, synthetic construct
  source 1..38: mol_type = other DNA
acacccatcctcggctgtctttccttatcctaagaaaa

SEQ ID NO: 174 - DNA, 40 bp, synthetic construct
  source 1..40: mol_type = other DNA
caattttatcctgaatccgcccagcaaaatcacacgtcac

SEQ ID NO: 175 - DNA, 42 bp, synthetic construct
  source 1..42: mol_type = other DNA
gactttaccgcagaatgcaaacaagtcagaccaactaatcag

SEQ ID NO: 176 - DNA, 47 bp, synthetic construct
  source 1..47: mol_type = other DNA
ccagagcctaatgtgaattttaacctccagacgacgacaaagtcctg

SEQ ID NO: 177 - DNA, 42 bp, synthetic construct
  source 1..42: mol_type = other DNA
aggtaagcagttaccgacgccgccccgccacaccctcaccag

SEQ ID NO: 178 - DNA, 38 bp, synthetic construct
  source 1..38: mol_type = other DNA
cgatttcgagaggtaaagtaattctgtccggagaggca

SEQ ID NO: 179 - DNA, 42 bp, synthetic construct
  source 1..42: mol_type = other DNA
tttaatacacctttagcgtcacatagccccctttgtgtttca

SEQ ID NO: 180 - DNA, 32 bp, synthetic construct
  source 1..32: mol_type = other DNA
tccaaataagaaacgaatattatttatcccaa

SEQ ID NO: 181 - DNA, 42 bp, synthetic construct
  source 1..42: mol_type = other DNA
aaaacaattcgtcaaaaatgattttcataatcacactattag

SEQ ID NO: 182 - DNA, 26 bp, synthetic construct
  source 1..26: mol_type = other DNA
ttacagagagaaaaagaacatttcat

SEQ ID NO: 183 - DNA, 46 bp, synthetic construct
  source 1..46: mol_type = other DNA
ctcccccgaaccgcctggccctgaacagctccgcctcttttgtcgt

SEQ ID NO: 184 - DNA, 21 bp, synthetic construct
  source 1..21: mol_type = other DNA
tcatttgtcaatatattcatt

SEQ ID NO: 185 - DNA, 42 bp, synthetic construct
  source 1..42: mol_type = other DNA
tagcaagcaaagccgttcgcaaagtaaaggtttagcaattaa

SEQ ID NO: 186 - DNA, 42 bp, synthetic construct
  source 1..42: mol_type = other DNA
aggaagtaagattagttgctaaacctcccgacttggggaatt

SEQ ID NO: 187 - DNA, 39 bp, synthetic construct
  source 1..39: mol_type = other DNA
aaccaagtcaataataatttaatcaacaaataacgcaga

SEQ ID NO: 188 - DNA, 35 bp, synthetic construct
  source 1..35: mol_type = other DNA
aagaacgtcatcgtaccgcgcgaggcgtttcaatt

SEQ ID NO: 189 - DNA, 26 bp, synthetic construct
  source 1..26: mol_type = other DNA
ataatattatattttgcacccagcta

SEQ ID NO: 190 - DNA, 42 bp, synthetic construct
  source 1..42: mol_type = other DNA
aacaatattgccagttacaaatattaccaacgctagaatcaa

SEQ ID NO: 191 - DNA, 39 bp, synthetic construct
  source 1..39: mol_type = other DNA
catgttccgacaaaccagtaatatttaaagcaagagaat

SEQ ID NO: 192 - DNA, 28 bp, synthetic construct
  source 1..28: mol_type = other DNA
gaggcatggaaataaacagccttttttg

SEQ ID NO: 193 - DNA, 35 bp, synthetic construct
  source 1..35: mol_type = other DNA
cttaccagtataaaaacatgtaatttactaacata

SEQ ID NO: 194 - DNA, 28 bp, synthetic construct
  source 1..28: mol_type = other DNA
attcttcaataagaacgtcaacccgaga

SEQ ID NO: 195 - DNA, 34 bp, synthetic construct
  source 1..34: mol_type = other DNA
ctaccggcgagaggtgccacccaaatcaagtttt

SEQ ID NO: 196 - DNA, 49 bp, synthetic construct
  source 1..49: mol_type = other DNA
ttttgctcatggaaatacctaagtcacataaaagggacattcaagcgta

SEQ ID NO: 197 - DNA, 49 bp, synthetic construct
  source 1..49: mol_type = other DNA
tataatcgcacttaggttgggttataccttttatcaaaatcatagtttt

SEQ ID NO: 198 - DNA, 32 bp, synthetic construct
  source 1..32: mol_type = other DNA
cttgaaatattaattaaccttgcttctgtttt

SEQ ID NO: 199 - DNA, 35 bp, synthetic construct
  source 1..35: mol_type = other DNA
tttttagattaagacgctgagaagagtctagaatc

SEQ ID NO: 200 - DNA, 30 bp, synthetic construct
  source 1..30: mol_type = other DNA
ttttaatgctgatgcaaatccttatcccaa

SEQ ID NO: 201 - DNA, 28 bp, synthetic construct
  source 1..28: mol_type = other DNA
aatttaattagttagcgagaaaactttt

SEQ ID NO: 202 - DNA, 30 bp, synthetic construct
  source 1..30: mol_type = other DNA
ttttccgaccgtgtgatctatcacctaaag

SEQ ID NO: 203 - DNA, 31 bp, synthetic construct
  source 1..31: mol_type = other DNA
ttttggggtcacgtggcgagaaaggaatttt

SEQ ID NO: 204 - DNA, 42 bp, synthetic construct
  source 1..42: mol_type = other DNA
ttttagaaagcgaaaggagcgccgccgcgcttaatgcgtttt

SEQ ID NO: 205 - DNA, 42 bp, synthetic construct
  source 1..42: mol_type = other DNA
tttttacagggcgcgtactataagggattttagacaggtttt

SEQ ID NO: 206 - DNA, 42 bp, synthetic construct
  source 1..42: mol_type = other DNA
ttttgtacgccagaatcctgagcaaattaaccgttgtatttt

SEQ ID NO: 207 - DNA, 43 bp, synthetic construct
  source 1..43: mol_type = other DNA
tttttacttctttgattagtaagccattgcaacaggaaatttt

SEQ ID NO: 208 - DNA, 36 bp, synthetic construct
  source 1..36: mol_type = other DNA
tttttaccttttacatcgatgaatatacagtatttt

SEQ ID NO: 209 - DNA, 36 bp, synthetic construct
  source 1..36: mol_type = other DNA
ttttattacctgagcaaaggcgaattattcattttt

SEQ ID NO: 210 - DNA, 38 bp, synthetic construct
  source 1..38: mol_type = other DNA
ttttaaacagtacataaaaattaccttttttaattttt

SEQ ID NO: 211 - DNA, 26 bp, synthetic construct
  source 1..26: mol_type = other DNA
ttttataacatcacaatattactttt

SEQ ID NO: 212 - DNA, 54 bp, synthetic construct
  source 1..54: mol_type = other DNA
ttttcgccagccattgcaactccagaacttgcctacttctttgattagtatttt

SEQ ID NO: 213 - DNA, 26 bp, synthetic construct
  source 1..26: mol_type = other DNA
ttttatcgtctgaggacattcttttt

SEQ ID NO: 214 - DNA, 54 bp, synthetic construct
  source 1..54: mol_type = other DNA
ttttggccaacagagatagaataaaagaatggattacattttgacgctcatttt

SEQ ID NO: 215 - DNA, 26 bp, synthetic construct
  source 1..26: mol_type = other DNA
ttttaatattttttaaaaatactttt

SEQ ID NO: 216 - DNA, 54 bp, synthetic construct
  source 1..54: mol_type = other DNA
ttttcgaacgaaccaccagctcgccatgaatggcaatacgtggcacagactttt

SEQ ID NO: 217 - DNA, 40 bp, synthetic construct
  source 1..40: mol_type = other DNA
ttttcctgcaacagtgccacgtcagtattaacaccgtttt

SEQ ID NO: 218 - DNA, 26 bp, synthetic construct
  source 1..26: mol_type = other DNA
ttttacctcaaatcaaatcaactttt

SEQ ID NO: 219 - DNA, 29 bp, synthetic construct
  source 1..29: mol_type = other DNA
ttttagttgaaaggagcactaacaatttt

SEQ ID NO: 220 - DNA, 29 bp, synthetic construct
  source 1..29: mol_type = other DNA
ttttctaatagattaggaagtattatttt

SEQ ID NO: 221 - DNA, 27 bp, synthetic construct
  source 1..27: mol_type = other DNA
ttttgactttacaccgaacgttatttt

SEQ ID NO: 222 - DNA, 29 bp, synthetic construct
  source 1..29: mol_type = other DNA
ttttaattttaaaagtaaccaccagtttt

SEQ ID NO: 223 - DNA, 29 bp, synthetic construct
  source 1..29: mol_type = other DNA
ttttaaggagcggggcaattcatcatttt

SEQ ID NO: 224 - DNA, 31 bp, synthetic construct
  source 1..31: mol_type = other DNA
ttttatataatcctgatgtttttataatttt

SEQ ID NO: 225 - DNA, 25 bp, synthetic construct
  source 1..25: mol_type = other DNA
ttttaaacatagcatagtgaatttt

SEQ ID NO: 226 - DNA, 52 bp, synthetic construct
  source 1..52: mol_type = other DNA
ttttatcaaaatcatattagagtcagatagctcccttagaatccttgatttt

SEQ ID NO: 227 - DNA, 25 bp, synthetic construct
  source 1..25: mol_type = other DNA
ttttaacctccggctgatgcatttt

SEQ ID NO: 228 - DNA, 28 bp, synthetic construct
  source 1..28: mol_type = other DNA
ttttaatccaatcatatattttagtttt

SEQ ID NO: 229 - DNA, 29 bp, synthetic construct
  source 1..29: mol_type = other DNA
tttttaatttcatcttcgtgtgatatttt

SEQ ID NO: 230 - DNA, 29 bp, synthetic construct
  source 1..29: mol_type = other DNA
ttttaataaggcgctagaaaaagcctttt

SEQ ID NO: 231 - DNA, 31 bp, synthetic construct
  source 1..31: mol_type = other DNA
ttttgtttagtatcagagcgggagctatttt

SEQ ID NO: 232 - DNA, 25 bp, synthetic construct
  source 1..25: mol_type = other DNA
ttttggaatcattaaggcttatttt

SEQ ID NO: 233 - DNA, 29 bp, synthetic construct
  source 1..29: mol_type = other DNA
ttttccggtattcacttgcgggaggtttt

SEQ ID NO: 234 - DNA, 21 bp, synthetic construct
  source 1..21: mol_type = other DNA
ttttgaagcctttacaatttt

SEQ ID NO: 235 - DNA, 29 bp, synthetic construct
  source 1..29: mol_type = other DNA
ttttatcctgaatctaatttgccagtttt

SEQ ID NO: 236 - DNA, 23 bp, synthetic construct
  source 1..23: mol_type = other DNA
ttttacaaaataaaaacgatttt

SEQ ID NO: 237 - DNA, 27 bp, synthetic construct
  source 1..27: mol_type = other DNA
ttttgtttaacgaataacataaatttt

SEQ ID NO: 238 - DNA, 32 bp, synthetic construct
  source 1..32: mol_type = other DNA
ttttaacagggaagcggcgccgctacagtttt

SEQ ID NO: 239 - DNA, 26 bp, synthetic construct
  source 1..26: mol_type = other DNA
ttttgacattcaaaaattattctttt

SEQ ID NO: 240 - DNA, 29 bp, synthetic construct
  source 1..29: mol_type = other DNA
ttttattaaaggtggaattagagcctttt

SEQ ID NO: 241 - DNA, 29 bp, synthetic construct
  source 1..29: mol_type = other DNA
ttttagcaaaatcacccgtcaccaatttt

SEQ ID NO: 242 - DNA, 27 bp, synthetic construct
  source 1..27: mol_type = other DNA
ttttgaaaccattcaagtttgcctttt

SEQ ID NO: 243 - DNA, 25 bp, synthetic construct
  source 1..25: mol_type = other DNA
ttttagcgtcagacagccccctttt

SEQ ID NO: 244 - DNA, 29 bp, synthetic construct
  source 1..29: mol_type = other DNA
ttttattagcgttcagagccaccactttt

SEQ ID NO: 245 - DNA, 32 bp, synthetic construct
  source 1..32: mol_type = other DNA
ttttcggaaccgcctcagcgggcgctagtttt

SEQ ID NO: 246 - DNA, 26 bp, synthetic construct
  source 1..26: mol_type = other DNA
ttttaagagaagggcggataagtttt

SEQ ID NO: 247 - DNA, 28 bp, synthetic construct
  source 1..28: mol_type = other DNA
ttttgccgtcgagtatcaccgtactttt

SEQ ID NO: 248 - DNA, 28 bp, synthetic construct
  source 1..28: mol_type = other DNA
ttttcaggaggtttaaccgccacctttt

SEQ ID NO: 249 - DNA, 29 bp, synthetic construct
  source 1..29: mol_type = other DNA
ttttctcagagcctaggaacccatgtttt

SEQ ID NO: 250 - DNA, 28 bp, synthetic construct
  source 1..28: mol_type = other DNA
ttttaccgtaacactgtagcattctttt

SEQ ID NO: 251 - DNA, 29 bp, synthetic construct
  source 1..29: mol_type = other DNA
ttttcacagacaggtcgtctttccatttt

SEQ ID NO: 252 - DNA, 32 bp, synthetic construct
  source 1..32: mol_type = other DNA
ttttgacgttagtaaagcccccgatttatttt

SEQ ID NO: 253 - DNA, 25 bp, synthetic construct
  source 1..25: mol_type = other DNA
ttttaaaatacgcagcgattatttt

SEQ ID NO: 254 - DNA, 28 bp, synthetic construct
  source 1..28: mol_type = other DNA
ttttaccaagcggacggtcaatcatttt

SEQ ID NO: 255 - DNA, 28 bp, synthetic construct
  source 1..28: mol_type = other DNA
ttttaagggaaccgagtaatcttgtttt

SEQ ID NO: 256 - DNA, 27 bp, synthetic construct
  source 1..27: mol_type = other DNA
ttttacaagaacccttgagatggtttt

SEQ ID NO: 257 - DNA, 28 bp, synthetic construct
  source 1..28: mol_type = other DNA
ttttaatttcaacttctacgttaatttt

SEQ ID NO: 258 - DNA, 28 bp, synthetic construct
  source 1..28: mol_type = other DNA
ttttaaaacgaagatacataacgctttt

SEQ ID NO: 259 - DNA, 32 bp, synthetic construct
  source 1..32: mol_type = other DNA
ttttcaaaaggaattagaaccatcaccctttt

SEQ ID NO: 260 - DNA, 46 bp, synthetic construct
  source 1..46: mol_type = other DNA
actacgtcgaggcaaagttttccctcataacgcctgagtttcgaca

SEQ ID NO: 261 - DNA, 35 bp, synthetic construct
  source 1..35: mol_type = other DNA
agtgttgagggcgaaaaaccgctatcattgagaat

SEQ ID NO: 262 - DNA, 49 bp, synthetic construct
  source 1..49: mol_type = other DNA
gatagactgctaaagccgccaccagatcccctcagggaagggtgcgcgt

SEQ ID NO: 263 - DNA, 42 bp, synthetic construct
  source 1..42: mol_type = other DNA
aggcctcagaacagagagtcaaaaaataagacagccattttt

SEQ ID NO: 264 - DNA, 42 bp, synthetic construct
  source 1..42: mol_type = other DNA
aagagttgcagcaaaatcctgtttgaaaaaccgccagcgcta

SEQ ID NO: 265 - DNA, 49 bp, synthetic construct
  source 1..49: mol_type = other DNA
tgagaccgaacaccttaattgagaatacattcttagtgctttagacagg

SEQ ID NO: 266 - DNA, 45 bp, synthetic construct
  source 1..45: mol_type = other DNA
cgcgtcacgcaagaaagggcgaacgaaccctcgaggtgatggccc

SEQ ID NO: 267 - DNA, 35 bp, synthetic construct
  source 1..35: mol_type = other DNA
aatcattagaataattattaaatataccgacctga

SEQ ID NO: 268 - DNA, 42 bp, synthetic construct
  source 1..42: mol_type = other DNA
aaaatcggccaacgagggtggtttttacccagtataattatt

SEQ ID NO: 269 - DNA, 49 bp, synthetic construct
  source 1..49: mol_type = other DNA
attaaagtgagaagttgtttgggtaataaggaaaaaaatacctatttac

SEQ ID NO: 270 - DNA, 42 bp, synthetic construct
  source 1..42: mol_type = other DNA
taatgcgataatggcaattccaatcatgccccgggcggccag

SEQ ID NO: 271 - DNA, 46 bp, synthetic construct
  source 1..46: mol_type = other DNA
accgttgaagagtcagaatccggattttcctcgttttgacgaccgc

SEQ ID NO: 272 - DNA, 26 bp, synthetic construct
  source 1..26: mol_type = other DNA
gagccggccgctcaaagggttagaac

SEQ ID NO: 273 - DNA, 21 bp, synthetic construct
  source 1..21: mol_type = other DNA
agaactcaaactaccaaatta

SEQ ID NO: 274 - DNA, 32 bp, synthetic construct
  source 1..32: mol_type = other DNA
ccttgctgattatagattatctatacaacgga

SEQ ID NO: 275 - DNA, 18 bp, synthetic construct
  source 1..18: mol_type = other DNA
acgctcgttgcggaatca

SEQ ID NO: 276 - DNA, 26 bp, synthetic construct
  source 1..26: mol_type = other DNA
tgccaagcacgacgagatgaatatac

SEQ ID NO: 277 - DNA, 46 bp, synthetic construct
  source 1..46: mol_type = other DNA
attggcatcacacgacattatattaaataaatttagaaaactatta

SEQ ID NO: 278 - DNA, 46 bp, synthetic construct
  source 1..46: mol_type = other DNA
tgaccatcatttgacgacaacaactataaaagaacattttgcacgc

SEQ ID NO: 279 - DNA, 35 bp, synthetic construct
  source 1..35: mol_type = other DNA
aagtattagtctttaatatagcccaatagattaaa

SEQ ID NO: 280 - DNA, 26 bp, synthetic construct
  source 1..26: mol_type = other DNA
ggaagggcgccatttcatttcaatta

SEQ ID NO: 281 - DNA, 21 bp, synthetic construct
  source 1..21: mol_type = other DNA
gcgcgcagaaagggggtgaaa

SEQ ID NO: 282 - DNA, 49 bp, synthetic construct
  source 1..49: mol_type = other DNA
gtgtttcattaaacaaaattccaacaataatcatacatagtatgtagtt

SEQ ID NO: 283 - DNA, 46 bp, synthetic construct
  source 1..46: mol_type = other DNA
aaaacgcatctggtggaaggtgctgagatacgagccaaatcagcga

SEQ ID NO: 284 - DNA, 28 bp, synthetic construct
  source 1..28: mol_type = other DNA
gcggctgagagccagcaaatctaacctc

SEQ ID NO: 285 - DNA, 35 bp, synthetic construct
  source 1..35: mol_type = other DNA
ccagttttgggcgcagtacatctgtaaacaaattg

SEQ ID NO: 286 - DNA, 21 bp, synthetic construct
  source 1..21: mol_type = other DNA
gcaaagtctccagccaagagg

SEQ ID NO: 287 - DNA, 17 bp, synthetic construct
  source 1..17: mol_type = other DNA
attctccgtgggaacaa

SEQ ID NO: 288 - DNA, 39 bp, synthetic construct
  source 1..39: mol_type = other DNA
acggcggtaaatgtaaataatttttgttaatcagaggta

SEQ ID NO: 289 - DNA, 42 bp, synthetic construct
  source 1..42: mol_type = other DNA
gataggtgaagccagctttcatcaacatattgaccgtaatgg

SEQ ID NO: 290 - DNA, 28 bp, synthetic construct
  source 1..28: mol_type = other DNA
aattaatttttagattaaagccgtccaa

SEQ ID NO: 291 - DNA, 35 bp, synthetic construct
  source 1..35: mol_type = other DNA
atcaagatgaattacctttattttccggcgaactg

SEQ ID NO: 292 - DNA, 14 bp, synthetic construct
  source 1..14: mol_type = other DNA
cctgagcaaaagaa

SEQ ID NO: 293 - DNA, 49 bp, synthetic construct
  source 1..49: mol_type = other DNA
tgaaatcaagaggcgaattatcaggctgcaccgctgatcgcagcatctg

SEQ ID NO: 294 - DNA, 28 bp, synthetic construct
  source 1..28: mol_type = other DNA
atattgggttccatcctgattagttagc

SEQ ID NO: 295 - DNA, 42 bp, synthetic construct
  source 1..42: mol_type = other DNA
gcgcatagtgctgcacaccaggattcgataccgagctcatgg

SEQ ID NO: 296 - DNA, 24 bp, synthetic construct
  source 1..24: mol_type = other DNA
agtaacagtaccaaagtaccgaca

SEQ ID NO: 297 - DNA, 49 bp, synthetic construct
  source 1..49: mol_type = other DNA
tcggggtcaaggtttaacgtcttgtaaaaaggcgaagctggcactgttg

SEQ ID NO: 298 - DNA, 33 bp, synthetic construct
  source 1..33: mol_type = other DNA
aaattgcgtagattttcttaattcgtacatcgg

SEQ ID NO: 299 - DNA, 35 bp, synthetic construct
  source 1..35: mol_type = other DNA
tattttgaaaagaataacaatccaatgaaaagcat

SEQ ID NO: 300 - DNA, 24 bp, synthetic construct
  source 1..24: mol_type = other DNA
ctaccatatcaaaaagccaacgct

SEQ ID NO: 301 - DNA, 49 bp, synthetic construct
  source 1..49: mol_type = other DNA
tgcacctcttctgagtacgcctgtccatcattgcgctcactggctgcat

SEQ ID NO: 302 - DNA, 41 bp, synthetic construct
  source 1..41: mol_type = other DNA
ggccttcctgtgtttgttaaaggaagagtaacaagagcatt

SEQ ID NO: 303 - DNA, 23 bp, synthetic construct
  source 1..23: mol_type = other DNA
taaacgtccttatcattaattac

SEQ ID NO: 304 - DNA, 49 bp, synthetic construct
  source 1..49: mol_type = other DNA
agtaccgtcgtcgctattaatcatttaatggaaacatcgtaacctgaaa

SEQ ID NO: 305 - DNA, 14 bp, synthetic construct
  source 1..14: mol_type = other DNA
cataacggaatacc

SEQ ID NO: 306 - DNA, 18 bp, synthetic construct
  source 1..18: mol_type = other DNA
atgcagaaccctgattgc

SEQ ID NO: 307 - DNA, 28 bp, synthetic construct
  source 1..28: mol_type = other DNA
acaatattcagaaacaataaccaaaatc

SEQ ID NO: 308 - DNA, 49 bp, synthetic construct
  source 1..49: mol_type = other DNA
caagcaagactgtaaatgcttaggtctttaggaattgacagttggatca

SEQ ID NO: 309 - DNA, 49 bp, synthetic construct
  source 1..49: mol_type = other DNA
cctcctttgcaacaattggatttaagccgtctaaaacaagaagattgag

SEQ ID NO: 310 - DNA, 40 bp, synthetic construct
  source 1..40: mol_type = other DNA
tatttaattcgagccagtaggtcgtaaaacagaaataaag

SEQ ID NO: 311 - DNA, 24 bp, synthetic construct
  source 1..24: mol_type = other DNA
caacagtagggcctgaacaaagtc

SEQ ID NO: 312 - DNA, 35 bp, synthetic construct
  source 1..35: mol_type = other DNA
atccgggagaagcctttgccgccaaaaatcatctg

SEQ ID NO: 313 - DNA, 49 bp, synthetic construct
  source 1..49: mol_type = other DNA
cgtagatgataattatcacaaagattgagtaaccagtaacccttcgcgt

SEQ ID NO: 314 - DNA, 28 bp, synthetic construct
  source 1..28: mol_type = other DNA
atttactcgcaaagaatagaattaccat

SEQ ID NO: 315 - DNA, 45 bp, synthetic construct
  source 1..45: mol_type = other DNA
atcaggtcatatgccgggtaggtatttttagaatacttgagcata

SEQ ID NO: 316 - DNA, 43 bp, synthetic construct
  source 1..43: mol_type = other DNA
gatgaacaaagccccaaaaacaattcgcattaaattcgcgtct

SEQ ID NO: 317 - DNA, 46 bp, synthetic construct
  source 1..46: mol_type = other DNA
tagaccagcgagggagggtattaattagcggtgaggaaactacgaa

SEQ ID NO: 318 - DNA, 23 bp, synthetic construct
  source 1..23: mol_type = other DNA
taggaataattgtataagcaaat

SEQ ID NO: 319 - DNA, 42 bp, synthetic construct
  source 1..42: mol_type = other DNA
gcaacagatgtagataatatcatagataagtcctgaagatga

SEQ ID NO: 320 - DNA, 45 bp, synthetic construct
  source 1..45: mol_type = other DNA
ggcgtaaatacaccgtcttgctcagatataatcatcttaagtaca

SEQ ID NO: 321 - DNA, 14 bp, synthetic construct
  source 1..14: mol_type = other DNA
caaaagaactggtg

SEQ ID NO: 322 - DNA, 46 bp, synthetic construct
  source 1..46: mol_type = other DNA
gaaccattacacttgagcaccctcagcccggaactttgcggaacga

SEQ ID NO: 323 - DNA, 42 bp, synthetic construct
  source 1..42: mol_type = other DNA
gcaataaagaaaaaaaccaatgaacgggtattaactacaaac

SEQ ID NO: 324 - DNA, 42 bp, synthetic construct
  source 1..42: mol_type = other DNA
tttaagaaaggaaaccgagctgccgacgacaataatttatca

SEQ ID NO: 325 - DNA, 45 bp, synthetic construct
  source 1..45: mol_type = other DNA
taaagcaaggagcaccgccgccactcattttgaccttccattacc

SEQ ID NO: 326 - DNA, 24 bp, synthetic construct
  source 1..24: mol_type = other DNA
atagcaatagctcacaaacaaata

SEQ ID NO: 327 - DNA, 42 bp, synthetic construct
  source 1..42: mol_type = other DNA
aagagcaattctgtccagagaatataagagaatatttttaca

SEQ ID NO: 328 - DNA, 35 bp, synthetic construct
  source 1..35: mol_type = other DNA
tttcgtctttcgttttccagtagcgtcaccagata

SEQ ID NO: 329 - DNA, 26 bp, synthetic construct
  source 1..26: mol_type = other DNA
gaattgagttaagcccaccaccgcca

SEQ ID NO: 330 - DNA, 24 bp, synthetic construct
  source 1..24: mol_type = other DNA
agagggtaattgaccctcagagcc

SEQ ID NO: 331 - DNA, 49 bp, synthetic construct
  source 1..49: mol_type = other DNA
atatcgcattaactaccacacgcacgtatacttttcaccagtctggccc

SEQ ID NO: 332 - DNA, 42 bp, synthetic construct
  source 1..42: mol_type = other DNA
atggtttcaatataaaagaaacatcgagaacaagcagaacca

SEQ ID NO: 333 - DNA, 43 bp, synthetic construct
  source 1..43: mol_type = other DNA
accgttctgagaaacattcaacgcaaggataaaaaaagattca

SEQ ID NO: 334 - DNA, 23 bp, synthetic construct
  source 1..23: mol_type = other DNA
caatatgtataaactagcaaacg

SEQ ID NO: 335 - DNA, 46 bp, synthetic construct
  source 1..46: mol_type = other DNA
cgatattcaattttgtcacaatcacaccacgaaaatacggctgtct

SEQ ID NO: 336 - DNA, 38 bp, synthetic construct
  source 1..38: mol_type = other DNA
aatcttgagttttgcggggcttgccaaaagacatcgcc

SEQ ID NO: 337 - DNA, 42 bp, synthetic construct
  source 1..42: mol_type = other DNA
tcagtgcgccccctgcctaaggttccttattacgcaaaggtg

SEQ ID NO: 338 - DNA, 14 bp, synthetic construct
  source 1..14: mol_type = other DNA
gtaatgacaacaac

SEQ ID NO: 339 - DNA, 42 bp, synthetic construct
  source 1..42: mol_type = other DNA
tttaacgcaacaggagtgtaccatgattaagactttggaaac

SEQ ID NO: 340 - DNA, 24 bp, synthetic construct
  source 1..24: mol_type = other DNA
aatcctcattaatctccaaaaaaa

SEQ ID NO: 341 - DNA, 42 bp, synthetic construct
  source 1..42: mol_type = other DNA
atggaatatggccttgatattatcttaccgaagagatataat

SEQ ID NO: 342 - DNA, 49 bp, synthetic construct
  source 1..49: mol_type = other DNA
gaacgatagcccggaaaagtagcacctcccgtaagaacgatatagaccg

SEQ ID NO: 343 - DNA, 26 bp, synthetic construct
  source 1..26: mol_type = other DNA
cgattcgtaagagagataacccacaa

SEQ ID NO: 344 - DNA, 38 bp, synthetic construct
  source 1..38: mol_type = other DNA
tttttttcataaactacagttagcttgggaaaacaaca

SEQ ID NO: 345 - DNA, 24 bp, synthetic construct
  source 1..24: mol_type = other DNA
accaccctcagacaactttcaaca

SEQ ID NO: 346 - DNA, 49 bp, synthetic construct
  source 1..49: mol_type = other DNA
aactgccatccggtcattgtagcgccagagccttaccaacccagcaaat

SEQ ID NO: 347 - DNA, 41 bp, synthetic construct
  source 1..41: mol_type = other DNA
aaagggtagctgataaattatgcctgagagtctggagaatc

SEQ ID NO: 348 - DNA, 28 bp, synthetic construct
  source 1..28: mol_type = other DNA
aaatcatacagcattgaggacaacgaaa

SEQ ID NO: 349 - DNA, 42 bp, synthetic construct
  source 1..42: mol_type = other DNA
gcgaaagtctgaaacatgaaagatttcggaacctaaattcat

SEQ ID NO: 350 - DNA, 23 bp, synthetic construct
  source 1..23: mol_type = other DNA
aaagggtagggccggagacagtc

SEQ ID NO: 351 - DNA, 26 bp, synthetic construct
  source 1..26: mol_type = other DNA
catcgcccacgcaacggtgacctgct

SEQ ID NO: 352 - DNA, 28 bp, synthetic construct
  source 1..28: mol_type = other DNA
atataatagagttgcgccgacaataagt

SEQ ID NO: 353 - DNA, 37 bp, synthetic construct
  source 1..37: mol_type = other DNA
tgaatttcttaaacagcagcttggaccaggaaagctg

SEQ ID NO: 354 - DNA, 42 bp, synthetic construct
  source 1..42: mol_type = other DNA
ccgataataaagcgcagtctcgcttttgatgatttcgccctt

SEQ ID NO: 355 - DNA, 42 bp, synthetic construct
  source 1..42: mol_type = other DNA
ccccctcagagtaccgcccatttggaattatttgacggccga

SEQ ID NO: 356 - DNA, 26 bp, synthetic construct
  source 1..26: mol_type = other DNA
aggctccaaaagttaagaactgacga

SEQ ID NO: 357 - DNA, 28 bp, synthetic construct
  source 1..28: mol_type = other DNA
taattgggttcacgttgaaaaagccaga

SEQ ID NO: 358 - DNA, 18 bp, synthetic construct
  source 1..18: mol_type = other DNA
gcatgtgaatagtagtaa

SEQ ID NO: 359 - DNA, 40 bp, synthetic construct
  source 1..40: mol_type = other DNA
tgcgaataataatttttacaaaccaccaccagaggtcaga

SEQ ID NO: 360 - DNA, 26 bp, synthetic construct
  source 1..26: mol_type = other DNA
gtttcagcggagaaccctcgttgaga

SEQ ID NO: 361 - DNA, 49 bp, synthetic construct
  source 1..49: mol_type = other DNA
agaaaaagggattttaaatcggtggcgagatggtggttccgaagcccga

SEQ ID NO: 362 - DNA, 24 bp, synthetic construct
  source 1..24: mol_type = other DNA
tatcgatctatagtaagattcaac

SEQ ID NO: 363 - DNA, 49 bp, synthetic construct
  source 1..49: mol_type = other DNA
agaagcctttatacaaattaagcaataacatccaataaatcaaataacc

SEQ ID NO: 364 - DNA, 30 bp, synthetic construct
  source 1..30: mol_type = other DNA
gaggcttcggaacgggccgctaacagtgcc

SEQ ID NO: 365 - DNA, 21 bp, synthetic construct
  source 1..21: mol_type = other DNA
gaaaagaattagcaggctaca

SEQ ID NO: 366 - DNA, 48 bp, synthetic construct
  source 1..48: mol_type = other DNA
ggcaccaaaacacgtttcggtcgctgagatcgtcaccctttaccgggg

SEQ ID NO: 367 - DNA, 42 bp, synthetic construct
  source 1..42: mol_type = other DNA
tgtttagtacatttaagtttcgtagctcaacatgtagagagt

SEQ ID NO: 368 - DNA, 30 bp, synthetic construct
  source 1..30: mol_type = other DNA
ttgtgtctacaggcaaggcggagggagtta

SEQ ID NO: 369 - DNA, 49 bp, synthetic construct
  source 1..49: mol_type = other DNA
tgataaaatccgcgtaactaaagtacggtgtctggcgcaaatggtcgaa

SEQ ID NO: 370 - DNA, 49 bp, synthetic construct
  source 1..49: mol_type = other DNA
acggagaacttagcaaagagggctggctcagtatcggtttatcttgata

SEQ ID NO: 371 - DNA, 49 bp, synthetic construct
  source 1..49: mol_type = other DNA
ccatgtttttgtatatacactaacctaataaagactttttcaggcagca

SEQ ID NO: 372 - DNA, 49 bp, synthetic construct
  source 1..49: mol_type = other DNA
ataacaggtttgaccattagactatattgcattaaagcctcattgcggg

SEQ ID NO: 373 - DNA, 42 bp, synthetic construct
  source 1..42: mol_type = other DNA
acctttactccaacgaagccctattatagtcagaacattgaa

SEQ ID NO: 374 - DNA, 37 bp, synthetic construct
  source 1..37: mol_type = other DNA
ctcattcagtgatttttaaatatgcacactttcgagg

SEQ ID NO: 375 - DNA, 47 bp, synthetic construct
  source 1..47: mol_type = other DNA
caaatcacagaacgtaccttacaggacggtggaacaactaaaggaat

SEQ ID NO: 376 - DNA, 35 bp, synthetic construct
  source 1..35: mol_type = other DNA
gaaacacacgtaaccgcatagacagatgataaccg

SEQ ID NO: 377 - DNA, 45 bp, synthetic construct
  source 1..45: mol_type = other DNA
attgggggatattatcaagaactgaccaataggtgagggttgtac

SEQ ID NO: 378 - DNA, 42 bp, synthetic construct
  source 1..42: mol_type = other DNA
ttcaaatagaccggaagcaaaattgctctaatgctattccat

SEQ ID NO: 379 - DNA, 35 bp, synthetic construct
  source 1..35: mol_type = other DNA
ttaagagaggtcaggaataaggcttgccctgcatc

SEQ ID NO: 380 - DNA, 49 bp, synthetic construct
  source 1..49: mol_type = other DNA
ggattggctcattaaagattcatgtcataaatattgcaaagcaaaaaga

SEQ ID NO: 381 - DNA, 28 bp, synthetic construct
  source 1..28: mol_type = other DNA
tcccccttggatagaccaaaatagcgag

SEQ ID NO: 382 - DNA, 42 bp, synthetic construct
  source 1..42: mol_type = other DNA
ttattacataccacagcaacatctatcaccgtaaagcggttg

SEQ ID NO: 383 - DNA, 35 bp, synthetic construct
  source 1..35: mol_type = other DNA
tttaggaaggtagataccagttgcgattgagcctt

SEQ ID NO: 384 - DNA, 39 bp, synthetic construct
  source 1..39: mol_type = other DNA
taatgcactaacgggaaaaattaatcatagcccaaacca

SEQ ID NO: 385 - DNA, 42 bp, synthetic construct
  source 1..42: mol_type = other DNA
aggcttttaaaatgtttagaccaaatgcccctgacgaaagac

SEQ ID NO: 386 - DNA, 42 bp, synthetic construct
  source 1..42: mol_type = other DNA
agacgacgataaaacgtccaatactgcggaatccagtttacc

SEQ ID NO: 387 - DNA, 50 bp, synthetic construct
  source 1..50: mol_type = other DNA
ttttgaacgtggactccaacgtcaattccagtttggaacaagagtctttt

SEQ ID NO: 388 - DNA, 37 bp, synthetic construct
  source 1..37: mol_type = other DNA
ttttaaattgttataagcataaagtgtaaagcctttt

SEQ ID NO: 389 - DNA, 42 bp, synthetic construct
  source 1..42: mol_type = other DNA
ttttggtcgactctagaggatgtcatagctgtttcctgtttt

SEQ ID NO: 390 - DNA, 24 bp, synthetic construct
  source 1..24: mol_type = other DNA
ttttcccagtcttgcatgcctttt

SEQ ID NO: 391 - DNA, 28 bp, synthetic construct
  source 1..28: mol_type = other DNA
ttttaagcgccattcgatcggtgctttt

SEQ ID NO: 392 - DNA, 28 bp, synthetic construct
  source 1..28: mol_type = other DNA
ttttcaagagcgagtaacaacccgtttt

SEQ ID NO: 393 - DNA, 27 bp, synthetic construct
  source 1..27: mol_type = other DNA
ttttatgtaccccggttgataaatttt

SEQ ID NO: 394 - DNA, 28 bp, synthetic construct
  source 1..28: mol_type = other DNA
ttttatcgtaaaactagcatgtcatttt

SEQ ID NO: 395 - DNA, 28 bp, synthetic construct
  source 1..28: mol_type = other DNA
ttttcaatgcctgagtaatgtagatttt

SEQ ID NO: 396 - DNA, 44 bp, synthetic construct
  source 1..44: mol_type = other DNA
ttttaaaaacattatgaccctgtaaccctcatatattttatttt

SEQ ID NO: 397 - DNA, 38 bp, synthetic construct
  source 1..38: mol_type = other DNA
ttttagagggggtaatagtgcaaaagaagttttgtttt

SEQ ID NO: 398 - DNA, 45 bp, synthetic construct
  source 1..45: mol_type = other DNA
ttttcctaatgagtgagctaactcacattaattgcgcgaacatac

SEQ ID NO: 399 - DNA, 31 bp, synthetic construct
  source 1..31: mol_type = other DNA
ttttctcttcgctattacgccttaagttcaa

SEQ ID NO: 400 - DNA, 29 bp, synthetic construct
  source 1..29: mol_type = other DNA
ttttagtatcggcctcaggaatctgtttt

SEQ ID NO: 401 - DNA, 37 bp, synthetic construct
  source 1..37: mol_type = other DNA
attatatgtcacgttggtgtagagaggggacgatttt

SEQ ID NO: 402 - DNA, 23 bp, synthetic construct
  source 1..23: mol_type = other DNA
tttgaatacgggtaacgcctttt

SEQ ID NO: 403 - DNA, 47 bp, synthetic construct
  source 1..47: mol_type = other DNA
tttaatattgaataaccttgcttaaatcaattaacaaccggaaacca

SEQ ID NO: 404 - DNA, 49 bp, synthetic construct
  source 1..49: mol_type = other DNA
aagctaatactaatagtagtattcatttggggcgcgagctgaaaatttt

SEQ ID NO: 405 - DNA, 32 bp, synthetic construct
  source 1..32: mol_type = other DNA
ttttaacgagtagatttattgattcttaattg

SEQ ID NO: 406 - DNA, 35 bp, synthetic construct
  source 1..35: mol_type = other DNA
ctgaatacttttgataagaggtcatttttgctttt

SEQ ID NO: 407 - DNA, 32 bp, synthetic construct
  source 1..32: mol_type = other DNA
ttttcttcaaagcgaaccatcgcgtaaatcag

SEQ ID NO: 408 - DNA, 35 bp, synthetic construct
  source 1..35: mol_type = other DNA
gtctttatttaaacagttcagaaaacgagaatttt

SEQ ID NO: 409 - DNA, 41 bp, synthetic construct
  source 1..41: mol_type = other DNA
ttttaaatcaagttttttggggtaaagggatgaatttccgg

SEQ ID NO: 410 - DNA, 27 bp, synthetic construct
  source 1..27: mol_type = other DNA
ttttgagcttgacggggaaagcccgaa

SEQ ID NO: 411 - DNA, 27 bp, synthetic construct
  source 1..27: mol_type = other DNA
ttttggcgctggcaagtgtagcggctt

SEQ ID NO: 412 - DNA, 34 bp, synthetic construct
  source 1..34: mol_type = other DNA
ttttggcgcgtactatggttgctagaatcatatg

SEQ ID NO: 413 - DNA, 16 bp, synthetic construct
  source 1..16: mol_type = other DNA
ttttaacaggaggccg

SEQ ID NO: 414 - DNA, 37 bp, synthetic construct
  source 1..37: mol_type = other DNA
ttttcagtgaggccaccgagtaatagcaatgagtaga

SEQ ID NO: 415 - DNA, 23 bp, synthetic construct
  source 1..23: mol_type = other DNA
aacagcatcaccttgctgatttt

SEQ ID NO: 416 - DNA, 23 bp, synthetic construct
  source 1..23: mol_type = other DNA
cgcttttattttcatcgtatttt

SEQ ID NO: 417 - DNA, 25 bp, synthetic construct
  source 1..25: mol_type = other DNA
caaaaggtctgagagactacctttt

SEQ ID NO: 418 - DNA, 23 bp, synthetic construct
  source 1..23: mol_type = other DNA
ttgccaaagacaaaagggctttt

SEQ ID NO: 419 - DNA, 30 bp, synthetic construct
  source 1..30: mol_type = other DNA
cagattaggagaggctgagactcctctttt

SEQ ID NO: 420 - DNA, 47 bp, synthetic construct
  source 1..47: mol_type = other DNA
ggcgcacgaaacatgacccctaatgccgtttccattaaacgggtttt

SEQ ID NO: 421 - DNA, 16 bp, synthetic construct
  source 1..16: mol_type = other DNA
tttttaaacactattt

SEQ ID NO: 422 - DNA, 36 bp, synthetic construct
  source 1..36: mol_type = other DNA
ggtataaatcaaaagaataatcggcaaaatccctga

SEQ ID NO: 423 - DNA, 36 bp, synthetic construct
  source 1..36: mol_type = other DNA
cctgccccagcaggcgaagcggtccacgctggttgc

SEQ ID NO: 424 - DNA, 36 bp, synthetic construct
  source 1..36: mol_type = other DNA
aagattgcccttcaccgcgagacgggcaacagctcg

SEQ ID NO: 425 - DNA, 36 bp, synthetic construct
  source 1..36: mol_type = other DNA
gcttgcgtattgggcgcccgcggggagaggcggtaa

SEQ ID NO: 426 - DNA, 35 bp, synthetic construct
  source 1..35: mol_type = other DNA
agaaacctgtcgtgccacccgctttccagtcggac

SEQ ID NO: 427 - DNA, 16 bp, synthetic construct
  source 1..16: mol_type = other DNA
gggagtaacgaccgtg

SEQ ID NO: 428 - DNA, 36 bp, synthetic construct
  source 1..36: mol_type = other DNA
tgttttgaatggctattagtggcacagacaatattg

SEQ ID NO: 429 - DNA, 36 bp, synthetic construct
  source 1..36: mol_type = other DNA
tgtgaggcggtcagtattgaagataaaacagaggca

SEQ ID NO: 430 - DNA, 36 bp, synthetic construct
  source 1..36: mol_type = other DNA
agaatatcaaaccctcaaaccttgctgaacctcagg

SEQ ID NO: 431 - DNA, 36 bp, synthetic construct
  source 1..36: mol_type = other DNA
ggtaatagattagagccgtaggagcactaacaacgc

SEQ ID NO: 432 - DNA, 36 bp, synthetic construct
  source 1..36: mol_type = other DNA
ggcccgaacgttattaatcgtattaaatcctttgca

SEQ ID NO: 433 - DNA, 36 bp, synthetic construct
  source 1..36: mol_type = other DNA
cgtcagatgatggcaattatcatattcctgattaac

SEQ ID NO: 434 - DNA, 36 bp, synthetic construct
  source 1..36: mol_type = other DNA
tcgaaataaagaaattgcatttgcacgtaaaacagg

SEQ ID NO: 435 - DNA, 34 bp, synthetic construct
  source 1..34: mol_type = other DNA
acccaataggaacgccacagctcattttttaaag

SEQ ID NO: 436 - DNA, 36 bp, synthetic construct
  source 1..36: mol_type = other DNA
atgcctgattgctttgaaaaacaataacggattcca

SEQ ID NO: 437 - DNA, 36 bp, synthetic construct
  source 1..36: mol_type = other DNA
ttgagatctacaaaggctgggtagctatttttgaca

SEQ ID NO: 438 - DNA, 36 bp, synthetic construct
  source 1..36: mol_type = other DNA
aatcaagaaaacaaaattgatgatgaaacaaacatg

SEQ ID NO: 439 - DNA, 28 bp, synthetic construct
  source 1..28: mol_type = other DNA
tatggcatcaattcatcggttgtaccgg

SEQ ID NO: 440 - DNA, 25 bp, synthetic construct
  source 1..25: mol_type = other DNA
ggaatcgtcgcacatagcgatagcg

SEQ ID NO: 441 - DNA, 25 bp, synthetic construct
  source 1..25: mol_type = other DNA
gtatggcttagagcccaattctgct

SEQ ID NO: 442 - DNA, 25 bp, synthetic construct
  source 1..25: mol_type = other DNA
ggctgagagactataactatatgag

SEQ ID NO: 443 - DNA, 25 bp, synthetic construct
  source 1..25: mol_type = other DNA
tcaccataaatcaatttaattcgta

SEQ ID NO: 444 - DNA, 25 bp, synthetic construct
  source 1..25: mol_type = other DNA
tgaaatatatttggtttgaaatacc

SEQ ID NO: 445 - DNA, 34 bp, synthetic construct
  source 1..34: mol_type = other DNA
atgaccataaatcgcctgataaatggagggaggg

SEQ ID NO: 446 - DNA, 34 bp, synthetic construct
  source 1..34: mol_type = other DNA
tgtgtcgaaatccctcagaaccgcggagggaggg

SEQ ID NO: 447 - DNA, 34 bp, synthetic construct
  source 1..34: mol_type = other DNA
caccctcagagcgcagcaccgtaaggagggaggg

SEQ ID NO: 448 - DNA, 42 bp, synthetic construct
  source 1..42: mol_type = other DNA
tttaggcagaggcattcaacgccaacatgtaaccagccagcc

SEQ ID NO: 449 - DNA, 42 bp, synthetic construct
  source 1..42: mol_type = other DNA
cgaacaaagttaccagaaagtaagcagatagcccagccagcc

SEQ ID NO: 450 - DNA, 42 bp, synthetic construct
  source 1..42: mol_type = other DNA
gtaagcgtcatacatgtgaatttaccgttccaccagccagcc

SEQ ID NO: 451 - DNA, 19 bp, synthetic construct
  source 1..19: mol_type = other DNA
ttgctttgacgagcacgta

SEQ ID NO: 452 - DNA, 24 bp, synthetic construct
  source 1..24: mol_type = other DNA
gccgctacagggcgcgtggtcaat

SEQ ID NO: 453 - DNA, 35 bp, synthetic construct
  source 1..35: mol_type = other DNA
taacgtgctttcaattctaccaccgagtaaaagtt

SEQ ID NO: 454 - DNA, 32 bp, synthetic construct
  source 1..32: mol_type = other DNA
aacctgtttagctagcttagtttgaccattag

SEQ ID NO: 455 - DNA, 48 bp, synthetic construct
  source 1..48: mol_type = other DNA
agggcgctgaacgtggcgagaaaggggagcccccgatttaggtcgagg

SEQ ID NO: 456 - DNA, 32 bp, synthetic construct
  source 1..32: mol_type = other DNA
ggtggcatcctcgttagaatcaaatactatgg

SEQ ID NO: 457 - DNA, 32 bp, synthetic construct
  source 1..32: mol_type = other DNA
gaaatattttcatttgagtacggtgctgaata

SEQ ID NO: 458 - DNA, 27 bp, synthetic construct
  source 1..27: mol_type = other DNA
atagtagtagcctaaatcgaaactatc

SEQ ID NO: 459 - DNA, 32 bp, synthetic construct
  source 1..32: mol_type = other DNA
gcaaggcaaagaaggagcttaattgtctggaa

SEQ ID NO: 460 - DNA, 43 bp, synthetic construct
  source 1..43: mol_type = other DNA
gcataaagattaacatcatgagtctgtccatcagcaaaatcac

SEQ ID NO: 461 - DNA, 32 bp, synthetic construct
  source 1..32: mol_type = other DNA
atcttagcaaaattaacaggattaattcgagc

SEQ ID NO: 462 - DNA, 48 bp, synthetic construct
  source 1..48: mol_type = other DNA
tgccgtaagtctatcagtgaaccattggaacaagagtccaaaagaata

SEQ ID NO: 463 - DNA, 27 bp, synthetic construct
  source 1..27: mol_type = other DNA
gtaccaaaaacaaaattttaataccta

SEQ ID NO: 464 - DNA, 32 bp, synthetic construct
  source 1..32: mol_type = other DNA
gggaacgtcaaagggcgcgttttagagagtac

SEQ ID NO: 465 - DNA, 43 bp, synthetic construct
  source 1..43: mol_type = other DNA
caaggataattatgacccgtgctggtaatatcgcgcagtctct

SEQ ID NO: 466 - DNA, 32 bp, synthetic construct
  source 1..32: mol_type = other DNA
gaacgtggactccagatagtcagacgagaatg

SEQ ID NO: 467 - DNA, 47 bp, synthetic construct
  source 1..47: mol_type = other DNA
aaccctcatataggccggagagggggtgcttttgctattcggttatt

SEQ ID NO: 468 - DNA, 32 bp, synthetic construct
  source 1..32: mol_type = other DNA
tgtggcaaaatccctttcagaaaaagcaaagc

SEQ ID NO: 469 - DNA, 48 bp, synthetic construct
  source 1..48: mol_type = other DNA
gcccgagagcaggcgaaaatcctgagagagttgcagcaagtttttctt

SEQ ID NO: 470 - DNA, 43 bp, synthetic construct
  source 1..43: mol_type = other DNA
ggtgagaaattttaaacagtgacgctcaatcggggatagcaag

SEQ ID NO: 471 - DNA, 24 bp, synthetic construct
  source 1..24: mol_type = other DNA
aaatcgtaagcgtccaccagacga

SEQ ID NO: 472 - DNA, 40 bp, synthetic construct
  source 1..40: mol_type = other DNA
agtcaaatcacctattttttatttttgatgtcaatcatat

SEQ ID NO: 473 - DNA, 32 bp, synthetic construct
  source 1..32: mol_type = other DNA
taggcccttcaccgccctcgtttaatactgcg

SEQ ID NO: 474 - DNA, 32 bp, synthetic construct
  source 1..32: mol_type = other DNA
gagggtagcatcaataagcgagagaatagtaa

SEQ ID NO: 475 - DNA, 48 bp, synthetic construct
  source 1..48: mol_type = other DNA
tgattctgctaatgcagagaatcggaagattgtattaactcacattaa

SEQ ID NO: 476 - DNA, 40 bp, synthetic construct
  source 1..40: mol_type = other DNA
ttcaccaggcggggagaggcggtttgcgtatttccagtcg

SEQ ID NO: 477 - DNA, 35 bp, synthetic construct
  source 1..35: mol_type = other DNA
agatctacaaaggctatcaaaactagcaatattta

SEQ ID NO: 478 - DNA, 40 bp, synthetic construct
  source 1..40: mol_type = other DNA
tctggagcaaaaatcggccaacgctgagacgggcaacagc

SEQ ID NO: 479 - DNA, 24 bp, synthetic construct
  source 1..24: mol_type = other DNA
taatcgtaggtcattgatgccgga

SEQ ID NO: 480 - DNA, 37 bp, synthetic construct
  source 1..37: mol_type = other DNA
gtaccccggttgataatcagaatattttgagatgcga

SEQ ID NO: 481 - DNA, 48 bp, synthetic construct
  source 1..48: mol_type = other DNA
ttgcgttggtcgtgccagctgcattaatgcaagatacataacaacatt

SEQ ID NO: 482 - DNA, 35 bp, synthetic construct
  source 1..35: mol_type = other DNA
aaacgttaaaagccccttcatcagttgagggccgc

SEQ ID NO: 483 - DNA, 32 bp, synthetic construct
  source 1..32: mol_type = other DNA
ctaatgagtgagcaagagtcaggaggtttaat

SEQ ID NO: 484 - DNA, 37 bp, synthetic construct
  source 1..37: mol_type = other DNA
taaatttttgttaaatcagcttaattcgcttggtaac

SEQ ID NO: 485 - DNA, 32 bp, synthetic construct
  source 1..32: mol_type = other DNA
gttatccgcatagctggcttgccctcttgaca

SEQ ID NO: 486 - DNA, 35 bp, synthetic construct
  source 1..35: mol_type = other DNA
atcaaaaacattttttgtgaattaccttaagaagc

SEQ ID NO: 487 - DNA, 48 bp, synthetic construct
  source 1..48: mol_type = other DNA
gtagccagctttcatcaacattccgtgggccgaactgcgcagacgacg

SEQ ID NO: 488 - DNA, 32 bp, synthetic construct
  source 1..32: mol_type = other DNA
tgccaagcacgacgttaacggtgtgacctgct

SEQ ID NO: 489 - DNA, 48 bp, synthetic construct
  source 1..48: mol_type = other DNA
ctgcaggtaattcgtaatcatggtctcacaattccacacatggggtgc

SEQ ID NO: 490 - DNA, 35 bp, synthetic construct
  source 1..35: mol_type = other DNA
tcggattctaaatgtgtacccaaatcaacctgcgg

SEQ ID NO: 491 - DNA, 37 bp, synthetic construct
  source 1..37: mol_type = other DNA
ttgaccgtaatgggataggtccatctgccgaccccca

SEQ ID NO: 492 - DNA, 32 bp, synthetic construct
  source 1..32: mol_type = other DNA
taaccgtgacgttggtgaacgaggaccaactt

SEQ ID NO: 493 - DNA, 48 bp, synthetic construct
  source 1..48: mol_type = other DNA
tgggaaggagctggcgaaagggggcagggttttcccagtcttgcatgc

SEQ ID NO: 494 - DNA, 45 bp, synthetic construct
  source 1..45: mol_type = other DNA
gacgacagtatcggcctcaggaagatcgcactccagcgcgcatcg

SEQ ID NO: 495 - DNA, 24 bp, synthetic construct
  source 1..24: mol_type = other DNA
cttctggtgccggaaagcaactgt

SEQ ID NO: 496 - DNA, 32 bp, synthetic construct
  source 1..32: mol_type = other DNA
atacatttgcaactaagggcgcgatcatacag

SEQ ID NO: 497 - DNA, 40 bp, synthetic construct
  source 1..40: mol_type = other DNA
gtttcattgagtagatgaaaggagccgccgcgcttaatgc

SEQ ID NO: 498 - DNA, 32 bp, synthetic construct
  source 1..32: mol_type = other DNA
taatgctgcaacaggtgcaataaaacttttgc

SEQ ID NO: 499 - DNA, 40 bp, synthetic construct
  source 1..40: mol_type = other DNA
ggcctgaagcaaactctagctcaaccaataaagctgaaaa

SEQ ID NO: 500 - DNA, 32 bp, synthetic construct
  source 1..32: mol_type = other DNA
ctttaattggcttagaaccctaaagaagggaa

SEQ ID NO: 501 - DNA, 48 bp, synthetic construct
  source 1..48: mol_type = other DNA
ccagccattcacttgcccatatttaaggcttacaatagcacgaattca

SEQ ID NO: 502 - DNA, 32 bp, synthetic construct
  source 1..32: mol_type = other DNA
ttcaaagcgactattaagcctttactgagtaa

SEQ ID NO: 503 - DNA, 40 bp, synthetic construct
  source 1..40: mol_type = other DNA
catttgtctttaccctgaaccagacctgtaatgcctcaga

SEQ ID NO: 504 - DNA, 32 bp, synthetic construct
  source 1..32: mol_type = other DNA
ggattgcacaaatatcgaaaaaccagcactaa

SEQ ID NO: 505 - DNA, 48 bp, synthetic construct
  source 1..48: mol_type = other DNA
tacattggtgcaacagtaattttcttaattgaaaagccaagaggacga

SEQ ID NO: 506 - DNA, 32 bp, synthetic construct
  source 1..32: mol_type = other DNA
accataaagactggatggtaaagaaaccgttc

SEQ ID NO: 507 - DNA, 32 bp, synthetic construct
  source 1..32: mol_type = other DNA
aatgtttatcaaaaattgcaatgctttcaacg

SEQ ID NO: 508 - DNA, 32 bp, synthetic construct
  source 1..32: mol_type = other DNA
gaatcgtctaaacagtataaatcactattaaa

SEQ ID NO: 509 - DNA, 48 bp, synthetic construct
  source 1..48: mol_type = other DNA
aacagagaagtaataaggattatatcgtcgctagtgaatatagccctc

SEQ ID NO: 510 - DNA, 32 bp, synthetic construct
  source 1..32: mol_type = other DNA
cgataaaaacattcaaataaattacctgagag

SEQ ID NO: 511 - DNA, 32 bp, synthetic construct
  source 1..32: mol_type = other DNA
ggaataccaccaaaattgatattcttcaaaag

SEQ ID NO: 512 - DNA, 36 bp, synthetic construct
  source 1..36: mol_type = other DNA
caaatcataacctggccctgtttgatggtggttccg

SEQ ID NO: 513 - DNA, 28 bp, synthetic construct
  source 1..28: mol_type = other DNA
ccctaatcctgacagatgatctattgat

SEQ ID NO: 514 - DNA, 24 bp, synthetic construct
  source 1..24: mol_type = other DNA
gcgaactgtacgtggcttctggcc

SEQ ID NO: 515 - DNA, 25 bp, synthetic construct
  source 1..25: mol_type = other DNA
agtcccaccagcttaaaattcgcat

SEQ ID NO: 516 - DNA, 32 bp, synthetic construct
  source 1..32: mol_type = other DNA
attacaggattatacccaaatattgtgaaatt

SEQ ID NO: 517 - DNA, 40 bp, synthetic construct
  source 1..40: mol_type = other DNA
ttttaagaactggctctagaaagaaaaaacagatgaacgg

SEQ ID NO: 518 - DNA, 32 bp, synthetic construct
  source 1..32: mol_type = other DNA
cggtcagtaaaaatacaaggccgcttgcgcat

SEQ ID NO: 519 - DNA, 32 bp, synthetic construct
  source 1..32: mol_type = other DNA
ttcaactttgaataagtttcctgtacggccag

SEQ ID NO: 520 - DNA, 40 bp, synthetic construct
  source 1..40: mol_type = other DNA
aaagctgctcattcagtaatcattaaccaatataaattgt

SEQ ID NO: 521 - DNA, 28 bp, synthetic construct
  source 1..28: mol_type = other DNA
atctatcattttaattttaataaaaatc

SEQ ID NO: 522 - DNA, 40 bp, synthetic construct
  source 1..40: mol_type = other DNA
ccctcaataaatgaaaccaccagattttgcggtttcttaa

SEQ ID NO: 523 - DNA, 32 bp, synthetic construct
  source 1..32: mol_type = other DNA
agaaccgggacagatggtaaaacgtcttcgct

SEQ ID NO: 524 - DNA, 32 bp, synthetic construct
  source 1..32: mol_type = other DNA
tgaaagagatattcatagcgagtaggaacgcc

SEQ ID NO: 525 - DNA, 40 bp, synthetic construct
  source 1..40: mol_type = other DNA
aggttatctcaacagttaaagactgcggaacagtatgcgt

SEQ ID NO: 526 - DNA, 40 bp, synthetic construct
  source 1..40: mol_type = other DNA
ccatgttacgaaacaatgcgggcccagctttccggcaccg

SEQ ID NO: 527 - DNA, 32 bp, synthetic construct
  source 1..32: mol_type = other DNA
gtcaacacctacgaagttttcatgtttttcac

SEQ ID NO: 528 - DNA, 40 bp, synthetic construct
  source 1..40: mol_type = other DNA
gcgattataccaagcgcttagccggtagatggacaacccg

SEQ ID NO: 529 - DNA, 48 bp, synthetic construct
  source 1..48: mol_type = other DNA
aatacattattcgacagcaccaacaagattgctttgaatatcatttca

SEQ ID NO: 530 - DNA, 17 bp, synthetic construct
  source 1..17: mol_type = other DNA
ggcaaaagaatatagat

SEQ ID NO: 531 - DNA, 15 bp, synthetic construct
  source 1..15: mol_type = other DNA
ttggtaaaatacgtt

SEQ ID NO: 532 - DNA, 48 bp, synthetic construct
  source 1..48: mol_type = other DNA
ctacagaggcttccattaagtcaatcatcatctttagtttgaggggac

SEQ ID NO: 533 - DNA, 15 bp, synthetic construct
  source 1..15: mol_type = other DNA
ttgtaacattggttt

SEQ ID NO: 534 - DNA, 37 bp, synthetic construct
  source 1..37: mol_type = other DNA
ggtagcaaaacctcaataagggaaaacaaacggcgga

SEQ ID NO: 535 - DNA, 35 bp, synthetic construct
  source 1..35: mol_type = other DNA
gaaagacagcatcggaaaaaatctaaaaggtgagg

SEQ ID NO: 536 - DNA, 48 bp, synthetic construct
  source 1..48: mol_type = other DNA
tgaggcttgcaccctcagctaaaacaggcatcaccgtctggccttcct

SEQ ID NO: 537 - DNA, 27 bp, synthetic construct
  source 1..27: mol_type = other DNA
aaccgataaaaagaagacagacaagag

SEQ ID NO: 538 - DNA, 37 bp, synthetic construct
  source 1..37: mol_type = other DNA
gacaacaaccatcgcccatttaagggacaggattatt

SEQ ID NO: 539 - DNA, 24 bp, synthetic construct
  source 1..24: mol_type = other DNA
accgatagccgtaacaattaccct

SEQ ID NO: 540 - DNA, 32 bp, synthetic construct
  source 1..32: mol_type = other DNA
acagcccatatatgtgtaatggaaagtgaatt

SEQ ID NO: 541 - DNA, 45 bp, synthetic construct
  source 1..45: mol_type = other DNA
ctttcgaggtgaagatcgtcagggagttacgaacgaatttaatgc

SEQ ID NO: 542 - DNA, 32 bp, synthetic construct
  source 1..32: mol_type = other DNA
gaaattcgacctttttctgagttttttagtac

SEQ ID NO: 543 - DNA, 32 bp, synthetic construct
  source 1..32: mol_type = other DNA
agattttcatttaacacatcaagattaggcgg

SEQ ID NO: 544 - DNA, 21 bp, synthetic construct
  source 1..21: mol_type = other DNA
ggagcctttaattaagacgag

SEQ ID NO: 545 - DNA, 32 bp, synthetic construct
  source 1..32: mol_type = other DNA
tccaaaaaagttttgtatttcattcccaaatc

SEQ ID NO: 546 - DNA, 32 bp, synthetic construct
  source 1..32: mol_type = other DNA
gttgacaggaaacaaaattacctgtgatgcaa

SEQ ID NO: 547 - DNA, 45 bp, synthetic construct
  source 1..45: mol_type = other DNA
tgcgaataataataggaagttttgaggactgaaaggaatatcaaa

SEQ ID NO: 548 - DNA, 32 bp, synthetic construct
  source 1..32: mol_type = other DNA
gcctggaataaacaaccgtctttcttgctcag

SEQ ID NO: 549 - DNA, 48 bp, synthetic construct
  source 1..48: mol_type = other DNA
tttcaacatccatcgcaagacaaagttaattttgaaacatccaagtcc

SEQ ID NO: 550 - DNA, 15 bp, synthetic construct
  source 1..15: mol_type = other DNA
tttctgtatggtttt

SEQ ID NO: 551 - DNA, 22 bp, synthetic construct
  source 1..22: mol_type = other DNA
agttagcgtaaagtaaatgaat

SEQ ID NO: 552 - DNA, 32 bp, synthetic construct
  source 1..32: mol_type = other DNA
aacgcctgtagcattcattgtttatcagcttg

SEQ ID NO: 553 - DNA, 27 bp, synthetic construct
  source 1..27: mol_type = other DNA
cattttcatctgaaatttttgccagac

SEQ ID NO: 554 - DNA, 37 bp, synthetic construct
  source 1..37: mol_type = other DNA
gccaccctcagagccaccaatgaaaaacgtattaccg

SEQ ID NO: 555 - DNA, 24 bp, synthetic construct
  source 1..24: mol_type = other DNA
agaaccgcaccagtatgaagccag

SEQ ID NO: 556 - DNA, 32 bp, synthetic construct
  source 1..32: mol_type = other DNA
cgccacttcatatgcgtactagaaaaagtacc

SEQ ID NO: 557 - DNA, 42 bp, synthetic construct
  source 1..42: mol_type = other DNA
cgtactcaggaggcgtcaccacccatgtattgcgccgacaat

SEQ ID NO: 558 - DNA, 32 bp, synthetic construct
  source 1..32: mol_type = other DNA
tatcaggagtactggtttatacaattgaggca

SEQ ID NO: 559 - DNA, 32 bp, synthetic construct
  source 1..32: mol_type = other DNA
ataggtctatcataatcgttaaatcatccctc

SEQ ID NO: 560 - DNA, 32 bp, synthetic construct
  source 1..32: mol_type = other DNA
ataagtgcaaataaggaataagttccaaaggt

SEQ ID NO: 561 - DNA, 32 bp, synthetic construct
  source 1..32: mol_type = other DNA
taccaggttttgaaatcatcttcttcaacaat

SEQ ID NO: 562 - DNA, 42 bp, synthetic construct
  source 1..42: mol_type = other DNA
gattagcggggttcagacgttcgatctaaaaaggctccaaaa

SEQ ID NO: 563 - DNA, 32 bp, synthetic construct
  source 1..32: mol_type = other DNA
atccatcatattattcaccgaccgctcagaac

SEQ ID NO: 564 - DNA, 15 bp, synthetic construct
  source 1..15: mol_type = other DNA
tttccctgcctattt

SEQ ID NO: 565 - DNA, 32 bp, synthetic construct
  source 1..32: mol_type = other DNA
gagtaacagtgcccgtgatcgtcgagagggtt

SEQ ID NO: 566 - DNA, 32 bp, synthetic construct
  source 1..32: mol_type = other DNA
gaatttaccgttccagcttcaccctcagaacc

SEQ ID NO: 567 - DNA, 27 bp, synthetic construct
  source 1..27: mol_type = other DNA
aatggaaacagaacaactcatggatag

SEQ ID NO: 568 - DNA, 37 bp, synthetic construct
  source 1..37: mol_type = other DNA
aaataaatcctcattaaatcgctgagtagtaaccgtt

SEQ ID NO: 569 - DNA, 24 bp, synthetic construct
  source 1..24: mol_type = other DNA
ttggccttagcaaggctccgggaa

SEQ ID NO: 570 - DNA, 32 bp, synthetic construct
  source 1..32: mol_type = other DNA
ggtcagcaaccgcgcctttattttagattagt

SEQ ID NO: 571 - DNA, 32 bp, synthetic construct
  source 1..32: mol_type = other DNA
gacaaccaaagccgttcggaaacgattgacgg

SEQ ID NO: 572 - DNA, 32 bp, synthetic construct
  source 1..32: mol_type = other DNA
aaagtaataccgcactccaagaaccgcaaatt

SEQ ID NO: 573 - DNA, 32 bp, synthetic construct
  source 1..32: mol_type = other DNA
agagccacactgtagccatcgagaacattttg

SEQ ID NO: 574 - DNA, 32 bp, synthetic construct
  source 1..32: mol_type = other DNA
cgccagttttatcattcaatcaatccagttac

SEQ ID NO: 575 - DNA, 32 bp, synthetic construct
  source 1..32: mol_type = other DNA
cagagccgccacctgtataaacagttaatgcc

SEQ ID NO: 576 - DNA, 32 bp, synthetic construct
  source 1..32: mol_type = other DNA
agataccgccatctttgcgttttccacaatca

SEQ ID NO: 577 - DNA, 32 bp, synthetic construct
  source 1..32: mol_type = other DNA
tgaacaaggtagaaactcataatccgcaaaca

SEQ ID NO: 578 - DNA, 15 bp, synthetic construct
  source 1..15: mol_type = other DNA
tttgcccccttattt

SEQ ID NO: 579 - DNA, 22 bp, synthetic construct
  source 1..22: mol_type = other DNA
agtttgccttttttcggtcata

SEQ ID NO: 580 - DNA, 32 bp, synthetic construct
  source 1..32: mol_type = other DNA
tagcagcaccgtaatcagcgagccgccgccag

SEQ ID NO: 581 - DNA, 22 bp, synthetic construct
  source 1..22: mol_type = other DNA
cagtagcaccagaaaccatcga

SEQ ID NO: 582 - DNA, 27 bp, synthetic construct
  source 1..27: mol_type = other DNA
ttagagccacgcaaataagaactcgtt

SEQ ID NO: 583 - DNA, 37 bp, synthetic construct
  source 1..37: mol_type = other DNA
caccgacttgagccatttggttttataataagggatt

SEQ ID NO: 584 - DNA, 24 bp, synthetic construct
  source 1..24: mol_type = other DNA
ttaaaggtataataagtaccgaag

SEQ ID NO: 585 - DNA, 42 bp, synthetic construct
  source 1..42: mol_type = other DNA
agggaaggtaaattcaccaatttaccattgatattcacaaac

SEQ ID NO: 586 - DNA, 32 bp, synthetic construct
  source 1..32: mol_type = other DNA
agacaaaagggcgacaagtagcgacagaatca

SEQ ID NO: 587 - DNA, 25 bp, synthetic construct
  source 1..25: mol_type = other DNA
atagataatacagagagtcaaaaat

SEQ ID NO: 588 - DNA, 42 bp, synthetic construct
  source 1..42: mol_type = other DNA
aagtttattttgtatcggcatagcgtcagcaccctcagagcc

SEQ ID NO: 589 - DNA, 24 bp, synthetic construct
  source 1..24: mol_type = other DNA
gccatatttgtttaacatacataa

SEQ ID NO: 590 - DNA, 29 bp, synthetic construct
  source 1..29: mol_type = other DNA
aggtggcacaaacgtagacaccacggaat

SEQ ID NO: 591 - DNA, 48 bp, synthetic construct
  source 1..48: mol_type = other DNA
ttttcagaatcctgaacttctttagatatagaacaacgccaacatttt

SEQ ID NO: 592 - DNA, 29 bp, synthetic construct
  source 1..29: mol_type = other DNA
ttttttgcccgactttaggagcacttttt

SEQ ID NO: 593 - DNA, 29 bp, synthetic construct
  source 1..29: mol_type = other DNA
tttttcatattccgcctgcaacagttttt

SEQ ID NO: 594 - DNA, 29 bp, synthetic construct
  source 1..29: mol_type = other DNA
ttttgaagggttagaacggcaattctttt

SEQ ID NO: 595 - DNA, 29 bp, synthetic construct
  source 1..29: mol_type = other DNA
tttttgcacgtaaaacaaattatcatttt

SEQ ID NO: 596 - DNA, 29 bp, synthetic construct
  source 1..29: mol_type = other DNA
ttttgatgaatatacagaagtttgatttt

SEQ ID NO: 597 - DNA, 53 bp, synthetic construct
  source 1..53: mol_type = other DNA
ttttgggagaaacaatataaatccttacaaacatgaggatttagaagtatttt

SEQ ID NO: 598 - DNA, 29 bp, synthetic construct
  source 1..29: mol_type = other DNA
ttttaagatgattaccttttacatctttt

SEQ ID NO: 599 - DNA, 25 bp, synthetic construct
  source 1..25: mol_type = other DNA
ttaattacaggtttaacgtcatttt

SEQ ID NO: 600 - DNA, 25 bp, synthetic construct
  source 1..25: mol_type = other DNA
tttttaaatcaatatcaaaattatt

SEQ ID NO: 601 - DNA, 29 bp, synthetic construct
  source 1..29: mol_type = other DNA
ttttgaaaacatagcgaaccttgcttttt

SEQ ID NO: 602 - DNA, 25 bp, synthetic construct
  source 1..25: mol_type = other DNA
ttttgagaagagtcaatacagtaca

SEQ ID NO: 603 - DNA, 25 bp, synthetic construct
  source 1..25: mol_type = other DNA
tttaacctccggcaaacaaaatttt

SEQ ID NO: 604 - DNA, 52 bp, synthetic construct
  source 1..52: mol_type = other DNA
ttttatatgtaaatgcagcaaaagcgaattatccaagttacaaaatcgtttt

SEQ ID NO: 605 - DNA, 26 bp, synthetic construct
  source 1..26: mol_type = other DNA
ttttaatggtgggttatataactttt

SEQ ID NO: 606 - DNA, 29 bp, synthetic construct
  source 1..29: mol_type = other DNA
ttttacaccggagagagactacctttttt

SEQ ID NO: 607 - DNA, 28 bp, synthetic construct
  source 1..28: mol_type = other DNA
tttttttagtatagattaagacgctttt

SEQ ID NO: 608 - DNA, 29 bp, synthetic construct
  source 1..29: mol_type = other DNA
ttttagtagggcccttagaatcctttttt

SEQ ID NO: 609 - DNA, 29 bp, synthetic construct
  source 1..29: mol_type = other DNA
tttttgtaatttaggcacgctcaactttt

SEQ ID NO: 610 - DNA, 29 bp, synthetic construct
  source 1..29: mol_type = other DNA
ttttaataagagaatataaagcctgtttt

SEQ ID NO: 611 - DNA, 29 bp, synthetic construct
  source 1..29: mol_type = other DNA
ttttcgacaataaacaaaagaataatttt

SEQ ID NO: 612 - DNA, 53 bp, synthetic construct
  source 1..53: mol_type = other DNA
ttttacgcgcctgtttagacctaaaatattttagaacgcgagaaaactttttt

SEQ ID NO: 613 - DNA, 29 bp, synthetic construct
  source 1..29: mol_type = other DNA
ttttgtctttcccagctaatgcagatttt

SEQ ID NO: 614 - DNA, 29 bp, synthetic construct
  source 1..29: mol_type = other DNA
ttttaaccaagttctgtccagacgatttt

SEQ ID NO: 615 - DNA, 29 bp, synthetic construct
  source 1..29: mol_type = other DNA
ttttgaatcattttttcgagccagttttt

SEQ ID NO: 616 - DNA, 29 bp, synthetic construct
  source 1..29: mol_type = other DNA
ttttttagcgaacctccagcaaatctttt

SEQ ID NO: 617 - DNA, 29 bp, synthetic construct
  source 1..29: mol_type = other DNA
ttttaagccttaaatcacatcgtagtttt

SEQ ID NO: 618 - DNA, 29 bp, synthetic construct
  source 1..29: mol_type = other DNA
tttttgaatcttaccaagggtattatttt

SEQ ID NO: 619 - DNA, 53 bp, synthetic construct
  source 1..53: mol_type = other DNA
ttttagagcctaatttgaatcggctacgagcataaaaataatatcccattttt

SEQ ID NO: 620 - DNA, 29 bp, synthetic construct
  source 1..29: mol_type = other DNA
ttttgcagccttcgagcgtctttcctttt

SEQ ID NO: 621 - DNA, 29 bp, synthetic construct
  source 1..29: mol_type = other DNA
ttttattaactgtacaattttatcctttt

SEQ ID NO: 622 - DNA, 29 bp, synthetic construct
  source 1..29: mol_type = other DNA
ttttagataaccgcgggaggttttgtttt

SEQ ID NO: 623 - DNA, 35 bp, synthetic construct
  source 1..35: mol_type = other DNA
ttttccctttttgccgattacagtgaggctatttt

SEQ ID NO: 624 - DNA, 41 bp, synthetic construct
  source 1..41: mol_type = other DNA
ttagacaggaacggtaatagcaataacgcgaggcgtttttt

SEQ ID NO: 625 - DNA, 52 bp, synthetic construct
  source 1..52: mol_type = other DNA
gtagcaatgaagtgttattctaagagctatctagcaagaaacaatgaatttt

SEQ ID NO: 626 - DNA, 15 bp, synthetic construct
  source 1..15: mol_type = other DNA
tttttagtaataaca

SEQ ID NO: 627 - DNA, 33 bp, synthetic construct
  source 1..33: mol_type = other DNA
cagattcatctgtaaacttctgaataatgtttt

SEQ ID NO: 628 - DNA, 34 bp, synthetic construct
  source 1..34: mol_type = other DNA
ttttgtcacacgacctagaacccatcaatataaa

SEQ ID NO: 629 - DNA, 27 bp, synthetic construct
  source 1..27: mol_type = other DNA
tttttgacctgaaagcgtaagaaatag

SEQ ID NO: 630 - DNA, 47 bp, synthetic construct
  source 1..47: mol_type = other DNA
ttttacatcgccattattaacacctgattataggagcgggaaataaa

SEQ ID NO: 631 - DNA, 29 bp, synthetic construct
  source 1..29: mol_type = other DNA
ttttgccacgctgagagccagcagccaat

SEQ ID NO: 632 - DNA, 47 bp, synthetic construct
  source 1..47: mol_type = other DNA
ttttcagttggcaaataaaatatacgttattactcgtatacggattc

SEQ ID NO: 633 - DNA, 25 bp, synthetic construct
  source 1..25: mol_type = other DNA
ttttaacaactaatagattagagcc

SEQ ID NO: 634 - DNA, 12 bp, synthetic construct
  source 1..12: mol_type = other DNA
ttttttagactt

SEQ ID NO: 635 - DNA, 12 bp, synthetic construct
  source 1..12: mol_type = other DNA
ttttcgcagagg

SEQ ID NO: 636 - DNA, 12 bp, synthetic construct
  source 1..12: mol_type = other DNA
tttttttcaaat

SEQ ID NO: 637 - DNA, 12 bp, synthetic construct
  source 1..12: mol_type = other DNA
ttttcctaattt

SEQ ID NO: 638 - DNA, 36 bp, synthetic construct
  source 1..36: mol_type = other DNA
aaattcttcacaagaaagcgctaatatcagagtttt

SEQ ID NO: 639 - DNA, 36 bp, synthetic construct
  source 1..36: mol_type = other DNA
cacccagcaacaccctcgcattagacgggagatttt

SEQ ID NO: 640 - DNA, 12 bp, synthetic construct
  source 1..12: mol_type = other DNA
ttttataagaaa

SEQ ID NO: 641 - DNA, 35 bp, synthetic construct
  source 1..35: mol_type = other DNA
gaaaatacgatttttatttatcccaatccaatttt

SEQ ID NO: 642 - DNA, 30 bp, synthetic construct
  source 1..30: mol_type = other DNA
ttttggaaagccggcggcaagtgtagtttt

SEQ ID NO: 643 - DNA, 30 bp, synthetic construct
  source 1..30: mol_type = other DNA
ttttaagttttttgggagcttgacggtttt

SEQ ID NO: 644 - DNA, 30 bp, synthetic construct
  source 1..30: mol_type = other DNA
ttttgttgttccagttcacccaaatctttt

SEQ ID NO: 645 - DNA, 30 bp, synthetic construct
  source 1..30: mol_type = other DNA
ttttggtttgccccatagggttgagttttt

SEQ ID NO: 646 - DNA, 30 bp, synthetic construct
  source 1..30: mol_type = other DNA
ttttcgccagggtggcggtccacgcttttt

SEQ ID NO: 647 - DNA, 40 bp, synthetic construct
  source 1..40: mol_type = other DNA
ttttcataaagtgtaaagccacatacgagccggaagtttt

SEQ ID NO: 648 - DNA, 40 bp, synthetic construct
  source 1..40: mol_type = other DNA
ttttccgggtaccgagctcgcgactctagaggatcctttt

SEQ ID NO: 649 - DNA, 40 bp, synthetic construct
  source 1..40: mol_type = other DNA
ttttttaagttgggtaacgcatgtgctgcaaggcgatttt

SEQ ID NO: 650 - DNA, 40 bp, synthetic construct
  source 1..40: mol_type = other DNA
tttttcgccattcaggctgcccaggcaaagcgccattttt

SEQ ID NO: 651 - DNA, 19 bp, synthetic construct
  source 1..19: mol_type = other DNA
ttttgagatcgttggtttt

SEQ ID NO: 652 - DNA, 19 bp, synthetic construct
  source 1..19: mol_type = other DNA
ttttgagtaatgacgtttt

SEQ ID NO: 653 - DNA, 19 bp, synthetic construct
  source 1..19: mol_type = other DNA
tttttccgcacagactttt

SEQ ID NO: 654 - DNA, 39 bp, synthetic construct
  source 1..39: mol_type = other DNA
ttttcggtcacgctgcgcgtaaccaccacaccgggcgct

SEQ ID NO: 655 - DNA, 28 bp, synthetic construct
  source 1..28: mol_type = other DNA
ggaaacctcgctcactgcccgctttttt

SEQ ID NO: 656 - DNA, 33 bp, synthetic construct
  source 1..33: mol_type = other DNA
attacgccgcgatcggagtacaacggagatttt

SEQ ID NO: 657 - DNA, 44 bp, synthetic construct
  source 1..44: mol_type = other DNA
ttaaatatcgcagagcgggagctaaacaggagaagaaaagtttt

SEQ ID NO: 658 - DNA, 14 bp, synthetic construct
  source 1..14: mol_type = other DNA
ttttaacggaacgc

SEQ ID NO: 659 - DNA, 45 bp, synthetic construct
  source 1..45: mol_type = other DNA
ttttgagaatagactaaaacgtaatgccataaaacacattgagga

SEQ ID NO: 660 - DNA, 42 bp, synthetic construct
  source 1..42: mol_type = other DNA
ttttggctgagacgtttcagcgattttgccaactaaaggaat

SEQ ID NO: 661 - DNA, 42 bp, synthetic construct
  source 1..42: mol_type = other DNA
ttttccagagccagaaagtattcggaaccagagaaggattag

SEQ ID NO: 662 - DNA, 44 bp, synthetic construct
  source 1..44: mol_type = other DNA
tgctattcggtaattgttgagttaagcagttaccagaaggtttt

SEQ ID NO: 663 - DNA, 44 bp, synthetic construct
  source 1..44: mol_type = other DNA
ttttcatatggtcagggaaggaacaaagtcaataacggaatacc

SEQ ID NO: 664 - DNA, 44 bp, synthetic construct
  source 1..44: mol_type = other DNA
aaaataaagaaaatacgaataacataaaagactccttatttttt

SEQ ID NO: 665 - DNA, 42 bp, synthetic construct
  source 1..42: mol_type = other DNA
tttttaaaagaaaaaaatcacagcgtttggaaccgcctccct

SEQ ID NO: 666 - DNA, 13 bp, synthetic construct
  source 1..13: mol_type = other DNA
ttttgtatgttag

SEQ ID NO: 667 - DNA, 34 bp, synthetic construct
  source 1..34: mol_type = other DNA
ttttgaactggcatgattaaattaccagcgccaa

SEQ ID NO: 668 - DNA, 34 bp, synthetic construct
  source 1..34: mol_type = other DNA
ttttgaggaaacgcaatagagaaccgattgaggg

SEQ ID NO: 669 - DNA, 34 bp, synthetic construct
  source 1..34: mol_type = other DNA
ttttagatagccgaacaaccagaattatcaccgt

SEQ ID NO: 670 - DNA, 45 bp, synthetic construct
  source 1..45: mol_type = other DNA
gcgtattgggcgccagggtggtttttcttttcaccagcttgcttc

SEQ ID NO: 671 - DNA, 32 bp, synthetic construct
  source 1..32: mol_type = other DNA
atcggccaggaaacagaatttatccagacgac

SEQ ID NO: 672 - DNA, 32 bp, synthetic construct
  source 1..32: mol_type = other DNA
tgtcgaaaatcctgtttgatggtgaaagaata

SEQ ID NO: 673 - DNA, 37 bp, synthetic construct
  source 1..37: mol_type = other DNA
atttgaattaccttttttaatacgcgcggccagctgc

SEQ ID NO: 674 - DNA, 48 bp, synthetic construct
  source 1..48: mol_type = other DNA
gcccgagaagtccactattaaagagtctatcagaaccatcgtaaagca

SEQ ID NO: 675 - DNA, 32 bp, synthetic construct
  source 1..32: mol_type = other DNA
aaacaaaaaagatgatatttacgatgaaaata

SEQ ID NO: 676 - DNA, 32 bp, synthetic construct
  source 1..32: mol_type = other DNA
ggaacaagtagggttgttcagctaagacgctg

SEQ ID NO: 677 - DNA, 45 bp, synthetic construct
  source 1..45: mol_type = other DNA
tttcaattacctgagcaaaagttaattacattctgtcaaaatcat

SEQ ID NO: 678 - DNA, 32 bp, synthetic construct
  source 1..32: mol_type = other DNA
tacaaaatgcctgatttgagcgcttcaccgac

SEQ ID NO: 679 - DNA, 48 bp, synthetic construct
  source 1..48: mol_type = other DNA
ctaaatcggacggggaaagccggcaaggagcgggcgctagtaaccacc

SEQ ID NO: 680 - DNA, 32 bp, synthetic construct
  source 1..32: mol_type = other DNA
gaggtgccacccaaatgaataacacaagaaaa

SEQ ID NO: 681 - DNA, 45 bp, synthetic construct
  source 1..45: mol_type = other DNA
gggagaaacaataacggattccgcgcagagtcaaaaagcatgtag

SEQ ID NO: 682 - DNA, 32 bp, synthetic construct
  source 1..32: mol_type = other DNA
gaatatacagattttcagcagcactaagtttt

SEQ ID NO: 683 - DNA, 32 bp, synthetic construct
  source 1..32: mol_type = other DNA
gaaagcgagaacgtggttagagccccctgaac

SEQ ID NO: 684 - DNA, 45 bp, synthetic construct
  source 1..45: mol_type = other DNA
acagaaataaagaaattgcgtagtaacagtatcaccgaatatcag

SEQ ID NO: 685 - DNA, 40 bp, synthetic construct
  source 1..40: mol_type = other DNA
acacccgcatggttgctttgacgagagcgggagctaaaca

SEQ ID NO: 686 - DNA, 40 bp, synthetic construct
  source 1..40: mol_type = other DNA
ctaccatatctgaataattaagagaggagcggccgaacgt

SEQ ID NO: 687 - DNA, 15 bp, synthetic construct
  source 1..15: mol_type = other DNA
tatttgcacgtaaat

SEQ ID NO: 688 - DNA, 32 bp, synthetic construct
  source 1..32: mol_type = other DNA
cgcgtactcgcgcttatgagtaacaacgtcac

SEQ ID NO: 689 - DNA, 45 bp, synthetic construct
  source 1..45: mol_type = other DNA
cctgattgtttggattatacttcaaaattactggtaacgtaatca

SEQ ID NO: 690 - DNA, 24 bp, synthetic construct
  source 1..24: mol_type = other DNA
ggaggccgattaatatctacaggg

SEQ ID NO: 691 - DNA, 24 bp, synthetic construct
  source 1..24: mol_type = other DNA
atggcaatccaccagagcttatac

SEQ ID NO: 692 - DNA, 29 bp, synthetic construct
  source 1..29: mol_type = other DNA
tcaagggattttagaccctattattagcg

SEQ ID NO: 693 - DNA, 34 bp, synthetic construct
  source 1..34: mol_type = other DNA
tcattttgcggaacaaagaaatcatcaatataat

SEQ ID NO: 694 - DNA, 48 bp, synthetic construct
  source 1..48: mol_type = other DNA
ccaccgagttgtagcaatacttctaagaactcaaactatccgccagcc

SEQ ID NO: 695 - DNA, 35 bp, synthetic construct
  source 1..35: mol_type = other DNA
tattaattgtattaaagaatcatgaggaagttcag

SEQ ID NO: 696 - DNA, 36 bp, synthetic construct
  source 1..36: mol_type = other DNA
attaaccgtaaaagagattaggattctgaaaccagt

SEQ ID NO: 697 - DNA, 37 bp, synthetic construct
  source 1..37: mol_type = other DNA
ttacaaacaattcgacaactcttaaaagtgaccccca

SEQ ID NO: 698 - DNA, 35 bp, synthetic construct
  source 1..35: mol_type = other DNA
tacatttgtagattagcagaggccgcttttgcaat

SEQ ID NO: 699 - DNA, 48 bp, synthetic construct
  source 1..48: mol_type = other DNA
attgcaacgacgctcaatcgtctgtcacacgaccagtaatccttctga

SEQ ID NO: 700 - DNA, 32 bp, synthetic construct
  source 1..32: mol_type = other DNA
aatattacggccttgcaggaggttgagggttg

SEQ ID NO: 701 - DNA, 37 bp, synthetic construct
  source 1..37: mol_type = other DNA
tttaggagcactaacaactaaaggatttaactaaaga

SEQ ID NO: 702 - DNA, 24 bp, synthetic construct
  source 1..24: mol_type = other DNA
aggaattgtcagttggcattgctt

SEQ ID NO: 703 - DNA, 32 bp, synthetic construct
  source 1..32: mol_type = other DNA
ttcaccagaaatggataacccatgcctcagag

SEQ ID NO: 704 - DNA, 37 bp, synthetic construct
  source 1..37: mol_type = other DNA
aaaccctcaatcaatatctggaggaaggttgcaggga

SEQ ID NO: 705 - DNA, 48 bp, synthetic construct
  source 1..48: mol_type = other DNA
cctgaaagaatggctattagtcttcattaaaaataccgaacgaaccac

SEQ ID NO: 706 - DNA, 24 bp, synthetic construct
  source 1..24: mol_type = other DNA
aagcatcaagccagcacagcggag

SEQ ID NO: 707 - DNA, 37 bp, synthetic construct
  source 1..37: mol_type = other DNA
ctgcaacagtgccacgctgagccttgctgcggtttat

SEQ ID NO: 708 - DNA, 48 bp, synthetic construct
  source 1..48: mol_type = other DNA
cagcagaaacagacaatatttttgcgtaagaaagttttgtctgtagca

SEQ ID NO: 709 - DNA, 29 bp, synthetic construct
  source 1..29: mol_type = other DNA
agaggtgaggcggtcagtattaacaccgc

SEQ ID NO: 710 - DNA, 40 bp, synthetic construct
  source 1..40: mol_type = other DNA
ccattcgcgaataataaaagctgcattcattaaacccacc

SEQ ID NO: 711 - DNA, 48 bp, synthetic construct
  source 1..48: mol_type = other DNA
cgcttctggcactccaagtgaatagccagaggagaggctttgcgaata

SEQ ID NO: 712 - DNA, 40 bp, synthetic construct
  source 1..40: mol_type = other DNA
gggacgcatagtaaaacggtgtcttgttttaagaaatccg

SEQ ID NO: 713 - DNA, 48 bp, synthetic construct
  source 1..48: mol_type = other DNA
gcatcgtataggtcacttcattccggtaaagaaatgcaattcagtttg

SEQ ID NO: 714 - DNA, 29 bp, synthetic construct
  source 1..29: mol_type = other DNA
gaaattgtcatggtcaaccgtgtgataaa

SEQ ID NO: 715 - DNA, 29 bp, synthetic construct
  source 1..29: mol_type = other DNA
ctcacatttggggtgcaagacaaagaacg

SEQ ID NO: 716 - DNA, 40 bp, synthetic construct
  source 1..40: mol_type = other DNA
attaatgatgtaaatccaatagtgtacataaacatcaaga

SEQ ID NO: 717 - DNA, 32 bp, synthetic construct
  source 1..32: mol_type = other DNA
cgggaaacgagactacctttttaattagtacc

SEQ ID NO: 718 - DNA, 48 bp, synthetic construct
  source 1..48: mol_type = other DNA
agaagagtgtcgctattgaataactgagacgggcaacagctgattgcc

SEQ ID NO: 719 - DNA, 29 bp, synthetic construct
  source 1..29: mol_type = other DNA
aggtctgactgtcgtgggagaggcggttt

SEQ ID NO: 720 - DNA, 24 bp, synthetic construct
  source 1..24: mol_type = other DNA
acggtccgcagaaaagtgagctaa

SEQ ID NO: 721 - DNA, 42 bp, synthetic construct
  source 1..42: mol_type = other DNA
tatataactatatgaggcattcaacgccaaagccgtttttat

SEQ ID NO: 722 - DNA, 32 bp, synthetic construct
  source 1..32: mol_type = other DNA
cgagaaaactttttatggcttaattgagaatc

SEQ ID NO: 723 - DNA, 24 bp, synthetic construct
  source 1..24: mol_type = other DNA
cggagaatttgttaaatcctgtgt

SEQ ID NO: 724 - DNA, 42 bp, synthetic construct
  source 1..42: mol_type = other DNA
tttcatcttctgaattcttactttagtatagaacgcgaggcg

SEQ ID NO: 725 - DNA, 22 bp, synthetic construct
  source 1..22: mol_type = other DNA
gaaaaagcctgcagtataaagc

SEQ ID NO: 726 - DNA, 32 bp, synthetic construct
  source 1..32: mol_type = other DNA
caacgctcaacagtagttcaccgcgcccaata

SEQ ID NO: 727 - DNA, 22 bp, synthetic construct
  source 1..22: mol_type = other DNA
gccatatttaattcgagccagt

SEQ ID NO: 728 - DNA, 32 bp, synthetic construct
  source 1..32: mol_type = other DNA
aataagagaatataaaagcatcattccaagaa

SEQ ID NO: 729 - DNA, 32 bp, synthetic construct
  source 1..32: mol_type = other DNA
gacaataaccatcctagaaacaaataccaagt

SEQ ID NO: 730 - DNA, 48 bp, synthetic construct
  source 1..48: mol_type = other DNA
gacaaaagcaataatcggctgtcttcgagaaacgatttttcccacaag

SEQ ID NO: 731 - DNA, 32 bp, synthetic construct
  source 1..32: mol_type = other DNA
ataatatcacaacatgagtgttgttcaatata

SEQ ID NO: 732 - DNA, 29 bp, synthetic construct
  source 1..29: mol_type = other DNA
aaaccaatgtaaagtaatttaacaatttc

SEQ ID NO: 733 - DNA, 32 bp, synthetic construct
  source 1..32: mol_type = other DNA
taacacttaataaagctttaggcagtaaatgc

SEQ ID NO: 734 - DNA, 32 bp, synthetic construct
  source 1..32: mol_type = other DNA
cgggtattaaaccgtcaaacagccatattatt

SEQ ID NO: 735 - DNA, 32 bp, synthetic construct
  source 1..32: mol_type = other DNA
cactcgaatgtaccaactcagagcatcgatga

SEQ ID NO: 736 - DNA, 32 bp, synthetic construct
  source 1..32: mol_type = other DNA
gaacaagcacatgtaagaagcctttcaagggt

SEQ ID NO: 737 - DNA, 32 bp, synthetic construct
  source 1..32: mol_type = other DNA
aacctgttttgcgggaaaacattatcacaaat

SEQ ID NO: 738 - DNA, 32 bp, synthetic construct
  source 1..32: mol_type = other DNA
aatggattaacgcaagttatacaacctaaatt

SEQ ID NO: 739 - DNA, 32 bp, synthetic construct
  source 1..32: mol_type = other DNA
ggcttcgaatattttagataaaaaattaatgc

SEQ ID NO: 740 - DNA, 24 bp, synthetic construct
  source 1..24: mol_type = other DNA
gtattctacatatgcgttctaaac

SEQ ID NO: 741 - DNA, 37 bp, synthetic construct
  source 1..37: mol_type = other DNA
ttttagcgaacctcccgaagtgttggtgttctccgtg

SEQ ID NO: 742 - DNA, 32 bp, synthetic construct
  source 1..32: mol_type = other DNA
aatcaagattagttgcgtaaactggcatgatt

SEQ ID NO: 743 - DNA, 32 bp, synthetic construct
  source 1..32: mol_type = other DNA
ttgccagttacaaaataggctttttaagaaaa

SEQ ID NO: 744 - DNA, 32 bp, synthetic construct
  source 1..32: mol_type = other DNA
tatcccaatccaaatacgtcaataataagagc

SEQ ID NO: 745 - DNA, 32 bp, synthetic construct
  source 1..32: mol_type = other DNA
gcagccttagggtaatgctttgaacgtcagat

SEQ ID NO: 746 - DNA, 32 bp, synthetic construct
  source 1..32: mol_type = other DNA
aaagtcagtacagagacaagtttttccagttt

SEQ ID NO: 747 - DNA, 29 bp, synthetic construct
  source 1..29: mol_type = other DNA
agagataatgtttaacggcgaattattca

SEQ ID NO: 748 - DNA, 32 bp, synthetic construct
  source 1..32: mol_type = other DNA
aattgagtcataattattcattaaagaatcaa

SEQ ID NO: 749 - DNA, 32 bp, synthetic construct
  source 1..32: mol_type = other DNA
attgcgcctttaattctccaacagaagtaccg

SEQ ID NO: 750 - DNA, 42 bp, synthetic construct
  source 1..42: mol_type = other DNA
aagaaacaatgaaaaccgattgccaaagacgtttgccatctt

SEQ ID NO: 751 - DNA, 32 bp, synthetic construct
  source 1..32: mol_type = other DNA
agctagtcaagcaaacgagcttcaagtagcat

SEQ ID NO: 752 - DNA, 32 bp, synthetic construct
  source 1..32: mol_type = other DNA
ccgaagccattagagactaacgagatctcaat

SEQ ID NO: 753 - DNA, 32 bp, synthetic construct
  source 1..32: mol_type = other DNA
gttcagaaaagaggtcgtacctttgctatcga

SEQ ID NO: 754 - DNA, 32 bp, synthetic construct
  source 1..32: mol_type = other DNA
gctttacataccaacgaatataatatatagaa

SEQ ID NO: 755 - DNA, 42 bp, synthetic construct
  source 1..42: mol_type = other DNA
gaaaccgaggaaaaagacaccgtggcaaccgccaccctcaga

SEQ ID NO: 756 - DNA, 32 bp, synthetic construct
  source 1..32: mol_type = other DNA
taacgggaaattgctgatttttgcatttcgca

SEQ ID NO: 757 - DNA, 24 bp, synthetic construct
  source 1..24: mol_type = other DNA
cccaaaaggctcaacaggacttgc

SEQ ID NO: 758 - DNA, 37 bp, synthetic construct
  source 1..37: mol_type = other DNA
aagactccttattacgcataaaggcgattagatgggc

SEQ ID NO: 759 - DNA, 32 bp, synthetic construct
  source 1..32: mol_type = other DNA
ttattttgtcacaatccatgaaccagagccac

SEQ ID NO: 760 - DNA, 22 bp, synthetic construct
  source 1..22: mol_type = other DNA
ggtttaccagcgagggagggaa

SEQ ID NO: 761 - DNA, 32 bp, synthetic construct
  source 1..32: mol_type = other DNA
ggtaaatattgacggacagtcagactgtagcg

SEQ ID NO: 762 - DNA, 32 bp, synthetic construct
  source 1..32: mol_type = other DNA
ttgagccaccatcgataggtttaagttagaac

SEQ ID NO: 763 - DNA, 32 bp, synthetic construct
  source 1..32: mol_type = other DNA
caatgaaatttgggaacgagaaagttggggtc

SEQ ID NO: 764 - DNA, 29 bp, synthetic construct
  source 1..29: mol_type = other DNA
gtagcgacaggtgaattaccttttacatc

SEQ ID NO: 765 - DNA, 32 bp, synthetic construct
  source 1..32: mol_type = other DNA
gtttgcctcctcttttgatgatacaaacaaag

SEQ ID NO: 766 - DNA, 32 bp, synthetic construct
  source 1..32: mol_type = other DNA
ataaagcgttgagattcgacattcatagcaat

SEQ ID NO: 767 - DNA, 21 bp, synthetic construct
  source 1..21: mol_type = other DNA
cgttttcatcggcccggaatt

SEQ ID NO: 768 - DNA, 32 bp, synthetic construct
  source 1..32: mol_type = other DNA
tcataatacagatacataggaatacaaagcgg

SEQ ID NO: 769 - DNA, 32 bp, synthetic construct
  source 1..32: mol_type = other DNA
cttattagcaaaagggagcaacaccgaaaaca

SEQ ID NO: 770 - DNA, 45 bp, synthetic construct
  source 1..45: mol_type = other DNA
ttcataatcaaaatcctcattccttgatattcggtcgaaacagct

SEQ ID NO: 771 - DNA, 32 bp, synthetic construct
  source 1..32: mol_type = other DNA
gtgaattaatagtaagtaacgccacagtctta

SEQ ID NO: 772 - DNA, 32 bp, synthetic construct
  source 1..32: mol_type = other DNA
tttaaccgaaccctcggaaacgcacgcaataa

SEQ ID NO: 773 - DNA, 21 bp, synthetic construct
  source 1..21: mol_type = other DNA
caccggaaccgccgacggagg

SEQ ID NO: 774 - DNA, 32 bp, synthetic construct
  source 1..32: mol_type = other DNA
agccggagaaatagcgtttaccagcctcaaat

SEQ ID NO: 775 - DNA, 24 bp, synthetic construct
  source 1..24: mol_type = other DNA
ctcagaacatataaaaggggtatg

SEQ ID NO: 776 - DNA, 37 bp, synthetic construct
  source 1..37: mol_type = other DNA
gccaccaccctcagagccttcgccagcttggggatgt

SEQ ID NO: 777 - DNA, 35 bp, synthetic construct
  source 1..35: mol_type = other DNA
gccgccagcattgacaaaggagcctttcaactaaa

SEQ ID NO: 778 - DNA, 37 bp, synthetic construct
  source 1..37: mol_type = other DNA
ttgaggcaaatttcttctgaggcttatctaaaatatc

SEQ ID NO: 779 - DNA, 48 bp, synthetic construct
  source 1..48: mol_type = other DNA
cagacgattggaaagccaggggatcgtctttgagggaagtattagact

SEQ ID NO: 780 - DNA, 35 bp, synthetic construct
  source 1..35: mol_type = other DNA
ggaaagcgcagtctctaactacagaggcaccctca

SEQ ID NO: 781 - DNA, 37 bp, synthetic construct
  source 1..37: mol_type = other DNA
taccgttctccattaatcatctttttgagtaacatta

SEQ ID NO: 782 - DNA, 35 bp, synthetic construct
  source 1..35: mol_type = other DNA
taagcgtcatacatgggcttaaaacacacgggtaa

SEQ ID NO: 783 - DNA, 20 bp, synthetic construct
  source 1..20: mol_type = other DNA
gcctatgcgccggaagggaa

SEQ ID NO: 784 - DNA, 40 bp, synthetic construct
  source 1..40: mol_type = other DNA
aacggggtatgaaagtatggaaggtcctgattatcagatg

SEQ ID NO: 785 - DNA, 16 bp, synthetic construct
  source 1..16: mol_type = other DNA
caagcgcgaggagtgt

SEQ ID NO: 786 - DNA, 48 bp, synthetic construct
  source 1..48: mol_type = other DNA
tacaacggagatttgtgaatacacccatgttataagggaaattttcgg

SEQ ID NO: 787 - DNA, 40 bp, synthetic construct
  source 1..40: mol_type = other DNA
gcgatgagactcctcaatagcccgtcctttgcatagataa

SEQ ID NO: 788 - DNA, 32 bp, synthetic construct
  source 1..32: mol_type = other DNA
atataagtagagaaggtctgtccaaattatca

SEQ ID NO: 789 - DNA, 40 bp, synthetic construct
  source 1..40: mol_type = other DNA
aatacgtagcaacggctgaccaaccaaataaatcatcatt

SEQ ID NO: 790 - DNA, 40 bp, synthetic construct
  source 1..40: mol_type = other DNA
ctttttaggtgtatcactcattttagccgtcacagttgaa

SEQ ID NO: 791 - DNA, 32 bp, synthetic construct
  source 1..32: mol_type = other DNA
ccaccaccccgtactctggtaatatcacgcaa

SEQ ID NO: 792 - DNA, 40 bp, synthetic construct
  source 1..40: mol_type = other DNA
gcagcgaaccgatatattcacaaagtaatctttccctcag

SEQ ID NO: 793 - DNA, 40 bp, synthetic construct
  source 1..40: mol_type = other DNA
gttaaggatagcaagcacagccctcaaatcaaaaaatcta

SEQ ID NO: 794 - DNA, 40 bp, synthetic construct
  source 1..40: mol_type = other DNA
acgcataaagacagcagtacagactttgaaagattgcccc

SEQ ID NO: 795 - DNA, 32 bp, synthetic construct
  source 1..32: mol_type = other DNA
ttccacagccaataggtatttacatccagaac

SEQ ID NO: 796 - DNA, 15 bp, synthetic construct
  source 1..15: mol_type = other DNA
tttcgaggtgggttt

SEQ ID NO: 797 - DNA, 32 bp, synthetic construct
  source 1..32: mol_type = other DNA
tgataccgtccaaaagaaccggattcagccac

SEQ ID NO: 798 - DNA, 40 bp, synthetic construct
  source 1..40: mol_type = other DNA
cagctagttagcgtaaaacagtttgcaaatgagataaaac

SEQ ID NO: 799 - DNA, 40 bp, synthetic construct
  source 1..40: mol_type = other DNA
aaaaaggcatagttgccatcaagacaggcgcaatttcaac

SEQ ID NO: 800 - DNA, 46 bp, synthetic construct
  source 1..46: mol_type = other DNA
gggattttgctaaacaactttccgatctaatacgtggcttggcaga

SEQ ID NO: 801 - DNA, 15 bp, synthetic construct
  source 1..15: mol_type = other DNA
tttgagaatagcctt

SEQ ID NO: 802 - DNA, 24 bp, synthetic construct
  source 1..24: mol_type = other DNA
ggaattgccattcaggtccggcac

SEQ ID NO: 803 - DNA, 48 bp, synthetic construct
  source 1..48: mol_type = other DNA
tttttcaggaagatcgtgccggaaacgtaacattttttcacgtttttt

SEQ ID NO: 804 - DNA, 48 bp, synthetic construct
  source 1..48: mol_type = other DNA
ttttccgtaatgggaaccgtgcactaaagtatgtttagactggatttt

SEQ ID NO: 805 - DNA, 29 bp, synthetic construct
  source 1..29: mol_type = other DNA
ttttctggccttaaaggccggagactttt

SEQ ID NO: 806 - DNA, 29 bp, synthetic construct
  source 1..29: mol_type = other DNA
ttttccaataggtgatattcaaccgtttt

SEQ ID NO: 807 - DNA, 29 bp, synthetic construct
  source 1..29: mol_type = other DNA
tttttaatatttttgagagatctactttt

SEQ ID NO: 808 - DNA, 29 bp, synthetic construct
  source 1..29: mol_type = other DNA
ttttattgtatacctgagagtctggtttt

SEQ ID NO: 809 - DNA, 29 bp, synthetic construct
  source 1..29: mol_type = other DNA
ttttagcaaacaagagaataaagcttttt

SEQ ID NO: 810 - DNA, 29 bp, synthetic construct
  source 1..29: mol_type = other DNA
ttttaaaggctatcaggtgaccctgtttt

SEQ ID NO: 811 - DNA, 29 bp, synthetic construct
  source 1..29: mol_type = other DNA
ttttttctagctgataatttttagatttt

SEQ ID NO: 812 - DNA, 29 bp, synthetic construct
  source 1..29: mol_type = other DNA
ttttagtcaaatcaccagcctgagttttt

SEQ ID NO: 813 - DNA, 29 bp, synthetic construct
  source 1..29: mol_type = other DNA
ttttaccctcatgtagatttagttttttt

SEQ ID NO: 814 - DNA, 29 bp, synthetic construct
  source 1..29: mol_type = other DNA
tttttaatactttagctatattttctttt

SEQ ID NO: 815 - DNA, 29 bp, synthetic construct
  source 1..29: mol_type = other DNA
ttttaaatcggtaaggtggcatcaatttt

SEQ ID NO: 816 - DNA, 50 bp, synthetic construct
  source 1..50: mol_type = other DNA
ttttcaaggcaaagaattagcaaaacctaatcgtaaaactagcatgtttt

SEQ ID NO: 817 - DNA, 29 bp, synthetic construct
  source 1..29: mol_type = other DNA
ttttttctactaatagtaagcgaactttt

SEQ ID NO: 818 - DNA, 29 bp, synthetic construct
  source 1..29: mol_type = other DNA
ttttatttggggcgcgaaattgctctttt

SEQ ID NO: 819 - DNA, 29 bp, synthetic construct
  source 1..29: mol_type = other DNA
ttttgaccattagatacggatggcttttt

SEQ ID NO: 820 - DNA, 29 bp, synthetic construct
  source 1..29: mol_type = other DNA
ttttttcccaattctgcatatgcaatttt

SEQ ID NO: 821 - DNA, 29 bp, synthetic construct
  source 1..29: mol_type = other DNA
tttttagagcttatcgtcataaatatttt

SEQ ID NO: 822 - DNA, 29 bp, synthetic construct
  source 1..29: mol_type = other DNA
ttttcttttgataacgagaatgacctttt

SEQ ID NO: 823 - DNA, 29 bp, synthetic construct
  source 1..29: mol_type = other DNA
ttttcagaccggtttaccctgactatttt

SEQ ID NO: 824 - DNA, 50 bp, synthetic construct
  source 1..50: mol_type = other DNA
ttttaagcccgaaagacttcaaatattctccaataaatcatacaggtttt

SEQ ID NO: 825 - DNA, 29 bp, synthetic construct
  source 1..29: mol_type = other DNA
ttttttatagtcagaagccacattctttt

SEQ ID NO: 826 - DNA, 29 bp, synthetic construct
  source 1..29: mol_type = other DNA
ttttataaatcaaaaataaaggaattttt

SEQ ID NO: 827 - DNA, 29 bp, synthetic construct
  source 1..29: mol_type = other DNA
ttttttcattgaatcccacgacgattttt

SEQ ID NO: 828 - DNA, 29 bp, synthetic construct
  source 1..29: mol_type = other DNA
tttttagcgtccaatacttgcaaaatttt

SEQ ID NO: 829 - DNA, 29 bp, synthetic construct
  source 1..29: mol_type = other DNA
ttttaaaaaccatagtaaattgggctttt

SEQ ID NO: 830 - DNA, 29 bp, synthetic construct
  source 1..29: mol_type = other DNA
tttttacgaggcccttatgcgattttttt

SEQ ID NO: 831 - DNA, 29 bp, synthetic construct
  source 1..29: mol_type = other DNA
ttttaactaatgccagtcaggacgttttt

SEQ ID NO: 832 - DNA, 50 bp, synthetic construct
  source 1..50: mol_type = other DNA
ttttattattacaggtagaaagatttaaatcaaaaagattaagaggtttt

SEQ ID NO: 833 - DNA, 29 bp, synthetic construct
  source 1..29: mol_type = other DNA
tttttgggaagaaaaatgaacgaggtttt

SEQ ID NO: 834 - DNA, 29 bp, synthetic construct
  source 1..29: mol_type = other DNA
tttttaagaactggctcaggacagatttt

SEQ ID NO: 835 - DNA, 29 bp, synthetic construct
  source 1..29: mol_type = other DNA
ttttttgagatggtttataggctggtttt

SEQ ID NO: 836 - DNA, 29 bp, synthetic construct
  source 1..29: mol_type = other DNA
ttttcgagaaacaccagcccaaatctttt

SEQ ID NO: 837 - DNA, 29 bp, synthetic construct
  source 1..29: mol_type = other DNA
ttttctgaccttgccgacaatgacatttt

SEQ ID NO: 838 - DNA, 19 bp, synthetic construct
  source 1..19: mol_type = other DNA
tttttgaacggttcgtttt

SEQ ID NO: 839 - DNA, 29 bp, synthetic construct
  source 1..29: mol_type = other DNA
ttttcgcagacgacgaaggcaccaatttt

SEQ ID NO: 840 - DNA, 50 bp, synthetic construct
  source 1..50: mol_type = other DNA
ttttaaattgtgtcgaaatccgcgattaacgaactaacggaacaactttt

SEQ ID NO: 841 - DNA, 42 bp, synthetic construct
  source 1..42: mol_type = other DNA
ttttcctaaaacgaaagaggcaaaaatcatcgcctgattttt

SEQ ID NO: 842 - DNA, 51 bp, synthetic construct
  source 1..51: mol_type = other DNA
ttttgaacgagggtaatgccactgtcaatcacttagccgctacgttatttt

SEQ ID NO: 843 - DNA, 15 bp, synthetic construct
  source 1..15: mol_type = other DNA
ttttcaggcaaagcg

SEQ ID NO: 844 - DNA, 41 bp, synthetic construct
  source 1..41: mol_type = other DNA
gctgacgacagtatcggaagttttaggcttgccctgatttt

SEQ ID NO: 845 - DNA, 15 bp, synthetic construct
  source 1..15: mol_type = other DNA
ttttgccagtttgag

SEQ ID NO: 846 - DNA, 52 bp, synthetic construct
  source 1..52: mol_type = other DNA
ggaacaaataacaaccagggtgagcctgtagccaaaaataattcgcgttttt

SEQ ID NO: 847 - DNA, 33 bp, synthetic construct
  source 1..33: mol_type = other DNA
cggcggataatgtgtaatataacagttgatttt

SEQ ID NO: 848 - DNA, 33 bp, synthetic construct
  source 1..33: mol_type = other DNA
tttttcaatcatatgtaccccggttgatccagt

SEQ ID NO: 849 - DNA, 36 bp, synthetic construct
  source 1..36: mol_type = other DNA
tgatgttgagcaaataccccaaaaacaggaagtttt

SEQ ID NO: 850 - DNA, 36 bp, synthetic construct
  source 1..36: mol_type = other DNA
agctattttgttaaaatttaaattgtaaacgttttt

SEQ ID NO: 851 - DNA, 36 bp, synthetic construct
  source 1..36: mol_type = other DNA
taatgataaacgccattcagctcattttttaatttt

SEQ ID NO: 852 - DNA, 49 bp, synthetic construct
  source 1..49: mol_type = other DNA
taaggcgttaaataagaaaaacgtcggatattaaatgtgagcgagtttt

SEQ ID NO: 853 - DNA, 17 bp, synthetic construct
  source 1..17: mol_type = other DNA
ttttacaaccatcgccc

SEQ ID NO: 854 - DNA, 17 bp, synthetic construct
  source 1..17: mol_type = other DNA
ttttgaaaatctccaaa

SEQ ID NO: 855 - DNA, 40 bp, synthetic construct
  source 1..40: mol_type = other DNA
ttttttgcagcaagcggtcccctggccctgagagagtttt

SEQ ID NO: 856 - DNA, 40 bp, synthetic construct
  source 1..40: mol_type = other DNA
ttttaatcccttataaatcagttccgaaatcggcaatttt

SEQ ID NO: 857 - DNA, 40 bp, synthetic construct
  source 1..40: mol_type = other DNA
ttttcaaagggcgaaaaaccacgtggactccaacgttttt

SEQ ID NO: 858 - DNA, 40 bp, synthetic construct
  source 1..40: mol_type = other DNA
ttttccccgatttagagcttgaaccctaaagggagctttt

SEQ ID NO: 859 - DNA, 40 bp, synthetic construct
  source 1..40: mol_type = other DNA
ttttgcggtcacgctgcgcgggcgctggcaagtgtatttt

SEQ ID NO: 860 - DNA, 40 bp, synthetic construct
  source 1..40: mol_type = other DNA
tttttcacttgcctgagtagttgattagtaataacatttt

SEQ ID NO: 861 - DNA, 40 bp, synthetic construct
  source 1..40: mol_type = other DNA
ttttgaaatacctacattttaggaaaaacgctcatgtttt

SEQ ID NO: 862 - DNA, 40 bp, synthetic construct
  source 1..40: mol_type = other DNA
ttttccaacagagatagaacaaaagggacattctggtttt

SEQ ID NO: 863 - DNA, 40 bp, synthetic construct
  source 1..40: mol_type = other DNA
ttttagccctaaaacatcgctaatgcgcgaactgattttt

SEQ ID NO: 864 - DNA, 35 bp, synthetic construct
  source 1..35: mol_type = other DNA
tttttctagaggatcaacgcatgcctgcaggtttt

SEQ ID NO: 865 - DNA, 25 bp, synthetic construct
  source 1..25: mol_type = other DNA
ttttattcgtaattatccgcttttt

SEQ ID NO: 866 - DNA, 25 bp, synthetic construct
  source 1..25: mol_type = other DNA
tttttgtaaagccaattgcgttttt

SEQ ID NO: 867 - DNA, 19 bp, synthetic construct
  source 1..19: mol_type = other DNA
ttttagattaatgcatttt

SEQ ID NO: 868 - DNA, 19 bp, synthetic construct
  source 1..19: mol_type = other DNA
ttttctgaataaaaatttt

SEQ ID NO: 869 - DNA, 19 bp, synthetic construct
  source 1..19: mol_type = other DNA
tttttgaacaagcaatttt

SEQ ID NO: 870 - DNA, 19 bp, synthetic construct
  source 1..19: mol_type = other DNA
ttttccggaagtgcctttt

SEQ ID NO: 871 - DNA, 30 bp, synthetic construct
  source 1..30: mol_type = other DNA
tttttcggaaaggaacggcagtgaggtttt

SEQ ID NO: 872 - DNA, 19 bp, synthetic construct
  source 1..19: mol_type = other DNA
ttttgtcgatagtactttt

SEQ ID NO: 873 - DNA, 19 bp, synthetic construct
  source 1..19: mol_type = other DNA
ttttcgccactaccgtttt

SEQ ID NO: 874 - DNA, 19 bp, synthetic construct
  source 1..19: mol_type = other DNA
ttttaacgccgtctttttt

SEQ ID NO: 875 - DNA, 50 bp, synthetic construct
  source 1..50: mol_type = other DNA
cttcaccgacgctggtttgccccagcagggagtaattaattttccctttt

SEQ ID NO: 876 - DNA, 12 bp, synthetic construct
  source 1..12: mol_type = other DNA
ttttttcctcgt

SEQ ID NO: 877 - DNA, 36 bp, synthetic construct
  source 1..36: mol_type = other DNA
aatcctgatagaatcagcacgtataacgtgcttttt

SEQ ID NO: 878 - DNA, 28 bp, synthetic construct
  source 1..28: mol_type = other DNA
ttttgaagtgtttttataattacgccag

SEQ ID NO: 879 - DNA, 47 bp, synthetic construct
  source 1..47: mol_type = other DNA
gggcgaccaccagagaaaggaaaattgtataacctcaaatatctttt

SEQ ID NO: 880 - DNA, 21 bp, synthetic construct
  source 1..21: mol_type = other DNA
ccagctcgtagaaaatatttt

SEQ ID NO: 881 - DNA, 21 bp, synthetic construct
  source 1..21: mol_type = other DNA
gttttcttgaagccttatttt

SEQ ID NO: 882 - DNA, 21 bp, synthetic construct
  source 1..21: mol_type = other DNA
ccagtgcataattactatttt

SEQ ID NO: 883 - DNA, 34 bp, synthetic construct
  source 1..34: mol_type = other DNA
ttttttccacacaacatatttatattttagttaa

SEQ ID NO: 884 - DNA, 34 bp, synthetic construct
  source 1..34: mol_type = other DNA
tttttcactgcccgctttaatgcttaggttgggt

SEQ ID NO: 885 - DNA, 44 bp, synthetic construct
  source 1..44: mol_type = other DNA
ccaatcgcctaatgagttcgcattaaacgagccggaagcatttt

SEQ ID NO: 886 - DNA, 44 bp, synthetic construct
  source 1..44: mol_type = other DNA
aaataccgtagctgttcagctttcatccccgggtaccgagtttt

SEQ ID NO: 887 - DNA, 33 bp, synthetic construct
  source 1..33: mol_type = other DNA
accggaatccaagcttgacgttgtaaaactttt

SEQ ID NO: 888 - DNA, 33 bp, synthetic construct
  source 1..33: mol_type = other DNA
gggaggttccagtcacaagttgggtaacgtttt

SEQ ID NO: 889 - DNA, 33 bp, synthetic construct
  source 1..33: mol_type = other DNA
ttagcaaaggcgaaagggcctcttcgctatttt

SEQ ID NO: 890 - DNA, 33 bp, synthetic construct
  source 1..33: mol_type = other DNA
cagaaccatcggtgcgctgcgcaactgtttttt

SEQ ID NO: 891 - DNA, 37 bp, synthetic construct
  source 1..37: mol_type = other DNA
gggtcccaattctgcgaacccatataacagttgataa

SEQ ID NO: 892 - DNA, 37 bp, synthetic construct
  source 1..37: mol_type = other DNA
ttaggtcatttttgcggatgctccttttgataagacg

SEQ ID NO: 893 - DNA, 37 bp, synthetic construct
  source 1..37: mol_type = other DNA
ccagaagcccgaaagactttcaaaaagattaagaggg

SEQ ID NO: 894 - DNA, 37 bp, synthetic construct
  source 1..37: mol_type = other DNA
gactccccctcaaatgcttataaatattcattgaagg

SEQ ID NO: 895 - DNA, 37 bp, synthetic construct
  source 1..37: mol_type = other DNA
tcgagtaagagcaacactaaggaattacgaggcatac

SEQ ID NO: 896 - DNA, 35 bp, synthetic construct
  source 1..35: mol_type = other DNA
ctcttaataaaacgaactgaagaaaaatctacgga

SEQ ID NO: 897 - DNA, 37 bp, synthetic construct
  source 1..37: mol_type = other DNA
cacgtagtaaattgggcttagaaacaccagaacgaaa

SEQ ID NO: 898 - DNA, 35 bp, synthetic construct
  source 1..35: mol_type = other DNA
taagctgaccttcatcaacaggcgcataggctgag

SEQ ID NO: 899 - DNA, 37 bp, synthetic construct
  source 1..37: mol_type = other DNA
tgcataaattgtgtcgaaatttgtatcatcgcctggc

SEQ ID NO: 900 - DNA, 35 bp, synthetic construct
  source 1..35: mol_type = other DNA
aaaacatagcgatagcttttagaatccttgaaaga

SEQ ID NO: 901 - DNA, 37 bp, synthetic construct
  source 1..37: mol_type = other DNA
ggacaacaatagataagtcgaacgcgcctgtttatgt

SEQ ID NO: 902 - DNA, 35 bp, synthetic construct
  source 1..35: mol_type = other DNA
taagacgggagaattaaccagggaagcgcattaga

SEQ ID NO: 903 - DNA, 37 bp, synthetic construct
  source 1..37: mol_type = other DNA
cggattaccattagcaaggaatcaccagtagcaccaa

SEQ ID NO: 904 - DNA, 35 bp, synthetic construct
  source 1..35: mol_type = other DNA
acatgccccctgcctattcgtataaacagttaata

SEQ ID NO: 905 - DNA, 37 bp, synthetic construct
  source 1..37: mol_type = other DNA
acgcaggcggataagtgccgggttttgctcagtacca

SEQ ID NO: 906 - DNA, 35 bp, synthetic construct
  source 1..35: mol_type = other DNA
caacgccaccctcagaaccgccaccctcagaacaa

SEQ ID NO: 907 - DNA, 37 bp, synthetic construct
  source 1..37: mol_type = other DNA
aaacaccagtacaaactactaacactgagtttcgtcc

SEQ ID NO: 908 - DNA, 35 bp, synthetic construct
  source 1..35: mol_type = other DNA
taaatgaattttctgtattccagacgttagtaagc

SEQ ID NO: 909 - DNA, 42 bp, synthetic construct
  source 1..42: mol_type = other DNA
gatataagtatagtgacacagacagccctcatggagggaggg

SEQ ID NO: 910 - DNA, 32 bp, synthetic construct
  source 1..32: mol_type = other DNA
cttttgatgatgtcagtgccttggagggaggg

SEQ ID NO: 911 - DNA, 42 bp, synthetic construct
  source 1..42: mol_type = other DNA
cattgacaggaggatttaagcgtcatacatggggagggaggg

SEQ ID NO: 912 - DNA, 42 bp, synthetic construct
  source 1..42: mol_type = other DNA
gcaagcaaatcaggcttattttgcacccagctccagccagcc

SEQ ID NO: 913 - DNA, 32 bp, synthetic construct
  source 1..32: mol_type = other DNA
acaattttatccagagcctaatccagccagcc

SEQ ID NO: 914 - DNA, 42 bp, synthetic construct
  source 1..42: mol_type = other DNA
gtaagcagatagctataatagaaaattcatatccagccagcc

SEQ ID NO: 915 - DNA, 40 bp, synthetic construct
  source 1..40: mol_type = other DNA
ttttcagtacaaactacaaccactgagtttcgtcactttt

SEQ ID NO: 916 - DNA, 29 bp, synthetic construct
  source 1..29: mol_type = other DNA
ttttaattttctcagctttccggcatttt

SEQ ID NO: 917 - DNA, 23 bp, synthetic construct
  source 1..23: mol_type = other DNA
tttttcacgttggagatcttttt

SEQ ID NO: 918 - DNA, 40 bp, synthetic construct
  source 1..40: mol_type = other DNA
ttttataccgatagttgcgctttcttaaacagcttgtttt

SEQ ID NO: 919 - DNA, 30 bp, synthetic construct
  source 1..30: mol_type = other DNA
ttttccattaaacggcaagcgcgaaatttt

SEQ ID NO: 920 - DNA, 52 bp, synthetic construct
  source 1..52: mol_type = other DNA
gattatacgtaaaatatgtttagagtcaccctgttaaaggccgctttttttt

SEQ ID NO: 921 - DNA, 30 bp, synthetic construct
  source 1..30: mol_type = other DNA
ttttcaaagtacaacaaccgaactgatttt

SEQ ID NO: 922 - DNA, 33 bp, synthetic construct
  source 1..33: mol_type = other DNA
cataagggggagatttaagaagttttgcctttt

SEQ ID NO: 923 - DNA, 30 bp, synthetic construct
  source 1..30: mol_type = other DNA
ttttccaactttgaaaacgtaacaaatttt

SEQ ID NO: 924 - DNA, 33 bp, synthetic construct
  source 1..33: mol_type = other DNA
cccaaatcagaggacaccctcgtttaccatttt

SEQ ID NO: 925 - DNA, 30 bp, synthetic construct
  source 1..30: mol_type = other DNA
ttttgctgctcattcatgcgattttatttt

SEQ ID NO: 926 - DNA, 33 bp, synthetic construct
  source 1..33: mol_type = other DNA
attaccttagtgaatatacgaggcatagttttt

SEQ ID NO: 927 - DNA, 22 bp, synthetic construct
  source 1..22: mol_type = other DNA
ttttagaactggctccggtttt

SEQ ID NO: 928 - DNA, 41 bp, synthetic construct
  source 1..41: mol_type = other DNA
taataaaacgaactaaattataccgatttaggaatactttt

SEQ ID NO: 929 - DNA, 48 bp, synthetic construct
  source 1..48: mol_type = other DNA
ttttaacaacattatgcttcaaattcaaatagagagtacctttatttt

SEQ ID NO: 930 - DNA, 40 bp, synthetic construct
  source 1..40: mol_type = other DNA
ttttcacattcaactaatgaaaaagattaagaggaatttt

SEQ ID NO: 931 - DNA, 50 bp, synthetic construct
  source 1..50: mol_type = other DNA
ttttaagagcaacactagactattaaatcaaaatcaacatgttttatttt

SEQ ID NO: 932 - DNA, 40 bp, synthetic construct
  source 1..40: mol_type = other DNA
ttttgacgacgataaaaacgacagttcagaaaacgatttt

SEQ ID NO: 933 - DNA, 48 bp, synthetic construct
  source 1..48: mol_type = other DNA
ttttagagggggtaataataaatatagcgtccagtagatttagttttt

SEQ ID NO: 934 - DNA, 22 bp, synthetic construct
  source 1..22: mol_type = other DNA
ttttgattcattgaatcctttt

SEQ ID NO: 935 - DNA, 40 bp, synthetic construct
  source 1..40: mol_type = other DNA
ttttccctcaaatgctttaaggtgtgtctggaagtttttt

SEQ ID NO: 936 - DNA, 30 bp, synthetic construct
  source 1..30: mol_type = other DNA
ttttgaatgaccatatagtcagaagctttt

SEQ ID NO: 937 - DNA, 40 bp, synthetic construct
  source 1..40: mol_type = other DNA
ttttaaagcggattgcatcaacaggtcatttttgcgtttt

SEQ ID NO: 938 - DNA, 22 bp, synthetic construct
  source 1..22: mol_type = other DNA
ttttgcccgaaagacgcgtttt

SEQ ID NO: 939 - DNA, 48 bp, synthetic construct
  source 1..48: mol_type = other DNA
ttttaaccagaccggacattatgaaagctaatcaacgcaaggattttt

SEQ ID NO: 940 - DNA, 40 bp, synthetic construct
  source 1..40: mol_type = other DNA
ttttattgctccttttgcataaattaagcaataaagtttt

SEQ ID NO: 941 - DNA, 50 bp, synthetic construct
  source 1..50: mol_type = other DNA
ttttgatggcttagagcccaataaatactaatatgagaaaggccggtttt

SEQ ID NO: 942 - DNA, 40 bp, synthetic construct
  source 1..40: mol_type = other DNA
ttttaatatgcaactaaaaacgcgagctgaaaaggttttt

SEQ ID NO: 943 - DNA, 37 bp, synthetic construct
  source 1..37: mol_type = other DNA
tttttcattccatataagtcaataaaccattagattt

SEQ ID NO: 944 - DNA, 22 bp, synthetic construct
  source 1..22: mol_type = other DNA
ttttttgcctgtttagcttttt

SEQ ID NO: 945 - DNA, 40 bp, synthetic construct
  source 1..40: mol_type = other DNA
ttttatattttcatttggggtccaatatgatattcatttt

SEQ ID NO: 946 - DNA, 30 bp, synthetic construct
  source 1..30: mol_type = other DNA
ttttggcatcaattctcatacaggcatttt

SEQ ID NO: 947 - DNA, 40 bp, synthetic construct
  source 1..40: mol_type = other DNA
ttttaggcaaagaattagcaagcatatattttaaattttt

SEQ ID NO: 948 - DNA, 15 bp, synthetic construct
  source 1..15: mol_type = other DNA
ttttcctcagagcat

SEQ ID NO: 949 - DNA, 44 bp, synthetic construct
  source 1..44: mol_type = other DNA
ttttccctgtacattttttcattaaatctggccttcctgttttt

SEQ ID NO: 950 - DNA, 40 bp, synthetic construct
  source 1..40: mol_type = other DNA
ttttaaaaatttttagatcctaaacgttaatatttttttt

SEQ ID NO: 951 - DNA, 50 bp, synthetic construct
  source 1..50: mol_type = other DNA
ttttgcaatgcctgagtaaacaggaggttgataattgaccgtaatgtttt

SEQ ID NO: 952 - DNA, 29 bp, synthetic construct
  source 1..29: mol_type = other DNA
ttttagacagtcaaatctgtacccctttt

SEQ ID NO: 953 - DNA, 37 bp, synthetic construct
  source 1..37: mol_type = other DNA
ttttaccgttctagctggagcaaacatcaggtcactc

SEQ ID NO: 954 - DNA, 32 bp, synthetic construct
  source 1..32: mol_type = other DNA
ttgaaaaatctcgcgaataataattttttttt

SEQ ID NO: 955 - DNA, 26 bp, synthetic construct
  source 1..26: mol_type = other DNA
ttttaaaggctaagagaatcgatttt

SEQ ID NO: 956 - DNA, 40 bp, synthetic construct
  source 1..40: mol_type = other DNA
tttttgaacggtaatcgtaaaactgcatctgccagttttt

SEQ ID NO: 957 - DNA, 15 bp, synthetic construct
  source 1..15: mol_type = other DNA
agattgtataatttt

SEQ ID NO: 958 - DNA, 40 bp, synthetic construct
  source 1..40: mol_type = other DNA
ttttgcaaatatttaaattgtttcccgtcggattcttttt

SEQ ID NO: 959 - DNA, 15 bp, synthetic construct
  source 1..15: mol_type = other DNA
ttttgttaaaattcg

SEQ ID NO: 960 - DNA, 44 bp, synthetic construct
  source 1..44: mol_type = other DNA
ttttaccaatagtcgactcagtgccaagaaattgttatcctttt

SEQ ID NO: 961 - DNA, 40 bp, synthetic construct
  source 1..40: mol_type = other DNA
ttttagccagctttcatatacagtcacgacgttgtatttt

SEQ ID NO: 962 - DNA, 50 bp, synthetic construct
  source 1..50: mol_type = other DNA
ttttccgtgggaacaaacaaggcgaagctggcgaactcacattaattttt

SEQ ID NO: 963 - DNA, 40 bp, synthetic construct
  source 1..40: mol_type = other DNA
ttttggataggtcacgtgctcggtgcgggcctcttctttt

SEQ ID NO: 964 - DNA, 48 bp, synthetic construct
  source 1..48: mol_type = other DNA
ttttttgaggggacgacgccattcacggaaacccgtattgggcgtttt

SEQ ID NO: 965 - DNA, 32 bp, synthetic construct
  source 1..32: mol_type = other DNA
cagcgtatgggacagacgttagtaaatgtttt

SEQ ID NO: 966 - DNA, 32 bp, synthetic construct
  source 1..32: mol_type = other DNA
ttttccgcttctggtgcggctgcgcaactttt

SEQ ID NO: 967 - DNA, 40 bp, synthetic construct
  source 1..40: mol_type = other DNA
tttttgttgggaagggcgatattgtcgtgccagctgtttt

SEQ ID NO: 968 - DNA, 30 bp, synthetic construct
  source 1..30: mol_type = other DNA
ttttgctattacgccttaagttgggttttt

SEQ ID NO: 969 - DNA, 40 bp, synthetic construct
  source 1..40: mol_type = other DNA
ttttaacgccagggttttccaaagtgtaaagcctggtttt

SEQ ID NO: 970 - DNA, 32 bp, synthetic construct
  source 1..32: mol_type = other DNA
ttttaaacgacggcctagaggatccccgtttt

SEQ ID NO: 971 - DNA, 41 bp, synthetic construct
  source 1..41: mol_type = other DNA
ttttggtaccgagctcgaattcgtacaaagggcattaaaga

SEQ ID NO: 972 - DNA, 33 bp, synthetic construct
  source 1..33: mol_type = other DNA
ttttgctcacaattccatgttgttcagaatagc

SEQ ID NO: 973 - DNA, 33 bp, synthetic construct
  source 1..33: mol_type = other DNA
ttttggtgcctaatgagcgaaatcggaaaatcc

SEQ ID NO: 974 - DNA, 33 bp, synthetic construct
  source 1..33: mol_type = other DNA
tttttgcgttgcgctcaagcggtcccctggccc

SEQ ID NO: 975 - DNA, 40 bp, synthetic construct
  source 1..40: mol_type = other DNA
ttttcattaatgaatcgagacgggcaacagctgatttttt

SEQ ID NO: 976 - DNA, 39 bp, synthetic construct
  source 1..39: mol_type = other DNA
ttttccagggtggtttttctttttaccgtaagcctgtag

SEQ ID NO: 977 - DNA, 30 bp, synthetic construct
  source 1..30: mol_type = other DNA
ttttgcccttcaccgacgctggtttgtttt

SEQ ID NO: 978 - DNA, 30 bp, synthetic construct
  source 1..30: mol_type = other DNA
ttttccccagcaggcgcaaaatcccttttt

SEQ ID NO: 979 - DNA, 30 bp, synthetic construct
  source 1..30: mol_type = other DNA
tttttataaatcaaacagtttggaactttt

SEQ ID NO: 980 - DNA, 15 bp, synthetic construct
  source 1..15: mol_type = other DNA
ttttaagagtccact

SEQ ID NO: 981 - DNA, 35 bp, synthetic construct
  source 1..35: mol_type = other DNA
ttttgaaaaaccgtctatcatccaacgtatcatgg

SEQ ID NO: 982 - DNA, 33 bp, synthetic construct
  source 1..33: mol_type = other DNA
ccaataggaacccatgataacgtgttagagagg

SEQ ID NO: 983 - DNA, 33 bp, synthetic construct
  source 1..33: mol_type = other DNA
cattccacagttttgtttaaaaatccatcagga

SEQ ID NO: 984 - DNA, 32 bp, synthetic construct
  source 1..32: mol_type = other DNA
tcgagaggtcagtaccaggcggattaacagtg

SEQ ID NO: 985 - DNA, 48 bp, synthetic construct
  source 1..48: mol_type = other DNA
aagtatagaccctcagagccaccaccctcattttcagggaaagtgccg

SEQ ID NO: 986 - DNA, 40 bp, synthetic construct
  source 1..40: mol_type = other DNA
cgatctaaagacagccaagggattctttcctcgctttgac

SEQ ID NO: 987 - DNA, 33 bp, synthetic construct
  source 1..33: mol_type = other DNA
aacaacttaacaactagaacctactaaggagag

SEQ ID NO: 988 - DNA, 48 bp, synthetic construct
  source 1..48: mol_type = other DNA
cccgtatagggtcagtgccttgagcacaaacaaataaatcgattggcc

SEQ ID NO: 989 - DNA, 32 bp, synthetic construct
  source 1..32: mol_type = other DNA
tagaaaggtcaacagtttcagcggtagcgtaa

SEQ ID NO: 990 - DNA, 48 bp, synthetic construct
  source 1..48: mol_type = other DNA
ttttaacgaacagttaatgcccccattagcggggttttgcgttgatat

SEQ ID NO: 991 - DNA, 34 bp, synthetic construct
  source 1..34: mol_type = other DNA
aggctccattgctttcattttagttgaattctgc

SEQ ID NO: 992 - DNA, 28 bp, synthetic construct
  source 1..28: mol_type = other DNA
ttatcagcaaaggagcaacagaaacata

SEQ ID NO: 993 - DNA, 48 bp, synthetic construct
  source 1..48: mol_type = other DNA
ttgatattcgcctccctcagagccgagccaccaccggaaccagtagcg

SEQ ID NO: 994 - DNA, 31 bp, synthetic construct
  source 1..31: mol_type = other DNA
acaacaacctgaggctcattaccgcttatcc

SEQ ID NO: 995 - DNA, 48 bp, synthetic construct
  source 1..48: mol_type = other DNA
cagaaccgttgaggcaggtcagacctcattaaagccagaagtaataag

SEQ ID NO: 996 - DNA, 28 bp, synthetic construct
  source 1..28: mol_type = other DNA
ttcggtcgcatcgccctaatggtttaat

SEQ ID NO: 997 - DNA, 24 bp, synthetic construct
  source 1..24: mol_type = other DNA
aaagacagctttgaggcactacga

SEQ ID NO: 998 - DNA, 40 bp, synthetic construct
  source 1..40: mol_type = other DNA
acagaatcatagcagcgtgaattatcaccgtcaaattatt

SEQ ID NO: 999 - DNA, 24 bp, synthetic construct
  source 1..24: mol_type = other DNA
ctttttcatgaggaaggcgggatc

SEQ ID NO: 1000 - DNA, 36 bp, synthetic construct
  source 1..36: mol_type = other DNA
tacagaggcatcggaaatagaaggcgcccaattttt

SEQ ID NO: 1001 - DNA, 48 bp, synthetic construct
  source 1..48: mol_type = other DNA
aaccatcgaagtttgcctttagcgaaaatcaccggaaccagccaccct

SEQ ID NO: 1002 - DNA, 29 bp, synthetic construct
  source 1..29: mol_type = other DNA
aggcaccaaaacactcgcgttttagcgaa

SEQ ID NO: 1003 - DNA, 24 bp, synthetic construct
  source 1..24: mol_type = other DNA
cgaaagagaccgtaatgcaacggc

SEQ ID NO: 1004 - DNA, 40 bp, synthetic construct
  source 1..40: mol_type = other DNA
atacactaacctaaaaaatcagatcgagggtaccgatata

SEQ ID NO: 1005 - DNA, 48 bp, synthetic construct
  source 1..48: mol_type = other DNA
cattaaagtttattttgtcacaatgacaccacggaataagtacccaaa

SEQ ID NO: 1006 - DNA, 40 bp, synthetic construct
  source 1..40: mol_type = other DNA
attgacggaccgacttgagccattgaaacgtcaccaatga

SEQ ID NO: 1007 - DNA, 29 bp, synthetic construct
  source 1..29: mol_type = other DNA
aattgtgtcggaacgattttgaagcctta

SEQ ID NO: 1008 - DNA, 29 bp, synthetic construct
  source 1..29: mol_type = other DNA
accaggcgttgacaagtatcctgaatctt

SEQ ID NO: 1009 - DNA, 48 bp, synthetic construct
  source 1..48: mol_type = other DNA
ggctgaccctccatgttacttagccgaaatccgcgacctggcaaaaga

SEQ ID NO: 1010 - DNA, 48 bp, synthetic construct
  source 1..48: mol_type = other DNA
agaactggcgcaataataacggaaagagcaagaaacaatggttaagcc

SEQ ID NO: 1011 - DNA, 48 bp, synthetic construct
  source 1..48: mol_type = other DNA
agactcctataaaagaaacgcaaacaatagaaaattcataaggtaaat

SEQ ID NO: 1012 - DNA, 36 bp, synthetic construct
  source 1..36: mol_type = other DNA
gaaacacctaatttcatttccagatattatttaacg

SEQ ID NO: 1013 - DNA, 48 bp, synthetic construct
  source 1..48: mol_type = other DNA
agatggttagaacgagtagtaaatttcatcaagagtaatccataggct

SEQ ID NO: 1014 - DNA, 48 bp, synthetic construct
  source 1..48: mol_type = other DNA
caataataaaaacagggaagcgcattagacgggagaattaaacccaca

SEQ ID NO: 1015 - DNA, 48 bp, synthetic construct
  source 1..48: mol_type = other DNA
agaattgaaaatagcaatagctataaggaaaccgaggaaacatgatta

SEQ ID NO: 1016 - DNA, 30 bp, synthetic construct
  source 1..30: mol_type = other DNA
gagagaaacagccagcctaatttgccagtt

SEQ ID NO: 1017 - DNA, 24 bp, synthetic construct
  source 1..24: mol_type = other DNA
tcagttgaagtcaggacattgtga

SEQ ID NO: 1018 - DNA, 24 bp, synthetic construct
  source 1..24: mol_type = other DNA
acaaaataataacatatgggcttg

SEQ ID NO: 1019 - DNA, 34 bp, synthetic construct
  source 1..34: mol_type = other DNA
cataagtcactttaatcgttgggaagactttaca

SEQ ID NO: 1020 - DNA, 23 bp, synthetic construct
  source 1..23: mol_type = other DNA
aaggaataggcttgcattcatta

SEQ ID NO: 1021 - DNA, 21 bp, synthetic construct
  source 1..21: mol_type = other DNA
accaacgctaacgatcctaat

SEQ ID NO: 1022 - DNA, 23 bp, synthetic construct
  source 1..23: mol_type = other DNA
acaatttaaccggatcctgacga

SEQ ID NO: 1023 - DNA, 26 bp, synthetic construct
  source 1..26: mol_type = other DNA
tttgcactaagatgaacgcggtcaat

SEQ ID NO: 1024 - DNA, 21 bp, synthetic construct
  source 1..21: mol_type = other DNA
aatcaagattagtataatcgg

SEQ ID NO: 1025 - DNA, 25 bp, synthetic construct
  source 1..25: mol_type = other DNA
tagcgaaggggcgcagagtgtacag

SEQ ID NO: 1026 - DNA, 22 bp, synthetic construct
  source 1..22: mol_type = other DNA
ttgcaagtatcatccccccagc

SEQ ID NO: 1027 - DNA, 42 bp, synthetic construct
  source 1..42: mol_type = other DNA
cctcccgacttgccactcatcctgtctttgtatcatatgcgt

SEQ ID NO: 1028 - DNA, 22 bp, synthetic construct
  source 1..22: mol_type = other DNA
cgcgagatctttgagcctgata

SEQ ID NO: 1029 - DNA, 25 bp, synthetic construct
  source 1..25: mol_type = other DNA
ggtattaaacgtaatgcactaaaga

SEQ ID NO: 1030 - DNA, 40 bp, synthetic construct
  source 1..40: mol_type = other DNA
catcaccgaccgaccggaatacgcgagaataactattttt

SEQ ID NO: 1031 - DNA, 19 bp, synthetic construct
  source 1..19: mol_type = other DNA
agccgtttttaagcaagca

SEQ ID NO: 1032 - DNA, 32 bp, synthetic construct
  source 1..32: mol_type = other DNA
gagaacaagaataaactgtgataaataaggcg

SEQ ID NO: 1033 - DNA, 21 bp, synthetic construct
  source 1..21: mol_type = other DNA
ttacgagcataaagccaacgc

SEQ ID NO: 1034 - DNA, 48 bp, synthetic construct
  source 1..48: mol_type = other DNA
tgtttatcacgccaactaataagaattaattaaccttgctctttttta

SEQ ID NO: 1035 - DNA, 36 bp, synthetic construct
  source 1..36: mol_type = other DNA
cgccaaacaacaaaagtaccgacaaaagagtgaata

SEQ ID NO: 1036 - DNA, 32 bp, synthetic construct
  source 1..32: mol_type = other DNA
taattcgatacaggtagaaagccaatctacgt

SEQ ID NO: 1037 - DNA, 31 bp, synthetic construct
  source 1..31: mol_type = other DNA
aggattatcgcgtttataagtcctgcagata

SEQ ID NO: 1038 - DNA, 32 bp, synthetic construct
  source 1..32: mol_type = other DNA
ggcattttcgagccagatgtaatttaggcaga

SEQ ID NO: 1039 - DNA, 31 bp, synthetic construct
  source 1..31: mol_type = other DNA
tttaacaaacaatagtaatgcagatcattca

SEQ ID NO: 1040 - DNA, 34 bp, synthetic construct
  source 1..34: mol_type = other DNA
agaatcagaagaaaaattttacccttcaccagct

SEQ ID NO: 1041 - DNA, 33 bp, synthetic construct
  source 1..33: mol_type = other DNA
tcaacagtagggcacgctgagattttcccaaac

SEQ ID NO: 1042 - DNA, 34 bp, synthetic construct
  source 1..34: mol_type = other DNA
ctgaatagtatgtagaaaatatcccagccgccaa

SEQ ID NO: 1043 - DNA, 29 bp, synthetic construct
  source 1..29: mol_type = other DNA
tgtagcatcaggtcttccaagaaccaaaa

SEQ ID NO: 1044 - DNA, 41 bp, synthetic construct
  source 1..41: mol_type = other DNA
tatacaaattcttctttttaaaaaatcattacaaaattgag

SEQ ID NO: 1045 - DNA, 29 bp, synthetic construct
  source 1..29: mol_type = other DNA
ctgtttaccttatcaaccaatcatgctat

SEQ ID NO: 1046 - DNA, 35 bp, synthetic construct
  source 1..35: mol_type = other DNA
actagaacgattaaaccgaatcgtcgtactaagaa

SEQ ID NO: 1047 - DNA, 15 bp, synthetic construct
  source 1..15: mol_type = other DNA
ttttttaaataagca

SEQ ID NO: 1048 - DNA, 35 bp, synthetic construct
  source 1..35: mol_type = other DNA
ttcccaaatgtaggaataagtaccgggggaggctt

SEQ ID NO: 1049 - DNA, 30 bp, synthetic construct
  source 1..30: mol_type = other DNA
gaacgaatactgcgtgcagggacagcagcg

SEQ ID NO: 1050 - DNA, 24 bp, synthetic construct
  source 1..24: mol_type = other DNA
cctaaattacgcataagtatcggt

SEQ ID NO: 1051 - DNA, 25 bp, synthetic construct
  source 1..25: mol_type = other DNA
ttcaaaacttttaattgcgtagatt

SEQ ID NO: 1052 - DNA, 15 bp, synthetic construct
  source 1..15: mol_type = other DNA
ttttttttcttctga

SEQ ID NO: 1053 - DNA, 15 bp, synthetic construct
  source 1..15: mol_type = other DNA
ggttgggttattttt

SEQ ID NO: 1054 - DNA, 15 bp, synthetic construct
  source 1..15: mol_type = other DNA
ttttaagagtcaata

SEQ ID NO: 1055 - DNA, 49 bp, synthetic construct
  source 1..49: mol_type = other DNA
tgcgggtactctgtaaataccaaaaaagcaaactccaatattgttcagc

SEQ ID NO: 1056 - DNA, 48 bp, synthetic construct
  source 1..48: mol_type = other DNA
atggaaacctaatagatttagaagaatcaacacaatcaatatctggtc

SEQ ID NO: 1057 - DNA, 32 bp, synthetic construct
  source 1..32: mol_type = other DNA
ctttattatcggttgcttgaaaaatagccata

SEQ ID NO: 1058 - DNA, 32 bp, synthetic construct
  source 1..32: mol_type = other DNA
atcaagattagaatctcgtcgctgaacaggtc

SEQ ID NO: 1059 - DNA, 36 bp, synthetic construct
  source 1..36: mol_type = other DNA
aaaattcatttgtttgaggattagagccaggaaggt

SEQ ID NO: 1060 - DNA, 32 bp, synthetic construct
  source 1..32: mol_type = other DNA
gatgaactcgatagcttattaacatttaattg

SEQ ID NO: 1061 - DNA, 32 bp, synthetic construct
  source 1..32: mol_type = other DNA
caaattttaaaaaacaattccaaaccctgttg

SEQ ID NO: 1062 - DNA, 32 bp, synthetic construct
  source 1..32: mol_type = other DNA
aggtaaagtaggtctgaagattaagttaattg

SEQ ID NO: 1063 - DNA, 32 bp, synthetic construct
  source 1..32: mol_type = other DNA
aaaaggggtagtagcgctgatgcagtaaaagc

SEQ ID NO: 1064 - DNA, 32 bp, synthetic construct
  source 1..32: mol_type = other DNA
ggattcgcctgattgccgggagaaattcatca

SEQ ID NO: 1065 - DNA, 32 bp, synthetic construct
  source 1..32: mol_type = other DNA
agtaccatgtaaatgagactacaccataatgc

SEQ ID NO: 1066 - DNA, 41 bp, synthetic construct
  source 1..41: mol_type = other DNA
acattttgaatagagcggaagcggaacatctaaagcatcac

SEQ ID NO: 1067 - DNA, 31 bp, synthetic construct
  source 1..31: mol_type = other DNA
atataatcaatcgcaacgcaaatgcagttga

SEQ ID NO: 1068 - DNA, 42 bp, synthetic construct
  source 1..42: mol_type = other DNA
ttcaggtttaacgtacttctgatataatcattaacaccgcct

SEQ ID NO: 1069 - DNA, 32 bp, synthetic construct
  source 1..32: mol_type = other DNA
aatgccagatcaaatatgacaaagacataatt

SEQ ID NO: 1070 - DNA, 31 bp, synthetic construct
  source 1..31: mol_type = other DNA
ggtagctatacatttgaggtgaacgacaatg

SEQ ID NO: 1071 - DNA, 23 bp, synthetic construct
  source 1..23: mol_type = other DNA
cacgtaactttaattagtgagaa

SEQ ID NO: 1072 - DNA, 25 bp, synthetic construct
  source 1..25: mol_type = other DNA
tcaaaataatgggcagaagataaaa

SEQ ID NO: 1073 - DNA, 15 bp, synthetic construct
  source 1..15: mol_type = other DNA
tttttttaattattt

SEQ ID NO: 1074 - DNA, 32 bp, synthetic construct
  source 1..32: mol_type = other DNA
catcaattggtcaatagaatcagctatacttt

SEQ ID NO: 1075 - DNA, 16 bp, synthetic construct
  source 1..16: mol_type = other DNA
tatctaaattgacgct

SEQ ID NO: 1076 - DNA, 32 bp, synthetic construct
  source 1..32: mol_type = other DNA
ttcgcgtttttgttagtattaaaaccacaaac

SEQ ID NO: 1077 - DNA, 14 bp, synthetic construct
  source 1..14: mol_type = other DNA
agttggcatatttt

SEQ ID NO: 1078 - DNA, 33 bp, synthetic construct
  source 1..33: mol_type = other DNA
aatgttatgacaactcataatacaaatagaagc

SEQ ID NO: 1079 - DNA, 30 bp, synthetic construct
  source 1..30: mol_type = other DNA
gtaacaagcccgaacagccccaaaatgtgt

SEQ ID NO: 1080 - DNA, 42 bp, synthetic construct
  source 1..42: mol_type = other DNA
cttgctgaacctccagagatagattcacctggtaatatccag

SEQ ID NO: 1081 - DNA, 29 bp, synthetic construct
  source 1..29: mol_type = other DNA
gaaaaaaagaaaccgttattaaagaagat

SEQ ID NO: 1082 - DNA, 34 bp, synthetic construct
  source 1..34: mol_type = other DNA
ccagccggatcagaaacaatcataacccagtaac

SEQ ID NO: 1083 - DNA, 25 bp, synthetic construct
  source 1..25: mol_type = other DNA
gcaacagtgccacagaatacggaac

SEQ ID NO: 1084 - DNA, 34 bp, synthetic construct
  source 1..34: mol_type = other DNA
gatgggagtctgattgtaccagaagccaagattc

SEQ ID NO: 1085 - DNA, 29 bp, synthetic construct
  source 1..29: mol_type = other DNA
taaccgtagcatgtagagtctgataaatt

SEQ ID NO: 1086 - DNA, 42 bp, synthetic construct
  source 1..42: mol_type = other DNA
cagaggtgaggcgactgatagtggcacagagtaaaagagtct

SEQ ID NO: 1087 - DNA, 33 bp, synthetic construct
  source 1..33: mol_type = other DNA
tcggccccaaagggttattggattatcagatga

SEQ ID NO: 1088 - DNA, 31 bp, synthetic construct
  source 1..31: mol_type = other DNA
agatcgcattgcctgaaggaattcaaaaaaa

SEQ ID NO: 1089 - DNA, 16 bp, synthetic construct
  source 1..16: mol_type = other DNA
tttacgaaaccgattt

SEQ ID NO: 1090 - DNA, 40 bp, synthetic construct
  source 1..40: mol_type = other DNA
ccctaaaaaggaacggcagtgaggatgcgccgtaaccacc

SEQ ID NO: 1091 - DNA, 36 bp, synthetic construct
  source 1..36: mol_type = other DNA
ccttgattagtaactatcggcggcgaacgatttaga

SEQ ID NO: 1092 - DNA, 32 bp, synthetic construct
  source 1..32: mol_type = other DNA
tcataggcctgaaatgggcctgcagggaacgc

SEQ ID NO: 1093 - DNA, 30 bp, synthetic construct
  source 1..30: mol_type = other DNA
ctgtgtgcttgcatgaccagtacaacatta

SEQ ID NO: 1094 - DNA, 29 bp, synthetic construct
  source 1..29: mol_type = other DNA
aacaatattaccgcactaaatttttgggg

SEQ ID NO: 1095 - DNA, 34 bp, synthetic construct
  source 1..34: mol_type = other DNA
tacgagtgcagtcacacattatttagaaaaataa

SEQ ID NO: 1096 - DNA, 33 bp, synthetic construct
  source 1..33: mol_type = other DNA
gcataaagggacattatgtgctgcggagcaaat

SEQ ID NO: 1097 - DNA, 31 bp, synthetic construct
  source 1..31: mol_type = other DNA
ttctttctgacctgctggccaaaaagagcga

SEQ ID NO: 1098 - DNA, 15 bp, synthetic construct
  source 1..15: mol_type = other DNA
tttttttacttgcct

SEQ ID NO: 1099 - DNA, 31 bp, synthetic construct
  source 1..31: mol_type = other DNA
ttgtagctaaagggggtttgaatgtggtgta

SEQ ID NO: 1100 - DNA, 29 bp, synthetic construct
  source 1..29: mol_type = other DNA
gtccatcacgcaacgctggcaaagcgaaa

SEQ ID NO: 1101 - DNA, 33 bp, synthetic construct
  source 1..33: mol_type = other DNA
ctttccccgacaatattaaagcgtagctgagag

SEQ ID NO: 1102 - DNA, 30 bp, synthetic construct
  source 1..30: mol_type = other DNA
gaaacctagtctttacgccattcgacagta

SEQ ID NO: 1103 - DNA, 35 bp, synthetic construct
  source 1..35: mol_type = other DNA
cgcggggaccatcgccaatgcgcgagtccgcatcg

SEQ ID NO: 1104 - DNA, 15 bp, synthetic construct
  source 1..15: mol_type = other DNA
tttttttaatcctga

SEQ ID NO: 1105 - DNA, 31 bp, synthetic construct
  source 1..31: mol_type = other DNA
cggtttgaggcaaagcgtctttcttttgcta

SEQ ID NO: 1106 - DNA, 25 bp, synthetic construct
  source 1..25: mol_type = other DNA
gagcacgtcgcgcttaccaagtcgg

SEQ ID NO: 1107 - DNA, 23 bp, synthetic construct
  source 1..23: mol_type = other DNA
tgagagagcaccagtggccaacg

SEQ ID NO: 1108 - DNA, 24 bp, synthetic construct
  source 1..24: mol_type = other DNA
acacccgccgctagggattaaccg

SEQ ID NO: 1109 - DNA, 23 bp, synthetic construct
  source 1..23: mol_type = other DNA
tgtttgatttgcagcactgcccg

SEQ ID NO: 1110 - DNA, 25 bp, synthetic construct
  source 1..25: mol_type = other DNA
ggagcggggggaaagccctccggaa

SEQ ID NO: 1111 - DNA, 26 bp, synthetic construct
  source 1..26: mol_type = other DNA
ccgagataggtggttctgagcaatac

SEQ ID NO: 1112 - DNA, 26 bp, synthetic construct
  source 1..26: mol_type = other DNA
gcttgacgccgtaaagccactgtttc

SEQ ID NO: 1113 - DNA, 23 bp, synthetic construct
  source 1..23: mol_type = other DNA
acgtggacgggttgagcacaaca

SEQ ID NO: 1114 - DNA, 40 bp, synthetic construct
  source 1..40: mol_type = other DNA
ttttcaccctcagaaccgcccccggaataggtgtattttt

SEQ ID NO: 1115 - DNA, 40 bp, synthetic construct
  source 1..40: mol_type = other DNA
ttttcaagagaaggattaggtgcctatttcggaacctttt

SEQ ID NO: 1116 - DNA, 40 bp, synthetic construct
  source 1..40: mol_type = other DNA
ttttatacaggagtgtactgtggaaagcgcagtctctttt

SEQ ID NO: 1117 - DNA, 40 bp, synthetic construct
  source 1..40: mol_type = other DNA
ttttcagcattgacaggaggccaccctcagagccactttt

SEQ ID NO: 1118 - DNA, 40 bp, synthetic construct
  source 1..40: mol_type = other DNA
ttttccatcttttcataatctcagactgtagcgcgttttt

SEQ ID NO: 1119 - DNA, 32 bp, synthetic construct
  source 1..32: mol_type = other DNA
ttttcaaggccgtgggaattagagccagtttt

SEQ ID NO: 1120 - DNA, 40 bp, synthetic construct
  source 1..40: mol_type = other DNA
ttttccgattgagggagggatggtttaccagcgccatttt

SEQ ID NO: 1121 - DNA, 40 bp, synthetic construct
  source 1..40: mol_type = other DNA
ttttataaaggtggcaacattattacgcagtatgtttttt

SEQ ID NO: 1122 - DNA, 40 bp, synthetic construct
  source 1..40: mol_type = other DNA
ttttcgaacaaagttaccagcttaccgaagcccttttttt

SEQ ID NO: 1123 - DNA, 40 bp, synthetic construct
  source 1..40: mol_type = other DNA
ttttctaatatcagagagatactgaacaccctgaactttt

SEQ ID NO: 1124 - DNA, 47 bp, synthetic construct
  source 1..47: mol_type = other DNA
ttttgaaaatagcagcaaaatccaaataagaaacgacgacaattttt

SEQ ID NO: 1125 - DNA, 24 bp, synthetic construct
  source 1..24: mol_type = other DNA
tttttccagacgattttttgtttt

SEQ ID NO: 1126 - DNA, 24 bp, synthetic construct
  source 1..24: mol_type = other DNA
ttttactaacaaagtacatatttt

SEQ ID NO: 1127 - DNA, 44 bp, synthetic construct
  source 1..44: mol_type = other DNA
ttttaaagcattggcacaatcgtcattgcaacaggaaaaatttt

SEQ ID NO: 1128 - DNA, 48 bp, synthetic construct
  source 1..48: mol_type = other DNA
ttttgagtagaagaactcaaataacatcagggaagaagtgtagctttt

SEQ ID NO: 1129 - DNA, 52 bp, synthetic construct
  source 1..52: mol_type = other DNA
gaagtgtttttataattacgccagctatggttgttagaatcagagcggtttt

SEQ ID NO: 1130 - DNA, 40 bp, synthetic construct
  source 1..40: mol_type = other DNA
ttttaacaggaggccgattactcatagttagcaagctttt

SEQ ID NO: 1131 - DNA, 24 bp, synthetic construct
  source 1..24: mol_type = other DNA
ttttgctgcgcgctacagggtttt

SEQ ID NO: 1132 - DNA, 24 bp, synthetic construct
  source 1..24: mol_type = other DNA
ttttgagcccccgtggcgagtttt

SEQ ID NO: 1133 - DNA, 28 bp, synthetic construct
  source 1..28: mol_type = other DNA
tcgaggtgcgatggcccactacgttttt

SEQ ID NO: 1134 - DNA, 24 bp, synthetic construct
  source 1..24: mol_type = other DNA
ttttatcaagttcggaaccctttt

SEQ ID NO: 1135 - DNA, 24 bp, synthetic construct
  source 1..24: mol_type = other DNA
ttttattgacggaccgactttttt

SEQ ID NO: 1136 - DNA, 24 bp, synthetic construct
  source 1..24: mol_type = other DNA
tttttttgggaaaccattagtttt

SEQ ID NO: 1137 - DNA, 24 bp, synthetic construct
  source 1..24: mol_type = other DNA
ttttcggaaacgatcagtagtttt

SEQ ID NO: 1138 - DNA, 24 bp, synthetic construct
  source 1..24: mol_type = other DNA
ttttaatcaagtatcggcattttt

SEQ ID NO: 1139 - DNA, 25 bp, synthetic construct
  source 1..25: mol_type = other DNA
tttttcatagccaaaatcacctttt

SEQ ID NO: 1140 - DNA, 53 bp, synthetic construct
  source 1..53: mol_type = other DNA
ttttgagccaccaccggaaccgagccgccaccgtaacagcaagccccagacgt

SEQ ID NO: 1141 - DNA, 44 bp, synthetic construct
  source 1..44: mol_type = other DNA
ttttcctcagagccaccaccctaccagaaccaccaccagatttt

SEQ ID NO: 1142 - DNA, 43 bp, synthetic construct
  source 1..43: mol_type = other DNA
ttttgccagcattgacaggaggttgagagatcagaaccgccac

SEQ ID NO: 1143 - DNA, 44 bp, synthetic construct
  source 1..44: mol_type = other DNA
ttttcaaacaaataaatcctcaaatggaaagcgcagtctctttt

SEQ ID NO: 1144 - DNA, 43 bp, synthetic construct
  source 1..43: mol_type = other DNA
tttttaccgttccagtaagcgtcatacagcggggttttgctca

SEQ ID NO: 1145 - DNA, 36 bp, synthetic construct
  source 1..36: mol_type = other DNA
ttttttttaacgaaacatgaaagtattatttcgagg

SEQ ID NO: 1146 - DNA, 28 bp, synthetic construct
  source 1..28: mol_type = other DNA
ttttggaacctattattctggggtcagt

SEQ ID NO: 1147 - DNA, 22 bp, synthetic construct
  source 1..22: mol_type = other DNA
ccgtactcttggccttgatttt

SEQ ID NO: 1148 - DNA, 22 bp, synthetic construct
  source 1..22: mol_type = other DNA
cccatgtaccctcagaactttt

SEQ ID NO: 1149 - DNA, 38 bp, synthetic construct
  source 1..38: mol_type = other DNA
cagcttgcagaggctgagactcctatacaggagttttt

SEQ ID NO: 1150 - DNA, 42 bp, synthetic construct
  source 1..42: mol_type = other DNA
ttttaaccatcgcccacgcatttttaagaactggctcatttt

SEQ ID NO: 1151 - DNA, 28 bp, synthetic construct
  source 1..28: mol_type = other DNA
tattcggtttaaacagcttgatactttt

SEQ ID NO: 1152 - DNA, 44 bp, synthetic construct
  source 1..44: mol_type = other DNA
ttttaaaaatctacgttaatgaattaccttatgcgaaaccgata

SEQ ID NO: 1153 - DNA, 44 bp, synthetic construct
  source 1..44: mol_type = other DNA
ttttgaacgagtagatttagttttgtaaacgttaatattttttt

SEQ ID NO: 1154 - DNA, 28 bp, synthetic construct
  source 1..28: mol_type = other DNA
agatacatggaagtttcattccattttt

SEQ ID NO: 1155 - DNA, 32 bp, synthetic construct
  source 1..32: mol_type = other DNA
ttttttcgcattaaatttttctattaaatttt

SEQ ID NO: 1156 - DNA, 40 bp, synthetic construct
  source 1..40: mol_type = other DNA
gcaacattaaagattcaaccgattgagggagggaagtttt

SEQ ID NO: 1157 - DNA, 56 bp, synthetic construct
  source 1..56: mol_type = other DNA
ttttgtgctgcaaggcgattaagttggggcgatcggtgcgggcctcttcgcttttt

SEQ ID NO: 1158 - DNA, 40 bp, synthetic construct
  source 1..40: mol_type = other DNA
tttttggtcatagctgtttcgcatgcctgcaggtcgtttt

SEQ ID NO: 1159 - DNA, 40 bp, synthetic construct
  source 1..40: mol_type = other DNA
ttttgctttccagtcgggaaagcctggggtgcctaatttt

SEQ ID NO: 1160 - DNA, 40 bp, synthetic construct
  source 1..40: mol_type = other DNA
ttttcgcctggccctgagaggcgccagggtggtttttttt

SEQ ID NO: 1161 - DNA, 40 bp, synthetic construct
  source 1..40: mol_type = other DNA
ttttgttccagtttggaacacgaaatcggcaaaatctttt

SEQ ID NO: 1162 - DNA, 54 bp, synthetic construct
  source 1..54: mol_type = other DNA
ttttgcgaaaaaccgtctatcaatggcccactacgtgaagagtccagttaaatc

SEQ ID NO: 1163 - DNA, 30 bp, synthetic construct
  source 1..30: mol_type = other DNA
gccttgagtaacagtgcccgtataaatttt

SEQ ID NO: 1164 - DNA, 31 bp, synthetic construct
  source 1..31: mol_type = other DNA
atagaaaaaataagttctggtcagaggttat

SEQ ID NO: 1165 - DNA, 40 bp, synthetic construct
  source 1..40: mol_type = other DNA
tcaccgtcaaattattagcgccataagaactctaataaca

SEQ ID NO: 1166 - DNA, 34 bp, synthetic construct
  source 1..34: mol_type = other DNA
acatataagaaaatacttgctttgttaatccccc

SEQ ID NO: 1167 - DNA, 19 bp, synthetic construct
  source 1..19: mol_type = other DNA
gcaccattttagagccgcc

SEQ ID NO: 1168 - DNA, 31 bp, synthetic construct
  source 1..31: mol_type = other DNA
tccttattcaaaagaaaaatatatatggttt

SEQ ID NO: 1169 - DNA, 29 bp, synthetic construct
  source 1..29: mol_type = other DNA
gcaccgtatcaccaatcagttcagaaaac

SEQ ID NO: 1170 - DNA, 31 bp, synthetic construct
  source 1..31: mol_type = other DNA
accgaggagccgaacaccaagaacacaagca

SEQ ID NO: 1171 - DNA, 29 bp, synthetic construct
  source 1..29: mol_type = other DNA
gcgttttcttgcctttcatcgcctgataa

SEQ ID NO: 1172 - DNA, 19 bp, synthetic construct
  source 1..19: mol_type = other DNA
tcataatccccttattact

SEQ ID NO: 1173 - DNA, 41 bp, synthetic construct
  source 1..41: mol_type = other DNA
aagactcagcctccattcagtacaaagcgtttgactgtagc

SEQ ID NO: 1174 - DNA, 47 bp, synthetic construct
  source 1..47: mol_type = other DNA
acacccccgcccagagtgacagggatactgagtttccctcataacgc

SEQ ID NO: 1175 - DNA, 41 bp, synthetic construct
  source 1..41: mol_type = other DNA
cagaggcaggtcagacgaaggaggttcggaatagatttttt

SEQ ID NO: 1176 - DNA, 46 bp, synthetic construct
  source 1..46: mol_type = other DNA
ccaaagccagttaaataagtatagcctagtaccgagtgagaaaaca

SEQ ID NO: 1177 - DNA, 34 bp, synthetic construct
  source 1..34: mol_type = other DNA
ttttgatgcaagagaaggattaggatacctttaa

SEQ ID NO: 1178 - DNA, 23 bp, synthetic construct
  source 1..23: mol_type = other DNA
ctacaaagcctaatttgcccaat

SEQ ID NO: 1179 - DNA, 23 bp, synthetic construct
  source 1..23: mol_type = other DNA
gtaccaggcggataacgaaaatc

SEQ ID NO: 1180 - DNA, 20 bp, synthetic construct
  source 1..20: mol_type = other DNA
atataagaaacgatccttta

SEQ ID NO: 1181 - DNA, 21 bp, synthetic construct
  source 1..21: mol_type = other DNA
aacgccccataacataactga

SEQ ID NO: 1182 - DNA, 23 bp, synthetic construct
  source 1..23: mol_type = other DNA
cctcagaaccgccgagatgaatt

SEQ ID NO: 1183 - DNA, 21 bp, synthetic construct
  source 1..21: mol_type = other DNA
cattttacaaagtcaacccac

SEQ ID NO: 1184 - DNA, 33 bp, synthetic construct
  source 1..33: mol_type = other DNA
agccgtcacgagttaagcaatagctccatcttt

SEQ ID NO: 1185 - DNA, 24 bp, synthetic construct
  source 1..24: mol_type = other DNA
acagttagcgtaacgatctaaagt

SEQ ID NO: 1186 - DNA, 28 bp, synthetic construct
  source 1..28: mol_type = other DNA
ctgtatttgtatagcgtcagcgatagca

SEQ ID NO: 1187 - DNA, 21 bp, synthetic construct
  source 1..21: mol_type = other DNA
tttgtcgtctttcaataggaa

SEQ ID NO: 1188 - DNA, 22 bp, synthetic construct
  source 1..22: mol_type = other DNA
tagtaacatttataccaagcgc

SEQ ID NO: 1189 - DNA, 32 bp, synthetic construct
  source 1..32: mol_type = other DNA
ggattttgctatagaaaggaacaactaaagga

SEQ ID NO: 1190 - DNA, 49 bp, synthetic construct
  source 1..49: mol_type = other DNA
acttcactacgaatacactaaaagaggaagggaaccagcgtccaatact

SEQ ID NO: 1191 - DNA, 21 bp, synthetic construct
  source 1..21: mol_type = other DNA
attgcgaataatagtgtatca

SEQ ID NO: 1192 - DNA, 22 bp, synthetic construct
  source 1..22: mol_type = other DNA
cacgttatgagtttccattaaa

SEQ ID NO: 1193 - DNA, 29 bp, synthetic construct
  source 1..29: mol_type = other DNA
tccaaacggctacaacagcatccaccaga

SEQ ID NO: 1194 - DNA, 23 bp, synthetic construct
  source 1..23: mol_type = other DNA
aaggctccaaaaggagtaaagcg

SEQ ID NO: 1195 - DNA, 32 bp, synthetic construct
  source 1..32: mol_type = other DNA
tgaatttccgctgaggcttgcaggcaacttta

SEQ ID NO: 1196 - DNA, 46 bp, synthetic construct
  source 1..46: mol_type = other DNA
ttgtatttgcgggatcgtcaccgatagtaaattgggcttagaaaga

SEQ ID NO: 1197 - DNA, 41 bp, synthetic construct
  source 1..41: mol_type = other DNA
aaaggaggcttttaaggctttaacaaagtatcataaccctc

SEQ ID NO: 1198 - DNA, 32 bp, synthetic construct
  source 1..32: mol_type = other DNA
aacatgagcagtaccgacaataaacaagtgcc

SEQ ID NO: 1199 - DNA, 15 bp, synthetic construct
  source 1..15: mol_type = other DNA
ttttggtagcaaaaa

SEQ ID NO: 1200 - DNA, 32 bp, synthetic construct
  source 1..32: mol_type = other DNA
aaagacagggacgacgacaaaaggtcacccag

SEQ ID NO: 1201 - DNA, 32 bp, synthetic construct
  source 1..32: mol_type = other DNA
tgaggattcagctattcagcggccagaggcgt

SEQ ID NO: 1202 - DNA, 65 bp, synthetic construct
  source 1..65: mol_type = other DNA
cgggtaaaatacgttacaagattcatggtaaaccaaacagaggggtaagaaagagccccaggaag

SEQ ID NO: 1203 - DNA, 32 bp, synthetic construct
  source 1..32: mol_type = other DNA
aaattccaatagatatgcagaagaaagggttg

SEQ ID NO: 1204 - DNA, 31 bp, synthetic construct
  source 1..31: mol_type = other DNA
tatgctgctcaacagttaatttacaccctca

SEQ ID NO: 1205 - DNA, 15 bp, synthetic construct
  source 1..15: mol_type = other DNA
ttttcgaaagagatg

SEQ ID NO: 1206 - DNA, 33 bp, synthetic construct
  source 1..33: mol_type = other DNA
catcttggatcccatccaagtcctgattctaag

SEQ ID NO: 1207 - DNA, 32 bp, synthetic construct
  source 1..32: mol_type = other DNA
cagcgagtagaaacacagacagcgtttttatt

SEQ ID NO: 1208 - DNA, 33 bp, synthetic construct
  source 1..33: mol_type = other DNA
gaaacaaagtacagccggaacccgcgaccgctt

SEQ ID NO: 1209 - DNA, 32 bp, synthetic construct
  source 1..32: mol_type = other DNA
tgtgatttatcattcaatcaatcaaccaccct

SEQ ID NO: 1210 - DNA, 33 bp, synthetic construct
  source 1..33: mol_type = other DNA
gaaatagagagcattccaagttaccatcttacc

SEQ ID NO: 1211 - DNA, 24 bp, synthetic construct
  source 1..24: mol_type = other DNA
attgtgtcgaaatgaggcgcagac

SEQ ID NO: 1212 - DNA, 42 bp, synthetic construct
  source 1..42: mol_type = other DNA
ggtcaatcatacagatgaaagttttgcatagcgaggcgaacc

SEQ ID NO: 1213 - DNA, 21 bp, synthetic construct
  source 1..21: mol_type = other DNA
aggcgcataggctacctaaaa

SEQ ID NO: 1214 - DNA, 33 bp, synthetic construct
  source 1..33: mol_type = other DNA
tattcattacccaaatcaacggccctgaccata

SEQ ID NO: 1215 - DNA, 27 bp, synthetic construct
  source 1..27: mol_type = other DNA
aaaggaacgagggccgcttcggtttat

SEQ ID NO: 1216 - DNA, 21 bp, synthetic construct
  source 1..21: mol_type = other DNA
acgagtagctttaggaacaaa

SEQ ID NO: 1217 - DNA, 48 bp, synthetic construct
  source 1..48: mol_type = other DNA
atcattgtaaaacgaactaacggactaaagtacggtgtctttcgcaaa

SEQ ID NO: 1218 - DNA, 16 bp, synthetic construct
  source 1..16: mol_type = other DNA
tttaatttgagttaaa

SEQ ID NO: 1219 - DNA, 37 bp, synthetic construct
  source 1..37: mol_type = other DNA
ttcatcaggatctgtataatgtataaaaggtggcatc

SEQ ID NO: 1220 - DNA, 32 bp, synthetic construct
  source 1..32: mol_type = other DNA
cgtcgcagattagattatcagtgaagaggact

SEQ ID NO: 1221 - DNA, 15 bp, synthetic construct
  source 1..15: mol_type = other DNA
tttttgcagatagag

SEQ ID NO: 1222 - DNA, 36 bp, synthetic construct
  source 1..36: mol_type = other DNA
acgctaccacatgctgaatagctcaacattttcatt

SEQ ID NO: 1223 - DNA, 32 bp, synthetic construct
  source 1..32: mol_type = other DNA
aggatctgataacttttgaaatacaggcgcct

SEQ ID NO: 1224 - DNA, 32 bp, synthetic construct
  source 1..32: mol_type = other DNA
ggcatacaaaatttatcagacgctgcaacgcc

SEQ ID NO: 1225 - DNA, 32 bp, synthetic construct
  source 1..32: mol_type = other DNA
caacacctgctcatcctccggctaagttatac

SEQ ID NO: 1226 - DNA, 23 bp, synthetic construct
  source 1..23: mol_type = other DNA
gtttaccagacgacaggaagcaa

SEQ ID NO: 1227 - DNA, 32 bp, synthetic construct
  source 1..32: mol_type = other DNA
tgaattctttttaaaaaatcatagttttttca

SEQ ID NO: 1228 - DNA, 32 bp, synthetic construct
  source 1..32: mol_type = other DNA
taacaataatgggttatcaactttgaaacact

SEQ ID NO: 1229 - DNA, 33 bp, synthetic construct
  source 1..33: mol_type = other DNA
taaaattgaaaatccaaataactattagtatca

SEQ ID NO: 1230 - DNA, 32 bp, synthetic construct
  source 1..32: mol_type = other DNA
ctggatgaactgacgttacttaacgccgaccg

SEQ ID NO: 1231 - DNA, 33 bp, synthetic construct
  source 1..33: mol_type = other DNA
gcggaatcgtcatgactattaaatcaaaaaatg

SEQ ID NO: 1232 - DNA, 35 bp, synthetic construct
  source 1..35: mol_type = other DNA
cattgattcactttttctcgcaagaacctgacccc

SEQ ID NO: 1233 - DNA, 20 bp, synthetic construct
  source 1..20: mol_type = other DNA
taaagaaaccatcaccagta

SEQ ID NO: 1234 - DNA, 30 bp, synthetic construct
  source 1..30: mol_type = other DNA
tcaaattgctccatctggcatgagaaggaa

SEQ ID NO: 1235 - DNA, 44 bp, synthetic construct
  source 1..44: mol_type = other DNA
gagaatgaccatatagtcagatttagaactatttcaaatattca

SEQ ID NO: 1236 - DNA, 22 bp, synthetic construct
  source 1..22: mol_type = other DNA
agaccgaaaagctaaatcggtt

SEQ ID NO: 1237 - DNA, 29 bp, synthetic construct
  source 1..29: mol_type = other DNA
actccagcaataaaaaggcaaagaatcga

SEQ ID NO: 1238 - DNA, 42 bp, synthetic construct
  source 1..42: mol_type = other DNA
agagagtacctttaattgctcgaggtcatttttgcggatggc

SEQ ID NO: 1239 - DNA, 37 bp, synthetic construct
  source 1..37: mol_type = other DNA
ttagagcttaatttcaactaaattacaggtgagatgg

SEQ ID NO: 1240 - DNA, 40 bp, synthetic construct
  source 1..40: mol_type = other DNA
tggtcaataaacaggaagattgtattttaaccaataggaa

SEQ ID NO: 1241 - DNA, 32 bp, synthetic construct
  source 1..32: mol_type = other DNA
tagctatatgttttaaatatgcaaacaacatt

SEQ ID NO: 1242 - DNA, 18 bp, synthetic construct
  source 1..18: mol_type = other DNA
tggggcgcgcgaccccgg

SEQ ID NO: 1243 - DNA, 32 bp, synthetic construct
  source 1..32: mol_type = other DNA
gaacaactgcagatgatattatactattacga

SEQ ID NO: 1244 - DNA, 32 bp, synthetic construct
  source 1..32: mol_type = other DNA
aattctactaataagagtaatcgtaaaactag

SEQ ID NO: 1245 - DNA, 32 bp, synthetic construct
  source 1..32: mol_type = other DNA
cattaataattgtttggggcaattctgtaaat

SEQ ID NO: 1246 - DNA, 32 bp, synthetic construct
  source 1..32: mol_type = other DNA
taaatctaatggaaacgtaaaacgatttcatt

SEQ ID NO: 1247 - DNA, 33 bp, synthetic construct
  source 1..33: mol_type = other DNA
cagaaaggctatgtagctatgcgcatcgtaacc

SEQ ID NO: 1248 - DNA, 32 bp, synthetic construct
  source 1..32: mol_type = other DNA
gacaacttatttgcgggttagaaatgtaagag

SEQ ID NO: 1249 - DNA, 32 bp, synthetic construct
  source 1..32: mol_type = other DNA
ttacaataataaagaaaatatacaggtaatag

SEQ ID NO: 1250 - DNA, 32 bp, synthetic construct
  source 1..32: mol_type = other DNA
gtaccaaaaacataagctagctgataaattaa

SEQ ID NO: 1251 - DNA, 32 bp, synthetic construct
  source 1..32: mol_type = other DNA
ctgtaatagtcagatgattgcgtagttacatt

SEQ ID NO: 1252 - DNA, 32 bp, synthetic construct
  source 1..32: mol_type = other DNA
ccttcctcatatagggtgaggtaatgtgccag

SEQ ID NO: 1253 - DNA, 29 bp, synthetic construct
  source 1..29: mol_type = other DNA
gcgggatacctttttttaccctaaatatt

SEQ ID NO: 1254 - DNA, 21 bp, synthetic construct
  source 1..21: mol_type = other DNA
taaaaattagcaaagcggatt

SEQ ID NO: 1255 - DNA, 32 bp, synthetic construct
  source 1..32: mol_type = other DNA
aggagctcgcctgaacatcgggtgagtttaga

SEQ ID NO: 1256 - DNA, 20 bp, synthetic construct
  source 1..20: mol_type = other DNA
caatagcaaaatgtgaatta

SEQ ID NO: 1257 - DNA, 33 bp, synthetic construct
  source 1..33: mol_type = other DNA
ctaaaattaatcaggtcatacataaattaagac

SEQ ID NO: 1258 - DNA, 43 bp, synthetic construct
  source 1..43: mol_type = other DNA
tgaaaaggccggcaccgctgatcgcaccagtgaggaatcctga

SEQ ID NO: 1259 - DNA, 21 bp, synthetic construct
  source 1..21: mol_type = other DNA
caccatcaatatgcgcaagga

SEQ ID NO: 1260 - DNA, 41 bp, synthetic construct
  source 1..41: mol_type = other DNA
accgttcatctcaggaatctggtgcttgattagaaactatc

SEQ ID NO: 1261 - DNA, 23 bp, synthetic construct
  source 1..23: mol_type = other DNA
tgccggagaggcaggtcattagg

SEQ ID NO: 1262 - DNA, 21 bp, synthetic construct
  source 1..21: mol_type = other DNA
ggagcaaacaagagaattagc

SEQ ID NO: 1263 - DNA, 41 bp, synthetic construct
  source 1..41: mol_type = other DNA
tgaacgagatgggaacagttggtgtggttgcttgaatcaga

SEQ ID NO: 1264 - DNA, 32 bp, synthetic construct
  source 1..32: mol_type = other DNA
agctcatttaagcaaatatttaaatgaccatt

SEQ ID NO: 1265 - DNA, 40 bp, synthetic construct
  source 1..40: mol_type = other DNA
cgccatcaaaaataatatcagaaaagccccaaaacctgtt

SEQ ID NO: 1266 - DNA, 32 bp, synthetic construct
  source 1..32: mol_type = other DNA
acgtggagctgatagccccaccagcgtagtag

SEQ ID NO: 1267 - DNA, 29 bp, synthetic construct
  source 1..29: mol_type = other DNA
cattaaatgtgagggagcgggtgcgcgta

SEQ ID NO: 1268 - DNA, 32 bp, synthetic construct
  source 1..32: mol_type = other DNA
caacccagacgaacgaactaaaacattttgcg

SEQ ID NO: 1269 - DNA, 32 bp, synthetic construct
  source 1..32: mol_type = other DNA
tctccgtaaaacaggatctacagcaacaattc

SEQ ID NO: 1270 - DNA, 21 bp, synthetic construct
  source 1..21: mol_type = other DNA
taatgggatgcctgagagtct

SEQ ID NO: 1271 - DNA, 28 bp, synthetic construct
  source 1..28: mol_type = other DNA
tcacaacggcggggtcacgccgctaggg

SEQ ID NO: 1272 - DNA, 32 bp, synthetic construct
  source 1..32: mol_type = other DNA
acacgacgcctgcaaggtgaggtatcatccaa

SEQ ID NO: 1273 - DNA, 32 bp, synthetic construct
  source 1..32: mol_type = other DNA
agattctggttttgagaaaaatctatatgacc

SEQ ID NO: 1274 - DNA, 29 bp, synthetic construct
  source 1..29: mol_type = other DNA
gtgcatctgccagtacgccagccaccgag

SEQ ID NO: 1275 - DNA, 32 bp, synthetic construct
  source 1..32: mol_type = other DNA
ggacgaacggcaaatgaacagtgccttagact

SEQ ID NO: 1276 - DNA, 32 bp, synthetic construct
  source 1..32: mol_type = other DNA
atcggccaccttgcgattcaaaatttatcttt

SEQ ID NO: 1277 - DNA, 32 bp, synthetic construct
  source 1..32: mol_type = other DNA
acaggaatcaatattgaacctccaatactttt

SEQ ID NO: 1278 - DNA, 40 bp, synthetic construct
  source 1..40: mol_type = other DNA
tgggaaggtaacgccaggccagtgccaagcttctgtgtga

SEQ ID NO: 1279 - DNA, 16 bp, synthetic construct
  source 1..16: mol_type = other DNA
caggctgcgcaactgt

SEQ ID NO: 1280 - DNA, 37 bp, synthetic construct
  source 1..37: mol_type = other DNA
ctgagtagtcgccattgctttccggagacagtcaaat

SEQ ID NO: 1281 - DNA, 24 bp, synthetic construct
  source 1..24: mol_type = other DNA
ggccttgcagggcgaccacaatca

SEQ ID NO: 1282 - DNA, 24 bp, synthetic construct
  source 1..24: mol_type = other DNA
tcacttgctttataattccagcca

SEQ ID NO: 1283 - DNA, 48 bp, synthetic construct
  source 1..48: mol_type = other DNA
aattgttagaagcataaagtgtaaacctgtcgtgccagctgcggtttg

SEQ ID NO: 1284 - DNA, 48 bp, synthetic construct
  source 1..48: mol_type = other DNA
tccgctcacaattccagacgttgtaaaacgacgggttttcccagtcac

SEQ ID NO: 1285 - DNA, 26 bp, synthetic construct
  source 1..26: mol_type = other DNA
taaaagagatacttctcggcattgca

SEQ ID NO: 1286 - DNA, 32 bp, synthetic construct
  source 1..32: mol_type = other DNA
gaagtgttcgtgctttcctcgttatgacgagc

SEQ ID NO: 1287 - DNA, 23 bp, synthetic construct
  source 1..23: mol_type = other DNA
gcgggagcaggaacggtttgagg

SEQ ID NO: 1288 - DNA, 48 bp, synthetic construct
  source 1..48: mol_type = other DNA
cgtattggagttgcagcaagcggttgtttgatggtggttcggtgccgt

SEQ ID NO: 1289 - DNA, 48 bp, synthetic construct
  source 1..48: mol_type = other DNA
caacgcgcggggagaggcattaatgaatcggccacaacatacgagccg

SEQ ID NO: 1290 - DNA, 24 bp, synthetic construct
  source 1..24: mol_type = other DNA
acgtataaagtgtagcattgaccg

SEQ ID NO: 1291 - DNA, 26 bp, synthetic construct
  source 1..26: mol_type = other DNA
accaccaccgtactatagaaccagtc

SEQ ID NO: 1292 - DNA, 24 bp, synthetic construct
  source 1..24: mol_type = other DNA
cgctggcagggagcccccgattta

SEQ ID NO: 1293 - DNA, 40 bp, synthetic construct
  source 1..40: mol_type = other DNA
aaagcactagttttttggggtcgaaccatcacccaaatca

SEQ ID NO: 1294 - DNA, 48 bp, synthetic construct
  source 1..48: mol_type = other DNA
aaatcggaaccctaaacagcaggcgaaaatccccacgctggtttgccc

SEQ ID NO: 1295 - DNA, 31 bp, synthetic construct
  source 1..31: mol_type = other DNA
gagcttgacggggaaaaagcgaaacgagtaa

SEQ ID NO: 1296 - DNA, 32 bp, synthetic construct
  source 1..32: mol_type = other DNA
catgtcaatcatatgtaaccagctttcatcaa

SEQ ID NO: 1297 - DNA, 40 bp, synthetic construct
  source 1..40: mol_type = other DNA
tttttaccagcgccaaagacaaatggtaatatccagtttt

SEQ ID NO: 1298 - DNA, 30 bp, synthetic construct
  source 1..30: mol_type = other DNA
ttttagacaccacggttcatatggtttttt

SEQ ID NO: 1299 - DNA, 30 bp, synthetic construct
  source 1..30: mol_type = other DNA
tttttagcaaacgtaaagaaacgcaatttt

SEQ ID NO: 1300 - DNA, 30 bp, synthetic construct
  source 1..30: mol_type = other DNA
ttttaacggaataccacgcagtatgttttt

SEQ ID NO: 1301 - DNA, 30 bp, synthetic construct
  source 1..30: mol_type = other DNA
ttttgtaagcagataaacgcaataattttt

SEQ ID NO: 1302 - DNA, 36 bp, synthetic construct
  source 1..36: mol_type = other DNA
gaagcccttgaaatagcccaataataagagcatttt

SEQ ID NO: 1303 - DNA, 27 bp, synthetic construct
  source 1..27: mol_type = other DNA
ttttagaaacaattttaagaaaatttt

SEQ ID NO: 1304 - DNA, 29 bp, synthetic construct
  source 1..29: mol_type = other DNA
ttttctaatatcgtaggaatcattatttt

SEQ ID NO: 1305 - DNA, 29 bp, synthetic construct
  source 1..29: mol_type = other DNA
ttttttagacggaatcagatatagatttt

SEQ ID NO: 1306 - DNA, 29 bp, synthetic construct
  source 1..29: mol_type = other DNA
ttttaaaatgaagaacctcccgacttttt

SEQ ID NO: 1307 - DNA, 29 bp, synthetic construct
  source 1..29: mol_type = other DNA
ttttcagccatattaaatcaagatttttt

SEQ ID NO: 1308 - DNA, 33 bp, synthetic construct
  source 1..33: mol_type = other DNA
ttttcaacgctaacgagcgtctttccagatggc

SEQ ID NO: 1309 - DNA, 29 bp, synthetic construct
  source 1..29: mol_type = other DNA
ttttagttgctattttgaaagtaattttt

SEQ ID NO: 1310 - DNA, 37 bp, synthetic construct
  source 1..37: mol_type = other DNA
gtcgaggccttatttatccagttacaaaataaatttt

SEQ ID NO: 1311 - DNA, 29 bp, synthetic construct
  source 1..29: mol_type = other DNA
tttttgcgggaggttttcgcgcctgtttt

SEQ ID NO: 1312 - DNA, 34 bp, synthetic construct
  source 1..34: mol_type = other DNA
tttagcaatagcagtttttgtttaacgtcatttt

SEQ ID NO: 1313 - DNA, 29 bp, synthetic construct
  source 1..29: mol_type = other DNA
ttttaggcttatccggtaacaagaatttt

SEQ ID NO: 1314 - DNA, 37 bp, synthetic construct
  source 1..37: mol_type = other DNA
gagccagcagagaattaaaaacagggaagcgcatttt

SEQ ID NO: 1315 - DNA, 29 bp, synthetic construct
  source 1..29: mol_type = other DNA
ttttccgcgcccaatagaatcggcttttt

SEQ ID NO: 1316 - DNA, 34 bp, synthetic construct
  source 1..34: mol_type = other DNA
ttcatcagagagatagagggtaattgagcgtttt

SEQ ID NO: 1317 - DNA, 53 bp, synthetic construct
  source 1..53: mol_type = other DNA
ttttaccgcactcatcgagagggtattagtctttccaaataaggcgttatttt

SEQ ID NO: 1318 - DNA, 16 bp, synthetic construct
  source 1..16: mol_type = other DNA
aaccaagttttttttt

SEQ ID NO: 1319 - DNA, 29 bp, synthetic construct
  source 1..29: mol_type = other DNA
ttttaaataataatcataattactatttt

SEQ ID NO: 1320 - DNA, 29 bp, synthetic construct
  source 1..29: mol_type = other DNA
tttttttatcaattaccagtataaatttt

SEQ ID NO: 1321 - DNA, 29 bp, synthetic construct
  source 1..29: mol_type = other DNA
tttttctgtccagcttaattgagaatttt

SEQ ID NO: 1322 - DNA, 50 bp, synthetic construct
  source 1..50: mol_type = other DNA
tttttttcgagccagtaataagagaattttttatcctgaatcttactttt

SEQ ID NO: 1323 - DNA, 29 bp, synthetic construct
  source 1..29: mol_type = other DNA
tttttcgccatatttaaagaagagttttt

SEQ ID NO: 1324 - DNA, 29 bp, synthetic construct
  source 1..29: mol_type = other DNA
ttttgccaacgctcaacaggtctgatttt

SEQ ID NO: 1325 - DNA, 29 bp, synthetic construct
  source 1..29: mol_type = other DNA
ttttgaaaaagcctgttatgtaaattttt

SEQ ID NO: 1326 - DNA, 29 bp, synthetic construct
  source 1..29: mol_type = other DNA
ttttaataagaataaaccaaagaactttt

SEQ ID NO: 1327 - DNA, 53 bp, synthetic construct
  source 1..53: mol_type = other DNA
ttttttctgacctaaatttatttagttagcgagaaaatttcaattaccttttt

SEQ ID NO: 1328 - DNA, 16 bp, synthetic construct
  source 1..16: mol_type = other DNA
atttcatctttttttt

SEQ ID NO: 1329 - DNA, 29 bp, synthetic construct
  source 1..29: mol_type = other DNA
ttttgctgatgcaacaaacatcaagtttt

SEQ ID NO: 1330 - DNA, 29 bp, synthetic construct
  source 1..29: mol_type = other DNA
ttttgagactacaccttttttaatgtttt

SEQ ID NO: 1331 - DNA, 29 bp, synthetic construct
  source 1..29: mol_type = other DNA
ttttcaatagtgtatatgtgagtgatttt

SEQ ID NO: 1332 - DNA, 50 bp, synthetic construct
  source 1..50: mol_type = other DNA
tttttagaatccttgaaaacatagcctctaatttaggcagaggcattttt

SEQ ID NO: 1333 - DNA, 29 bp, synthetic construct
  source 1..29: mol_type = other DNA
ttttataaccttgcttcatcaatattttt

SEQ ID NO: 1334 - DNA, 29 bp, synthetic construct
  source 1..29: mol_type = other DNA
ttttgaaacagtacataacctaccatttt

SEQ ID NO: 1335 - DNA, 29 bp, synthetic construct
  source 1..29: mol_type = other DNA
ttttaaaacaaaattaaattttcagtttt

SEQ ID NO: 1336 - DNA, 29 bp, synthetic construct
  source 1..29: mol_type = other DNA
ttttgagcaaaagaagaagaaacaatttt

SEQ ID NO: 1337 - DNA, 53 bp, synthetic construct
  source 1..53: mol_type = other DNA
ttttatcgcgcagaggcgaaaataccaataacggatactaacaactaattttt

SEQ ID NO: 1338 - DNA, 16 bp, synthetic construct
  source 1..16: mol_type = other DNA
gttacaaatttttttt

SEQ ID NO: 1339 - DNA, 29 bp, synthetic construct
  source 1..29: mol_type = other DNA
ttttgtttaacgataatacatttgatttt

SEQ ID NO: 1340 - DNA, 29 bp, synthetic construct
  source 1..29: mol_type = other DNA
tttttatcaaaatcgtattaaatcctttt

SEQ ID NO: 1341 - DNA, 29 bp, synthetic construct
  source 1..29: mol_type = other DNA
ttttaatcctgattttaaaagtttgtttt

SEQ ID NO: 1342 - DNA, 50 bp, synthetic construct
  source 1..50: mol_type = other DNA
ttttcggaattatcatcatattcctttgtattaattaattttcccttttt

SEQ ID NO: 1343 - DNA, 29 bp, synthetic construct
  source 1..29: mol_type = other DNA
ttttagtaacattatcatcgccatttttt

SEQ ID NO: 1344 - DNA, 29 bp, synthetic construct
  source 1..29: mol_type = other DNA
tttttttgcccgaacgtcggtcagttttt

SEQ ID NO: 1345 - DNA, 29 bp, synthetic construct
  source 1..29: mol_type = other DNA
ttttggatttagaagtaacgctgagtttt

SEQ ID NO: 1346 - DNA, 29 bp, synthetic construct
  source 1..29: mol_type = other DNA
ttttagattagagccgtaaatatcatttt

SEQ ID NO: 1347 - DNA, 53 bp, synthetic construct
  source 1..53: mol_type = other DNA
tttttgaaaggaattgaggattggcaaaaaccctcaaaaacgctcatggtttt

SEQ ID NO: 1348 - DNA, 16 bp, synthetic construct
  source 1..16: mol_type = other DNA
tcaacagttttttttt

SEQ ID NO: 1349 - DNA, 29 bp, synthetic construct
  source 1..29: mol_type = other DNA
ttttagccagcactcaatcgtctgatttt

SEQ ID NO: 1350 - DNA, 29 bp, synthetic construct
  source 1..29: mol_type = other DNA
ttttattaacacccagtaataaaagtttt

SEQ ID NO: 1351 - DNA, 29 bp, synthetic construct
  source 1..29: mol_type = other DNA
ttttaaaaatacgatagaacccttctttt

SEQ ID NO: 1352 - DNA, 47 bp, synthetic construct
  source 1..47: mol_type = other DNA
ttgatatcgcgtctggccttcctgtcacagacaatatttttgatttt

SEQ ID NO: 1353 - DNA, 50 bp, synthetic construct
  source 1..50: mol_type = other DNA
ttttatggctattagtctttaatgcgagagaaaccaccagaaggagtttt

SEQ ID NO: 1354 - DNA, 29 bp, synthetic construct
  source 1..29: mol_type = other DNA
tttttgacctgaaagcgacgtggcgtttt

SEQ ID NO: 1355 - DNA, 29 bp, synthetic construct
  source 1..29: mol_type = other DNA
ttttggacattctggcccgcttaattttt

SEQ ID NO: 1356 - DNA, 29 bp, synthetic construct
  source 1..29: mol_type = other DNA
ttttaatggattatttaaggccgattttt

SEQ ID NO: 1357 - DNA, 29 bp, synthetic construct
  source 1..29: mol_type = other DNA
ttttaaatacctacatttcacgcaatttt

SEQ ID NO: 1358 - DNA, 49 bp, synthetic construct
  source 1..49: mol_type = other DNA
ttttaacaatattaccgccagcaaataggtaaatattttgtaggtggca

SEQ ID NO: 1359 - DNA, 38 bp, synthetic construct
  source 1..38: mol_type = other DNA
ttttattaaccgttgtagcatctgtccattgcgacagt

SEQ ID NO: 1360 - DNA, 35 bp, synthetic construct
  source 1..35: mol_type = other DNA
tttttaaagggattttagactaaacaggcattggc

SEQ ID NO: 1361 - DNA, 38 bp, synthetic construct
  source 1..38: mol_type = other DNA
ttttgcgccgctacagggcgacccgccgaacgtcggat

SEQ ID NO: 1362 - DNA, 35 bp, synthetic construct
  source 1..35: mol_type = other DNA
ttttagaaaggaagggaagagccggcgataagaat

SEQ ID NO: 1363 - DNA, 24 bp, synthetic construct
  source 1..24: mol_type = other DNA
gtataccgccaccctcagaaccgc

SEQ ID NO: 1364 - DNA, 48 bp, synthetic construct
  source 1..48: mol_type = other DNA
gagtattaagaggctgagactcctcaccgtactcaggaggtttagaat

SEQ ID NO: 1365 - DNA, 48 bp, synthetic construct
  source 1..48: mol_type = other DNA
caacgtcatacatggcttttgatgtattattctgaaacatgaaagcca

SEQ ID NO: 1366 - DNA, 48 bp, synthetic construct
  source 1..48: mol_type = other DNA
cgaaaccaccaccagagccgccgctgaatttaccgttccagtaagggc

SEQ ID NO: 1367 - DNA, 48 bp, synthetic construct
  source 1..48: mol_type = other DNA
ttttagcccccttattagcgtttgcaccctcagagccgccaccagcag

SEQ ID NO: 1368 - DNA, 47 bp, synthetic construct
  source 1..47: mol_type = other DNA
ggaagtagcaccattaccattagtttcatcggcattttcggtcacgg

SEQ ID NO: 1369 - DNA, 28 bp, synthetic construct
  source 1..28: mol_type = other DNA
cgcgcgacattcaacaaaatcaccacca

SEQ ID NO: 1370 - DNA, 28 bp, synthetic construct
  source 1..28: mol_type = other DNA
gccaaaatacatacaagacaaaaggcac

SEQ ID NO: 1371 - DNA, 28 bp, synthetic construct
  source 1..28: mol_type = other DNA
tataagcagatagcagcaaacgtaggcc

SEQ ID NO: 1372 - DNA, 28 bp, synthetic construct
  source 1..28: mol_type = other DNA
tgagtaattgagcgttaagaaaagttca

SEQ ID NO: 1373 - DNA, 15 bp, synthetic construct
  source 1..15: mol_type = other DNA
cagaacgtcaaaaat

SEQ ID NO: 1374 - DNA, 28 bp, synthetic construct
  source 1..28: mol_type = other DNA
gtactggtaataagaaagtcagaggatt

SEQ ID NO: 1375 - DNA, 48 bp, synthetic construct
  source 1..48: mol_type = other DNA
tttttaatgccccctgcctatttccgatagttgcgccgacaatgactg

SEQ ID NO: 1376 - DNA, 30 bp, synthetic construct
  source 1..30: mol_type = other DNA
ttatcaatatatgtggtaaagtaattcaac

SEQ ID NO: 1377 - DNA, 48 bp, synthetic construct
  source 1..48: mol_type = other DNA
aataccagtcaggacgttgggaagataacagttgattcccaattcagc

SEQ ID NO: 1378 - DNA, 28 bp, synthetic construct
  source 1..28: mol_type = other DNA
tgtatacctacattatatctttaggtgc

SEQ ID NO: 1379 - DNA, 16 bp, synthetic construct
  source 1..16: mol_type = other DNA
cgctcatggaataaaa

SEQ ID NO: 1380 - DNA, 16 bp, synthetic construct
  source 1..16: mol_type = other DNA
attacgccagctgcta

SEQ ID NO: 1381 - DNA, 40 bp, synthetic construct
  source 1..40: mol_type = other DNA
gaggcgaaagggggatactctagaggatccccgggtagta

SEQ ID NO: 1382 - DNA, 48 bp, synthetic construct
  source 1..48: mol_type = other DNA
cgcccgagctcgaattcgtaatcatgagtgagctaactcacattacac

SEQ ID NO: 1383 - DNA, 48 bp, synthetic construct
  source 1..48: mol_type = other DNA
ggtattgcgttgcgctcactgccctcttttcaccagtgagacggggga

SEQ ID NO: 1384 - DNA, 48 bp, synthetic construct
  source 1..48: mol_type = other DNA
aaacaacagctgattgcccttcacccttataaatcaaaagaatagagg

SEQ ID NO: 1385 - DNA, 48 bp, synthetic construct
  source 1..48: mol_type = other DNA
taacccgagatagggttgagtgttgaacgtggactccaacgtcaacaa

SEQ ID NO: 1386 - DNA, 15 bp, synthetic construct
  source 1..15: mol_type = other DNA
tgaaccatcaccagg

SEQ ID NO: 1387 - DNA, 46 bp, synthetic construct
  source 1..46: mol_type = other DNA
attcatttcaattacccgcgcagaggcgaattttttggagggaggg

SEQ ID NO: 1388 - DNA, 35 bp, synthetic construct
  source 1..35: mol_type = other DNA
tcagatgatggcaacaataacttttggagggaggg

SEQ ID NO: 1389 - DNA, 46 bp, synthetic construct
  source 1..46: mol_type = other DNA
attatcattttttatcatcatattcctgattattttggagggaggg

SEQ ID NO: 1390 - DNA, 65 bp, synthetic construct
  source 1..65: mol_type = other DNA
ttctgtgcaaaagaaggcaccaggctgaccgtaatcttgacaagaaccggattttccagccagcc

SEQ ID NO: 1391 - DNA, 35 bp, synthetic construct
  source 1..35: mol_type = other DNA
gcaaaagacggtgtacagaccttttccagccagcc

SEQ ID NO: 1392 - DNA, 56 bp, synthetic construct
  source 1..56: mol_type = other DNA
gcatcaaaaagattaagaggaacttcaaatatcgcgttttaattttccagccagcc

SEQ ID NO: 1393 - DNA, 15 bp, synthetic construct
  source 1..15: mol_type = other DNA
  misc_binding 1: bound_moiety = Cy3-HRP, Cy3-GOx, Cy3-G6pDH, Cy3-betaGal, or ThioC6
tttttccctccctcc

SEQ ID NO: 1394 - DNA, 15 bp, synthetic construct
  source 1..15: mol_type = other DNA
  misc_binding 1: bound_moiety = Alexa Fluor 647-MDH, Alexa Fluor 647-LDH, ThioC6
tttttggctggctgg

SEQ ID NO: 1395 - DNA, 15 bp, synthetic construct
  source 1..15: mol_type = other DNA
  misc_binding 1: bound_moiety = biotin
tttttggagggaggg